(19) United States
(12) Patent Application Publication
(10) Pub. No.: US 2023/0101046 A1
(43) Pub. Date: Mar. 30, 2023

(54) ANAPLASTIC LYMPHOMA KINASE CHIMERIC ANTIGEN RECEPTORS AND METHODS OF USE

(71) Applicant: The Children's Medical Center Corporation, Boston, MA (US)

(72) Inventors: Roberto CHIARLE, Boston, MA (US); Wei-Tien TAI, Boston, MA (US); Elisa BERGAGGIO, Boston, MA (US)

(73) Assignee: The Children's Medical Center Corporation, Boston, MA (US)

(21) Appl. No.: 17/794,895

(22) PCT Filed: Jan. 28, 2021
(86) PCT No.: PCT/US2021/015519

§ 371 Date: Jul. 22, 2022

Related U.S. Application Data
(60) Provisional application No. 62/966,748, filed on Jan. 28, 2020.

(51) Int. Cl. C07K 16/40; C07K 14/725; C07K 14/54; C07K 14/545; C07K 14/55; C07K 14/535; C07K 14/525; C07K 14/52; C07K 14/57; A61K 35/17; A61K 39/00; A61K 31/675; C12N 15/62; C12N 5/0783; C12N 15/86

(52) U.S. Cl. CPC C07K 16/40; C07K 14/7051; C07K 14/5406; C07K 14/5412; C07K 14/5418; C07K 14/5434; C07K 14/5443; C07K 14/545; C07K 14/55; C07K 14/535; C07K 14/525; C07K 14/523; C07K 14/57; A61K 35/17; A61K 39/0005; A61K 31/675; C12N 15/625; C12N 5/0636; C12N 15/86; A61K 38/00

(57) ABSTRACT

Provided herein are anaplastic lymphoma kinase chimeric antigen receptors (ALK CARs). The invention also provides polynucleotides encoding ALK CARs, engineered immune cells comprising an ALK CAR, pharmaceutical compositions thereof, and kits for administering the same. Methods of treating a subject with a disease by administering the ALK CAR or engineered immune cell comprising an ALK CAR, or pharmaceutical compositions thereof, are also provided.

### CROSS-REFERENCE TO RELATED APPLICATION

This application claims the benefit of the following U.S. Provisional Application No. 62/966,748, filed Jan. 28, 2020, the entire contents of which are incorporated herein by reference.

### BACKGROUND OF THE INVENTION

Anaplastic lymphoma kinase (ALK) is a receptor tyrosine kinase in the insulin receptor superfamily, which plays an important role in the development of the brain and nervous system. ALK is processed into peptides by the proteasome, transported to the endoplasmic reticulum by transporters associated with antigen processing-1 and -2 (TAP1 and TAP2), and binds to the HLA class I molecules. ALK is minimally expressed by normal tissues during adulthood. However, ALK is aberrantly expressed by tumors, such as non-small cell lung cancer (NSCLC), anaplastic large cell lymphoma (ALCL), and neuroblastoma. More rarely, ALK is expressed by B-cell lymphoma, thyroid cancer, colon cancer, breast cancer, inflammatory myofibroblastic tumors (IMT), renal carcinoma, esophageal cancer, and melanoma. Thus, ALK is an ideal shared antigen across different type of cancers. ALK may become oncogenic by forming a fusion gene with other genes, by gaining additional gene copies, or by genetic mutations.

Several ALK tyrosine kinase inhibitors (TKIs) are available for the treatment of ALK-rearranged NSCLC, including crizotinib, ceritinib, alectinib, brigatinib, and lorlatinib. Unfortunately, resistance to these drugs occurs within 1-2 years through a variety of mechanisms. Once patients develop resistance to available ALK inhibitors, they are typically treated with cytotoxic chemotherapy rather than immunotherapy since response rates to PD-1 pathway inhibitors in this population are very low. Although PD-1 inhibitors such as pembrolizumab (Keytruda®) and nivolumab (Opdivo®) have revolutionized the treatment of lung cancer in general, particularly in smoking-associated cancers, most patients with ALK-positive lung cancer do not respond to these immunotherapies. Thus, the development of additional ALK-targeted therapy in ALK-positive cancers is clearly a need.

### INCORPORATION BY REFERENCE

All publications, patents, and patent applications mentioned in this specification are herein incorporated by reference to the same extent as if each individual publication, patent, or patent application was specifically and individually indicated to be incorporated by reference. Absent any indication otherwise, publications, patents, and patent applications mentioned in this specification are incorporated herein by reference in their entireties

### SUMMARY OF THE INVENTION

As described below, the present invention features anaplastic lymphoma kinase chimeric antigen receptors (ALK CARs). The invention also provides engineered immune cells comprising an ALK CAR, polynucleotides encoding ALK CARs, pharmaceutical compositions thereof, and kits for administering the same. Methods of treating a subject with a disease by administering the ALK CAR, engineered immune cell comprising an ALK CAR, or polynucleotides encoding ALK CARs, or pharmaceutical compositions thereof, are also provided.

One aspect of the invention provides an anaplastic lymphoma kinase chimeric antigen receptor (ALK CAR) comprising: an extracellular binding domain comprising a heavy chain complementarity determining region 1 (HCDR1), a heavy chain complementarity determining region 2 (HCDR2), and a heavy chain complementarity determining region 3 (HCDR3) each comprising an amino acid sequence that is at least 80% identical to the HCDR1, HCDR2, and HCDR3 sequences of an anti-ALK antibody in Table 4, wherein the extracellular binding domain specifically binds to an anaplastic lymphoma kinase (ALK) polypeptide or antibody-binding fragment thereof; a transmembrane domain; and at least one signaling domain.

In some embodiments, the extracellular binding domain comprises the HCDR1, HCDR2, and HCDR3 amino acid sequences of an anti-ALK antibody in Table 4. In some embodiments, the extracellular binding domain further comprising a light chain complementarity determining region 1 (LCDR1), a light chain complementarity determining region 2 (LCDR2), and a light chain complementarity determining region 3 (LCDR3) each comprising an amino acid sequence that is at least 80% identical to the LCDR1, LCDR2 and LCDR3 sequences of an anti-ALK antibody in Table 3. In some embodiments, the extracellular binding domain comprises the LCDR1, LCDR2 and LCDR3 amino acid sequences of an anti-ALK antibody in Table 3.

In another aspect, the invention provides an anaplastic lymphoma kinase chimeric antigen receptor (ALK CAR) comprising: an extracellular binding domain comprising a heavy chain variable region (VH) comprising an amino acid sequence that is at least 80% identical to the VH of an anti-ALK antibody in Table 2, wherein the extracellular binding domain specifically binds to an anaplastic lymphoma kinase (ALK) polypeptide or antibody-binding fragment thereof; and a transmembrane domain; and at least one signaling domain.

In some embodiments, the extracellular binding domain comprises the VH of an anti-ALK antibody in Table 2. In some embodiments, the extracellular binding domain further comprises a light chain variable region (VL) comprising an amino acid sequence that is at least 80% identical to the VL of an anti-ALK antibody in Table 1. In some embodiments, the extracellular binding domain comprises the VL of an anti-ALK antibody in Table 1. In some embodiments, the VH comprises human framework regions. In some embodiments, the VL comprises human framework regions.

In some embodiments, the ALK CAR includes a linker. In one embodiment, the linker is a flexible peptide linker. In one embodiment, the linker is (Gly₄Ser)ₙ. In some embodiments, the ALK CAR includes a reporter gene. In one embodiment, the reporter gene is green fluorescent protein (GFP). In some embodiments, the extracellular binding domain is an scFv.

In some embodiments, the anti-ALK antibody comprises VH CDR amino acid sequences SYWMN, QIYPGDGDTNYNGKFKG, and YYYGSKAY, and VL CDR amino acid sequences RASENIYYSLA, NANSLED, KQAYDVPFT.

In some embodiments, the anti-ALK antibody comprises VH CDR amino acid sequences SYWMH, RIDPNSGGTKYNEKFKS, and DYYGSSYRFAY, and VL CDR amino acid sequences SVSQGISNSLN, YTSSLHS, and QQYSKLPLT.

In some embodiments, the anti-ALK antibody comprises VH CDR amino acid sequences NYWMH, YINPSSGYTKYNQKFKD, and DYYGSSSWFAY, and VL CDR amino acid sequences KASQNVGTNVA, SASYRYS, and QQYNSYPYMYT.

In some embodiments, the anti-ALK antibody comprises VH CDR amino acid sequences SYWVN, QIYPGDGDTNYNGKFKG, and SRGYFYGSTYDS, and VL CDR amino acid sequences RASESVDNYGISFMN, AASNQGS, and QQSKEVPWT.

In some embodiments, the anti-ALK antibody comprises VH CDR amino acid sequences SYWMH, YIKPSSGYTKYNQKFKD, and DYYGSSSWFAY, and VL CDR amino acid sequences KASQNVGTNVA, SASYRYS, and QQYNSYPYMYT.

In some embodiments, the anti-ALK antibody comprises VH CDR amino acid sequences SYAMS, YISSGGDYIYYADTVKG, and ERIWLRRFFDV, and VL CDR amino acid sequences KASQNVGTAVA, SASNRFT, and QQYSSYPLT.

In some embodiments, the anti-ALK antibody comprises VH CDR amino acid sequences SYWMH, YINPSSGYTKYNQKFKD, and DYYGSSSWFAY, and VL CDR amino acid sequences KASQNVGTNVA, SASYRYS, and QRYNSYPYMFT.

In some embodiments, the anti-ALK antibody comprises VH amino acid sequence QVQLQQSGAELVKPGASVKISCKASGYAFSSYWMNWVKQRPGKGLEWIGQIYPGDGD TNYNGKFKGKATLTADKSSSTAYMQLSSLTSEDSAVYFCASYYYGSKAYWGQGTLVT VSA, and VL amino acid sequence DIQMTQSPASLAASVGETVTITCRASENIYYSLAWYQQKQGKSPQLLIYNANSLEDGVPS RFSGSGSGTQYSMKINSMQPEDTATYFCKQAYDVPFTFGSGTKLEIKR

In some embodiments, the anti-ALK antibody comprises VH amino acid sequence QVQLQQPGAEFVKPGASVKLSCKASGYTFTSYWMIHWVKQRPGRGLEWIGRIDPNSGG TKYNEKFKSKATLTVDKPSSTAYMQLSSLTSEDSAVYYCARDYYGSSYRFAYWGQGTL VTVSA, and VL amino acid sequence AIQMTQTTSSLSASLGDRVTISCSVSQGISNSLNWYQQKPDGTVKLLIYYTSSLHSGVPSR FSGSGSGTDYSLTISNLEPEDIATYYCQQYSKLPLTFGAGTKLELKR.

In some embodiments, the anti-ALK antibody comprises VH amino acid sequence QVQLQQSGAELAKPGASVKLSCKASGYTFTNYWMHWVKQRPGQGLEWIGYINPSSGY TKYNQKFKDKATLTADKSSSTAYMQLSSLTYEDSAVYYCARDYYGSSSWFAYWGQGT LVTVSA, and VL amino acid sequence DIVMTQSQRFMSTSVGDRVSVTCKASQNVGTNVAWYQQKPGQSPKALIYSASYRYSGV PDRFTGSGSGTDFTLTVSNVQSEDLAEYFCQQYNSYPYMYTFGGGTKLEIKR.

In some embodiments, the anti-ALK antibody comprises VH amino acid sequence QVQLQQSGAELVKPGASVKISCKASGYAFSSYWVNWVKQRPGKGLEWIGQIYPGDGDT NYNGKFKGKATLTADKSSSTAYMQLSSLTSEDSAVYFCARSRGYFYGSTYDSWGQGTT LTVSS, and VL amino acid sequence DIVLTQSPASLAVSLGQRATISCRASESVDNYGISFMNWFQQKPGQPPKLLIYAASNQGS GVPARFSGSGSGTDFSLNIHPMEEDDTAMYFCQQSKEVPWTFGGGTKLEIKR.

In some embodiments, the anti-ALK antibody comprises VH amino acid sequence QVQLQQSGAELAKPGASVKLSCKASGYTFTSYWMHWVKQRPGQGLEWIGYIKPSSGY TKYNQKFKDKATLTADKSSSTAYMQLSSLTYEDSAVYYCARDYYGSSSWFAYWGQGT

LVTVSA, and VL amino acid sequence DIVMTQSQRFMSTSVGDRVSVTCKASQNVGTNVAWYQQKPGQSPKALIYSASYRYSGV PDRFTGSGSGTDFTLTISNVQSEDLAEYFCQQYNSYPYMYTFGGGTKLEIKR.

In some embodiments, the anti-ALK antibody comprises VH amino acid sequence DVKLVESGEGLVKPGGSLKLSCAASGFTFSSYAMSWVRQTPEKRLEWVTYISSGGDYIY YADTVKGRFTISRDNARNTLYLQMSSLKSEDTAMYYCTRERIWLRRFFDVWGTGTTVT VSS, and VL amino acid sequence DIVMTQSQKFMSTSVGDRVSITCKASQNVGTAVAWYQLKPGQSPKLLIYSASNRFTGVP DRFTGSGSGTDFTLTISNMQSEDLADYFCQQYSSYPLTFGSGTKLEIKR.

In some embodiments, the anti-ALK antibody comprises VH amino acid sequence QVQLQQSGAELAKPGASVKLSCKASGYTFTSYWMHWVKQRPGQGLEWIGYINPSSGY TKYNQKFKDKATLTADKSSSTAYMQLSSLTFEDSAVYYCARDYYGSSSWFAYWGQGT LVTVSA, and VL amino acid sequence DIVMTQSQKFMSTSVGDRVSVTCKASQNVGTNVAWYQQKPGHSPKALIYSASYRYSGV PDRFTGSGSGTDFTLTISNVQSEDLAEYFCQRYNSYPYMFTFGGGTKLEIKR.

In some embodiments, the transmembrane domain is selected from the group consisting of CD8, CD137 (4-1BB), and CD28 in one embodiment, the transmembrane domain is CD8. In some embodiments, the at least one signaling domain is selected from the group consisting of CD8, CD28, CD134 (OX40), CD137 (4-1BB), and CD3ζ. In one embodiment, the at least one signaling domain is CD28 and CD3ζ.

In some embodiments, the structure of the ALK CAR from 5′ to 3′ includes: the extracellular binding domain, a CD8 transmembrane domain, a CD28 signaling domain, and a CD3ζ signaling domain. In some embodiments, the ALK CAR includes a signal peptide. In one embodiment, the signal peptide is mCD8, CD8α, or GM-CSF. In some embodiments, the ALK

CAR includes a splice donor and/or splice acceptor site. In some embodiments, the ALK CAR includes a packaging signal. In some embodiments, the ALK CAR includes the backbone structure and domains of the m1928z CAR. In some embodiments, the extracellular binding domain specifically binds to an extracellular domain of an anaplastic lymphoma kinase (ALK) polypeptide or antibody-binding fragment thereof.

One aspect of the invention provides a polynucleotide encoding an ALK CAR as provided herein.

In another aspect, the invention provides a vector comprising a polynucleotide as provided herein. In some embodiments, the vector is a viral vector. In some embodiments, the vector is a lentiviral, a retroviral, an adenoviral, an Adeno-Associated Virus (AAV), a plasmid, a transposon, and insertion sequence, or an artificial chromosomal vector. In some embodiments, the vector includes a promoter operably linked to the polynucleotide sequence encoding the ALK CAR.

One aspect of the invention provides an engineered immune cell expressing at the cell surface membrane an ALK CAR as provided herein.

In another aspect, the invention provides an engineered immune cell produced by transforming an immune cell with a polynucleotide as provided herein or transducing with a vector as provided herein. In some embodiments, the engineered immune cell is derived from inflammatory T-lymphocytes, cytotoxic T-lymphocytes, regulatory T-lymphocytes, natural killer T (NKT) cells, natural killer (NK) cells, or helper T-lymphocytes. In some embodiments, the engineered immune cell further expresses one or more cytokines. In some embodiments, the cytokine is selected from the group consisting of: interleukin-1 (IL-1), interleukin-2 (IL-2), interleukin-4 (IL-4), interleukin-6 (IL-6), interleukin-7 (IL-7), interleukin-12 (IL-12), interleukin-15 (IL-15), interleukin-21 (IL-21), the protein memory T-cell attractant “Regulated on Activation, Normal Expressed and Secreted” (RANTES), granulocyte-macrophage-colony stimulating factor (GM-CSF), tumor necrosis factor-alpha (TNF-α), or interferon-gamma (IFN-γ), and macrophage inflammatory protein 1 alpha (MIP-1α). In one embodiment, the cytokine is a human cytokine.

In some embodiments, the engineered immune cell is for use in the treatment of an ALK-positive cancer. In some embodiments, the ALK-positive cancer is selected from the group consisting of non-small cell lung cancer (NSCLC), anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, and melanoma. In one embodiment, the ALK-positive cancer is neuroblastoma or melanoma. In another embodiment, the ALK-positive cancer is neuroblastoma. In another embodiment, the ALK-positive cancer is melanoma. In some embodiments, the ALK-positive cancer has an ALK^{F1174L} activating point mutation.

One aspect of the invention provides a method of engineering an immune cell comprising: providing an immune cell; and expressing at the surface of the immune cell at least one ALK CAR as provided herein.

In another aspect, the invention provides a method of engineering an immune cell comprising: providing an immune cell; introducing into the immune cell a polynucleotide as provided herein; and expressing the polynucleotide in the immune cell. In some embodiments, the immune cell is isolated from a subject. In some embodiments, the immune cell is selected from an inflammatory T-lymphocyte, cytotoxic T-lymphocyte, regulatory T-lymphocyte, natural killer T (NKT) cell, natural killer (NK) cell, or helper T-lymphocyte.

One aspect of the invention provides a pharmaceutical composition comprising an ALK CAR as provided herein, a polynucleotide as provided herein, or an engineered immune as provided herein, and a pharmaceutically acceptable carrier, diluent, or excipient. In some embodiments, the composition comprises an effective amount of a ALK CAR as provided herein, a polynucleotide as provided herein, or an engineered immune cell as provided herein.

In another aspect, the invention provides a method of treating a subject with an ALK-positive cancer comprising administrating a pharmaceutical composition as provided herein to the subject.

In yet another aspect, the invention provides a method of treating a subject with an ALK-positive cancer comprising administering to the subject an ALK CAR as provided herein, a polynucleotide as provided herein, or an engineered immune cell as provided herein.

In another aspect, the invention provides a method of treating a subject with an ALK-positive cancer, the method comprising: transforming immune cells with a vector as provided herein to obtain an engineered immune cell, wherein the immune cell comprises a polynucleotide as provided herein; and administering an effective amount of the engineered immune cell to the subject. In some embodiments, the immune cells are derived from the subject. In some embodiments, the immune cells are derived from a donor. In some embodiments, the method includes administering an effective amount of an ALK vaccine to the subject, wherein the ALK vaccine comprises at least one isolated ALK polypeptide or polynucleotide.

One aspect of the invention provides a method of treating a subject with an ALK-positive cancer, the method comprising administering an effective amount of an engineered immune cell comprising an ALK CAR and an effective amount of an ALK vaccine comprising at least one isolated ALK polypeptide or polynucleotide to the subject. In some embodiments, the engineered immune cell is administered simultaneously or sequentially with the ALK vaccine to the subject. In some embodiments, the ALK polypeptide or polynucleotide is conjugated to an amphiphile. In one embodiment, the amphiphile is N-hydroxy succinimidyl ester-end-functionalized poly(ethylene glycol)-lipid (NHS-PEG2KDa-DSPE). In some embodiments, the method includes administering, simultaneously or sequentially, an effective amount of one or more of an ALK inhibitor, immune checkpoint inhibitor, and/or tyrosine kinase inhibitor (TKI). In some embodiments, the method includes administering, simultaneously or sequentially, an effective amount of a tyrosine kinase inhibitor (TKI). In one embodiment, the TKI is lorlatinib. In some embodiments, the method includes administering, simultaneously or sequentially, an effective amount of an immunosuppressor. In one embodiment, the immunosuppressor is cyclophosphamide (CTX).

In some embodiments, the subject is a mammal. In some embodiments, the subject is a human or rodent. In some embodiments, the ALK-positive cancer is selected from the group consisting of non-small cell lung cancer (NSCLC), anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, and melanoma. In some embodiments, the ALK-positive cancer is neuroblastoma or melanoma. In one embodiment, the ALK-positive cancer has an ALK^{F1174L} activating point mutation.

One aspect of the invention provides a kit comprising an agent for administration to a subject. In some embodiments, the agent is an ALK CAR as provided herein, a polynucleotide as provided herein, an engineered immune cell as provided herein, a pharmaceutical composition as provided herein, or a vector as provided herein. In some embodiments, the kit includes instructions for using the kit.

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention pertains or relates. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); Benjamin Lewin, Genes V, published by Oxford University Press, 1994 (ISBN 0-19-854287-9); Kendrew et al. (eds.); The Encyclopedia of Molecular Biology, published by Blackwell Science Ltd., 1994 (ISBN 0-632-02182-9); Molecular Biology and Biotechnology: a Comprehensive Desk Reference, Robert A. Meyers (ed.), published by VCH Publishers, Inc., 1995 (ISBN 1-56081-569-8); and Hale & Marham, The Harper Collins Dictionary of Biology (1991). As used herein, the following terms have the meanings ascribed to them below, unless specified otherwise.

By “adjuvant” is meant a substance or vehicle that non-specifically enhances the immune response to an antigen. Adjuvants may include a suspension of minerals (e.g., alum, aluminum hydroxide, or phosphate) on which antigen is adsorbed; or water-in-oil emulsion in which antigen solution is emulsified in mineral oil (e.g., Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (see, e.g., U.S. Pat. Nos. 6,194,388; 6,207,646; 6,214,806; 6,218,371; 6,239,116; 6,339,068; 6,406,705; and 6,429,199). Adjuvants also include biological molecules, such as costimulatory molecules. In some embodiments, biological adjuvants include cytokines. Exemplary biological adjuvants include, without limitation, interleukin-1 (IL-2), the protein memory T-cell attractant “Regulated on Activation, Normal T Expressed and Secreted” (RANTES), granulocyte-macrophage-colony stimulating factor (GM-CSF), tumor necrosis factor-alpha (TNF-α), interferon-gamma (IFN-γ), granulocyte-colony stimulation factor (G-CSF), lymphocyte function-associated antigen 3 (LFA-3, also called CD58), cluster of differentiation antigen 72 (CD72), (a negative regulator of B-cell responsiveness), peripheral membrane protein, B7-1 (B7-1, also called CD80), peripheral membrane protein, B7-2 (B7-2, also called CD86), the TNF ligand superfamily member 4 ligand (OX40L) or the type 2 transmembrane glycoprotein receptor belonging to the TNF superfamily (4-1BBL). In some embodiments, the adjuvant may be conjugated to an amphiphile as described in H. Liu et al., Structure-based programming of lymph-node targeting in molecular vaccines. Nature 507, 5199522 (2014). In some embodiments, the amphiphile conjugated to the adjuvant is N-hydroxy succinimidyl ester-end-functionalized poly(ethylene glycol)-lipid (NHS-PEG2KDa-DSPE).

By “administer” is meant giving, supplying, or dispensing a composition, agent, therapeutic and the like to a subject, or applying or bringing the composition and the like into contact with the subject. Administering or administration may be accomplished by any of a number of routes, such as, for example, without limitation, topical, oral, subcutaneous, intramuscular, intraperitoneal, intravenous (IV), injection, intrathecal, intramuscular, dermal, intradermal, intracranial, inhalation, rectal, intravaginal, or intraocular.

By “adoptive cell transfer” or “ACT” is meant a process in which immune effector cells (e.g. T cells) are isolated and engineered to recognize a specific antigen (i.e., “engineered immune cells”), then expanded and reintroduced to a subject. Immune effector cells (e.g., T cells) used for ACT may be “autologous,” derived from the subject to be treated, or “allogeneic” (sometimes called “homologous”), derived from a donor subject with an immunogenic profile similar enough not to be rejected by the subject receiving ACT. In some embodiments, cells to be transferred in ACT are CAR-T cells.

By “agent” is meant any small molecule chemical compound, antibody, nucleic acid molecule, peptide, polypeptide, or fragments thereof.

By “anaplastic lymphoma kinase” or “ALK” is meant a receptor tyrosine kinase belonging to the insulin receptor superfamily.

By “ALK antibody” or “anti-ALK antibody” is meant an antibody or an antigen-binding portion thereof that specifically binds to an ALK polypeptide. In some embodiments, the anti-ALK antibody binds to a murine ALK protein or an antibody-binding portion thereof. In some embodiments, the anti-ALK antibody binds to a human ALK protein or an antibody-binding portion thereof. In some embodiments, the anti-ALK antibody binds to a portion of the extracellular domain of the ALK receptor. In some embodiments, the anti-ALK antibody binds to a portion of the extracellular domain of a murine ALK receptor. In some embodiments, the anti-ALK antibody binds to a portion of the extracellular domain of a human ALK receptor. In some embodiments, the anti-ALK antibody is a murine antibody. In some embodiments, the anti-ALK antibody is a human antibody. In some embodiments, the anti-ALK antibody is a humanized antibody. In some embodiments, the anti-ALK antibody is a chimeric antibody. In some embodiments, the anti-ALK antibody modulates ALK activity (e.g., ALK signaling) and/or ALK expression.

In some embodiments, the anti-ALK antibody is an antibody selected from ALK Antibody #1 (ALK #1), ALK Antibody #2 (ALK #2), ALK Antibody #3 (ALK #3), ALK Antibody #4 (ALK #4), ALK Antibody #5 (ALK #5), ALK Antibody #6 (ALK #6), or ALK Antibody #7 (ALK #7). In some embodiments, the anti-ALK antibody is ALK #1. In some embodiments, the anti-ALK antibody is ALK #2. In some embodiments, the anti-ALK antibody is ALK #3. In some embodiments, the anti-ALK antibody is ALK #4. In some embodiments, the anti-ALK antibody is ALK #5. In some embodiments, the anti-ALK antibody is ALK #6. In some embodiments, the anti-ALK antibody is ALK #7.

By “ALK inhibitor” is meant an agent that that inhibits or decreases ALK activity, such as ALK tyrosine kinase activity. In some embodiments, an ALK inhibitor can be a small molecule, a protein (e.g., an antibody), or a nucleic acid (e.g., an antisense molecule). An ALK inhibitor may inhibit or decrease binding of a ligand (e.g., pleiotrophin) to ALK and thus decrease ALK tyrosine kinase activity. An ALK inhibitor may also directly inhibit or decrease ALK tyrosine kinase activity, for example, an ATP-competitive inhibitor (e.g., crizotinib). Molecules that decrease or inhibit expression of ALK (e.g., antisense molecules) are also ALK inhibitors. The ALK inhibitor may specifically inhibit ALK tyrosine kinase activity or may inhibit other receptor tyrosine kinase activity (e.g., c-Met/HGFR activity), in addition to inhibiting ALK tyrosine kinase activity. Nonlimiting examples of ALK inhibitors include the following: crizotinib, ceritinib, alectinib, brigatinib, and lorlatinib. PKIs or other agents that affect ALK may render ALK-positive cancers more susceptible to immune targeting with anti-ALK antibody or with CAR-expressing T cells specific for ALK.

By “ALK polypeptide,” “ALK peptide” or “ALK protein” is meant an anaplastic lymphoma kinase (ALK) protein or fragment thereof. The full-length ALK protein includes an extracellular domain, a hydrophobic stretch corresponding to a single pass transmembrane region, and an intracellular kinase domain. In some embodiments, the ALK polypeptide is at least 85%, at least 90%, at least 95%, at least 98%, at least 99%, or 100% identical to a full-length ALK protein. In some embodiments, the ALK polypeptide is at least 85%, at least 90%, at least 95%, at least 98%, at least 99%, or 100% identical to a full-length ALK protein in Homo Sapiens. In some embodiments, the ALK polypeptide is at least 85%, at least 90%, at least 95%, at least 98%, at least 99%, or 100% identical to a full-length murine ALK protein. In some embodiments, the ALK polypeptide comprises an ALK extracellular domain. In some embodiments, the ALK polypeptide is at least 85%, at least 90%, at least 95%, at least 98%, at least 99%, or 100% identical to an ALK extracellular domain in Homo Sapiens. In some embodiments, the ALK polypeptide is at least 85%, at least 90%, at least 95%, at least 98%, at least 99%, or 100% identical to a murine ALK extracellular domain. In some embodiments, the ALK polypeptide comprises an ALK intracellular domain. In some embodiments, the ALK polypeptide is at least 85%, at least 90%, at least 95%, at least 98%, at least 99%, or 100% identical to an ALK intracellular domain in Homo Sapiens. In some embodiments, the ALK polypeptide is at least 85%, at least 90%, at least 95%, at least 98%, at least 99%, or 100% identical to a murine ALK intracellular domain.

In some embodiments, the ALK polypeptide comprises an amino acid sequence that is at least 85%, at least 90%, at least 95%, at least 98%, at least 99%, or 100% identical to an ALK amino acid sequence associated with GenBank™ Accession NOs.: BAD92714.1, ACY79563, NP_004295, ACI47591, or EDL38401.1). Human and murine ALK protein sequences are publicly available. One of ordinary skill in the art can identify additional ALK protein sequences, including ALK variants.

An exemplary ALK full-length amino acid sequence from Homo Sapiens is provided below (ALK cytoplasmic portion in bold font):

TASSGGMGAIGLLWLLPLLLSTAAVGSGMGTGQRAGSPAAGPPLQPREPL
SYSRLQRKSLAVDEVVPSLFRVYARDLLLPPSSSELKAGRPEARGSLALD
CAPLLRLLGPAPGVSWTAGSPAPAEARTLSRVLKGGSVRKLRRAKQLVLE
LGEEAILEGCVGPPGEAAVGLLQFNLSELFSWWIRQGEGRLRIRLMPEKK
ASEVGREGRLSAAIRASQPRLLFQIFGTGHSSLESPTNMPSPSPDYFTWN
LTWIMKDSFPFLSHRSRYGLECSFDFPCELEYSPPLHDLRNQSWSWRRIP
SEEASQMDLLDGPGAERSKEMPRGSFLLLNTSADSKHTILSPWMRSSSEH
CTLAVSVHRHLQPSGRYIAQLLPHNEAAREILLMPTPGKHGWTVLQGRIG
RPDNPFRVALEYISSGNRSLSAVDFFALKNCSEGTSPGSKMALQSSFTCW
NGTVLQLGQACDFHQDCAQGEDESQMCRKLPVGFYCNFEDGFCGWTQGTL
SPHTPQWQVRTLKDARFQDHQDHALLLSTTDVPASESATVTSATFPAPIK
SSPCELRMSWLIRGVLRGNVSLVLVENKTGKEQGRMVWHVAAYEGLSLWQ
WMVLPLLDVSDRFWLQMVAWWGQGSRAIVAFDNISISLDCYLTISGEDKI
LQNTAPKSRNLFERNPNKELKPGENSPRQTPIFDPTVHWLFTTCGASGPH
GPTQAQCNNAYQNSNLSVEVGSEGPLKGIQIWKVPATDTYSISGYGAAGG
KGGKNTMMRSHGVSVLGIFNLEKDDMLYILVGQQGEDACPSTNQLIQKVC
IGENNVIEEEIRVNRSVHEWAGGGGGGGGATYVFKMKDGVPVPLIIAAGG
GGRAYGAKTDTFHPERLENNSSVLGLNGNSGAAGGGGGWNDNTSLLWAGK
SLQEGATGGHSCPQAMKKWGWETRGGFGGGGGGCSSGGGGGGYIGGNAAS
NNDPEMDGEDGVSFISPLGILYTPALKVMEGHGEVNIKHYLNCSHCEVDE
CHMDPESHKVICFCDHGTVLAEDGVSCIVSPTPEPHLPLSLILSVVTSAL
VAALVLAFSGIMIVYRRKHQELQAMQMELQSPEYKLSKLRTSTIMTDYNP
NYCFAGKTSSISDLKEVPRKNITLIRGLGHGAFGEVYEGQVSGMPNDPSP
LQVAVKTLPEVCSEQDELDFLMEALIISKFNHQNIVRCIGVSLQSLPRFI
LLELMAGGDLKSFLRETRPRPSQPSSIAMLDLLHVARDIACGCQYLEENH
FIHRDIAARNCLLTCPGPGRVAKIGDFGMARDIYRASYYRKGGCAMLPVK
WMPPEAFMEGIFTSKTDTWSFGVLLWEIFSLGYMPYPSKSNQEVLEFVTS
GGRMDPPKNCPGPVYRIMTQCWQHQPEDRPNFAIILERIEYCTQDPDVIN
TALPIEYGPLVEEEEKVPVRPKDPEGVPPLLVSQQAKREEERSPAAPPPL
PTTSSGKAAKKPTAAEISVRVPRGPAVEGGHVNMAFSQSNPPSELHKVHG
SRNKPTSLWNPTYGSWFTEKPTKKNNPIAKKEPHDRGNLGLEGSCTVPPN
VATGRLPGASLLLEPSSLTANMKEVPLFRLRHFPCGNVNYGYQQQGLPLE
AATAPGAGHYEDTILKSKNSMNQPGP

An exemplary full-length ALK amino acid sequence from Homo Sapiens is provided below:

G A A A V V A A G T S R R L C S E G R G
A P R C F P A A L W S A T Q S R G R * *
W V R R G R Q D F G R P C P E R P Q L L
P P G P L Q C L R T L R S R G A G E S K
D A A N L R S A G A G I H A Q K F S R Q
T V R S L P A A E R * L E G A Q D G S L
R P R F P P R P G R R A W R S Q K E R K
R R P G Q R A A A G S R R S Q P * K L Q
R L E A A P R G D R P Q L R L R G A G E
D G T Q L P P P F N H S S S S V P S A A
S Y R R G R G T R R G E R E A Q G P S Q
* A Q C A * V S L D S P L S F Q V C F I
* T P A R L R A V G G K Q E T C A H A Q
S S G D Q V E G A A G Y Q G L F R A S S
H L G E S E G * G W A R R A V * T A S S
G G M G A I G L L W L L P L L L S T A A
V G S G M G T G Q R A G S P A A G P P L
Q P R E P L S Y S R L Q R K S L A V D F
V V P S L F R V Y A R D L L L P P S S S
E L K A G R P E A R G S L A L D C A P L
L R L L G P A P G V S W T A G S P A P A
E A R T L S R V L K G G S V R K L R R A
K Q L V L E L G E E A I L E G C V G P P
G E A A V G L L Q F N L S E L F S W W I
R Q G E G R L R I R L M P E K K A S E V
G R E G R L S A A I R A S Q P R L L F Q
I F G T G H S S L E S P T N M P S P S P
D Y F T W N L T W I M K D S F P F L S H
R S R Y G L E C S F D F P C E L E Y S P
P L H D L R N Q S W S W R R I P S E E A
S Q M D L L D G P G A E R S K E M P R G
S F L L L N T S A D S K H T I L S P W M
R S S S E H C T L A V S V H R H L Q P S
G R Y I A Q L L P H N E A A R E I L L M
P T P G K H G W T V L Q G R I G R P D N
P F R V A L E Y I S S G N R S L S A V D
F F A L K N C S E G T S P G S K M A L Q
S S F T C W N G T V L Q L G Q A C D F H
Q D C A Q G E D E S Q M C R K L P V G F
Y C N F E D G F C G W T Q G T L S P H T
P Q W Q V R T L K D A R F Q D H Q D H A
L L L S T T D V P A S E S A T V T S A T
F P A P I K S S P C E L R M S W L I R G
V L R G N V S L V L V E N K T G K E Q G
R M V W H V A A Y E G L S L W Q W M V L
P L L D V S D R F W L Q M V A W W G Q G
S R A I V A F D N I S I S L D C Y L T I
S G E D K I L Q N T A P K S R N L F E R
N P N K E L K P G E N S P R Q T P I F D
P T V H W L F T T C G A S G P H G P T Q
A Q C N N A Y Q N S N L S V E V G S E G
P L K G I Q I W K V P A T D T Y S I S G
Y G A A G G K G G K N T M M R S H G V S
V L G I F N L E K D D M L Y I L V G Q Q
G E D A C P S T N Q L I Q K V C I G E N
N V I E E E I R V N R S V H E W A G G G
G G G G G A T Y V F K M K D G V P V P L
I I A A G G G G R A Y G A K T D T F H P
E R L E N N S S V L G L N G N S G A A G
G G G G W N D N T S L L W A G K S L Q E
G A T G G H S C P Q A M K K W G W E T R
G G F G G G G G G C S S G G G G G G Y I
G G N A A S N N D P E M D G E D G V S F
I S P L G I L Y T P A L K V M E G H G E
V N I K H Y L N C S H C E V D E C H M D
P E S H K V I C F C D H G T V L A E D G
V S C I V S P T P E P H L P L S L I L S
V V T S A L V A A L V L A F S G I M I V
Y R R K H Q E L Q A M Q M E L Q S P E Y
K L S K L R T S T I M T D Y N P N Y C F
A G K T S S I S D L K E V P R K N I T L
I R G L G H G A F G E V Y E G Q V S G M
P N D P S P L Q V A V K T L P E V C S E
Q D E L D F L M E A L I I S K F N H Q N
I V R C I G V S L Q S L P R F I L L E L
M A G G D L K S F L R E T R P R P S Q P
S S L A M L D L L H V A R D I A C G C Q
Y L E E N H F I H R D I A A R N C L L T
C P G P G R V A K I G D F G M A R D I Y
R A S Y Y R K G G C A M L P V K W M P P
E A F M E G I F T S K T D T W S F G V L
L W E I F S L G Y M P Y P S K S N Q E V
L E F V T S G G R M D P P K N C P G P V
Y R I M T Q C W Q H Q P E D R P N F A I
I L E R I E Y C T Q D P D V I N T A L P
I E Y G P L V E E E E K V P V R P K D P
E G V P P L L V S Q Q A K R E E E R S P
A A P P P L P T T S S G K A A K K P T A
A E I S V R V P R G P A V E G G H V N M
A F S Q S N P P S E L H K V H G S R N K
P T S L W N P T Y G S W F T E K P T K K
N N P I A K K E P H D R G N L G L E G S
C T V P P N V A T G R L P G A S L L L E
P S S L T A N M K E V P L F R L R H F P
C G N V N Y G Y Q Q Q G L P L E A A T A
P G A G H Y E D T I L K S K N S M N Q P
G P * A R S H T H F S S L G S L R P W R
R E R Q W L L H K P E T K C H V L F C A
N L F * S T T K K A V F * K C F R K V L
S M G S S Y S F E R R K Y H K N E * * I
Q G P D V V A * G F Y A C L L Y T S L C
F F Q I V C A L L Q C S Q N * L L L C F
I V G V I D V S L P C * C G H E P F E G
R G N G N K G V I C N D *

An exemplary Homo Sapiens ALK amino acid sequence from GenBank™ accession no. NP_004295 is provided below:

| | |
|---|---|
| 1 | MGAIGLLWLL PLLLSTAAVG SGMGTGQRAG SPAAGPPLQP REPLSYSRLQ RKSLAVDFVV |
| 61 | PSLFRVYARD LLLPPSSSEL KAGRPEARGS LALDCAPLLR LLGPAPGVSW TAGSPAPAEA |
| 121 | RTLSRVLKGG SVRKLRRAKQ LVLELGEEAI LEGCVGPPGE AAVGLLQFNL SELFSWWIRQ |
| 181 | GEGRLRIRLM PEKKASEVGR EGRLSAAIRA SQPRLLFQIF GTGHSSLESP TNMPSPSPDY |
| 241 | FTWNLTWIMK DSFPFLSHRS RYGLECSFDF PCELEYSPPL HDLRNQSWSW RRIPSEEASQ |
| 301 | MDLLDGPGAE RSKEMPRGSF LLLNTSADSK HTILSPWMRS SSEHCTLAVS VHRHLQPSGR |
| 361 | YIAQLLPHNE AAREILLMPT PGKHGWTVLQ GRIGRPDNPF RVALEYISSG NRSLSAVDFF |
| 421 | ALKNCSEGTS PGSKMALQSS FTCWNGTVLQ LGQACDFHQD CAQGEDESQM CRKLPVGFYC |
| 481 | NFEDGFCGWT QGTLSPHTPQ WQVRTLKDAR FQDHQDHALL LSTTDVPASE SATVTSATFP |
| 541 | APIKSSPCEL RMSWLIRGVL RGNVSLVLVE NKTGKEQGRM VWHVAAYEGL SLWQWMVLPL |
| 601 | LDVSDRFWLQ MVAWWGQGSR AIVAFDNISI SLDCYLTISG EDKILQNTAP KSRNLFERNP |
| 661 | NKELKPGENS PRQTPIFDPT VHWLFTTCGA SGPHGPTQAQ CNNAYQNSNL SVEVGSEGPL |
| 721 | KGIQIWKVPA TDTYSISGYG AAGGKGGKNT MMRSHGVSVL GIFNLEKDDM LYILVGQQGE |
| 781 | DACPSTNQLI QKVCIGENNV IEEEIRVNRS VHEWAGGGGG GGGATYVFKM KDGVPVPLII |
| 841 | AAGGGGRAYG AKTDTFHPER LENNSSVLGL NGNSGAAGGG GGWNDNTSLL WAGKSLQEGA |
| 901 | TGGHSCPQAM KKWGWETRGG FGGGGGGCSS GGGGGGYIGG NAASNNDPEM DGEDGVSFIS |
| 961 | PLGILYTPAL KVMEGHGEVN IKHYLNCSHC EVDECHMDPE SHKVICFCDH GTVLAEDGVS |
| 1021 | CIVSPTPEPH LPLSLILSVV TSALVAALVL AFSGIMIVYR RKHQELQAMQ MELQSPEYKL |
| 1081 | SKLRTSTIMT DYNPNYCFAG KTSSISDLKE VPRKNITLIR GLGHGAFGEV YEGQVSGMPN |
| 1141 | DPSPLQVAVK TLPEVCSEQD ELDFLMEALI ISKFNHQNIV RCIGVSLQSL PRFILLELMA |
| 1201 | GGDLKSFLRE TRPRPSQPSS LAMLDLLHVA RDIACGCQYL EENHFIHRDI AARNCLLTCP |
| 1261 | GPGRVAKIGD FGMARDIYRA SYYRKGGCAM LPVKWMPPEA FMEGIFTSKT DTWSFGVLLW |
| 1321 | EIFSLGYMPY PSKSNQEVLE FVTSGGRMDP PKNCPGPVYR IMTQCWQHQP EDRPNFAIIL |
| 1381 | ERIEYCTQDP DVINTALPIE YGPLVEEEEK VPVRPKDPEG VPPLLVSQQA KREEERSPAA |
| 1441 | PPPLPTTSSG KAAKKPTAAE ISVRVPRGPA VEGGHVNMAF SQSNPPSELH KVHGSRNKPT |
| 1501 | SLWNPTYGSW FTEKPTKKNN PIAKKEPHDR GNLGLEGSCT VPPNVATGRL PGASLLLEPS |
| 1561 | SLTANMKEVP LFRLRHFPCG NVNYGYQQQG LPLEAATAPG AGHYEDTILK SKNSMNQPGP |

An exemplary Homo Sapiens ALK polypeptide sequence from UniProt Accession No. Q9UM73 is provided below (extracellular domain (amino acids 19-1038) provided in bold font):

MGAIGLLWLLPLLLSTAAVGSGMGTGQRAGSPAAGPPLQPREPLSYSRLQ
RKSLAVDFVVPSLFRVYARDLLLPPSSSELKAGRPEARGSLALDCAPLLR
LLGPAPGVSWTAGSPAPAEARTLSRVLKGGSVRKLRRAKQLVLELGEEAI
LEGCVGPPGEAAVGLLQFNLSELFSWWIRQGEGRLRIRLMPEKKASEVGR
EGRLSAAIRASQPRLLFQIFGTGHSSLESPTNMPSPSPDYFTWNLTWIMK
DSFPFLSHRSRYGLECSFDFPCELEYSPPLHDLRNQSWSWRRIPSEEASQ
MDLLDGPGAERSKEMPRGSFLLLNTSADSKHTILSPWMRSSSEHCTLAVS
VHRHLQPSGRYIAQLLPHNEAAREILLMPTPGKHGWTVLQGRIGRPDNPF
RVALEYISSGNRSLSAVDFFALKNCSEGTSPGSKMALQSSFTCWNGTVLQ
LGQACDFHQDCAQGEDESQMCRKLPVGFYCNFEDGFCGWTQGTLSPHTPQ
WQVRTLKDARFQDHQDHALLLSTTDVPASESATVTSATFPAPIKSSPCEL
RMSWLIRGVLRGNVSLVLVENKTGKEQGRMVWHVAAYEGLSLWQWMVLPL
LDVSDRFWLQMVAWWGQGSRAIVAFDNISISLDCYLTISGEDKILQNTAP
KSRNLFERNPNKELKPGENSPRQTPIFDPTVHWLFTTCGASGPHGPTQAQ
CNNAYQNSNLSVEVGSEGPLKGIQIWKVPATDTYSISGYGAAGGKGGKNT
MMRSHGVSVLGIFNLEKDDMLYILVGQQGEDACPSTNQLIQKVCIGENNV
IEEEIRVNRSVHEWAGGGGGGGGATYVFKMKDGVPVPLIIAAGGGGRAYG
AKTDTFHPERLENNSSVLGLNGNSGAAGGGGGWNDNTSLLWAGKSLQEGA
TGGHSCPQAMKKWGWETRGGFGGGGGGCSSGGGGGGYIGGNAASNNDPEM
DGEDGVSFISPLGILYTPALKVMEGHGEVNIKHYLNCSHCEVDECHMDPE
SHKVICFCDHGTVLAEDGVSCIVSPTPEPHLPLSLILSVVTSALVAALVL
AFSGIMIVYRRKHQELQAMQMELQSPEYKLSKLRTSTIMTDYNPNYCFAG
KTSSISDLKEVPRKNITLIRGLGHGAFGEVYEGQVSGMPNDPSPLQVAVK
TLPEVCSEQDELDFLMEALIISKFNHQNIVRCIGVSLQSLPRFILLELMA
GGDLKSFLRETRPRPSQPSSLAMLDLLHVARDIACGCQYLEENHFIHRDI
AARNCLLTCPGPGRVAKIGDFGMARDIYRASYYRKGGCAMLPVKWMPPEA
FMEGIFTSKTDTWSFGVLLWEIFSLGYMPYPSKSNQEVLEFVTSGGRMDP
PKNCPGPVYRIMTQCWQHQPEDRPNFAIILERIEYCTQDPDVINTALPIE
YGPLVEEEEKVPVRPKDPEGVPPLLVSQQAKREEERSPAAPPPLPTTSSG
KAAKKPTAAEISVRVPRGPAVEGGHVNMAFSQSNPPSELHKVHGSRNKPT
SLWNPTYGSWFTEKPTKKNNPIAKKEPHDRGNLGLEGSCTVPPNVATGRL
PGASLLLEPSSLTANMKEVPLFRLRHFPCGNVNYGYQQQGLPLEAATAPG
AGHYEDTILKSKNSMNQPGP

An exemplary ALK full-length amino acid sequence from Mus musculus is provided below:

| | |
|---|---|
| 1 | MGQRQSILKR CPEGSFLLLN TSADSKHTIL SPWMRSSSDH CTLAVSVHRH LQPSGRYVAQ |
| 61 | LLPHNEAGRE ILLVPTPGKH GWTVLQGRVG RPANPFRVAL EYISSGNRSL SAVDFFALKN |
| 121 | CSEGTSPGSK MALQSSFTCW NGTVLQLGQA CDFHQDCAQG EDEGQLCSKL PAGFYCNFEN |
| 181 | GFCGWTQSPL SPHMPRWQVR TLRDAHSQGH QGRALLLSTT DILASEGATV TSATFPAPMK |
| 241 | NSPCELRMSW LIRGVLRGNV SLVLVENKTG KEQSRTVWHV ATDEGLSLWQ HTVLSLLDVT |
| 301 | DRFWLQIVTW WGPGSRATVG FDNISISLDC YLTISGEEKM SLNSVPKSRN LFEKNPNKES |
| 361 | KSWANISGPT PIFDPTVHWL FTTCGASGPH GPTQAQCNNA YQNSNLSVVV GSEGPLKGVQ |
| 421 | IWKVPATDTY SISGYGAAGG KGGKNTMMRS HGVSVLGIFN LEKGDTLYIL VGQQGEDACP |
| 481 | RANQLIQKVC VGENNVIEEE IRVNRSVHEW AGGGGGGGGA TYVFKMKDGV PVPLIIAAGG |
| 541 | GGRAYGAKTE TFHPERLESN SSVLGLNGNS GAAGGGGGWN DNTSLLWAGK SLLEGAAGGH |
| 601 | SCPQAMKKWG WETRGGFGGG GGGGAPQVEE AEDI |

By “ALK polynucleotide” is meant any nucleic acid molecule encoding an ALK polypeptide or fragment thereof (e.g., antigen or antigen protein). In some embodiments, the ALK polynucleotide is at least 85%, at least 90%, at least 95%, at least 98%, at least 99%, or 100% identical to a polynucleotide encoding full-length ALK protein. In some embodiments, the ALK polynucleotide is at least 85%, at least 90%, at least 95%, at least 98%, at least 99%, or 100% identical to a polynucleotide encoding full-length ALK protein in Homo Sapiens. In some embodiments, the ALK polynucleotide is at least 85%, at least 90%, at least 95%, at least 98%, at least 99%, or 100% identical to a polynucleotide encoding a full-length murine ALK protein. In some embodiments, the ALK polynucleotide encodes an ALK extracellular domain. In some embodiments, the ALK polynucleotide is at least 85%, at least 90%, at least 95%, at least 98%, at least 99%, or 100% identical to a polypeptide encoding an ALK extracellular domain in Homo Sapiens. In some embodiments, the ALK polynucleotide is at least 85%, at least 90%, at least 95%, at least 98%, at least 99%, or 100% identical to a polypeptide encoding a murine ALK extracellular domain. In some embodiments, the ALK polynucleotide encodes an ALK intracellular domain. In some embodiments, the ALK polynucleotide is at least 85%, at least 90%, at least 95%, at least 98%, at least 99%, or 100% identical to a polynucleotide encoding an ALK intracellular domain in Homo Sapiens. In some embodiments, the ALK polynucleotide is at least 85%, at least 90%, at least 95%, at least 98%, at least 99%, or 100% identical to a polynucleotide encoding a murine ALK intracellular domain. In some embodiments, the ALK polynucleotide is at least 85%, at least 90%, at least 95%, at least 98%, at least 99%, or 100% identical to a polynucleotide encoding an ALK amino acid sequence associated with GenBank™ Accession Nos.: BAD92714.1, ACY79563, NP_004295, EDL38401.1 or ACI47591. Human and murine ALK polynucleotide sequences are publicly available. One of ordinary skill in the art can identify additional ALK polynucleotide sequences, including ALK variants.

An exemplary Homo Sapiens ALK amino acid sequence from GenBank™ accession no. NM_004304 is provided below:

| | |
|---|---|
| 1 | agatgcgatc cagcggctct gggggcggca gcggtggtag cagctggtac ctcccgccgc |
| 61 | ctctgttcgg agggtcgcgg ggcaccgagg tgctttccgg ccgccctctg gtcggccacc |
| 121 | caaagccgcg ggcgctgatg atgggtgagg agggggcggc aagatttcgg gcgcccctgc |
| 181 | cctgaacgcc ctcagctgct gccgccgggg ccgctccagt gcctgcgaac tctgaggagc |
| 241 | cgaggcgccg gtgagagcaa ggacgctgca aacttgcgca gcgcgggggc tgggattcac |
| 301 | gcccagaagt tcagcaggca gacagtccga agccttcccg cagcggagag atagcttgag |
| 361 | ggtgcgcaag acggcagcct ccgccctcgg ttcccgccca gaccgggcag aagagcttgg |
| 421 | aggagccaaa aggaacgcaa aaggcggcca ggacagcgtg cagcagctgg gagccgccgt |
| 481 | tctcagcctt aaaagttgca gagattggag gctgccccga gaggggacag accccagctc |
| 541 | cgactgcggg gggcaggaga ggacggtacc caactgccac ctcccttcaa ccatagtagt |
| 601 | tcctctgtac cgagcgcagc gagctacaga cgggggcgcg gcactcggcg cggagagcgg |
| 661 | gaggctcaag gtcccagcca gtgagcccag tgtgcttgag tgtctctgga ctcgcccctg |
| 721 | agcttccagg tctgtttcat ttagactcct gctcgcctcc gtgcagttgg gggaaagcaa |
| 781 | gagacttgcg cgcacgcaca gtcctctgga gatcaggtgg aaggagccgc tgggtaccaa |
| 841 | ggactgttca gagcctcttc ccatctcggg gagagcgaag ggtgaggctg ggcccggaga |
| 901 | gcagtgtaaa cggcctcctc cggcgggatg ggagccatcg ggctcctgtg gctcctgccg |
| 961 | ctgctgcttt ccacggcagc tgtgggctcc gggatgggga ccggccagcg cgcgggctcc |
| 1021 | ccagctgcgg ggccgccgct gcagccccgg gagccactca gctactcgcg cctgcagagg |
| 1081 | aagagtctgg cagttgactt cgtggtgccc tcgctcttcc gtgtctacgc ccgggaccta |
| 1141 | ctgctgccac catcctcctc ggagctgaag gctggcaggc ccgaggcccg cggctcgcta |
| 1201 | gctctggact gcgccccgct gctcaggttg ctggggccgg cgccgggggt ctcctggacc |
| 1261 | gccggttcac cagccccggc agaggcccgg acgctgtcca gggtgctgaa gggcggctcc |
| 1321 | gtgcgcaagc tccggcgtgc caagcagttg gtgctggagc tgggcgagga ggcgatcttg |
| 1381 | gagggttgcg tcgggccccc cggggaggcg gctgtggggc tgctccagtt caatctcagc |
| 1441 | gagctgttca gttggtggat tcgccaaggc gaagggcgac tgaggatccg cctgatgccc |
| 1501 | gagaagaagg cgtcggaagt gggcagagag ggaaggctgt ccgcggcaat tcgcgcctcc |
| 1561 | cagccccgcc ttctcttcca gatcttcggg actggtcata gctccttgga atcaccaaca |
| 1621 | aacatgcctt ctccttctcc tgattatttt acatggaatc tcacctggat aatgaaagac |
| 1681 | tccttccctt tcctgtctca tcgcagccga tatggtctgg agtgcagctt tgacttcccc |
| 1741 | tgtgagctgg agtattcccc tccactgcat gacctcagga accagagctg gtcctggcgc |
| 1801 | cgcatcccct ccgaggaggc ctcccagatg gacttgctgg atgggcctgg ggcagagcgt |
| 1861 | tctaaggaga tgcccagagg ctcctttctc cttctcaaca cctcagctga ctccaagcac |
| 1921 | accatcctga gtccgtggat gaggagcagc agtgagcact gcacactggc cgtctcggtg |
| 1981 | cacaggcacc tgcagccctc tggaaggtac attgcccagc tgctgcccca caacgaggct |
| 2041 | gcaagagaga tcctcctgat gcccactcca gggaagcatg gttggacagt gctccaggga |
| 2101 | agaatcgggc gtccagacaa cccatttcga gtggccctgg aatacatctc cagtggaaac |
| 2161 | cgcagcttgt ctgcagtgga cttctttgcc ctgaagaact gcagtgaagg aacatcccca |
| 2221 | ggctccaaga tggccctgca gagctccttc acttgttgga atgggacagt cctccagctt |
| 2281 | gggcaggcct gtgacttcca ccaggactgt gcccagggag aagatgagag ccagatgtgc |
| 2341 | cggaaactgc ctgtgggttt ttactgcaac tttgaagatg gcttctgtgg ctggacccaa |
| 2401 | ggcacactgt caccccacac tcctcaatgg caggtcagga ccctaaagga tgcccggttc |
| 2461 | caggaccacc aagaccatgc tctattgctc agtaccactg atgtccccgc ttctgaaagt |
| 2521 | gctacagtga ccagtgctac gtttcctgca ccgatcaaga gctctccatg tgagctccga |
| 2581 | atgtcctggc tcattcgtgg agtcttgagg ggaaacgtgt ccttggtgct agtggagaac |
| 2641 | aaaaccggga aggagcaagg caggatggtc tggcatgtcg ccgcctatga aggcttgagc |
| 2701 | ctgtggcagt ggatggtgtt gcctctcctc gatgtgtctg acaggttctg gctgcagatg |
| 2761 | gtcgcatggt ggggacaagg atccagagcc atcgtggctt ttgacaatat ctccatcagc |
| 2821 | ctggactgct acctcaccat tagcggagag gacaagatcc tgcagaatac agcacccaaa |
| 2881 | tcaagaaacc tgtttgagag aaacccaaac aaggagctga aacccgggga aaattcacca |
| 2941 | agacagaccc ccatctttga ccctacagtt cattggctgt tcaccacatg tggggccagc |
| 3001 | gggccccatg gccccaccca ggcacagtgc aacaacgcct accagaactc caacctgagc |
| 3061 | gtggaggtgg ggagcgaggg ccccctgaaa ggcatccaga tctggaaggt gccagccacc |
| 3121 | gacacctaca gcatctcggg ctacggagct gctggcggga aaggcgggaa gaacaccatg |
| 3181 | atgcggtccc acggcgtgtc tgtgctgggc atcttcaacc tggagaagga tgacatgctg |
| 3241 | tacatcctgg ttgggcagca gggagaggac gcctgcccca gtacaaacca gttaatccag |
| 3301 | aaagtctgca ttggagagaa caatgtgata gaagaagaaa tccgtgtgaa cagaagcgtg |
| 3361 | catgagtggg caggaggcgg aggaggaggg ggtggagcca cctacgtatt taagatgaag |
| 3421 | gatggagtgc cggtgcccct gatcattgca gccggaggtg gtggcagggc ctacggggcc |
| 3481 | aagacagaca cgttccaccc agagagactg gagaataact cctcggttct agggctaaac |
| 3541 | ggcaattccg gagccgcagg tggtggaggt ggctggaatg ataacacttc cttgctctgg |
| 3601 | gccggaaaat ctttgcagga gggtgccacc ggaggacatt cctgccccca ggccatgaag |
| 3661 | aagtgggggt gggagacaag agggggtttc ggagggggtg gaggggggtg ctcctcaggt |
| 3721 | ggaggaggcg gaggatatat aggcggcaat gcagcctcaa acaatgaccc cgaaatggat |
| 3781 | ggggaagatg gggtttcctt catcagtcca ctgggcatcc tgtacacccc agctttaaaa |
| 3841 | gtgatggaag gccacgggga agtgaatatt aagcattatc taaactgcag tcactgtgag |
| 3901 | gtagacgaat gtcacatgga ccctgaaagc cacaaggtca tctgcttctg tgaccacggg |
| 3961 | acggtgctgg ctgaggatgg cgtctcctgc attgtgtcac ccaccccgga gccacacctg |
| 4021 | ccactctcgc tgatcctctc tgtggtgacc tctgccctcg tggccgccct ggtcctggct |
| 4081 | ttctccggca tcatgattgt gtaccgccgg aagcaccagg agctgcaagc catgcagatg |
| 4141 | gagctgcaga gccctgagta caagctgagc aagctccgca cctcgaccat catgaccgac |
| 4201 | tacaacccca actactgctt tgctggcaag acctcctcca tcagtgacct gaaggaggtg |
| 4261 | ccgcggaaaa acatcaccct cattcggggt ctgggccatg gcgcctttgg ggaggtgtat |
| 4321 | gaaggccagg tgtccggaat gcccaacgac ccaagccccc tgcaagtggc tgtgaagacg |
| 4381 | ctgcctgaag tgtgctctga acaggacgaa ctggatttcc tcatggaagc cctgatcatc |
| 4441 | agcaaattca accaccagaa cattgttcgc tgcattgggg tgagcctgca atccctgccc |
| 4501 | cggttcatcc tgctggagct catggcgggg ggagacctca agtccttcct ccgagagacc |
| 4561 | cgccctcgcc cgagccagcc ctcctccctg gccatgctgg accttctgca cgtggctcgg |
| 4621 | gacattgcct gtggctgtca gtatttggag gaaaaccact tcatccaccg agacattgct |
| 4681 | gccagaaact gcctcttgac ctgtccaggc cctggaagag tggccaagat tggagacttc |
| 4741 | gggatggccc gagacatcta cagggcgagc tactatagaa agggaggctg tgccatgctg |
| 4801 | ccagttaagt ggatgccccc agaggccttc atggaaggaa tattcacttc taaaacagac |
| 4861 | acatggtcct ttggagtgct gctatgggaa atcttttctc ttggatatat gccatacccc |
| 4921 | agcaaaagca accaggaagt tctggagttt gtcaccagtg gaggccggat ggacccaccc |
| 4981 | aagaactgcc ctgggcctgt ataccggata atgactcagt gctggcaaca tcagcctgaa |
| 5041 | gacaggccca actttgccat cattttggag aggattgaat actgcaccca ggacccggat |
| 5101 | gtaatcaaca ccgctttgcc gatagaatat ggtccacttg tggaagagga agagaaagtg |
| 5161 | cctgtgaggc ccaaggaccc tgagggggtt cctcctctcc tggtctctca acaggcaaaa |
| 5221 | cgggaggagg agcgcagccc agctgcccca ccacctctgc ctaccacctc ctctggcaag |
| 5281 | gctgcaaaga aacccacagc tgcagagatc tctgttcgag tccctagagg gccggccgtg |
| 5341 | gaagggggac acgtgaatat ggcattctct cagtccaacc ctccttcgga gttgcacaag |
| 5401 | gtccacggat ccagaaacaa gcccaccagc ttgtggaacc caacgtacgg ctcctggttt |
| 5461 | acagagaaac ccaccaaaaa gaataatcct atagcaaaga aggagccaca cgacaggggt |
| 5521 | aacctggggc tggagggaag ctgtactgtc ccacctaacg ttgcaactgg gagacttccg |
| 5581 | ggggcctcac tgctcctaga gccctcttcg ctgactgcca atatgaagga ggtacctctg |
| 5641 | ttcaggctac gtcacttccc ttgtgggaat gtcaattacg gctaccagca acagggcttg |
| 5701 | cccttagaag ccgctactgc ccctggagct ggtcattacg aggataccat tctgaaaagc |
| 5761 | aagaatagca tgaaccagcc tgggccctga gctcggtcgc acactcactt ctcttccttg |
| 5821 | ggatccctaa gaccgtggag gagagagagg caatggctcc ttcacaaacc agagaccaaa |
| 5881 | tgtcacgttt tgttttgtgc caacctattt tgaagtacca ccaaaaaagc tgtattttga |
| 5941 | aaatgcttta gaaaggtttt gagcatgggt tcatcctatt ctttcgaaag aagaaaatat |
| 6001 | cataaaaatg agtgataaat acaaggccca gatgtggttg cataaggttt ttatgcatgt |
| 6061 | ttgttgtata cttccttatg cttctttcaa attgtgtgtg ctctgcttca atgtagtcag |
| 6121 | aattagctgc ttctatgttt catagttggg gtcatagatg tttccttgcc ttgttgatgt |
| 6181 | ggacatgagc catttgaggg gagagggaac ggaaataaag gagttatttg taatgactaa |

An exemplary full-length ALK nucleic acid sequence from Homo Sapiens is provided below:

| | | |
|---|---|---|
| 2 | ggggcggcagcggtggtagcagctggtacctcccgccgcctctgttcggagggtcgcggg | 61 |
| 62 | gcaccgaggtgctttccggccgccctctggtcggccacccaaagccgcgggcgctgatga | 121 |
| 122 | tgggtgaggagggggcggcaagatttcgggcgcccctgccctgaacgccctcagctgctg | 181 |
| 182 | ccgccggggccgctccagtgcctgcgaactctgaggagccgaggcgccggtgagagcaag | 241 |
| 242 | gacgctgcaaacttgcgcagcgcgggggctgggattcacgcccagaagttcagcaggcag | 301 |
| 302 | acagtccgaagccttcccgcagcggagagatagcttgagggtgcgcaagacggcagcctc | 361 |
| 362 | cgccctcggttcccgcccagaccgggcagaagagcttggaggagccaaaaggaacgcaaa | 421 |
| 422 | aggcggccaggacagcgtgcagcagctgggagccgccgttctcagccttaaaagttgcag | 481 |
| 482 | agattggaggctgccccgagaggggacagaccccagctccgactgcggggggcaggagag | 541 |
| 542 | gacggtacccaactgccacctcccttcaaccatagtagttcctctgtaccgagcgcagcg | 601 |
| 602 | agctacagacgggggcgcggcactcggcgcggagagcgggaggctcaaggtcccagccag | 661 |
| 662 | tgagcccagtgtgcttgagtgtctctggactcgcccctgagcttccaggtctgtttcatt | 721 |
| 722 | tagactcctgctcgcctccgtgcagttgggggaaagcaagagacttgcgcgcacgcacag | 781 |
| 782 | tcctctggagatcaggtggaaggagccgctgggtaccaaggactgttcagagcctcttcc | 841 |
| 842 | catctcggggagagcgaagggtgaggctgggcccggagagcagtgtaaacggcctcctcc | 901 |
| 902 | ggcgggatgggagccatcgggctcctgtggctcctgccgctgctgctttccacggcagct | 961 |
| 962 | gtgggctccgggatggggaccggccagcgcgcgggctccccagctgcggggccgccgctg | 1021 |
| 1022 | cagccccgggagccactcagctactcgcgcctgcagaggaagagtctggcagttgacttc | 1081 |
| 1082 | gtggtgccctcgctcttccgtgtctacgcccgggacctactgctgccaccatcctcctcg | 1141 |
| 1142 | gagctgaaggctggcaggcccgaggcccgcggctcgctagctctggactgcgccccgctg | 1201 |
| 1202 | ctcaggttgctggggccggcgccgggggtctcctggaccgccggttcaccagccccggca | 1261 |
| 1262 | gaggcccggacgctgtccagggtgctgaagggcggctccgtgcgcaagctccggcgtgcc | 1321 |
| 1322 | aagcagttggtgctggagctgggcgaggaggcgatcttggagggttgcgtcgggcccccc | 1381 |
| 1382 | ggggaggcggctgtggggctgctccagttcaatctcagcgagctgttcagttggtggatt | 1441 |
| 1442 | cgccaaggcgaagggcgactgaggatccgcctgatgcccgagaagaaggcgtcggaagtg | 1501 |
| 1502 | ggcagagagggaaggctgtccgcggcaattcgcgcctcccagccccgccttctcttccag | 1561 |
| 1562 | atcttcgggactggtcatagctccttggaatcaccaacaaacatgccttctccttctcct | 1621 |
| 1622 | gattattttacatggaatctcacctggataatgaaagactccttccctttcctgtctcat | 1681 |
| 1682 | cgcagccgatatggtctggagtgcagctttgacttcccctgtgagctggagtattcccct | 1741 |
| 1742 | ccactgcatgacctcaggaaccagagctggtcctggcgccgcatcccctccgaggaggcc | 1801 |
| 1802 | tcccagatggacttgctggatgggcctggggcagagcgttctaaggagatgcccagaggc | 1861 |
| 1862 | tcctttctccttctcaacacctcagctgactccaagcacaccatcctgagtccgtggatg | 1921 |
| 1922 | aggagcagcagtgagcactgcacactggccgtctcggtgcacaggcacctgcagccctct | 1981 |
| 1982 | ggaaggtacattgcccagctgctgccccacaacgaggctgcaagagagatcctcctgatg | 2041 |
| 2042 | cccactccagggaagcatggttggacagtgctccagggaagaatcgggcgtccagacaac | 2101 |
| 2102 | ccatttcgagtggccctggaatacatctccagtggaaaccgcagcttgtctgcagtggac | 2161 |
| 2162 | ttctttgccctgaagaactgcagtgaaggaacatccccaggctccaagatggccctgcag | 2221 |
| 2222 | agctccttcacttgttggaatgggacagtcctccagcttgggcaggcctgtgacttccac | 2281 |
| 2282 | caggactgtgcccagggagaagatgagagccagatgtgccggaaactgcctgtgggtttt | 2341 |
| 2342 | tactgcaactttgaagatggcttctgtggctggacccaaggcacactgtcaccccacact | 2401 |
| 2402 | cctcaatggcaggtcaggaccctaaaggatgcccggttccaggaccaccaagaccatgct | 2461 |
| 2462 | ctattgctcagtaccactgatgtccccgcttctgaaagtgctacagtgaccagtgctacg | 2521 |
| 2522 | tttcctgcaccgatcaagagctctccatgtgagctccgaatgtcctggctcattcgtgga | 2581 |
| 2582 | gtcttgaggggaaacgtgtccttggtgctagtggagaacaaaaccgggaaggagcaaggc | 2641 |
| 2642 | aggatggtctggcatgtcgccgcctatgaaggcttgagcctgtggcagtggatggtgttg | 2701 |
| 2702 | cctctcctcgatgtgtctgacaggttctggctgcagatggtcgcatggtggggacaagga | 2761 |
| 2762 | tccagagccatcgtggcttttgacaatatctccatcagcctggactgctacctcaccatt | 2821 |
| 2822 | agcggagaggacaagatcctgcagaatacagcacccaaatcaagaaacctgtttgagaga | 2881 |
| 2882 | aacccaaacaaggagctgaaacccggggaaaattcaccaagacagacccccatctttgac | 2941 |
| 2942 | cctacagttcattggctgttcaccacatgtggggccagcgggccccatggccccacccag | 3001 |
| 3002 | gcacagtgcaacaacgcctaccagaactccaacctgagcgtggaggtggggagcgagggc | 3061 |
| 3062 | cccctgaaaggcatccagatctggaaggtgccagccaccgacacctacagcatctcgggc | 3121 |
| 3122 | tacggagctgctggcgggaaaggcgggaagaacaccatgatgcggtcccacggcgtgtct | 3181 |
| 3182 | gtgctgggcatcttcaacctggagaaggatgacatgctgtacatcctggttgggcagcag | 3241 |
| 3242 | ggagaggacgcctgccccagtacaaaccagttaatccagaaagtctgcattggagagaac | 3301 |
| 3302 | aatgtgatagaagaagaaatccgtgtgaacagaagcgtgcatgagtgggcaggaggcgga | 3361 |
| 3362 | ggaggagggggtggagccacctacgtatttaagatgaaggatggagtgccggtgcccctg | 3421 |
| 3422 | atcattgcagccggaggtggtggcagggcctacggggccaagacagacacgttccaccca | 3481 |
| 3482 | gagagactggagaataactcctcggttctagggctaaacggcaattccggagccgcaggt | 3541 |
| 3542 | ggtggaggtggctggaatgataacacttccttgctctgggccggaaaatctttgcaggag | 3601 |
| 3602 | ggtgccaccggaggacattcctgcccccaggccatgaagaagtgggggtgggagacaaga | 3661 |
| 3662 | gggggtttcggagggggtggaggggggtgctcctcaggtggaggaggcggaggatatata | 3721 |
| 3722 | ggcggcaatgcagcctcaaacaatgaccccgaaatggatggggaagatggggtttccttc | 3781 |
| 3782 | atcagtccactgggcatcctgtacaccccagctttaaaagtgatggaaggccacggggaa | 3841 |
| 3842 | gtgaatattaagcattatctaaactgcagtcactgtgaggtagacgaatgtcacatggac | 3901 |
| 3902 | cctgaaagccacaaggtcatctgcttctgtgaccacgggacggtgctggctgaggatggc | 3961 |
| 3962 | gtctcctgcattgtgtcacccaccccggagccacacctgccactctcgctgatcctctct | 4021 |
| 4022 | gtggtgacctctgccctcgtggccgccctggtcctggctttctccggcatcatgattgtg | 4081 |
| 4082 | taccgccggaagcaccaggagctgcaagccatgcagatggagctgcagagccctgagtac | 4141 |
| 4142 | aagctgagcaagctccgcacctcgaccatcatgaccgactacaaccccaactactgcttt | 4201 |
| 4202 | gctggcaagacctcctccatcagtgacctgaaggaggtgccgcggaaaaacatcaccctc | 4261 |
| 4262 | attcggggtctgggccatggcgcctttggggaggtgtatgaaggccaggtgtccggaatg | 4321 |
| 4322 | cccaacgacccaagccccctgcaagtggctgtgaagacgctgcctgaagtgtgctctgaa | 4381 |
| 4382 | caggacgaactggatttcctcatggaagccctgatcatcagcaaattcaaccaccagaac | 4441 |
| 4442 | attgttcgctgcattggggtgagcctgcaatccctgccccggttcatcctgctggagctc | 4501 |
| 4502 | atggcggggggagacctcaagtccttcctccgagagacccgccctcgcccgagccagccc | 4561 |
| 4562 | tcctccctggccatgctggaccttctgcacgtggctcgggacattgcctgtggctgtcag | 4621 |
| 4622 | tatttggaggaaaaccacttcatccaccgagacattgctgccagaaactgcctcttgacc | 4681 |
| 4682 | tgtccaggccctggaagagtggccaagattggagacttcgggatggcccgagacatctac | 4741 |
| 4742 | agggcgagctactatagaaagggaggctgtgccatgctgccagttaagtggatgccccca | 4801 |
| 4802 | gaggccttcatggaaggaatattcacttctaaaacagacacatggtcctttggagtgctg | 4861 |
| 4862 | ctatgggaaatcttttctcttggatatatgccataccccagcaaaagcaaccaggaagtt | 4921 |
| 4922 | ctggagtttgtcaccagtggaggccggatggacccacccaagaactgccctgggcctgta | 4981 |
| 4982 | taccggataatgactcagtgctggcaacatcagcctgaagacaggcccaactttgccatc | 5041 |
| 5042 | attttggagaggattgaatactgcacccaggacccggatgtaatcaacaccgctttgccg | 5101 |
| 5102 | atagaatatggtccacttgtggaagaggaagagaaagtgcctgtgaggcccaaggaccct | 5161 |
| 5162 | gagggggttcctcctctcctggtctctcaacaggcaaaacgggaggaggagcgcagccca | 5221 |
| 5222 | gctgccccaccacctctgcctaccacctcctctggcaaggctgcaaagaaacccacagct | 5281 |
| 5282 | gcagagatctctgttcgagtccctagagggccggccgtggaagggggacacgtgaatatg | 5341 |
| 5342 | gcattctctcagtccaaccctccttcggagttgcacaaggtccacggatccagaaacaag | 5401 |
| 5402 | cccaccagcttgtggaacccaacgtacggctcctggtttacagagaaacccaccaaaaag | 5461 |
| 5462 | aataatcctatagcaaagaaggagccacacgacaggggtaacctggggctggagggaagc | 5521 |
| 5522 | tgtactgtcccacctaacgttgcaactgggagacttccgggggcctcactgctcctagag | 5581 |
| 5582 | ccctcttcgctgactgccaatatgaaggaggtacctctgttcaggctacgtcacttccct | 5641 |
| 5642 | tgtgggaatgtcaattacggctaccagcaacagggcttgcccttagaagccgctactgcc | 5701 |
| 5702 | cctggagctggtcattacgaggataccattctgaaaagcaagaatagcatgaaccagcct | 5761 |
| 5762 | gggccctgagctcggtcgcacactcacttctcttccttgggatccctaagaccgtggagg | 5821 |
| 5822 | agagagaggcaatggctccttcacaaaccagagaccaaatgtcacgttttgttttgtgcc | 5881 |
| 5882 | aacctattttgaagtaccaccaaaaaagctgtattttgaaaatgctttagaaaggttttg | 5941 |
| 5942 | agcatgggttcatcctattctttcgaaagaagaaaatatcataaaaatgagtgataaata | 6001 |
| 6002 | caaggcccagatgtggttgcataaggtttttatgcatgtttgttgtatacttccttatgc | 6061 |
| 6062 | ttctttcaaattgtgtgtgctctgcttcaatgtagtcagaattagctgcttctatgtttc | 6121 |
| 6122 | atagttggggtcatagatgtttccttgccttgttgatgtggacatgagccatttgagggg | 6181 |
| 6182 | agagggaacggaaataaaggagttatttgtaatgactaaaa | 6222 |

An exemplary Mus musculus ALK nucleic acid sequence from GenBank™ accession no. NM_007439.2 is provided below:

| | |
|---|---|
| 1 | gtgttcacgc ccagaagttc agcgggcagg gtgatcgatc cgaagacttc ctgcagcgga |
| 61 | ggtcacttga gggggcgcta gaaagcagcc ccctccggtg gtccttgcct agacctggga |
| 121 | aggagcgcag aggaggtgac aggagcggag gacgtgggca agacagtgac cgactcggag |
| 181 | ccacggttca cagcctggaa agttgcagaa gattggaagc taagaggaga gctctggtcg |
| 241 | ccgagggctc cttgaacggt acctaattgc cacctccctg gtccctgagc aaaggcctct |
| 301 | acaaatgggg cgcagcacgg cgagaggcgc aggatccagc tgttgagccc agggtgtctc |
| 361 | actgtctccg aactaccccc tgactttgtc ttccgttttg ctgagaaccc ttctcgcctc |
| 421 | cttgtagctt gggaaaagca agggcgctct atagtgtaca cacagtccct gagatctagt |
| 481 | ggaaggagcc attcaggacc aaggactatt tggagccctt tcctgtttgg gggagagtga |
| 541 | agggcgaggc tggaccagca agggaaggga gactagtgta aactcgccct ccagcgggat |
| 601 | gggagctgct gggttcctgt ggctgctgcc tccactgctt ttggcagcag cctcgtactc |
| 661 | cggagctgca accgatcagc gcgcgggttc cccagcctca gggcctcctc tgcagccccg |
| 721 | ggagccgctc agttattcgc gcctgcagag gaagagtctg gcagtggact tcgttgtacc |
| 781 | ctcgctcttc cgcgtctatg cccgagacct gctgctaccg cagccacggt ccccctcgga |
| 841 | gcccgaggct ggcgggctgg aggcgcgggg atcactggcc ctggattgtg agcctctgct |
| 901 | caggctgctg gggccactgc ctggaatctc ctgggcagat ggagccagtt ctcctagtcc |
| 961 | cgaggcgggt ccgacgctgt ccagggtgct gaagggaggc tcggtgcgca agctcaggcg |
| 1021 | tgccaaacag ctggtgctgg agctgggcga ggagacgatt cttgaaggct gtattggtcc |
| 1081 | cccagaggag gtagcggctg tggggatact ccagttcaac ctcagcgagc tgttcagctg |
| 1141 | gtggattctc cacggcgaag ggaggctgag gatccgcctg atgcctgaga agaaggcatc |
| 1201 | ggaagtgggc agggagggaa ggctatccag tgcgatccga gcctcccagc cccgccttct |
| 1261 | cttccagatc ttcgggacgg gacacagctc catggagtca ccctcagaaa cgccttctcc |
| 1321 | tcctggtacc ttcatgtgga atctgacctg gacgatgaaa gactccttcc ctttcctttc |
| 1381 | ccaccgcagt cgatatggtc tggagtgcag ctttgacttc ccctgtgagc tggaatattc |
| 1441 | tcctcccctg cacaaccacg ggaatcagag ctggtcctgg cgccatgtgc cctccgagga |
| 1501 | ggcctcgagg atgaacttgc tggatgggcc agaggcagag cattctcaag agatgcccag |
| 1561 | aggctccttc ctcctcctga acacctctgc agattccaag cataccattc tgagcccatg |
| 1621 | gatgaggagc agtagtgatc actgcacact ggctgtctcc gtgcacagac atctacagcc |
| 1681 | ttcggggaga tatgttgccc agctcctacc ccacaacgaa gctggaagag agattctttt |
| 1741 | ggtgcccacc ccagggaagc atggctggac agtgctgcag gggagagtcg ggcgcccagc |
| 1801 | aaacccattt cgagtggctc tggaatacat ctccagtggc aaccggagct tgtcggcggt |
| 1861 | ggatttcttt gccctgaaga actgcagtga agggacatcc ccaggctcca agatggcatt |
| 1921 | gcagagttcc ttcacttgtt ggaatgggac cgtcctccag ctcgggcaag cctgtgattt |
| 1981 | ccaccaggac tgtgcccaag gagaagatga gggccagctg tgcagtaaac ttcctgctgg |
| 2041 | attttactgt aactttgaaa atggcttctg tggctggacc caaagtccac tctcacccca |
| 2101 | tatgccccgg tggcaagtga ggaccctaag agatgcccat tcccagggcc accaaggccg |
| 2161 | tgccctgttg ctcagcacca ctgacatcct cgcttctgaa ggtgcaacag tgaccagtgc |
| 2221 | caccttccct gcaccaatga aaaattctcc ttgtgagctc cgcatgtcct ggctcatccg |
| 2281 | cggggttttg agaggaaacg tatctctggt gctggtggag aacaaaaccg gaaaggagca |
| 2341 | aagccggact gtctggcatg tcgccactga cgaaggctta agcctgtggc agcatacagt |
| 2401 | gctgtccctc ctcgatgtga ctgacaggtt ctggctgcag atagtcacat ggtggggtcc |
| 2461 | aggatccagg gcaaccgtgg gatttgacaa catttccatc agcctcgact gctaccttac |
| 2521 | catcagtgga gaggagaaaa tgtccctgaa ttcagtaccc aaatctagaa atctgtttga |
| 2581 | gaaaaaccca aacaaggagt caaaatcctg ggcaaacata tcaggaccaa ctcccatctt |
| 2641 | cgaccctaca gttcactggc tgttcaccac gtgtggggcc agtggacctc atggccccac |
| 2701 | ccaggcacag tgcaacaacg cctaccagaa ttccaacttg agcgtggtgg tgggaagtga |
| 2761 | agggcccttg aagggagtcc agatttggaa agtgccagct actgacacct acagtatctc |
| 2821 | gggctacgga gcagctggcg ggaaaggtgg gaaaaacacc atgatgcggt cccatggcgt |
| 2881 | gtctgtcctg ggcatcttca atctggagaa aggtgacaca ctctacatcc ttgtcggtca |
| 2941 | gcaaggggag gatgcctgtc ccagggcaaa ccaactaatc cagaaagtct gtgtgggtga |
| 3001 | gaacaatgtc atagaagaag agatccgagt gaacagaagc gtgcacgagt gggcaggagg |
| 3061 | aggaggaggt gggggtggag ccacctacgt gtttaagatg aaagatggcg tgcctgtacc |
| 3121 | cctgatcatt gcagctggtg gtggtggcag ggcctatggg gccaagacag aaacgttcca |
| 3181 | cccagagaga ctggagagta actcctcggt tctagggctg aacggcaatt ccggagccgc |
| 3241 | aggtggtgga ggcggctgga atgataacac ttccttgctc tgggccggaa agtctttgct |
| 3301 | ggagggtgcc gccggaggac attcctgccc ccaggccatg aagaagtggg ggtgggagac |
| 3361 | aagagggggt ttcggagggg gtggaggggg gtgctcctca ggtggaggag gcggaggata |
| 3421 | tataggtggc aacgcagcat caaacaatga ccccgaaatg gatggggaag atggggtttc |
| 3481 | cttcatcagt ccattgggta tcctgtacac cccggcctta aaagtgatgg agggccacgg |
| 3541 | ggaagtgaat atcaagcatt atctaaactg cagtcactgc gaggtagacg aatgtcacat |
| 3601 | ggaccccgag agccacaaag tcatctgctt ctgtgatcat gggaccgtgc tggctgatga |
| 3661 | tggtgtctcc tgcattgtgt cacccacccc ggagccccac ctgccgctct cattgatcct |
| 3721 | ctccgtcgtg acctctgccc tggtggctgc ccttgttctg gcattctccg gcatcatgat |
| 3781 | tgtgtaccgt cggaagcacc aggagttgca ggctatgcag atggaactgc agagccccga |
| 3841 | gtataagctg agcaagctac ggacctcgac catcatgacc gactacaacc ccaactactg |
| 3901 | cttcgctggc aagacttcct ccatcagtga cctgaaagaa gtgccacgga aaaacatcac |
| 3961 | actcatccgg ggcctaggcc atggcgcatt tggggaggtg tatgaaggcc aggtgtctgg |
| 4021 | aatgcccaat gacccaagcc ctctacaagt ggctgtaaag acgctgccag aagtgtgttc |
| 4081 | agaacaagat gagctggact ttctcatgga agctctgatc atcagcaaat tcaaccacca |
| 4141 | gaatattgtt cgctgcatcg gggtgagtct acaagccctg ccccgcttca tcctgctgga |
| 4201 | actcatggct ggcggagacc tcaagtcctt cctcagggag acacgccctc gcccgaacca |
| 4261 | acccacctcc ctggccatgc tggaccttct gcatgtggct cgggacattg cctgtggctg |
| 4321 | tcagtaccta gaggagaatc actttatcca ccgggatatt gctgctagaa actgtctgtt |
| 4381 | gacctgccca ggagctggaa gaatagcaaa gattggagac tttgggatgg cccgagatat |
| 4441 | ctacagggcc agctactacc gaaagggagg ctgcgccatg ctgccggtca agtggatgcc |
| 4501 | ccctgaagcc ttcatggaag ggatatttac ctctaaaaca gacacatggt cttttggagt |
| 4561 | gttgctatgg gaaatatttt ctcttggata tatgccgtac cccagcaaga gcaaccagga |
| 4621 | agttctggag tttgtcacca gcggaggacg gatggacccg cctaagaact gccccgggcc |
| 4681 | tgtataccgg ataatgacgc agtgctggca gcatcagcct gaagacagac ccaacttcgc |
| 4741 | catcattttg gagaggatcg aatactgcac ccaggacccc gatgtgatca acacagctct |
| 4801 | gcccatcgaa tacggtccag tagtagaaga ggaggagaaa gtgcccatgc gccccaaaga |
| 4861 | ccccgagggg atgccacctt tgctggtgtc tccccagcct gcgaagcacg aggaggcgtc |
| 4921 | cgcagctccc cagcccgcag ccctgacggc accaggccca tcggtgaaga agcccccggg |
| 4981 | tgcgggtgcg ggcgcgggcg cgggtgcggg tgccggcccg gtgccccgag gtgcggccga |
| 5041 | tcggggccac gtgaacatgg ctttctctca gcccaaccct cccccggagc tgcacaaagg |
| 5101 | cccgggatcc agaaacaagc cgaccagcct gtggaacccc acctacggct cgtggttcac |
| 5161 | cgagaagcct gccaaaaaga cccatcctcc gccaggcgcc gagccgcagg cgcgggcagg |
| 5221 | agcggccgag ggtggctgga ccgggccggg cgcggggccc cgcagagccg aggcagcgct |
| 5281 | gctgctagag ccatcggcgc tgagcgccac catgaaggag gtgccgctgt tcaggctgcg |
| 5341 | ccacttcccc tgcggcaatg tcaactatgg ttaccagcaa cagggtctcc ccttggaagc |
| 5401 | cacagccgcg ccaggggaca ccatgctgaa aagcaagaat aaggtcaccc agccggggcc |
| 5461 | ctgagccctg tactccacta gcttctcctc ctggcggagc cggagcccac ccagagggag |
| 5521 | atggacagga tggctccacc acaaacccaa gaccaaaact ttcatttttg tgccaacttg |
| 5581 | ttttgaagtg ccacatttta aaaaaaggaa acttgtgttt ttaagatgtg ttagaaggtt |
| 5641 | ttttgagcat gggttcatct atcctctcaa aagaagaaaa tgccattctt taaaaaagaa |
| 5701 | aaaaaagcaa tcagtgcaag gcccagattg gttgcgccaa gttttcgtgc atggtctgct |
| 5761 | gtacagtccc ctaaggcttc tttccgattt ttgtgtgcgc tctgcttccg cgtagtcaga |
| 5821 | aatagctgct tccatgtctc atagggggag tcctaggtgt ttcctttgcc ttatgaatat |
| 5881 | gaaccactcg aggggcgggc gagggaacag aaataaag |

By “alteration” is meant a change (increase or decrease) in the expression levels or activity of a gene or polypeptide as detected by standard art known methods such as those described herein. As used herein, an alteration includes a 5% change in expression levels, a 10% change in expression levels, preferably a 25% change, more preferably a 40% change, and most preferably a 50% or greater change in expression levels.

By “ameliorate” is meant decrease, reduce, delay diminish, suppress, attenuate, arrest, or stabilize the development or progression of a disease or pathological condition.

By “antibody” is meant an immunoglobulin (Ig) molecule produced by B lymphoid cells and having a specific amino acid sequence with an antigen binding site that specifically binds an antigen. “Antibody” may be used interchangeably herein with “immunoglobulin” or “Ig.”

Antibodies are evoked or elicited in subjects (e.g., humans, mammals, or other animals) following exposure to a specific antigen. A subject capable of generating antibodies (i.e., an immune response) directed against a specific antigen is said to be immunocompetent.

Typically, an immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of an antibody molecule:

IgM, IgD, IgG, IgA and IgE.

Each heavy and light chain contains a constant region (or constant domain) and a variable region (or variable domain) (see, e.g., Kindt et al. Kuby Immunology, 6.sup.th ed., W.H. Freeman and Co., page 91 (2007)). In several embodiments, the heavy and the light chain variable regions combine to specifically bind the antigen (e.g., an ALK protein or fragment thereof). Reference to “VH” refers to the variable region of an antibody heavy chain or an antigen binding fragment thereof, including Fv, scFv, dsFv or Fab. Reference to “VL” refers to the variable domain of an antibody light chain or an antigen binding fragment thereof, including Fv, scFv, dsFv or Fab.

Light and heavy chain variable regions contain a framework region (FR) interrupted by three hypervariable regions, also called complementarity-determining regions (CDRs) (see, e.g., Kabat et al., Sequences of Proteins of Immunological Interest, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRS in three-dimensional space. In some embodiments, the spatial orientation of CDRs and FRs are as follows, from N-terminus to C-terminus: FR1-CDR1-FR2-CDR2-FR3-CDR3-FR4.

In some embodiments, the variable region is a primate (e.g., human or non-human primate) variable region. In some embodiments, the variable region is a human variable region. In some embodiments, the variable region comprises murine (e.g., mouse or rat) CDRs and primate (e.g., human or non-human primate) framework regions (FRs). In some embodiments, the variable region comprises murine (e.g., mouse or rat) CDRs and human framework regions (FRs). In one embodiment, a variable region described herein is obtained from assembling two or more fragments of human sequences into a composite human sequence.

In some embodiments, the anti-ALK antibody or an antigen binding fragment thereof comprises a VL region selected from ALK Antibody #1 (ALK #1), ALK Antibody #2 (ALK #2), ALK Antibody #3 (ALK #3), ALK Antibody #4 (ALK #4), ALK Antibody #5 (ALK #5), ALK Antibody #6 (ALK #6), or ALK Antibody #7 (ALK #7). In some embodiments, the anti-ALK antibody VL region is selected from ALK #1. In some embodiments, the anti-ALK antibody VL region is selected from ALK #2. In some embodiments, the anti-ALK antibody VL region is selected from ALK #3. In some embodiments, the anti-ALK antibody VL region is selected from ALK #4. In some embodiments, the anti-ALK antibody VL region is selected from ALK #5. In some embodiments, the anti-ALK antibody VL region is selected from ALK #6. In some embodiments, the anti-ALK antibody VL region is selected from ALK #7.

In some embodiments, the anti-ALK antibody VL region is at least 85%, at least 90%, at least 95%, at least 98%, at least 99%, or 100% identical to an exemplary anti-ALK antibody VL amino acid sequence as provided below:

DIQMTQSPASLAASVGETVTITCRASENIYYSLAWYQQKQGKSPQLLIYN
ANSLEDGVPSRFSGSGSGTQYSMKINSMQPEDTATYFCKQAYDVPFTFGS
GTKLEIKR

In some embodiments, the anti-ALK antibody VL region is at least 85%, at least 90%, at least 95%, at least 98%, at least 99%, or 100% identical to an exemplary anti-ALK antibody VL amino acid sequence as provided below:

AIQMTQTTSSLSASLGDRVTISCSVSQGISNSLNWYQQKPDGTVKLLIYY
TSSLHSGVPSRFSGSGSGTDYSLTISNLEPEDIATYYCQQYSKLPLTFGA
GTKLELKR

In some embodiments, the anti-ALK antibody VL region is at least 85%, at least 90%, at least 95%, at least 98%, at least 99%, or 100% identical to an exemplary anti-ALK antibody VL amino acid sequence as provided below:

DIVMTQSQRFMSTSVGDRVSVTCKASQNVGTNVAWYQQKPGQSPKALI
YSASYRYSGVPDRFTGSGSGTDFTLTVSNVQSEDLAEYFCQQYNSYPY
MYTFGGGTKLEIKR

In some embodiments, the anti-ALK antibody VL region is at least 85%, at least 90%, at least 95%, at least 98%, at least 99%, or 100% identical to an exemplary anti-ALK antibody VL amino acid sequence as provided below:

DIVLTQSPASLAVSLGQRATISCRASESVDNYGISFMNWFQQKPGQPP
KLLIYAASNQGSGVPARFSGSGSGTDFSLNIHPMEEDDTAMYFCQQSK
EVPWTFGGGTKLEIKR

In some embodiments, the anti-ALK antibody VL region is at least 85%, at least 90%, at least 95%, at least 98%, at least 99%, or 100% identical to an exemplary anti-ALK antibody VL amino acid sequence as provided below:

DIVMTQSQREMSTSVGDRVSVTCKASQNVGTNVAWYQQKPGQSPKALI
YSASYRYSGVPDRETGSGSGTDFTLTISNVQSEDLAEYFCQQYNSYPY
MYTFGGGTKLEIKR

In some embodiments, the anti-ALK antibody VL region is at least 85%, at least 90%, at least 95%, at least 98%, at least 99%, or 100% identical to an exemplary anti-ALK antibody VL amino acid sequence as provided below:

DIVMTQSQKFMSTSVGDRVSITCKASQNVGTAVAWYQLKPGQSPKLLIY
SASNRFTGVPDRFTGSGSGTDFTLTISNMQSEDLADYFCQQYSSYPLTF
GSGTKLEIKR

In some embodiments, the anti-ALK antibody VL region is at least 85%, at least 90%, at least 95%, at least 98%, at least 99%, or 100% identical to an exemplary anti-ALK antibody VL amino acid sequence as provided below:

DIVMTQSQKFMSTSVGDRVSVTCKASQNVGTNVAWYQQKPGHSPKALIYS
ASYRYSGVPDRFTGSGSGTDFTLTISNVQSEDLAEYFCQRYNSYPYMFTF
GGGTKLEIKR

In some embodiments, the anti-ALK antibody VL region is encoded by a polynucleotide that is at least 85%, at least 90%, at least 95%, at least 98%, at least 99%, or 100% identical to an exemplary nucleic acid sequence as provided below:

gacatccaga tgactcagtc tccagcctcc ctggctgcat ctgtgggaga aactgtcacc 60
atcacatgtc gagcaagtga gaacatttac tacagtttag catggtatca gcagaagcaa 120
gggaaatctc ctcagctcct gatctataat gcaaacagct tggaagatgg tgtcccatcg 180
aggttcagtg gcagtggatc tgggacacag tattctatga agatcaacag catgcagcct 240
gaagataccg caacttattt ctgtaaacag gcttatgacg ttccattcac gttcggctcg 300
gggacaaagt tggaaataaa acgg 324

In some embodiments, the anti-ALK antibody VL region is encoded by a polynucleotide that is at least 85%, at least 90%, at least 95%, at least 98%, at least 99%, or 100% identical to an exemplary nucleic acid sequence as provided below:

gctatccaga tgacacagac tacatcctcc ctgtctgcct ctctgggaga cagagtcacc 60
atcagttgca gtgtaagtca gggcattagc aattctttaa actggtatca gcagaaacca 120
gatggaactg ttaaactcct gatctattac acatcaagtt tacactcagg agtcccatca 180
aggttcagtg gcagtgggtc tgggacagat tattctctca ccatcagcaa cctggaacct 240
gaagatattg ccacttacta ttgtcagcag tatagtaagc ttccgctcac gttcggtgct 300
gggaccaagc tggagctgaa acgg 324

In some embodiments, the anti-ALK antibody VL region is encoded by a polynucleotide that is at least 85%, at least 90%, at least 95%, at least 98%, at least 99%, or 100% identical to an exemplary nucleic acid sequence as provided below:

gacattgtga tgacccagtc tcaaagattc atgtccacat cagtaggaga cagggtcagc 60
gtcacctgca aggccagtca gaatgtgggt actaatgtag cctggtatca acagaaacca 120
gggcaatctc ctaaagcact gatttactcg gcatcctacc ggtacagtgg agtccctgat 180
cgcttcacag gcagtggatc tgggacagat ttcactctca ccgtcagcaa tgtgcagtct 240
gaagacttgg cagagtattt ctgtcagcaa tataacagct atccgtacat gtacacgttc 300
ggagggggga ccaagctgga aataaaacgg 330

In some embodiments, the anti-ALK antibody VL region is encoded by a polynucleotide that is at least 85%, at least 90%, at least 95%, at least 98%, at least 99%, or 100% identical to an exemplary nucleic acid sequence as provided below:

gacattgtgc tgacccaatc tccagcttct ttggctgtgt ctctagggca gagggccacc 60
atctcctgca gagccagcga aagtgttgat aattatggca ttagttttat gaactggttc 120
caacagaaac caggacagcc acccaaactc ctcatctatg ctgcatccaa ccaaggatcc 180
ggggtccctg ccaggtttag tggcagtggg tctgggacag acttcagcct caacatccat 240
cctatggagg aggatgatac tgcaatgtat ttctgtcagc aaagtaagga ggttccgtgg 300
acgttcggtg gaggcaccaa gctggaaatc aaacgg 336

In some embodiments, the anti-ALK antibody VL region is encoded by a polynucleotide that is at least 85%, at least 90%, at least 95%, at least 98%, at least 99%, or 100% identical to an exemplary nucleic acid sequence as provided below:

gacattgtga tgacccagtc tcaaagattc atgtccacat cagtaggaga cagggtcagc 60
gtcacctgca aggccagtca gaatgtgggt actaatgtag cctggtatca acagaaacca 120
gggcaatctc ctaaagcact gatttactcg gcatcctacc ggtacagtgg agtccctgat 180
cgcttcacag gcagtggatc tgggacagat ttcactctca ccatcagcaa tgtgcagtct 240
gaagacttgg cagagtattt ctgtcaacaa tataacagct atccgtacat gtacacgttc 300
ggagggggga ccaagctgga aataaaacgg 330

In some embodiments, the anti-ALK antibody VL region is encoded by a polynucleotide that is at least 85%, at least 90%, at least 95%, at least 98%, at least 99%, or 100% identical to an exemplary nucleic acid sequence as provided below:

gacattgtga tgacccagtc tcaaaaattc atgtccacat cagtaggaga cagggtcagc 60
atcacctgta aggccagtca gaatgtgggt actgctgtag cctggtatca actgaaacca 120
ggacaatctc ctaaactact gatttactcg gcatccaatc ggttcactgg agtccctgat 180
cgcttcacag gcagtggatc tgggacagat ttcactctca ccatcagcaa tatgcagtct 240
gaagacctgg cagattattt ctgccagcaa tatagcagct atcctctcac gttcggctcg 300
gggacaaagt tggaaataaa acgg 324

In some embodiments, the anti-ALK antibody VL region is encoded by a polynucleotide that is at least 85%, at least 90%, at least 95%, at least 98%, at least 99%, or 100% identical to an exemplary nucleic acid sequence as provided below:

gacattgtga tgacccagtc tcaaaaattc atgtccacat cagtaggaga cagggtcagc 60
gtcacctgca aggccagtca gaatgtgggt actaatgtag cctggtatca acagaaacca 120
gggcactctc ctaaagcact gatttactcg gcatcctacc ggtacagtgg agtccctgat 180
cgcttcacag gcagtggatc tgggacagat ttcactctca ccatcagcaa tgtgcagtct 240
gaagacttgg cagagtattt ctgtcagcga tataacagct atccgtacat gttcacgttc 300
ggagggggga ccaagctgga aataaaacgg 330

In some embodiments, the anti-ALK antibody or an antigen binding fragment thereof comprises a VH region selected from ALK Antibody #1 (ALK #1), ALK Antibody #2 (ALK #2), ALK Antibody #3 (ALK #3), ALK Antibody #4 (ALK #4), ALK Antibody #5 (ALK #5), ALK Antibody #6 (ALK #6), or ALK Antibody #7 (ALK #7). In some embodiments, the anti-ALK antibody VH region is selected from ALK #1. In some embodiments, the anti-ALK antibody VH region is selected from ALK #2. In some embodiments, the anti-ALK antibody VH region is selected from ALK #3. In some embodiments, the anti-ALK antibody VH region is selected from ALK #4. In some embodiments, the anti-ALK antibody VH region is selected from ALK #5. In some embodiments, the anti-ALK antibody VH region is selected from ALK #6. In some embodiments, the anti-ALK antibody VH region is selected from ALK #7.

In some embodiments, the anti-ALK antibody VH region is at least 85%, at least 90%, at least 95%, at least 98%, at least 99%, or 100% identical to an exemplary amino acid sequence as provided below:

QVQLQQSGAELVKPGASVKISCKASGYAFSSYWMNWKQRPGKG
LEWIGQIYPGDGDTNYNGKFKGKATLTADKSSSTAYMQLSSLT
SEDSAVYFCASYYYGSKAYWGQGTLVTVSA

In some embodiments, the anti-ALK antibody VH region is at least 85%, at least 90%, at least 95%, at least 98%, at least 99%, or 100% identical to an exemplary amino acid sequence as provided below:

QVQLQQPGAEFVKPGASVKLSCKASGYTFTSYWMHWKQRPGRG
LEWIGRIDPNSGGTKYNEKFKSKATLTVDKPSSTAYMQLSSLT
SEDSAVYYCARDYYGSSYRFAYWGQGTLVTVSA

In some embodiments, the anti-ALK antibody VH region is at least 85%, at least 90%, at least 95%, at least 98%, at least 99%, or 100% identical to an exemplary amino acid sequence as provided below:

QVQLQQSGAELAKPGASVKLSCKASGYTFTNYWMHWKQRPGQG
LEWIGYINPSSGYTKYNQKFKDKATLTADKSSSTAYMQLSSLT
YEDSAVYYCARDYYGSSSWFAYWGQGTLVTVSA

In some embodiments, the anti-ALK antibody VH region is at least 85%, at least 90%, at least 95%, at least 98%, at least 99%, or 100% identical to an exemplary amino acid sequence as provided below:

QVQLQQSGAELVKPGASVKISCKASGYAFSSYWVNWKQRPG
KGLEWIGQIYPGDGDTNYNGKFKGKATLTADKSSSTAYMQL
SSLTSEDSAVYFCARSRGYFYGSTYDSWGQGTTLTVSS

In some embodiments, the anti-ALK antibody VH region is at least 85%, at least 90%, at least 95%, at least 98%, at least 99%, or 100% identical to an exemplary amino acid sequence as

QVQLQQSGAELAKPGASVKLSCKASGYTFTSYWMHWVKQRP
GQGLEWIGYIKPSSGYTKYNQKFKDKATLTADKSSSTAYMQ
LSSLTYEDSAVYYCARDYYGSSSWFAYWGQGTLVTVSA

In some embodiments, the anti-ALK antibody VH region is at least 85%, at least 90%, at least 95%, at least 98%, at least 99%, or 100% identical to an exemplary amino acid sequence as provided below:

DVKLVESGEGLVKPGGSLKLSCAASGFTFSSYAMSWRQTPEK
RLEWTYISSGGDYIYYADTVKGRFTISRDNARNTLYLQMSSL
KSEDTAMYYCTRERIWLRRFFDVWGTGTTVTVSS

In some embodiments, the anti-ALK antibody VH region is at least 85%, at least 90%, at least 95%, at least 98%, at least 99%, or 100% identical to an exemplary amino acid sequence as provided below:

QVQLQQSGAELAKPGASVKLSCKASGYTFTSYWMHWVKQRPG
QGLEWIGYINPSSGYTKYNQKFKDKATLTADKSSSTAYMQLS
SLTFEDSAVYYCARDYYGSSSWFAYWGQGTLVTVSA

In some embodiments, the anti-ALK antibody VH region is encoded by a polynucleotide that is at least 85%, at least 90%, at least 95%, at least 98%, at least 99%, or 100% identical to an exemplary nucleic acid sequence as provided below:

caggttcagc tgcagcagtc tggggctgag ctggtgaagc ctggggcctc agtgaagatt 60
tcctgcaaag cttctggcta cgcattcagt agctactgga tgaactgggt gaagcagagg 120
cctggaaagg gtcttgagtg gattggacag atttatcctg gagatggtga tactaactac 180
aacggaaagt tcaagggcaa ggccacactg actgcagaca aatcctccag cacagcctac 240
atgcagctca gcagcctgac ctctgaggac tctgcggtct atttctgtgc ctcttattac 300
tacggtagta aggcttactg gggccaaggg actctggtca ctgtctctgc a 351

In some embodiments, the anti-ALK antibody VH region is encoded by a polynucleotide that is at least 85%, at least 90%, at least 95%, at least 98%, at least 99%, or 100% identical to an exemplary nucleic acid sequence as provided below:

caggtccaac tgcagcagcc tggggctgag tttgtgaagc ctggggcttc agtgaagctg 60
tcctgcaagg cttctggcta caccttcacc agctactgga tgcactgggt gaagcagagg 120
cctggacgag gccttgagtg gattggaagg attgatccta atagtggtgg tactaagtac 180
aatgagaagt tcaagagcaa ggccacactg actgtagaca aaccctccag cacagcctac 240
atgcagctca gcagcctgac atctgaggac tctgcggtct attattgtgc aagagattac 300
tacggtagta gctaccggtt tgcttactgg ggccaaggga ctctggtcac tgtctctgca 360

In some embodiments, the anti-ALK antibody VH region is encoded by a polynucleotide that is at least 85%, at least 90%, at least 95%, at least 98%, at least 99%, or 100% identical to an exemplary nucleic acid sequence as provided below:

caggtccagc tgcagcagtc tggggctgaa ctggcaaaac ctggggcctc agtgaagctg 60

tcctgcaagg cttctggcta cacctttact aactactgga tgcactgggt aaaacagagg 120
cctggacagg gtctggaatg gattggatac attaatccta gcagtggtta tactaagtac 180
aatcagaagt tcaaggacaa ggccacattg actgcagaca aatcctccag cacagcctac 240
atgcagctga gcagcctgac atatgaggac tctgcagtct attactgtgc aagagattac 300
tacggtagta gctcctggtt tgcttactgg ggccaaggga ctctggtcac tgtctctgca 360

In some embodiments, the anti-ALK antibody VH region is encoded by a polynucleotide that is at least 85%, at least 90%, at least 95%, at least 98%, at least 99%, or 100% identical to an exemplary nucleic acid sequence as provided below:

caggttcagc tgcaacagtc tggggctgag ctggtgaagc ctggggcctc agtgaagatt 60
tcctgcaaag cttctggcta cgcattcagt agctactggg tgaactgggt gaagcagagg 120
cctggaaagg gtcttgagtg gattggacag atttatcctg gagatggtga tactaactac 180
aacggaaagt tcaagggcaa ggccacactg actgcagaca aatcctccag cacagcctac 240
atgcagctca gcagcctgac ctctgaggac tctgcggtct atttctgtgc aagatcaaga 300
gggtatttct acggtagtac ctacgactcc tggggccaag gcaccactct cacagtctcc 360
tca 363

In some embodiments, the anti-ALK antibody VH region is encoded by a polynucleotide that is at least 85%, at least 90%, at least 95%, at least 98%, at least 99%, or 100% identical to an exemplary nucleic acid sequence as provided below:

caggtccagc tgcagcagtc tggggctgaa ctggcaaaac ctggggcctc agtgaagctg 60
tcctgcaagg cttctggcta cacctttact agctactgga tgcactgggt aaaacagagg 120
cctggacagg gtctggaatg gattggatac attaagccta gcagtggtta tactaagtac 180
aatcagaagt tcaaggacaa ggccacattg actgcagaca aatcctccag cacagcctac 240
atgcagctga gcagcctgac atatgaggac tctgcagtct attactgtgc aagagattac 300
tacggtagta gctcctggtt tgcttactgg ggccaaggga ctctggtcac tgtctctgca 360

In some embodiments, the anti-ALK antibody VH region is encoded by a polynucleotide that is at least 85%, at least 90%, at least 95%, at least 98%, at least 99%, or 100% identical to an exemplary nucleic acid sequence as provided below:

gacgtgaagc tggtggagtc tggggaaggc ttagtgaagc ctggagggtc cctgaaactc 60
tcctgtgcag cctctggatt cactttcagt agctatgcca tgtcttgggt tcgccagact 120
ccagagaaga ggctggagtg ggtcacatac attagtagtg gtggtgatta catctactat 180
gcagacactg tgaagggccg attcaccatc tccagagaca atgccaggaa caccctgtac 240
ctgcaaatga gcagtctgaa gtctgaggac acagccatgt attactgtac aagagagcgg 300
atatggttac gacggttctt cgatgtctgg ggcacaggga ccacggtcac cgtctcctca 360

In some embodiments, the anti-ALK antibody VH region is encoded by a polynucleotide that is at least 85%, at least 90%, at least 95%, at least 98%, at least 99%, or 100% identical to an exemplary nucleic acid sequence as provided below:

caggtccagc tgcagcagtc tggggctgaa ctggcaaaac ctggggcctc agtgaagctg 60
tcctgcaagg cttctggcta cacctttact agctactgga tgcactgggt aaaacagagg 120
cctggacagg gtctggaatg gattggatac attaatccta gcagtggtta tactaagtac 180
aatcagaagt tcaaggacaa ggccacattg actgcagaca aatcctccag cacagcctac 240
atgcagctga gcagcctgac atttgaggac tctgcagtct attactgtgc aagagattac 300
tacggtagta gctcctggtt tgcttactgg ggccaaggga ctctggtcac tgtctctgca 360

In some embodiments, an anti-ALK antibody provided herein, or an antigen-binding fragment thereof, comprises a VL region and a VH region that is at least 85%, at least 90%, at least 95%, at least 98%, at least 99%, or 100% identical to the VL and VH amino acid sequences of any one of antibodies ALK #1, ALK #2, ALK #3, ALK #4, ALK #5, ALK #6, or ALK #7 as provided in Tables 1 and 2, respectively. In some embodiments, an anti-ALK antibody provided herein, or an antigen-binding fragment thereof, comprises a VL region and a VH region of any one of antibodies ALK #1, ALK #2, ALK #3, ALK #4, ALK #5, ALK #6, or ALK #7 as provided in Tables 1 and 2, respectively.

The CDRs are primarily responsible for binding to an epitope of an antigen. The amino acid sequence positions of a given CDR can be readily determined using any methods known in the art, including those described by Kabat et al. (“Sequences of Proteins of Immunological Interest, 5th Ed. Public Health Service, National Institutes of Health, Bethesda, Md., 1991; “Kabat” numbering scheme), Al-Lazikani et al., (JMB 273,927-948, 1997: “Chothia” numbering scheme), and Lefranc et al. (“IMGT unique numbering for immunoglobulin and T cell receptor variable domains and Ig superfamily V-like domains.” Dev. Comp. Immunol., 27:55-77, 2003: “IMGT” numbering scheme). The CDRs of each chain are typically referred to as CDR1, CDR2, and CDR3 (from the N-terminus to C-terminus), and are also typically identified by the chain in which the particular CDR is located. Thus, herein a VH-CDR3 is the CDR3 from the variable domain of the heavy chain of the antibody in which it is found, and a VL-CDR1 is the CDR1 from the variable domain of the light chain of the antibody in which it is found. Light chain CDRs are referred herein as LCDR1, LCDR2, and LCDR3. Heavy chain CDRs are referred herein as HCDR1, HCDR2, and HCDR3.

In some embodiments, the CDRs of an anti-ALK antibody specifically bind ALK (e.g., human ALK). In some embodiments, the CDRs of an anti-ALK antibody specifically bind the extracellular domain (ECD) of ALK (e.g., human ALK ECD). In some embodiments, an anti-ALK antibody or an antigen binding fragment thereof comprises one or more CDRs of a VL region selected from ALK Antibody #1 (ALK #1), ALK Antibody #2 (ALK #2), ALK Antibody #3 (ALK #3), ALK Antibody #4 (ALK #4), ALK Antibody #5 (ALK #5), ALK Antibody #6 (ALK #6), or ALK Antibody #7 (ALK #7). In some embodiments, the anti-ALK antibody comprises one or more CDRs of a VL region selected from ALK #1. In some embodiments, the anti-ALK antibody comprises one or more CDRs of a VL region selected from ALK #2. In some embodiments, the anti-ALK antibody comprises one or more CDRs of a VL region selected from ALK #3. In some embodiments, the anti-ALK antibody comprises one or more CDRs of a VL region selected from ALK #4. In some embodiments, the anti-ALK antibody comprises one or more CDRs of a VL region selected from ALK #5. In some embodiments, the anti-ALK antibody comprises one or more CDRs of a VL region selected from ALK #6. In some embodiments, the anti-ALK antibody comprises one or more CDRs of a VL region selected from ALK #7.

In some embodiments, the anti-ALK antibody LCDR1 is at least 85%, at least 90%, at least 95%, at least 98%, at least 99%, or 100% identical to an exemplary amino acid sequence as provided below:

RASENIYYSLA

In some embodiments, the anti-ALK antibody LCDR2 is at least 85%, at least 90%, at least 95%, at least 98%, at least 99%, or 100% identical to an exemplary amino acid sequence as provided below:

NANSLED

In some embodiments, the anti-ALK antibody LCDR3 is at least 85%, at least 90%, at least 95%, at least 98%, at least 99%, or 100% identical to an exemplary amino acid sequence as provided below:

KQAYDVPFT

In some embodiments, the anti-ALK antibody LCDR1 is at least 85%, at least 90%, at least 95%, at least 98%, at least 99%, or 100% identical to an exemplary amino acid sequence as provided below:

SVSQGISNSLN

In some embodiments, the anti-ALK antibody LCDR2 is at least 85%, at least 90%, at least 95%, at least 98%, at least 99%, or 100% identical to an exemplary amino acid sequence as provided below:

YTSSLHS

In some embodiments, the anti-ALK antibody LCDR3 is at least 85%, at least 90%, at least 95%, at least 98%, at least 99%, or 100% identical to an exemplary amino acid sequence as provided below:

QQYSKLPLT

In some embodiments, the anti-ALK antibody LCDR1 is at least 85%, at least 90%, at least 95%, at least 98%, at least 99%, or 100% identical to an exemplary amino acid sequence as provided below:

KASQNVGTNVA

In some embodiments, the anti-ALK antibody LCDR2 is at least 85%, at least 90%, at least 95%, at least 98%, at least 99%, or 100% identical to an exemplary amino acid sequence as provided below:

SASYRYS

In some embodiments, the anti-ALK antibody LCDR3 is at least 85%, at least 90%, at least 95%, at least 98%, at least 99%, or 100% identical to an exemplary amino acid sequence as provided below:

QQYNSYPYMYT

In some embodiments, the anti-ALK antibody LCDR1 is at least 85%, at least 90%, at least 95%, at least 98%, at least 99%, or 100% identical to an exemplary amino acid sequence as provided below:

RASESVDNYGISFMN

In some embodiments, the anti-ALK antibody LCDR2 is at least 85%, at least 90%, at least 95%, at least 98%, at least 99%, or 100% identical to an exemplary amino acid sequence as provided below:

AASNQGS

In some embodiments, the anti-ALK antibody LCDR3 is at least 85%, at least 90%, at least 95%, at least 98%, at least 99%, or 100% identical to an exemplary amino acid sequence as provided below:

QQSKEVPWT

In some embodiments, the anti-ALK antibody LCDR1 is at least 85%, at least 90%, at least 95%, at least 98%, at least 99%, or 100% identical to an exemplary amino acid sequence as provided below:

KASQNVGTAVA

In some embodiments, the anti-ALK antibody LCDR2 is at least 85%, at least 90%, at least 95%, at least 98%, at least 99%, or 100% identical to an exemplary amino acid sequence as provided below:

SASNRFT

In some embodiments, the anti-ALK antibody LCDR3 is at least 85%, at least 90%, at least 95%, at least 98%, at least 99%, or 100% identical to an exemplary amino acid sequence as provided below:

QQYSSYPLT

In some embodiments, the anti-ALK antibody LCDR3 is at least 85%, at least 90%, at least 95%, at least 98%, at least 99%, or 100% identical to an exemplary amino acid sequence as provided below:

QRYNSYPYMFT

In some embodiments, an anti-ALK antibody or an antigen binding fragment thereof comprises one or more CDRs of a VH region selected from ALK Antibody #1 (ALK #1), ALK Antibody #2 (ALK #2), ALK Antibody #3 (ALK #3), ALK Antibody #4 (ALK #4), ALK Antibody #5 (ALK #5), ALK Antibody #6 (ALK #6), or ALK Antibody #7 (ALK #7). In some embodiments, the anti-ALK antibody comprises one or more CDRs of a VH region selected from ALK #1. In some embodiments, the anti-ALK antibody comprises one or more CDRs of a VH region selected from ALK #2. In some embodiments, the anti-ALK antibody comprises one or more CDRs of a VH region selected from ALK #3. In some embodiments, the anti-ALK antibody comprises one or more CDRs of a VH region selected from ALK #4. In some embodiments, the anti-ALK antibody comprises one or more CDRs of a VH region selected from ALK #5. In some embodiments, the anti-ALK antibody comprises one or more CDRs of a VH region selected from ALK #6. In some embodiments, the anti-ALK antibody comprises one or more CDRs of a VH region selected from ALK #7.

In some embodiments, the anti-ALK antibody HCDR1 is at least 85%, at least 90%, at least 95%, at least 98%, at least 99%, or 100% identical to an exemplary amino acid sequence as provided below:

SYWMN

In some embodiments, the anti-ALK antibody HCDR2 is at least 85%, at least 90%, at least 95%, at least 98%, at least 99%, or 100% identical to an exemplary amino acid sequence as provided below:

QIYPGDGDTNYNGKFKG

In some embodiments, the anti-ALK antibody HCDR3 is at least 85%, at least 90%, at least 95%, at least 98%, at least 99%, or 100% identical to an exemplary amino acid sequence as provided below:

YYYGSKAY

In some embodiments, the anti-ALK antibody HCDR1 is at least 85%, at least 90%, at least 95%, at least 98%, at least 99%, or 100% identical to an exemplary amino acid sequence as provided below:

SYWMH

In some embodiments, the anti-ALK antibody HCDR2 is at least 85%, at least 90%, at least 95%, at least 98%, at least 99%, or 100% identical to an exemplary amino acid sequence as provided below:

RIDPNSGGTKYNEKFKS

In some embodiments, the anti-ALK antibody HCDR3 is at least 85%, at least 90%, at least 95%, at least 98%, at least 99%, or 100% identical to an exemplary amino acid sequence as provided below:

DYYGSSYRFAY

In some embodiments, the anti-ALK antibody HCDR1 is at least 85%, at least 90%, at least 95%, at least 98%, at least 99%, or 100% identical to an exemplary amino acid sequence as provided below:

NYWMH

In some embodiments, the anti-ALK antibody HCDR2 is at least 85%, at least 90%, at least 95%, at least 98%, at least 99%, or 100% identical to an exemplary amino acid sequence as provided below:

YINPSSGYTKYNQKFKD

In some embodiments, the anti-ALK antibody HCDR3 is at least 85%, at least 90%, at least 95%, at least 98%, at least 99%, or 100% identical to an exemplary amino acid sequence as provided below:

DYYGSSSWFAY

In some embodiments, the anti-ALK antibody HCDR1 is at least 85%, at least 90%, at least 95%, at least 98%, at least 99%, or 100% identical to an exemplary amino acid sequence as provided below:

SYWVN

In some embodiments, the anti-ALK antibody HCDR3 is at least 85%, at least 90%, at least 95%, at least 98%, at least 99%, or 100% identical to an exemplary amino acid sequence as provided below:

SRGYFYGSTYDS

In some embodiments, the anti-ALK antibody HCDR2 is at least 85%, at least 90%, at least 95%, at least 98%, at least 99%, or 100% identical to an exemplary amino acid sequence as provided below:

YIKPSSGYTKYNQKFKD

In some embodiments, the anti-ALK antibody HCDR1 is at least 85%, at least 90%, at least 95%, at least 98%, at least 99%, or 100% identical to an exemplary amino acid sequence as provided below:

SYAMS

In some embodiments, the anti-ALK antibody HCDR2 is at least 85%, at least 90%, at least 95%, at least 98%, at least 99%, or 100% identical to an exemplary amino acid sequence as provided below:

YISSGGDYIYYADTVKG

In some embodiments, the anti-ALK antibody HCDR3 is at least 85%, at least 90%, at least 95%, at least 98%, at least 99%, or 100% identical to an exemplary amino acid sequence as provided below:

ERIWLRRFFDV

In some embodiments, an anti-ALK antibody provided herein, or an antigen-binding fragment thereof, comprises one or more CDRs from VL region and one or more CDRs from a VH region that is at least 85%, at least 90%, at least 95%, at least 98%, at least 99%, or 100% identical to the CDRs of the VL and VH amino acid sequences of any one of antibodies ALK #1, ALK #2, ALK #3, ALK #4, ALK #5, ALK #6, or ALK #7 as provided in Tables 3 and 4, respectively. In some embodiments, an anti-ALK antibody provided herein, or an antigen-binding fragment thereof, comprises one or more CDRs from a VL region and one or more CDRs from a VH region of any one of antibodies ALK #1, ALK #2, ALK #3, ALK #4, ALK #5, ALK #6, or ALK #7 as provided in Tables 3 and 4, respectively. In some embodiments, an anti-ALK antibody provided herein, or an antigen-binding fragment thereof, comprises three CDRs from a VL region of any one of antibodies ALK #1, ALK #2, ALK #3, ALK #4, ALK #5, ALK #6, or ALK #7 as provided in Table 3. In some embodiments, an anti-ALK antibody provided herein, or an antigen-binding fragment thereof, comprises three CDRs from a VH region of any one of antibodies ALK #1, ALK #2, ALK #3, ALK #4, ALK #5, ALK #6, or ALK #7 as provided in Table 4. In some embodiments, an anti-ALK antibody provided herein, or an antigen-binding fragment thereof, comprises three CDRs from a VL region and three CDRs from a VH region of any one of antibodies ALK #1, ALK #2, ALK #3, ALK #4, ALK #5, ALK #6, or ALK #7 as provided in Tables 3 and 4, respectively.

Antibodies can include, for example, monoclonal antibodies, recombinantly produced antibodies, monospecific antibodies, multispecific antibodies (including bispecific antibodies), human antibodies, humanized antibodies, such as composite human antibodies or deimmunized antibodies, murine antibodies (e.g., mouse or rat antibodies), chimeric antibodies, synthetic antibodies, and tetrameric antibodies comprising two heavy chain and two light chain molecules. In specific embodiments, antibodies can include, but are not limited to an antibody light chain monomer, an antibody heavy chain monomer, an antibody light chain dimer, an antibody heavy chain dimer, an antibody light chain/antibody heavy chain pair, an antibody with two light chain/heavy chain pairs (e.g., identical pairs), intrabodies, heteroconjugate antibodies, single domain antibodies, monovalent antibodies, bivalent antibodies, single chain antibodies or single-chain Fvs (scFv) (e.g., including monospecific, bispecific, etc.), camelized antibodies, and affybodies. Antigen-binding fragments can include antigen-binding fragments or epitope binding fragments such as, but not limited to, Fab fragments, F(ab′) fragments, F(ab′) 2 fragments, and disulfide-linked Fvs (sdFv). In certain embodiments, antibodies described herein refer to polyclonal antibody populations. Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2) of immunoglobulin molecule. In certain embodiments, antibodies described herein are IgG antibodies (e.g., human IgG), or a class (e.g., human IgG1, IgG2, IgG3, or IgG4) or subclass thereof.

“Eliciting an antibody response” refers to the ability of an antigen, immunogen or other molecule to induce the production of antibodies. Antibodies are of different classes, e.g., IgM, IgG, IgA, IgE, IgD and subtypes or subclasses, e.g., IgG1, IgG2, IgG2a, IgG2b, IgG3, IgG4. An antibody/immunoglobulin response elicited in a subject can neutralize a pathogenic (e.g., disease-causing) agent by binding to epitopes (antigenic determinants) on the agent and blocking or inhibiting the activity of the agent, and/or by forming a binding complex with the agent that is cleared from the system of the subject, e.g., via the liver.

By “amphiphile” is meant a chemical compound possessing both hydrophilic and lipophilic properties. Such a compound is called amphiphilic or amphipathic. The amphiphile may be conjugated or linked to an antigen or adjuvant cargo by a solubility-promoting polar polymer chain. In some embodiments, the amphiphile is conjugated or linked to an adjuvant. In some embodiments, the adjuvant is Freund's adjuvant. In some embodiments, the amphiphile is conjugated or linked to an ALK polypeptide. In some embodiments, the amphiphile is a lipophilic albumin-binding tail. In some embodiments, the amphiphile is N-hydroxy succinimidyl ester-end-functionalized poly(ethylene glycol)-lipid (NHS-PEG2KDa-DSPE).

By “antigen” is meant a moiety or molecule (e.g., polypeptide, peptide) that contains an epitope to which an antibody can specifically bind. As such, an antigen is also specifically bound by an antibody. In one embodiment, the antigen to which an antibody described herein binds is an anaplastic lymphoma kinase (ALK) protein or a fragment thereof. In one embodiment, the antigen to which an antibody described herein binds an ALK extracellular domain. In some embodiments, the antigen to which an antibody described herein binds is human ALK or the human ALK extracellular domain. Binding of an antigen by an antibody can stimulate an immune response in a subject, including compositions that are injected or absorbed into a subject. An antigen that elicits or stimulates an immune response in a subject is termed an “immunogen.” An antigen reacts with the products of specific humoral or cellular immunity, including those induced by heterologous immunogens.

By “antigen binding fragment” is meant a portion of a full-length antibody that retains the ability to specifically recognize an antigen (e.g., ALK protein), as well as various combinations of such portions. Non-limiting examples of antigen binding fragments include Fv, Fab, Fab′, Fab′-SH, F(ab)₂; diabodies; linear antibodies; single-chain antibody molecules (e.g., scFv); and multispecific antibodies formed from antibody fragments. Antigen binding fragments may be produced by the modification of whole antibodies or those synthesized de novo using recombinant DNA methodologies (see, e.g., Kontermann and Dubel (Ed), Antibody Engineering, Vols. 1-2, 2″ Ed., Springer Press, 2010).

A “chimeric antibody” is an antibody which includes sequences derived from two different antibodies, which typically are of different species. In some embodiments, a chimeric antibody includes one or more CDRs and/or framework regions from one antibody and CDRs and/or framework regions from another antibody. For example, a chimeric antibody can contain a variable region of a mouse or rat monoclonal antibody fused to a constant region of a human antibody. Methods for producing chimeric antibodies are known in the art (see e.g., Morrison, 1985, Science 229:1202; Oi et al., 1986, BioTechniques 4:214; Gillies et al., 1989, J. Immunol. Methods 125:797-202; and U.S. Pat. Nos. 5,807,715, 4,816,567, 4,816,397, and 6,331,415).

By “chimeric antigen receptor” or “CAR” is meant an engineered receptor comprising an extracellular antigen binding domain (e.g., scFv) joined to one or more intracellular signaling domains (e.g., T cell signaling domain) that confers specificity for an antigen onto an immune effector cell. In some embodiments, the CAR includes a transmembrane domain. In some embodiments, the CAR construct is derived from or comprises the m1928z CAR construct as provided in Davila et al., CD19 CAR-Targeted T Cells Induce Long-Term Remission and B Cell Aplasia in an Immunocompetent Mouse Model of B Cell Acute Lymphoblastic Leukemia, PLoS ONE (2013), which is incorporated by reference in its entirety herein. In some embodiments, the CAR is an anaplastic lymphoma kinase chimeric antigen receptor (ALK CAR) that specifically binds to an ALK polypeptide or antibody-binding fragment thereof.

By “chimeric antigen receptor T cell” or “CAR-T cell” is meant a T cell expressing a CAR that has antigen specificity determined by the antibody-derived targeting domain of the CAR. As used herein, “CAR-T cells” includes T cells or NK cells. As used herein, “CAR-T cells” includes cells engineered to express a CAR or a T cell receptor (TCR). In some embodiments, CAR-T cells can be T helper CD4+ and/or T effector CD8+ cells, optionally in defined proportions. In some embodiments, CAR-T cells may comprise total CD3+ cells. Methods of making CARS (e.g., for treatment of cancer) are publicly available (see, e.g., Park et al., Trends Biotechnol., 29:550-557, 2011; Grupp et al., N Engl J Med., 368:1509-1518, 2013; Han et al., J. Hematol Oncol. 6:47, 2013; Haso et al., (2013) Blood, 121, 1165-1174; PCT Pubs. WO2012/079000, WO2013/059593; and U.S. Pub. 2012/0213783, each of which is incorporated by reference herein in its entirety). In some embodiments, the CAR-T cell expresses an ALK CAR.

A “codon-optimized” nucleic acid (polynucleotide) refers to a nucleic acid sequence that has been altered such that the codons are optimal for expression in a particular system (such as a particular species of group of species). For example, a nucleic acid sequence can be optimized for expression in mammalian cells. Codon optimization does not alter the amino acid sequence of the encoded protein.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. “Detect” refers to identifying the presence, absence or amount of an analyte, compound, agent, or substance to be detected.

By “detectable label” is meant a composition that, when linked to a molecule of interest, renders the latter detectable, e.g., via spectroscopic, photochemical, biochemical, immunochemical, or chemical means. Nonlimiting examples of useful detectable labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition, disorder, or pathology that damages or interferes with the normal function of a cell, tissue, or organ. Examples of diseases include those caused by oncogenic ALK gene fusions, rearrangements, duplications or mutations (e.g., ALK-positive cancers). In some embodiments, the cancer is an ALK-positive cancer. By “ALK-positive cancer” is meant a cancer or tumor that expresses the ALK protein. Nonlimiting examples of ALK-positive cancers include non-small cell lung cancer (NSCLC), anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, and melanoma. In some embodiments, the ALK-positive cancer is neuroblastoma.

The ALK-positive cancer may be caused by an oncogenic ALK gene that either forms a fusion gene with other genes, gains additional gene copies, or is genetically mutated. In some embodiments, the ALK-positive cancer is caused by an ALK fusion gene encoding an ALK fusion protein. In some embodiments, the ALK-positive cancer is caused by a fusion between the ALK gene and the nucleophosmin (NPM) gene encoding a NPM-ALK fusion protein. In some embodiments, the ALK-positive cancer is caused by a fusion between the ALK gene and the echinoderm microtubule-associated protein-like 4 (EML4) gene encoding an ELM4-ALK fusion protein. In some embodiments, the ALK-positive cancer is caused by a point mutation. In some embodiments, the point mutation is F1174L (ALK^{F1174L}). In some embodiments, the ALK-positive cancer is neuroblastoma.

By “effective amount” is meant the amount of an active therapeutic agent, composition, compound, biologic (e.g., a vaccine or therapeutic peptide, polypeptide, or polynucleotide) required to ameliorate, reduce, delay, improve, abrogate, diminish, or eliminate the symptoms and/or effects of a disease, condition, or pathology relative to an untreated patient. In some embodiments, an effective amount of an ALK peptide is the amount required to induce an ALK-specific immune response in a subject immunized with the peptide. The effective amount of an immunogen or a composition comprising an immunogen, as used to practice the methods of therapeutic treatment of a disease, condition, or pathology, varies depending upon the manner of administration, the age, body weight, and general health of the subject. Ultimately, the attending physician or veterinarian will decide the appropriate amount and dosage regimen. Such amount is referred to as an “effective” amount.

The inventions herein provide a number of targets that are useful for the development of highly specific drugs to treat a disease or disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

A “therapeutically effective amount” refers to a quantity of a specified agent sufficient to achieve a desired effect in a subject being treated with that agent. For example, this may be the amount of an ALK-specific antigen, immunogen, immunogenic composition or vaccine useful for eliciting an immune response in a subject, treating and/or for preventing a disease caused by oncogenic ALK gene fusions, rearrangements, duplications or mutations (e.g., ALK-positive cancers). Ideally, in the context of the present disclosure, a therapeutically effective amount of an ALK-specific vaccine or immunogenic composition is an amount sufficient to prevent, ameliorate, reduce, delay and/or treat a disease caused by oncogenic ALK gene fusions, rearrangements, duplications or mutations (e.g., ALK-positive cancers) in a subject without causing a substantial cytotoxic effect in the subject. The effective amount of an ALK-specific vaccine or immunogenic composition useful for preventing, delaying, ameliorating, reducing, and/or treating a disease caused by oncogenic ALK gene fusions, rearrangements, duplications or mutations (e.g., ALK-positive cancers) in a subject depends on, for example, the subject being treated, the manner of administration of the therapeutic composition and other factors, as noted supra.

By “Epitope,” as used herein, means an antigenic determinant. An epitope is the part of an antigen molecule that by its structure determines the specific antibody molecule that will recognize and specifically bind to elicit a specific immune response. In some embodiments, a disclosed antibody specifically binds to an epitope on ALK.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 3, 4, 5, 6, 7, 8, 9, 10, 20, 30, 40, 50, 60, 70, 80, 90, or 100, 200, 300, 400, 500, 600, 700, 800, 900, or 1000 nucleotides or amino acids. A portion or fragment of a polypeptide may be a peptide. In the case of an antibody or immunoglobulin fragment, the fragment typically binds to the target antigen.

By “fusion protein” is meant a protein generated by expression of a nucleic acid (polynucleotide) sequence engineered from nucleic acid sequences encoding at least a portion of two different (heterologous) proteins or peptides. To create a fusion protein, the nucleic acid sequences must be in the same open reading frame and contain no internal stop codons. One protein can be located at the amino-terminal (N-terminal) portion of the fusion protein or at the carboxy-terminal (C-terminal) protein thus forming an amino-terminal fusion protein or a carboxy-terminal fusion protein, respectively.

For example, a fusion protein includes an ALK protein fused to a heterologous protein. In some embodiments, the fusion protein is an ALK protein fused to a nucleophosmin (NPM) protein. In some embodiments, the NPM-ALK fusion protein is at least 85%, at least 90%, at least 95%, at least 98%, at least 99%, or 100% identical to a NPM-ALK fusion protein in Homo Sapiens. In some embodiments, the NPM-ALK fusion protein is at least 85%, at least 90%, at least 95%, at least 98%, at least 99%, or 100% identical to an exemplary NPM-ALK fusion protein amino acid sequence from Homo Sapiens as provided below (ALK cytoplasmic portion in bold font):

MEDSMDMDMSPLRPQNYLFGCELKADKDYHFKVDN
DENEHQLSLRTVSLGAGAKDELHIVEAEAMNYEGS
PIKVTLATLKMSVQPTVSLGGFEITPPVVLRLKCG
SGPVHISGQHLVVYRRKHQELQAMOMELQSPEYKL
SKLRTSTIMTDYNPNYCFAGKTSSISDLKEVPRKN
NTLIRGLGHGAFGEVYEGQVSGMPNDPSPLQVAVK
TLPEVCSEQDELDFLMEALIISKFNHQNIVRCIGV
SLQSLPRFILLELMAGGDLKSFLRETRPRPSQPSS
IAMLDLLHVARDIACGCQYLEENHFIHRDIAARNC
LLTCPGPGRVAKIGDFGMARDIYRASYYRKGGCAM
LPVKWMPPEAFMEGIFTSKTDTWSFGVLLWEIFSL
GYMPYPSKSNQEVLEFVTSGGRMDPPKNCPGP
VYRIMTQCWQHQPEDRPNFAIILERIEYCTQDPDV
INTALPIEYGPLVEEEEKVPVRPKDPEGVPPLLVS
QQAKREEERSPAAPPPLPTTSSGKAAKKPTAAEVS
VRVPRGPAVEGGHVNMAFSQSNPPSELHRVHGSRN
KPTSLWNPTYGSWFTEKPTKKNNPIAKKEPHERGN
LGLEGSCTVPPNVATGRLPGASLLLEPSSLTANMK
EVPLFRLRHFPCG

In some embodiments, the NPM-ALK fusion protein is at least 85%, at least 90%, at least 95%, at least 98%, at least 99%, or 100% identical to an exemplary NPM-ALK fusion protein amino acid sequence from Homo Sapiens (GenBank: AAA58698.1) as provided below:

| | |
|---|---|
| 1 | MEDSMDMDMS PLRPQNYLFG CELKADKDYH |
| | FKVDNDENEH QLSLRTVSLG AGAKDELHIV |
| 61 | EAEAMNYEGS PIKVTLATLK MSVQPTVSLG |
| | GFEITPPVVL RLKCGSGPVH ISGQHLVVYR |
| 121 | RKHQELQAMQ MELQSPEYKL SKLRTSTIMT |
| | DYNPNYCFAG KTSSISDLKE VPRKNITLIR |
| 181 | GLGHGAFGEV YEGQVSGMPN DPSPLQVAVK |
| | TLPEVCSEQD ELDFLMEALI ISKFNHQNIV |
| 241 | RCIGVSLQSL PRFILLELMA GGDLKSFLRE |
| | TRPRPSQPSS LAMLDLLHVA RDIACGCQYL |
| 301 | EENHFIHRDI AARNCLLTCP GPGRVAKIGD |
| | FGMARDIYRA SYYRKGGCAM LPVKWMPPEA |
| 361 | FMEGIFTSKT DTWSFGVLLW EIFSLGYMPY |
| | PSKSNQEVLE FVTSGGRMDP PKNCPGPVYR |
| 421 | IMTQCWQHQP EDRPNFAIIL ERIEYCTQDP |
| | DVINTALPIE YGPLVEEEEK VPVRPKDPEG |
| 481 | VPPLLVSQQA KREEERSPAA PPPLPTTSSG |
| | KAAKKPTAAE VSVRVPRGPA VEGGHVNMAF |
| 541 | SQSNPPSELH KVHGSRNKPT SLWNPTYGSW |
| | FTEKPTKKNN PIAKKEPHDR GNLGLEGSCT |
| 601 | VPPNVATGRL PGASLLLEPS SLTANMKEVP |
| | LFRLRHFPCG NVNYGYQQQG LPLEAATAPG |
| 661 | AGHYEDTILK SKNSMNQPGP |

In some embodiments, the NPM-ALK fusion protein is at least 85%, at least 90%, at least 95%, at least 98%, at least 99%, or 100% identical to an exemplary NPM-ALK fusion protein amino acid sequence from Homo Sapiens as provided below:

M E D S M D M D M S P L R P Q N Y L F G
C E L K A D K D Y H F K V D N D E N E H
Q L S L R T V S L G A G A K D E L H I V
E A E A M N Y E G S P I K V T L A T L K
M S V Q P T V S L G G F E I T P P V V L
R L K C G S G P V H I S G Q H L V V Y R
R K H Q E L Q A M Q M E L Q S P E Y K L
S K L R T S T I M T D Y N P N Y C F A G
K T S S I S D L K E V P R K N I T L I R
G L G H G A F G E V Y E G Q V S G M P N
D P S P L Q V A V K T L P E V C S E Q D
E L D F L M E A L I I S K F N H Q N I V
R C I G V S L Q S L P R F I L L E L M A
G G D L K S F L R E T R P R P S Q P S S
L A M L D L L H V A R D I A C G C Q Y L
E E N H F I H R D I A A R N C L L T C P
G P G R V A K I G D F G M A R D I Y R A
S Y Y R K G G C A M L P V K W M P P E A
F M E G I F T S K T D T W S F G V L L W
E I F S L G Y M P Y P S K S N Q E V L E
F V T S G G R M D P P K N C P G P V Y R
I M T Q C W Q H Q P E D R P N F A I I L
E R I E Y C T Q D P D V I N T A L P I E
Y G P L V E E E E K V P V R P K D P E G
V P P L L V S Q Q A K R E E E R S P A A
P P P L P T T S S G K A A K K P T A A E
V S V R V P R G P A V E G G H V N M A E
S Q S N P P S E L H K V H G S R N K P T
S L W N P T Y G S W F T E K P T K K N N
P I A K K E P H D R G N L G L E G S C T
V P P N V A T G R L P G A S L L L E P S
S L T A N M K E V P L F R L R H F P C G
N V N Y G Y Q Q Q G L P L E A A T A P G
A G H Y E D T I L K S K N S M N Q P G P
*

In some embodiments, the NPM-ALK fusion protein is encoded by a nucleic acid sequence that is at least 85%, at least 90%, at least 95%, at least 98%, at least 99%, or 100% identical to the exemplary nucleic acid sequence from Homo Sapiens as provided below:

| | |
|---|---|
| 1 | atggaagattcgatggacatggacatgagccccctgaggccccagaactatcttttcggt 60 |
| 61 | tgtgaactaaaggccgacaaagattatcactttaaggtggataatgatgaaaatgagcac 120 |
| 121 | cagttatctttaagaacggtcagtttaggggctggtgcaaaggatgagttgcacattgtt 180 |
| 181 | gaagcagaggcaatgaattacgaaggcagtccaattaaagtaacactggcaactttgaaa 240 |
| 241 | atgtctgtacagccaacggtttcccttgggggctttgaaataacaccaccagtggtctta 300 |
| 301 | aggttgaagtgtggttcagggccagtgcatattagtggacagcacttagtagtgtaccgc 360 |
| 361 | cggaagcaccaggagctgcaagccatgcagatggagctgcagagccctgagtacaagctg 420 |
| 421 | agcaagctccgcacctcgaccatcatgaccgactacaaccccaactactgctttgctggc 480 |
| 481 | aagacctcctccatcagtgacctgaaggaggtgccgcggaaaaacatcaccctcattcgg 540 |
| 541 | ggtctgggccatggcgcctttggggaggtgtatgaaggccaggtgtccggaatgcccaac 600 |
| 601 | gacccaagccccctgcaagtggctgtgaagacgctgcctgaagtgtgctctgaacaggac 660 |
| 661 | gaactggatttcctcatggaagccctgatcatcagcaaattcaaccaccagaacattgtt 720 |
| 721 | cgctgcattggggtgagcctgcaatccctgccccggttcatcctgctggagctcatggcg 780 |
| 781 | gggggagacctcaagtccttcctccgagagacccgccctcgcccgagccagccctcctcc 840 |
| 841 | ctggccatgctggaccttctgcacgtggctcgggacattgcctgtggctgtcagtatttg 900 |
| 901 | gaggaaaaccacttcatccaccgagacattgctgccagaaactgcctcttgacctgtcca 960 |
| 961 | ggccctggaagagtggccaagattggagacttcgggatggcccgagacatctacagggcg 1020 |
| 1021 | agctactatagaaagggaggctgtgccatgctgccagttaagtggatgcccccagaggcc 1080 |
| 1081 | ttcatggaaggaatattcacttctaaaacagacacatggtcctttggagtgctgctatgg 1140 |
| 1141 | gaaatcttttctcttggatatatgccataccccagcaaaagcaaccaggaagttctggag 1200 |
| 1201 | tttgtcaccagtggaggccggatggacccacccaagaactgccctgggcctgtataccgg 1260 |
| 1261 | ataatgactcagtgctggcaacatcagcctgaagacaggcccaactttgccatcattttg 1320 |
| 1321 | gagaggattgaatactgcacccaggacccggatgtaatcaacaccgctttgccgatagaa 1380 |
| 1381 | tatggtccacttgtggaagaggaagagaaagtgcctgtgaggcccaaggaccctgagggg 1440 |
| 1441 | gttcctcctctcctggtctctcaacaggcaaaacgggaggaggagcgcagcccagctgcc 1500 |
| 1501 | ccaccacctctgcctaccacctcctctggcaaggctgcaaagaaacccacagctgcagag 1560 |
| 1561 | gtctctgttcgagtccctagagggccggccgtggaagggggacacgtgaatatggcattc 1620 |
| 1621 | tctcagtccaaccctccttcggagttgcacaaggtccacggatccagaaacaagcccacc 1680 |
| 1681 | agcttgtggaacccaacgtacggctcctggtttacagagaaacccaccaaaaagaataat 1740 |
| 1741 | cctatagcaaagaaggagccacacgacaggggtaacctggggctggagggaagctgtact 1800 |
| 1801 | gtcccacctaacgttgcaactgggagacttccgggggcctcactgctcctagagccctct 1860 |
| 1861 | tcgctgactgccaatatgaaggaggtacctctgttcaggctacgtcacttcccttgtggg 1920 |
| 1921 | aatgtcaattacggctaccagcaacagggcttgcccttagaagccgctactgcccctgga 1980 |
| 1981 | gctggtcattacgaggataccattctgaaaagcaagaatagcatgaaccagcctgggccc 2040 |
| 2041 | tga 2043 |

In some embodiments, the fusion protein is an ALK protein fused to an echinoderm microtubule-associated protein-like 4 (EML4) protein. In some embodiments, the ELM4-ALK fusion protein is at least 85%, at least 90%, at least 95%, at least 98%, at least 99%, or 100% identical to a ELM4-ALK fusion protein in Homo Sapiens or a variant thereof. In some embodiments, the ELM4-ALK fusion protein is at least 85%, at least 90%, at least 95%, at least 98%, at least 99%, or 100% identical to an exemplary ELM4-ALK fusion protein amino acid sequence from Homo Sapiens (GenBank: BAM37627.1) as provided below:

| | |
|---|---|
| 1 | MDGFAGSLDD SISAASTSDV QDRLSALESR |
| | VQQQEDEITV LKAALADVLR RLAISEDHVA |
| 61 | SVKKSVSSKG QPSPRAVIPM SCITNGSGAN |
| | RKPSHTSAVS IAGKETLSSA AKSGTEKKKE |
| 121 | KPQGQREKKE ESHSNDQSPQ IRASPSPQPS |
| | SQPLQIHRQT PESKNATPTK SIKRPSPAEK |
| 181 | SHNSWENSDD SRNKLSKIPS TPKLIPKVTK |
| | TADKHKDVII NQEGEYIKMF MRGRPITMFI |
| 241 | PSDVDNYDDI RTELPPEKLK LEWAYGYRGK |
| | DCRANVYLLP TGEIVYFIAS VVVLFNYEER |
| 301 | TQRHYLGHTD CVKCLAIHPD KIRIATGQIA |
| | GVDKDGRPLQ PHVRVWDSVT LSTLQIIGLG |
| 361 | TFERGVGCLD FSKADSGVHL CVIDDSNEHM |
| | LTVWDWQRKA KGAEIKTTNE VVLAVEFHPT |
| 421 | DANTIITCGK SHIFFWTWSG NSLTRKQGIF |
| | GKYEKPKFVQ CLAFLGNGDV LTGDSGGVML |
| 481 | IWSKTTVEPT PGKGPKGVYQ ISKQIKAHDG |
| | SVFTLCQMRN GMLLTGGGKD RKIILWDHDL |
| 541 | NPEREIEFSA SRARLPGHVA ADHPPAVYRR |
| | KHQELQAMQM ELQSPEYKLS KLRTSTIMTD |
| 601 | YNPNYCFAGK TSSISDLKEV PRKNITLIRG |
| | LGHGAFGEVY EGQVSGMPND PSPLQVAVKT |
| 661 | LPEVCSEQDE LDFLMEALII SKFNHQNIVR |
| | CIGVSLQSLP RFILLELMAG GDLKSFLRET |
| 721 | RPRPSQPSSL AMLDLLHVAR DIACGCQYLE |
| | ENHFIHRDIA ARNCLLTCPG PGRVAKIGDF |
| 781 | GMARDIYRAS YYRKGGCAML PVKWMPPEAF |
| | MEGIFTSKTD TWSFGVLLWE IFSLGYMPYP |
| 841 | SKSNQEVLEF VTSGGRMDPP KNCPGPVYRI |
| | MTQCWQHQPE DRPNFAIILE RIEYCTQDPD |
| 901 | VINTALPIEY GPLVEEEEKV PVRPKDPEGV |
| | PPLLVSQQAK REEERSPAAP PPLPTTSSGK |
| 961 | AAKKPTAAEI SVRVPRGPAV EGGHVNMAFS |
| | QSNPPSELHK VHGSRNKPTS LWNPTYGSWF |
| 1021 | TEKPTKKNNP IAKKEPHDRG NLGLEGSCTV |
| | PPNVATGRLP GASLLLEPSS LTANMKEVPL |
| 1081 | FRLRHFPCGN VNYGYQQQGL PLEAATAPGA |
| | GHYEDTILKS KNSMNQPGP |

In some embodiments, the ELM4-ALK fusion protein is encoded by a nucleic acid sequence that is at least 85%, at least 90%, at least 95%, at least 98%, at least 99%, or 100% identical to an exemplary nucleic acid sequence from Homo Sapiens (GenBank: AB274722.1) as provided below:

| | |
|---|---|
| 1 | ggcggcgcgg cgcggcgctc gcggctgctg cctgggaggg aggccgggca ggcggctgag |
| 61 | cggcgcggct ctcaacgtga cggggaagtg gttcgggcgg ccgcggctta ctaccccagg |
| 121 | gcgaacggac ggacgacgga ggcgggagcc ggtagccgag ccgggcgacc tagagaacga |
| 181 | gcgggtcagg ctcagcgtcg gccactctgt cggtccgctg aatgaagtgc ccgcccctct |
| 241 | gagcccggag cccggcgctt tccccgcaag atggacggtt tcgccggcag tctcgatgat |
| 301 | agtatttctg ctgcaagtac ttctgatgtt caagatcgcc tgtcagctct tgagtcacga |
| 361 | gttcagcaac aagaagatga aatcactgtg ctaaaggcgg ctttggctga tgttttgagg |
| 421 | cgtcttgcaa tctctgaaga tcatgtggcc tcagtgaaaa aatcagtctc aagtaaaggc |
| 481 | caaccaagcc ctcgagcagt tattcccatg tcctgtataa ccaatggaag tggtgcaaac |
| 541 | agaaaaccaa gtcataccag tgctgtctca attgcaggaa aagaaactct ttcatctgct |
| 601 | gctaaaagtg gtacagaaaa aaagaaagaa aaaccacaag gacagagaga aaaaaaagag |
| 661 | gaatctcatt ctaatgatca aagtccacaa attcgagcat caccttctcc ccagccctct |
| 721 | tcacaacctc tccaaataca cagacaaact ccagaaagca agaatgctac tcccaccaaa |
| 781 | agcataaaac gaccatcacc agctgaaaag tcacataatt cttgggaaaa ttcagatgat |
| 841 | agccgtaata aattgtcgaa aataccttca acacccaaat taataccaaa agttaccaaa |
| 901 | actgcagaca agcataaaga tgtcatcatc aaccaagaag gagaatatat taaaatgttt |
| 961 | atgcgcggtc ggccaattac catgttcatt ccttccgatg ttgacaacta tgatgacatc |
| 1021 | agaacggaac tgcctcctga gaagctcaaa ctggagtggg catatggtta tcgaggaaag |
| 1081 | gactgtagag ctaatgttta ccttcttccg accggggaaa tagtttattt cattgcatca |
| 1141 | gtagtagtac tatttaatta tgaggagaga actcagcgac actacctggg ccatacagac |
| 1201 | tgtgtgaaat gccttgctat acatcctgac aaaattagga ttgcaactgg acagatagct |
| 1261 | ggcgtggata aagatggaag gcctctacaa ccccacgtca gagtgtggga ttctgttact |
| 1321 | ctatccacac tgcagattat tggacttggc acttttgagc gtggagtagg atgcctggat |
| 1381 | ttttcaaaag cagattcagg tgttcattta tgtgttattg atgactccaa tgagcatatg |
| 1441 | cttactgtat gggactggca gaagaaagca aaaggagcag aaataaagac aacaaatgaa |
| 1501 | gttgttttgg ctgtggagtt tcacccaaca gatgcaaata ccataattac atgcggtaaa |
| 1561 | tctcatattt tcttctggac ctggagcggc aattcactaa caagaaaaca gggaattttt |
| 1621 | gggaaatatg aaaagccaaa atttgtgcag tgtttagcat tcttggggaa tggagatgtt |
| 1681 | cttactggag actcaggtgg agtcatgctt atatggagca aaactactgt agagcccaca |
| 1741 | cctgggaaag gacctaaagt gtaccgccgg aagcaccagg agctgcaagc catgcagatg |
| 1801 | gagctgcaga gccctgagta caagctgagc aagctccgca cctcgaccat catgaccgac |
| 1861 | tacaacccca actactgctt tgctggcaag acctcctcca tcagtgacct gaaggaggtg |
| 1921 | ccgcggaaaa acatcaccct cattcggggt ctgggccatg gagcctttgg ggaggtgtat |
| 1981 | gaaggccagg tgtccggaat gcccaacgac ccaagccccc tgcaagtggc tgtgaagacg |
| 2041 | ctgcctgaag tgtgctctga acaggacgaa ctggatttcc tcatggaagc cctgatcatc |
| 2101 | agcaaattca accaccagaa cattgttcgc tgcattgggg tgagcctgca atccctgccc |
| 2161 | cggttcatcc tgctggagct catggcgggg ggagacctca agtccttcct ccgagagacc |
| 2221 | cgccctcgcc cgagccagcc ctcctccctg gccatgctgg accttctgca cgtggctcgg |
| 2281 | gacattgcct gtggctgtca gtatttggag gaaaaccact tcatccaccg agacattgct |
| 2341 | gccagaaact gcctcttgac ctgtccaggc cctggaagag tggccaagat tggagacttc |
| 2401 | gggatggccc gagacatcta cagggcgagc tactatagaa agggaggctg tgccatgctg |
| 2461 | ccagttaagt ggatgccccc agaggccttc atggaaggaa tattcacttc taaaacagac |
| 2521 | acatggtcct ttggagtgct gctatgggaa atcttttctc ttggatatat gccatacccc |
| 2581 | agcaaaagca accaggaagt tctggagttt gtcaccagtg gaggccggat ggacccaccc |
| 2641 | aagaactgcc ctgggcctgt ataccggata atgactcagt gctggcaaca tcagcctgaa |
| 2701 | gacaggccca actttgccat cattttggag aggattgaat actgcaccca ggacccggat |
| 2761 | gtaatcaaca ccgctttgcc gatagaatat ggtccacttg tggaagagga agagaaagtg |
| 2821 | cctgtgaggc ccaaggaccc tgagggggtt cctcctctcc tggtctctca acaggcaaaa |
| 2881 | cgggaggagg agcgcagccc agctgcccca ccacctctgc ctaccacctc ctctggcaag |
| 2941 | gctgcaaaga aacccacagc tgcagaggtc tctgttcgag tccctagagg gccggccgtg |
| 3001 | gaagggggac acgtgaatat ggcattctct cagtccaacc ctccttcgga gttgcacagg |
| 3061 | gtccacggat ccagaaacaa gcccaccagc ttgtggaacc caacgtacgg ctcctggttt |
| 3121 | acagagaaac ccaccaaaaa gaataatcct atagcaaaga aggagccaca cgagaggggt |
| 3181 | aacctggggc tggagggaag ctgtactgtc ccacctaacg ttgcaactgg gagacttccg |
| 3241 | ggggcctcac tgctcctaga gccctcttcg ctgactgcca atatgaagga ggtacctctg |
| 3301 | ttcaggctac gtcacttccc ttgtgggaat gtcaattacg gctaccagca acagggcttg |
| 3361 | cccttagaag ccgctactgc ccctggagct ggtcattacg aggataccat tctgaaaagc |
| 3421 | aagaatagca tgaaccagcc tgggccctga gctcggtcac acactcactt ctcttccttg |
| 3481 | ggatccctaa gaccgtggag gagagagagg caatcaatgg ctccttcaca aaccagagac |
| 3541 | caaatgtcac gttttgtttt gtgccaacct attttgaagt accaccaaaa aagctgtatt |
| 3601 | ttgaaaatgc tttagaaagg ttttgagcat gggttcatcc tattctttcg aaagaagaaa |
| 3661 | atatcataaa aatgagtgat aaatacaagg cccagatgtg gttgcataag gtttttatgc |
| 3721 | atgtttgttg tatacttcct tatgcttctt ttaaattgtg tgtgctctgc ttcaatgtag |
| 3781 | tcagaattag ctgcttctat gtttcatagt tggggtcata gatgtttcct tgccttgttg |
| 3841 | atgtggacat gagccatttg aggggagagg gaacggaaat aaaggagtta tttgtaatga |
| 3901 | aaaaaaaaaa aaaaaaaaaa aaaaaa |

In some embodiments, the ELM4-ALK fusion protein is at least 85%, at least 90%, at least 95%, at least 98%, at least 99%, or 100% identical to an exemplary ELM4-ALK variant 1 fusion protein amino acid sequence from Homo Sapiens (GenBank: BAF73611.1) as provided below:

```
      1 mdgfagsldd sisaastsdv qdrlsalesr
          vqqqedeitv ikaaladvlr rlaisedhva
    61 svkksvsskg qpspravipm scitngsgan
          rkpshtsavs iagketlssa aksgtekkke
  121 kpqgqrekke eshsndqspq iraspspqps
          sqplqihrqt pesknatptk sikrpspaek
  181 shnswensdd srnklskips tpklipkvtk
          tadkhkdvii nqegeyikmf mrgrpitmfi
  241 psdvdnyddi rtelppeklk lewaygyrgk
          dcranvyllp tgeivyfias vvvlfnyeer
  301 tqrhylghtd cvkclaihpd kiriatgqia
          gvdkdgrplq phvrvwdsvt istlqiiglg
  361 tfergvgcld fskadsgvhl cviddsnehm
          itvwdwqkka kgaeikttne vvlavefhpt
  421 dantiitcgk shiffwtwsg nsltrkqgif
          gkyekpkfvq clafigngdv ltgdsggvml
  481 iwskttvept pgkgpkvyrr khqelqamqm
          elqspeykls klrtstimtd ynpnycfagk
  541 tssisdlkev prknitlirg ighgafgevy
          egqvsgmpnd psplqvavkt Ipevcseqde
  601 ldflmealii skfnhqnivr cigvslqslp
          rfillelmag gdlksfIret rprpsqpssl
  661 amldllhvar diacgcqyle enhfihrdia
          arnclltcpg pgrvakigdf gmardiyras
  721 yyrkggcaml pvkwmppeaf megiftsktd
          twsfgvilwe ifsigympyp sksnqevlef
  781 vtsggrmdpp kncpgpvyri mtqcwqhqpe
          drpnfaiile rieyctqdpd vintalpiey
  841 gplveeeekv pvrpkdpegv ppllvsqqak
          reeerspaap pplpttssgk aakkptaaev
  901 svrvprgpav egghvnmafs qsnppselhr
          vhgsrnkpts Iwnptygswf tekptkknnp
  961 iakkepherg nlglegsctv ppnvatgrlp
          gaslllepss itanmkevpl frlrhfpcgn
1021 vnygyqqqgl pleaatapga ghyedtilks
          knsmnqpgp
```

By “genetic vaccine” is meant an immunogenic composition comprising a polynucleotide encoding an antigen.

A “human antibody” is an antibody which includes sequences from (or derived from) the human genome, and does not include sequence from another species. In some embodiments, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1 Ed. New York: Cold Spring Harbor Laboratory Press, 2004. Print.: Lonberg, Nat. Biotech., 23: 1117-1125, 2005; Lonenberg, Curr. Opin. Immunol. 20:450-459, 2008).

By “humanized antibody” is meant a human framework region and one or more CDRs from a non-human (e.g., a mouse, rat, or synthetic) antibody or antigen binding fragment (e.g., ALK antibody or antigen binding fragment). In one embodiment, all the CDRs of an ALK humanized antibody are from a non-human (e.g., a mouse, rat, or synthetic) antibody. In some embodiments, the humanized antibody further comprises constant regions. In some embodiments, the constant regions are substantially identical (e.g., at least 85%) to human immunoglobulin constant regions. Humanized antibodies can be produced using a variety of techniques known in the art.

“Hybridization” means hydrogen bonding, which may be Watson-Crick, Hoogsteen, or reversed Hoogsteen hydrogen bonding, between complementary nucleobases. For example, in DNA, adenine and thymine, and cytosine and guanine, are, respectively, complementary nucleobases that pair through the formation of hydrogen bonds. By “hybridize” is meant pairing to form a double-stranded molecule between complementary polynucleotide sequences (e.g., a gene), or portions thereof, under various conditions of stringency. (See, e.g., Wahl, G. M. and S. L. Berger, (1987), Methods Enzymol., 152:399; Kimmel, A. R., (1987), Methods Enzymol.

By way of example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “immune effector cell” is meant a lymphocyte, once activated, capable of effecting an immune response upon a target cell. In some embodiments, immune effector cells are effector T cells. In some embodiments, the effector T cell is a naïve CD8⁺ T cell, a cytotoxic T cell, a natural killer T (NKT) cell, a natural killer (NK) cell, or a regulatory T (Treg) cell. In some embodiments, the effector T cells are thymocytes, immature T lymphocytes, mature T lymphocytes, resting T lymphocytes, or activated T lymphocytes. In some embodiments the immune effector cell is a CD4⁺ CD8⁺ T cell or a CD4⁻ CD8⁻ T cell. In some embodiments the immune effector cell is a T helper cell. In some embodiments the T helper cell is a T helper 1 (Th1), a T helper 2 (Th2) cell, or a helper T cell expressing CD4 (CD4+ T cell).

By “immunogen” is meant agent which is capable, under appropriate conditions, of eliciting or stimulating an immune response, such as the production a T-cell response, in an animal, including compositions that are injected or absorbed into an animal. As used herein, an “immunogenic composition” is a composition comprising an immunogen (such as an ALK polypeptide) or a vaccine comprising an immunogen (such as an ALK polypeptide). As will be appreciated by the skilled person in the art, if administered to a subject in need prior to the subject's contracting disease or experiencing full-blown disease, an immunogenic composition can be prophylactic and result in the subject's eliciting an immune response, e.g., a cellular immune response, to protect against disease, or to prevent more severe disease or condition, and/or the symptoms thereof. If administered to a subject in need following the subject's contracting disease, an immunogenic composition can be therapeutic and result in the subject's eliciting an immune response, e.g., a cellular immune response, to treat the disease, e.g., by reducing, diminishing, abrogating, ameliorating, or eliminating the disease, and/or the symptoms thereof. In some embodiments, the immune response is a B-cell response, which results in the production of antibodies, e.g., neutralizing antibodies, directed against the immunogen or immunogenic composition comprising the antigen or antigen sequence. In some embodiments, the immune response is a T-cell response, which results in the production of T-lymphocytes. In a manner similar to the foregoing, in some embodiments, an immunogenic composition or vaccine can be prophylactic. In some embodiments, an immunogenic composition or vaccine can be therapeutic. In some embodiments, the disease is caused by oncogenic ALK gene fusions, rearrangements, duplications or mutations (e.g., ALK-positive cancers). In some embodiments, the cancer is an ALK-positive cancer. In some embodiments, the ALK-positive cancer is non-small cell lung cancer (NSCLC), anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, melanoma, or a combination thereof.

The term “immune response” is meant any response mediated by an immunoresponsive cell. In one example of an immune response, leukocytes are recruited to carry out a variety of different specific functions in response to exposure to an antigen (e.g., a foreign entity). Immune responses are multifactorial processes that differ depending on the type of cells involved. Immune responses include cell-mediated responses (e.g., T-cell responses), humoral responses (B-cell/antibody responses), innate responses and combinations thereof.

By “immunogenic composition” is meant a composition comprising an antigen, antigen sequence, or immunogen, wherein the composition elicits an immune response in an immunized subject.

The term “immunize” (or immunization) refers to rendering a subject protected from a disease or pathology, or the symptoms thereof, caused by oncogenic ALK gene fusions, rearrangements, duplications or mutations (e.g., ALK-positive cancers), such as by vaccination.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid, protein, or peptide is purified if it is substantially free of cellular material, debris, non-relevant viral material, or culture medium when produced by recombinant DNA techniques, or of chemical precursors or other chemicals when chemically synthesized. Purity and homogeneity are typically determined using standard purification methods and analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. The term “isolated” also embraces recombinant nucleic acids or proteins, as well as chemically synthesized nucleic acids or peptides.

By “isolated polynucleotide” is meant a nucleic acid (e.g., a DNA molecule) that is free of the genes which flank the gene, in the naturally-occurring genome of the organism from which the nucleic acid molecule of the invention is derived. The term includes, for example, a recombinant DNA that is incorporated into a vector; into an autonomously replicating plasmid or virus; into the genomic DNA of a prokaryote or eukaryote; or that exists as a separate molecule independent of other sequences (for example, a cDNA or a genomic or cDNA fragment produced by PCR or restriction endonuclease digestion). In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 40%, by weight, at least 50%, by weight, at least 60%, by weight, free from the proteins and naturally-occurring organic molecules with which it is naturally associated.

Preferably, an isolated polypeptide preparation is at least 75%, more preferably at least 90%, and most preferably at least 99%, by weight, free from the proteins and naturally-occurring organic molecules with which it is naturally associated. An isolated polypeptide may be obtained, for example, by extraction from a natural source; by expression of a recombinant nucleic acid encoding such a polypeptide; or by chemically synthesizing the protein. Purity can be measured by any standard, appropriate method, for example, column chromatography, polyacrylamide gel electrophoresis, or by HPLC analysis. An isolated polypeptide can refer to an ALK antigen or immunogen polypeptide generated by the methods described herein.

By “K_{D}” is mean the dissociation constant for a given interaction (e.g., an antibody antigen interaction). For example, for the bimolecular interaction of an antibody or antigen binding fragment (e.g., an ALK antibody or an antigen binding fragment thereof) and an antigen (e.g., an ALK protein) it is the concentration of the individual components of the bimolecular interaction divided by the concentration of the complex.

By “linker” is meant a bond (e.g., covalent bond), chemical group, or a molecule (e.g., one or more amino acids) linking two molecules or moieties, e.g., two domains of a fusion protein (e.g., an ALK domain and a domain from ELM4 or NPM) or in the context of a chimeric antigen receptor, a linker linking an antibody variable heavy (VH) region to a constant heavy (CH) region.

Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 25, 35, 45, 50, 55, 60, 60, 65, 70, 70, 75, 80, 85, 90, 90, 95, 100, 101, 102, 103, 104, 105, 110, 120, 130, 140, 150, 160, 175, 180, 190, or 200 amino acids in length. Longer or shorter linkers are also contemplated.

In some embodiments, the linker joins two domains of a fusion protein, such as, for example, an ALK domain and a domain from ELM4 or NPM. In some embodiments, the linker joins an antibody variable heavy (VH) region to a constant heavy (CH) region. In some embodiments, the chimeric antigen receptor (CAR) comprises at least one linker. The at least one linker joins, or links, a variable heavy (VH) region to a constant heavy (CH) region of the extracellular binding domain of the chimeric antigen receptor. Linkers can also link a variable light (VL) region to a variable constant (VC) region of the extracellular binding domain.

In some embodiments, the linker is a flexible protein linker. In some embodiments, the linker is a (Gly₄Ser)ₙ linker. In some embodiments, the linker is (Gly₄Ser₁)₃.

By “marker” is meant any protein or polynucleotide having an alteration in expression level or activity that is associated with a disease, condition, pathology, or disorder.

By “monoclonal antibody” is meant an antibody obtained from a population of homogenous or substantially homogeneous antibodies. Monoclonal antibodies are highly specific, being directed against a single antigenic epitope. In some embodiments, a “monoclonal antibody,” as used herein, is an antibody produced by a single cell or cell line wherein the antibody specifically binds to an ALK epitope (e.g., an epitope of the extracellular domain of ALK) as determined, e.g., by ELISA or other antigen-binding or competitive binding assay known in the art. In some embodiments, a monoclonal antibody can be a chimeric antibody or a humanized antibody. In some embodiments, a monoclonal antibody can be a human antibody.

The term “monoclonal” is not limited to any particular method for making the antibody. Generally, a population of monoclonal antibodies can be generated by cells, a population of cells, or a cell line. Methods of producing monoclonal antibodies include but are not limited to hybridoma technology, recombinant technology, or phage display methods. In some embodiments, monoclonal antibodies are isolated from a subject. In some embodiments, monoclonal antibodies can be produced recombinantly from host cells engineered to express an antibody described herein (e.g., anti-ALK antibody comprising the CDRs of any one of antibodies ALK #1, ALK #2, ALK #3, ALK #4, ALK #5, ALK #6, or ALK #7 as provided in Tables 3 and 4, respectively) or a fragment thereof, for example, a light chain and/or heavy chain of such an antibody. Methods of generating monoclonal antibodies are known and are described in the art.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)).

“Neoplasia” refers to cells or tissues exhibiting abnormal growth or proliferation. The term neoplasia encompasses cancer and solid tumors.

“Neuroblastoma” refers to a solid cancerous tumor that usually originates in the abdomen in adrenal gland tissue, but can also originate from nerve tissue in the neck, chest, abdomen, and pelvis. Neuroblastoma is derived from the neural crest and is characterized by a marked clinical heterogeneity (aggressive, unremitting growth to spontaneous remission). Neuroblastoma may metastasize to the lymph nodes, liver, lungs, bones and bone marrow. Neuroblastoma is the most common heterogenous and malignant tumor of early childhood, and two thirds of individuals with neuroblastoma are diagnosed when they are younger than 5 years. About 10% of neuroblastoma cases have activating point mutation in the ALK protein (e.g., ALK^{F1174L}).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′—e.g., fluororibose, ribose, 2′-deoxyribose, arabinose, and hexose); and/or modified phosphate groups (e.g., phosphorothioates and 5′-N-phosphoramidite linkages).

As used herein, “obtaining” as in “obtaining an agent” includes synthesizing, isolating, purchasing, or otherwise acquiring the agent.

The term “operably linked” refers to nucleic acid sequences as used herein. By way of example, a first nucleic acid sequence is operably linked to a second nucleic acid sequence when the first nucleic acid sequence is placed in a functional relationship with the second nucleic acid sequence. For instance, a promoter is operably linked to a coding sequence if the promoter affects (allows) the transcription or expression of the coding sequence. Generally, operably linked DNA sequences are contiguous and, where necessary to join two protein-coding regions, are in the same open reading frame.

The nucleic acid sequence encoding an ALK protein (antigen protein) generated by the described methods can be optimized for expression in mammalian cells via codon-optimization and RNA optimization (such as to increase RNA stability) using procedures and techniques practiced in the art.

By “open reading frame (ORF)” is meant a series of nucleotide triplets (codons) that code for amino acids without any termination codons. These sequences are usually translatable into a peptide or polypeptide.

The term “pharmaceutically acceptable vehicle” refers to conventional carriers (vehicles) and excipients that are physiologically and pharmaceutically acceptable for use, particularly in mammalian, e.g., human, subjects. Such pharmaceutically acceptable vehicles are known to the skilled practitioner in the pertinent art and can be readily found in Remington's Pharmaceutical Sciences, by E. W. Martin, Mack Publishing Co., Easton, Pa., 15th Edition (1975) and its updated editions, which describes compositions and formulations suitable for pharmaceutical delivery of one or more therapeutic or immunogenic compositions, such as one or more vaccines, and additional pharmaceutical agents. In general, the nature of a pharmaceutically acceptable carrier depends on the particular mode of administration being employed. For instance, parenteral formulations usually comprise injectable fluids/liquids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers may include, for example, pharmaceutical grades of mannitol, lactose, starch, or magnesium stearate, which typically stabilize and/or increase the half-life of a composition or drug. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

By “plasmid” is meant a circular nucleic acid molecule capable of autonomous replication in a host cell.

By “polyclonal antibodies” is meant an antibody population obtained from different cell lineages that includes a variety of different antibodies that specifically bind to the same and/or to different epitopes within an antigen or antigens (e.g., ALK protein).

The terms “protein,” “peptide,” “polypeptide,” and their grammatical equivalents are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three (3) amino acids long. A protein, peptide, or polypeptide can refer to an individual protein or a collection of proteins. One or more of the amino acids in a protein, peptide, or polypeptide can be modified, such as glycoproteins, for example, by the addition of a chemical entity such as a carbohydrate group, a hydroxyl group, a phosphate group, a farnesyl group, an isofarnesyl group, a fatty acid group, a linker for conjugation, functionalization, or other modifications, etc. A protein, peptide, or polypeptide can also be a single molecule or can be a multi-molecular complex. A protein, peptide, or polypeptide can be just a fragment of a naturally occurring protein or peptide. A protein, peptide, or polypeptide can be naturally occurring, recombinant, or synthetic, or any combination thereof.

In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA or DNA. Any of the proteins provided herein can be produced by any method known in the art. For example, the proteins provided herein can be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

Conservative amino acid substitutions are those substitutions that, when made, least interfere with the properties of the original protein, that is, the structure and especially the function of the protein is conserved and is not significantly changed by such substitutions. Examples of conservative amino acid substitutions are known in the art, e.g., as set forth in, for example, U.S. Publication No. 2015/0030628. Conservative substitutions generally maintain (a) the structure of the polypeptide backbone in the area of the substitution, for example, as a sheet or helical conformation; (b) the charge or hydrophobicity of the molecule at the target site; and/or (c) the bulk of the side chain

The substitutions that are generally expected to produce the greatest changes in protein properties are non-conservative, for instance, changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

By “promoter” is meant an array of nucleic acid control sequences, which direct transcription of a nucleic acid. A promoter includes necessary nucleic acid sequences near the start site of transcription. A promoter also optionally includes distal enhancer or repressor sequence elements. A “constitutive promoter” is a promoter that is continuously active and is not subject to regulation by external signals or molecules. In contrast, the activity of an “inducible promoter” is regulated by an external signal or molecule (for example, a transcription factor). By way of example, a promoter may be a CMV promoter.

As will be appreciated by the skilled practitioner in the art, the term “purified” does not require absolute purity; rather, it is intended as a relative term. Thus, for example, a purified peptide, protein, or other active compound is one that is isolated in whole or in part from naturally associated proteins and other contaminants. In certain embodiments, the term “substantially purified” refers to a peptide, protein, or other active compound that has been isolated from a cell, cell culture medium, or other crude preparation and subjected to routine methods, such as fractionation, chromatography, or electrophoresis, to remove various components of the initial preparation, such as proteins, cellular debris, and other components.

A “recombinant” nucleic acid or protein is one that has a sequence that is not naturally occurring or that has a sequence that is made by an artificial combination of two otherwise separated segments of sequence. Such an artificial combination is often accomplished by chemical synthesis or by the artificial manipulation of isolated segments of nucleic acids, for example, by genetic engineering techniques. A “non-naturally occurring” nucleic acid or protein is one that may be made via recombinant technology, artificial manipulation, or genetic or molecular biological engineering procedures and techniques, such as those commonly practiced in the art.

By “reduces” is meant a negative alteration of at least 5%, 10%, 25%, 30%, 40%, 50%, 75%, 80%, 85%, 90%, 95%, 98%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “single-chain antibody” or “scFv” is meant a genetically engineered molecule containing the VH and VL domains of one or more antibodies linked by a suitable polypeptide linker as a genetically fused single chain molecule (see, e.g., Bird et al., Science, 242:423-426, 1988; Huston et al., Proc. Natl. Acad. Sci., 85:5879-5883, 1988: Ahmad et al., Clin. Dev. Immunol., 2012, doi:10.1155/2012/980250: Marbry, IDrugs, 13:543-549, 2010). In some embodiments, the intramolecular orientation of the VH-domain and the VL-domain in an scFv is VH-domain-linker domain-VL-domain. In some embodiments, the intramolecular orientation of the VH-domain and the VL-domain in an scFv is VL-domain-linker domain-VH-domain.

By “signal peptide” or “leader peptide” is meant a short amino acid sequence (e.g., approximately 16-30 amino acids in length) that directs newly synthesized secretory or membrane proteins to and through membranes (e.g., the endoplasmic reticulum membrane). Signal peptides are typically located at the N-terminus of a polypeptide and can be removed by signal peptidases after the polypeptide has crossed the membrane. Signal peptide sequences typically contain three common structural features: N-terminal polar basic region (n-region), a hydrophobic core, and a hydrophilic c-region). In some embodiments, a CAR of the present invention includes a signal peptide sequence (e.g., N-terminal to the antigen binding domain). In some embodiments, the signal peptide sequence is mCD8. In some embodiments the leader peptide is CD8α.

By “simultaneous” or “simultaneously” is meant at approximately the same time. For example, the terms “simultaneous” or “simultaneously” include where one or more agents is administered within minutes or hours of another agent.

By “specifically binds” is meant a compound, nucleic acid molecule, polypeptide, antibody, or complex thereof (e.g., a chimeric antigen receptor) that recognizes and binds a polypeptide (e.g., an ALK polypeptide) or vaccine product, but which does not substantially recognize and bind other molecules in a sample, for example, a biological sample, which naturally includes a polypeptide of the invention, such as an ALK polypeptide. For example, a chimeric antigen receptor that specifically binds to a particular marker (e.g., an ALK polypeptide) expressed on the surface of a cell, but does not bind to other polypeptides, carbohydrates, lipids, or any other compound on the surface of the cell.

Nucleic acid molecules useful in the methods described herein include any nucleic acid molecule that encodes a polypeptide as described, or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, or at least 80% or 85%, or at least or equal to 90%, 95%, 98% or even 99% identical at the amino acid level or nucleic acid to the sequence used for comparison.

“Sequence identity” refers to the similarity between amino acid or nucleic acid sequences that is expressed in terms of the similarity between the sequences. Sequence identity is frequently measured in terms of percentage identity (or similarity or homology); the higher the percentage, the more similar the sequences are. Homologs or variants of a given gene or protein will possess a relatively high degree of sequence identity when aligned using standard methods. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence. In addition, other programs and alignment algorithms are described in, for example, Smith and Waterman, 1981, Adv. Appl. Math. 2:482; Needleman and Wunsch, 1970, J. Mol. Biol. 48:443; Pearson and Lipman, 1988, Proc. Natl. Acad. Sci. U.S.A. 85:2444; Higgins and Sharp, 1988, Gene 73:237-244; Higgins and Sharp, 1989, CABIOS 5:151-153; Corpet et al., 1988, Nucleic Acids Research 16:10881-10890; Pearson and Lipman, 1988, Proc. Natl. Acad. Sci. U.S.A. 85:2444; and Altschul et al., 1994, Nature Genet. 6:119-129. The NCBI Basic Local Alignment Search Tool (BLAST™) (Altschul et al. 1990, J. Mol. Biol. 215:403-410) is readily available from several sources, including the National Center for Biotechnology Information (NCBI, Bethesda, Md.) and on the Internet, for use in connection with the sequence analysis programs blastp, blastn, blastx, tblastn and tblastx.

By “subject” is meant an animal, e.g., a mammal, including, but not limited to, a human, a non-human primate, or a non-human mammal, such as a bovine, equine, canine, ovine, or feline mammal, or a sheep, goat, llama, camel, or a rodent (rat, mouse), gerbil, or hamster. In a nonlimiting example, a subject is one who has, is at risk of developing, or who is susceptible to a disease caused by oncogenic ALK gene fusions, rearrangements, duplications or mutations (e.g., ALK-positive cancers). In particular aspects as described herein, the subject is a human subject, such as a patient.

Ranges provided herein are understood to be shorthand for all of the values within the range, inclusive of the first and last stated values. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or greater, consecutively, such as to 100 or greater.

As used herein, the terms “treat,” “treating,” “treatment,” and the like refer to reducing, diminishing, decreasing, delaying, abrogating, ameliorating, or eliminating, a disease, condition, disorder, or pathology, and/or symptoms associated therewith. While not intending to be limiting, “treating” typically relates to a therapeutic intervention that occurs after a disease, condition, disorder, or pathology, and/or symptoms associated therewith, have begun to develop to reduce the severity of the disease, etc., and the associated signs and symptoms. It will be appreciated that, although not precluded, treating a disorder or condition does not require that the disease, condition, disorder, pathology, or the symptoms associated therewith, be completely eliminated.

As used herein, the terms “prevent,” “preventing,” “prevention,” “prophylactic treatment” and the like, refer to inhibiting or blocking a disease state, or the full development of a disease in a subject, or reducing the probability of developing a disease, disorder or condition in a subject, who does not have, but is at risk of developing, or is susceptible to developing, a disease, disorder, or condition.

By “T Cell” is meant a white blood cell critical for immune response. T cells include, but are not limited to, CD4+ T cells and CD8+ T cells. A CD4+ T lymphocyte is an immune cell that carries a marker on its surface known as “cluster of differentiation 4 (CD4).” These cells, also known as helper T cells, help orchestrate the immune response, including antibody responses as well as killer T cell responses. CD8+ T cells carry the “cluster of differentiation 8” (CD8) marker. In one embodiment, a CD8+ T cell is a cytotoxic T lymphocyte. In another embodiment, a CD8+ cell is a suppressor T cell. An effector function of a T cell is a specialized function of the T cell, such as cytolytic activity or helper activity including the secretion of cytokines.

By “T Cell Signaling Domain” is meant an intracellular portion of a protein expressed in a T cell that transduces a T cell effector function signal (e.g., an activation signal) and directs the T cell to perform a specialized function. T cell activation can be induced by a number of factors, including binding of cognate antigen to the T cell receptor on the surface of T cells and binding of cognate ligand to costimulatory molecules on the surface of the T cell. A T cell co-stimulatory molecule is a cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule. Activation of a T cell leads to immune response, Such as T cell proliferation and differentiation (see, e.g., Smith-Garvin et al., Annu. Rev. Immunol., 27:591-619, 2009).

Exemplary T cell signaling domains are known in the art. Non-limiting examples include the CD3ζ, CD8, CD28, CD27, CD154, GITR (TNFRSF18), CD134 (OX40), and CD137 (4-1BB) signaling domains.

In some embodiments, the CD3ζ signaling domain is at least 85%, at least 90%, at least 95%, at least 98%, at least 99%, or 100% identical to the CD3ζ signaling domain of the m1928z CAR construct (see Davila et al., PlosOne 2013).

In some embodiments, the CD3ζ signaling domain is at least 85%, at least 90%, at least 95%, at least 98%, at least 99%, or 100% identical to an exemplary amino acid sequence as provided below:

RVKFSRSADAPAYQQGQNQLYNELNLGRREEYDVLDK
RRGRDPEMGGKPRRKNPQEGLYNELQKDKMAEAYSEI
GMKGERRRGKGHDGLYQGLSTATKDTYDALHMQALPP
R

In some embodiments, the CD8 signaling domain is at least 85%, at least 90%, at least 95%, at least 98%, at least 99%, or 100% identical to the CD8 signaling domain of the m1928z CAR construct (see Davila et al., PlosOne 2013).

In some embodiments, the CD8 signaling domain is at least 85%, at least 90%, at least 95%, at least 98%, at least 99%, or 100% identical to an exemplary amino acid sequence as provided below:

FVPVFLPARPTTTPAPRPPTPAPTIASQPLSLRPEA
CRPAAGGAVHTRGLDFACDIYIWAPLAGTCGVLLLS
LVITLYCNHRNR

In some embodiments, the CD28 signaling domain is at least 85%, at least 90%, at least 95%, at least 98%, at least 99%, or 100% identical to the CD28 signaling domain of the m1928z CAR construct (see Davila et al., PlosOne 2013).

In some embodiments, the CD28 signaling domain is at least 85%, at least 90%, at least 95%, at least 98%, at least 99%, or 100% identical to an exemplary amino acid sequence as provided below:

SKRSRLLHSDYMNMTPRRPGPTRKHYQPYAPPRTAAYR

In some embodiments, the CD137 (4-1BB) signaling domain is at least 85%, at least 90%, at least 95%, at least 98%, at least 99%, or 100% identical to an exemplary amino acid sequence as provided below:

KRGRKKLLYIFKQPEMRPVQTTQEEDGCSCRFPEEEEGGCEL

In some embodiments, the CD137 (4-1BB) signaling domain is at least 85%, at least 90%, at least 95%, at least 98%, at least 99%, or 100% identical to an exemplary amino acid sequence as provided below:

RFSVVKRGRKKLLYIFKQPEMRPVQTTQEEDGCSCRFPEEEEGGCEL

In some embodiments, the CD134 (OX40) signaling domain is at least 85%, at least 90%, at least 95%, at least 98%, at least 99%, or 100% identical to an exemplary amino acid sequence as provided below:

RRDQRLPPDAHKPPGGGSFRTPIQEEQADAHSTLAKI

As referred to herein, a “transformed” or “transfected” cell is a cell into which a nucleic acid molecule or polynucleotide sequence has been introduced by molecular biology techniques. As used herein, the term “transfection” encompasses all techniques by which a nucleic acid molecule or polynucleotide may be introduced into such a cell, including transfection with viral vectors, transformation with plasmid vectors, and introduction of naked nucleic acid (DNA or RNA) by electroporation, lipofection, and particle gun acceleration.

By “transmembrane domain” is meant an amino acid sequence that inserts into a lipid bilayer, such as the lipid bilayer of a cell or virus or virus-like particle. A transmembrane domain can be used to anchor a protein of interest (e.g., a CAR) to a membrane. The transmembrane domain may be derived either from a natural or from a synthetic source. Where the source is natural, the domain may be derived from any membrane-bound or transmembrane protein.

Transmembrane domains for use in the disclosed CARs can include at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154.

In some embodiments, the CD28 transmembrane domain is at least 85%, at least 90%, at least 95%, at least 98%, at least 99%, or 100% identical to an exemplary amino acid sequence as provided below:

IEVMYPPPYLDNEKSNGTIIHVKGKHLCPSPLFPGP
SKPEWVLVVVGGVLACYSLLVTVAFIIEWVR

In some embodiments, the CD8 transmembrane domain is at least 85%, at least 90%, at least 95%, at least 98%, at least 99%, or 100% identical to the CD8 transmembrane domain of the m1928z CAR construct (see Davila et al., PlosOne 2013).

In some embodiments, the CD8 transmembrane domain is at least 85%, at least 90%, at least 95%, at least 98%, at least 99%, or 100% identical to an exemplary amino acid sequence as provided below:

TTTPAPRPPTPAPTIASQPLSLRPEACRPAAGGAVH
TRGLDFACDIYIWAPLAGTCGVLLLSLVITLYC

By “vaccine” is meant a preparation of immunogenic material (e.g., protein or nucleic acid) capable of stimulating (eliciting) an immune response, administered to a subject to treat a disease, condition, or pathology, or to prevent a disease, condition, or pathology (e.g., ALK-positive cancer (e.g., neuroblastoma)). The immunogenic material may include, for example, antigenic proteins, peptides or DNA derived from tumors or cell lines (e.g., ALK-expressing tumors or cell lines). In some embodiments, the immunogenic material is an ALK polypeptide or fragment thereof. Vaccines may elicit a prophylactic (preventative) immune response in the subject; they may also elicit a therapeutic response immune response in a subject. Methods of vaccine administration vary according to the vaccine, and can include routes or means, such as inoculation (intravenous or subcutaneous injection), ingestion, inhalation, or other forms of administration. Inoculations can be delivered by any number of routes, including parenteral, such as intravenous, subcutaneous or intramuscular. Vaccines may also be administered with an adjuvant to boost the immune response.

As used herein, a “vector” refers to a nucleic acid (polynucleotide) molecule into which foreign nucleic acid can be inserted without disrupting the ability of the vector to replicate in and/or integrate into a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains the necessary regulatory sequences to allow transcription and translation of inserted gene or genes in a host cell. In some embodiments of the present disclosure, the vector encodes an ALK CAR. In some embodiments, the vector is the pTR600 expression vector (U.S. Patent Application Publication No. 2002/0106798; Ross et al., 2000, Nat Immunol. 1(2):102-103; and Green et al., 2001, Vaccine 20:242-248). In some embodiments, the vector is a viral vector (e.g., lentiviral vector).

Unless specifically stated or obvious from context, as used herein, the term “or” is understood to be inclusive. Unless specifically stated or obvious from context, as used herein, the terms “a,” “an,” and “the” are understood to be singular or plural. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description.

Unless specifically stated or obvious from context, as used herein, the term “about” is understood as within a range of normal tolerance in the art, for example within two (2) standard deviations (SD) of the mean. About may be understood as being within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of some embodiments for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1C depict a cloning strategy of variable heavy (VH) and variable light (VL) chains of anaplastic lymphoma kinase (ALK) antibodies into chimeric antigen receptor (CAR) constructs. FIG. 1A provides a schematic of the cloning strategy of ALK CAR using overlap PCT to create a VDJ-H followed by mCD8 signal peptide and VJκ followed by partial (Gly4Ser1)3 linker sequence. After the second-round PCR, mCD8SP, VDJ-H, linker and VJκ can be fused. The efficacy of gene transfer can be evaluated by GFP expression. FIG. 1B provides a schematic of a mouse CAR-T construct, GL-2A-m1928z for the reporter gene (GFP) and CAR (m1928z) using a 2A peptide sequence. Depicted are the packaging signal, splice donor (SD), splice acceptor (SA), the VH and VL regions of the ALK scFv, and the extracellular (EC), transmembrane (TM), and cytosolic (C) regions. FIG. 1C provides a schematic representation of murine CD19 CAR and ALK-CAR constructs.

FIG. 2 provides graphical depictions of the transduction efficiency of T cells with ALK CAR constructs (CAR-ALK #1-#7) using fluorescence-activated cell sorting (FACS) analysis. Mouse T cells were purified from spleen, activated with anti-CD3/CD28+IL2, and transduced with a CAR retroviral construct. Efficiency of transduction was evaluated 48 hours after viral infection, by GFP reporter expression. Activated non-transduced T cells were used as a negative control. CD19-directed CAR-T cells were used as a positive control.

FIGS. 3A-3C depict cytokine release by ALK CAR constructs. Since human neuroblastoma cells do not express CD19, CD19 CAR-T cells were used as a negative control.

FIG. 3A is a graphical depiction measuring IFNγ production by ALK CAR-T cells. Retrovirally transduced CAR-T cells were incubated at a 1:1 E:T ratio (effector=GFP+CAR-T cells) with target cells. Target cells used were NIH3T3 and Eμ-myc leukemia cells transduced with retroviral vector encoding for the full-length ALK receptor or mock vector. ELISA was used to evaluate production of IFNγ in the supernatant of cells after 24 hours incubation. FIG. 3B is a graphical depiction measuring IFNγ production by ALK CAR-T cells. Retrovirally transduced CAR-T cells were incubated at a 1:1 E:T ratio (effector=GFP+CAR-T cells) with target cells. Target cells used were human neuroblastoma cells SH-SY5Y and SK-N-BE. ELISA was used to evaluate production of IFNγ in the supernatant of cells after 24 hours incubation. Error bars indicate standard deviations from 5 independent experiments. FIG. 3C is a graphical depiction measuring GM-CSF production by ALK CAR-T cells. Retrovirally transduced CAR-T cells were incubated at a 1:1 E:T ratio (effector=GFP+CAR-T cells) with target cells. Target cells used were human neuroblastoma cells SH-SY5Y and SK-N-BE. ELISA was used to evaluate production of GM-CSF in the supernatant of cells after 24 hours incubation. Error bars indicate standard deviations from 5 independent experiments.

FIG. 4 provides a graphical depiction measuring ALK-specific cytolytic activity of ALK CAR-T cell constructs compared to CD19 CAR-19 cells. Eμ-myc expressing mock vector or with a vector encoding for the full-length ALK receptor were stained with CFSE and incubated with effector CAR-T cells at E:T=10:1 ratio. The cell numbers of CAR-T cells were normalized base on the percentage of GFP positive cells transduced with the CAR construct. After 18 hours, the cytolytic activity was calculated by determining the fraction alive target cells with the formula: cytolytic activity=100−% of CSFE+/CD19+ alive cells. CD19 CAR-T cells were used as golden standard control as they efficiently target CD19+Eμ-Myc cells. Eμ-Myc vector (ALK−) cells were used as control to determine the specificity of ALK-directed cytolytic activity. Error bars are standard deviations from 5 independent experiments.

FIGS. 5A-5C depict adoptive transfer of ALK CAR-T cells into mice with Eμ-Myc/AK systemic tumors. FIG. 5A is a graphical depiction of a survival curve of mice treated with cytophspamide (CTX, 100 mg/kg) alone, cytophosphamide plus CAR-CD19 (15×10⁶ based on GFP+), and cytophosphamide plus CAR-ALK #5 (15×10⁶ based on GFP+). Untreated mice were used as a negative control. FIG. 5B is a graphical depiction of FACS analysis. CD19+/ALK+ cells were found in one mouse treated with CTX alone. The circulating CD19+/ALK+ tumor cells were found in peripheral blood (Left) and the ALK+ tumor mass was isolated close to lymph node (Right). FIG. 5C is a graphical depiction of FACS analysis conducted on the 6 out of 8 mice that survived more than two months. No tumor cells were found in peripheral blood.

FIGS. 6A-6D depict the anti-tumor activity of ALK CAR-T cells with ALK Antibody #5 (ALK #5) in a neuroblastoma model. FIG. 6A is a schematic illustration of an experimental design to evaluate the anti-tumor efficacy of ALK CAR-T cells in a subcutaneous ALK_{F1174L}/MYCN neuroblastoma model. NSG mice were transplanted s.c. with 1×10⁶ ALK^{F1174L}/MYCN cells in both flanks. FIG. 6B is a graphical depiction of neuroblastoma growth delay induced by ALK CAR-T cells with tumor volume measured daily (two-tailed p value <0.0001, unpaired t test). FIG. 6C is a graphical depiction of survival curves of neuroblastoma-bearing mice treated with ALK #5 CAR-T cells. CD19 CAR-T cells were used as a control. FIG. 6D is a graphical depiction of survival curves of neuroblastoma-bearing mice treated with ALK #5 CAR-T cells. CD19 CAR-T cells were used as a control.

FIGS. 7A-7C depict the anti-tumor activity of ALK CAR-T cells with ALK #5 compared to lorlatinib in an immunocompetent model of metastatic neuroblastoma. FIG. 7A is a graphical depiction of ALK^{F1174L}/MYCN neuroblastoma transplanted s.c. into BALB/c mice. ALK #5 CAR-T cells or CD19 CAR-T cells were generated from BALB/c purified T cells and injected i.v. weekly for three weeks. Lorlatinib was administered by oral gavage (4 mg/kg/day) for three weeks. Tumor volumes were measured at day 23. FIG. 7B depicts MRI images in immunocompetent mice injected i.v. with 1×10⁶ ALK^{F1174L}/MYCN neuroblastoma cells to induce multiple metastatic tumor formation, and treated with CD19 CAR-T cells or ALK CAR-T cells. Metastatic tumors are highlighted by dashed-lined circles. FIG. 7C is a graphical depiction of survival curves of immunocompetent mice in a metastatic model of neuroblastoma treated with the indicated CAR-T cells or lorlatinib.

FIGS. 8A-8D depict in vitro validation of human ALK CAR-T cells. FIG. 8A is a graphical depiction of ALK CAR expression in human T cells at day 4 after transduction by flow cytometry analysis. FIG. 8B is a graphical depiction of IFN-γ released by human T cells in co-culture with the human neuroblastoma cells IMR-32 at the indicated ration of effector:target (E:T) cells. FIG. 8C is a graphical depiction of proliferation by human T cells in co-culture with the human neuroblastoma cells IMR-32 at the indicated ration of E:T cells. FIG. 8D is a graphical depiction quantifying in vitro killing activity of ALK CAR-T cells evaluated by the number of residual IMR-32 neuroblastoma cells after 3 days of co-culture at the indicated ratio.

FIGS. 9A and 9B depict generation of NK cells to target ALK+ cells. FIG. 9A depicts schematic representations of hALK #5 CAR constructs to generate NK cells. FIG. 9B are graphical depictions quantifying in vitro killing activity of NK-92 cells transduced with an hALK CAR construct after 24 hours incubation with HT1080 cells expressing the human ALK receptor.

FIGS. 10A and 10B depict the effects of lorlatinib on ALK viability and expression in neuroblastoma cells. FIG. 10A is a graphical depiction of several neuroblastoma cell lines with various ALK genetic alterations (NB-1 (ALK WT amplified), IMR-32 (ALK WT), NBL-S (ALK WT), SH-SY5Y (mutated ALKF1174L), Kelly (mutated ALKF1174L)) treated with increasing doses of lorlatinib. Viability was measured at 48 hours. FIG. 10B is a graphical depiction measuring expression of surface ALK on Kelly and IMR-32 cells by flow cytometry on Kelly and IMR-32 cells treated with 10 nM lorlatinib for 24 hours.

FIGS. 11A and 11B depict the addition of ALK vaccine improves survival rate in a syngeneic model of neuroblastoma. FIG. 11A depicts a schematic illustration of an administration schedule for mice treated with a combination of an ALK vaccine, ALK CAR-T cells, and lorlatinib. BALB/c mice were injected s.c. with 1×10⁶ cells of syngeneic ALK^{F1174L}/MYCN neuroblastoma cells. Mice were vaccinated with an ALK vaccine and injected with ALK CAR-T cells at the indicated times. The ALK TKI lorlatinib was administered at 4 mg/Kg BID for the indicated time. FIG. 11B is a graphical depiction of survival curves of mice treated with a combination of an ALK vaccine, ALK CAR-T cells, and lorlatinib or a combination of ALK CAR-T cells and lorlatinib. Follow-up curves were assessed up to a cut-off of 34 days. Addition of the ALK vaccine to ALK CAR-T cells further increased survival of mice.

FIGS. 12A and 12B depict in vitro validation of hALK CAR-T cells. FIG. 12A provides a western blot showing ALK expression in a set of human neuroblastoma cell lines (LAN-1, SK-N-FI, NGP, SK-N-SH, SH-SY5Y, Kelly, LAN-5, NBL-S, Felix, IMR-32, and NB-1). FIG. 12B is a graph depicting the killing efficacy of hALK CAR-T cells against human neuroblastoma cell lines (NBL-S, SK-N-FI, IMR-32, NGP, NB-1, LAN-5, SK-N-SH, Kelly, SH-SY5Y). Data are from triplicates of CAR-T cells obtained from two independent donors. CD19 CAR-T cells and untransduced T cells were used as negative controls, and GD2 CAR-T cells were used as positive control.

FIGS. 13A-13E depict the lack of toxicity of ALK CAR-T cells. FIG. 13A are graphs depicting change in body weight in mice with (left) and without (right) tumors injected with ALK5 CAR-T cells alone and in combination with lorlatinib. CD19 CAR-T cells in combination with lorlatinib, lorlatinib alone, and untransduced T cells were used as controls. FIG. 13B are graphs depicting change in body temperature in mice with (left) and without tumors (right) injected with ALK5 CAR-T cells alone and in combination with lorlatinib. CD19 CAR-T cells in combination with lorlatinib, lorlatinib alone, and untransduced T cells were used as controls.

FIG. 13C are graphs depicting Interferon gamma (IFNγ) production (pg/ml) in mice with and without tumors injected with ALK5 CAR-T cells alone (left/right) and in combination with lorlatinib (right). CD19 CAR-T cells, CD19 CAR-T cells in combination with lorlatinib, lorlatinib alone, and untransduced T cells were used as controls. FIG. 13D are graphs depicting interleukin 6 (IL-6) production (pg/ml) in mice with and without tumors following injection of ALK5 CAR-T cells alone (left/right) and in combination with lorlatinib (right). CD19 CAR-T cells, CD19 CAR-T cells in combination with lorlatinib, lorlatinib alone, and untransduced T cells were used as controls. FIG. 13E are graphs depicting serum amyloid A 3 (mSAA3) production (μg/ml) in mice with and without tumors following injection of ALK5 CAR-T cells alone (left/right) and in combination with lorlatinib (right). CD19 CAR-T cells, CD19 CAR-T cells in combination with lorlatinib, lorlatinib alone, and untransduced T cells were used as controls.

FIGS. 14A and 14B depict killing activity of human ALK CAR-T cells against several cell lines (NBL-S, SK-N-FI, IMR-32, NGP, NB-1, LANS, SK-N-SH, Kelly, SH-SY5Y, Raji) of human neuroblastoma at 1:1 tumor:CAR-T ratio (FIG. 14A) or 1:5 tumor:CAR-T ratio (FIG. 14B). CD19 CAR-T cells and untransduced T cells were used as negative controls, and GD2 CAR-T cells were used as positive control.

FIGS. 15A-15F depict killing activity of human ALK CAR-T cells in combination with ALK inhibitor lorlatinib. FIG. 15A is a graph depicting killing activity via residual tumor cells of human ALK CAR-T cells against Kelly and SH-SY5Y cell lines of human neuroblastoma alone or in combination with lorlatinib at 10 nM and 100 nM. ALK CAR-T cells in combination with DMSO, GD2 CAR-T cells, and untransduced T cells were used as controls. FIG. 15B is a graph depicting killing activity via residual tumor cells of human ALK CAR-T cells against Kelly and SH-SY5Y cell lines of human neuroblastoma alone or in combination with lorlatinib at 10 nM and 100 nM. CD19 CAR-T cells, CD19 CAR-T cells in combination with lorlatinib at 10 nM and 100 nM, ALK CAR-T cells in combination with DMSO, GD2 CAR-T cells, and untransduced T cells were used as controls. FIG. 15C is a graph depicting killing activity via residual tumor cells of human ALK CAR-T cells against several cell lines of human neuroblastoma (LANS, SK-N-FI, IMR-32, and NGP) alone or in combination with lorlatinib at 10 nM and 100 nM. CD19 CAR-T cells, CD19 CAR-T cells in combination with lorlatinib at 10 nM and 100 nM, ALK CAR-T cells in combination with DMSO, GD2 CAR-T cells, and untransduced T cells were used as controls. FIG. 15D is a schematic depicting the mechanisms by which the ALK inhibitor lorlatinib enhances expression of ALK on the surface of neuroblastoma cells and increases the targeting by ALK CAR-T cells. FIG. 15E is a western blot showing expression of ALK in neuroblastoma cells that have a mutation of the ALK gene (LAN-5 (R1275Q), SH-SY5Y (F1174L), SK-N-SH (F1174L), NGP (D1529E), NBL-S(WT), IMR-32 (WT), SK-N-FI (WT), Kelly (WT)) when used in combination with 10 nM and 100 nM of lorlatinib. DMSO treated and untransduced cells were used as controls. FIG. 15F is a graph depicting the relative ALK mRNA expression in SH-SY5Y neuroblastoma cells after treatment with 10 nM and 100 nM of lorlatinib at 24, 48, 72, and 96 hours. DMSO treated and untransduced cells were used as controls.

FIGS. 16A and 16B depict in vivo anti-tumor activity of human ALK CAR-T cells against the human neuroblastoma cell line NB-1 that expresses high levels of ALK. FIG. 16A are heat map images of NSG mice injected with NB-1 cells and then treated with one single injection of ALK CAR-T cells. CD19 CAR-T cells and non-transduced (NT) cells were used as negative controls, and GD2 CAR-T cells were used as a positive control. Tumor growth was monitored over time by luciferase luminescence detected with IVIS instrumentation. FIG. 16B is a graph depicting the treatment-free survival (TFS) of mice treated as described in FIG. 16A.

FIGS. 17A-17D depict in vivo anti-tumor activity of human ALK CAR-T cells against the human neuroblastoma cell line SK-N-SH that expresses low levels of mutated ALK. FIG. 17A is a schematic depicting an experimental procedure for combining ALK CAR-T cells with lorlatinib in vivo in NSG mice. FIG. 17B are heat map images depicting NSG mice injected with SK-N-SH cells and then treated with one single injection of ALK CAR-T cells. CD19 CAR-T cells were used as a negative control, and GD2 CAR-T cells were used as a positive control. Lorlatinib was administered according to the procedure depicted in FIG. 17A. Tumor growth was monitored by luciferase luminescence detected with IVIS instrumentation. FIG. 17C is a graph depicting the survival of mice injected with the human neuroblastoma cell line SK-N-SH and treated with ALK CAR-T cells as described in FIG. 17B. FIG. 17D is a graph depicting the survival of mice injected with the human neuroblastoma cell line SK-N-SH and treated with ALK CAR-T cells in combination with lorlatinib as described in FIG. 17B.

FIG. 18 depicts a schematic of a hALK CAR-T construct. Depicted are the 5′ and 3′ long terminal repeat (LTR) promoters, ALK scFv, CD8a transmembrane domain (TMCD8α), CD28 signaling domain, and CD3ζ signaling domain.

### DETAILED DESCRIPTION OF THE INVENTION

As described below, the present invention features anaplastic lymphoma kinase chimeric antigen receptors (ALK CARs) and engineered immune cells comprising ALK CARs (e.g., ALK CAR-T cells). The ALK CARs of the present invention feature ALK antibody sequences that specifically bind to an ALK protein (e.g., ALK extracellular domain). The present invention also features polynucleotides encoding for ALK CARs. The ALK CAR, polynucleotide encoding an ALK CAR, or engineered immune cell comprising an ALK CAR may be used in methods to treat and/or reduce disease in a subject (e.g. ALK-positive cancer (e.g., neuroblastoma)).

The ALK CARs, polynucleotides encoding an ALK CARs, or engineered immune cells comprising ALK CARs described herein may also be used in pharmaceutical compositions that treat ALK-positive cancers (e.g., neuroblastoma) in a subject, particularly a human subject, to whom the pharmaceutical composition, is administered. ALK CARs, polynucleotides encoding an ALK CARs, or engineered immune cells comprising ALK CARs, and pharmaceutical compositions thereof, of the invention provide an additional treatment option for patients that have either become resistant to or have failed to respond to prior and traditional therapies for ALK-positive cancers.

### Anaplastic Lymphoma Kinase Chimeric Antigen Receptors (ALK CARs) and CAR-T Cells

The invention provides anaplastic lymphoma kinase chimeric antigen receptors (ALK CARs) and immune effector cells that express ALK CARs. Immune effector cells expressing a chimeric antigen receptor (CAR) can enhance an immune effector cell's immunoreactive activity, wherein the CAR has an affinity for an epitope on an antigen (e.g., ALK), wherein the antigen is associated with an altered fitness of an organism. For example, the CAR can have an affinity for an epitope on a protein expressed in a neoplastic cell (e.g., ALK-positive cancer (e.g., neuroblastoma)). Because the CAR-T cells can act independently of major histocompatibility complex (MHC), activated CAR-T cells can kill the neoplastic cell expressing the antigen. The direct action of the CAR-T cell evades neoplastic cell defensive mechanisms that have evolved in response to WIC presentation of antigens to immune effector cells.

Some embodiments comprise autologous immune effector cell immunotherapy, wherein immune effector cells are obtained from a subject having a disease or altered fitness characterized by cancerous or otherwise altered cells expressing a surface marker (e.g., ALK-positive cancer (e.g., neuroblastoma)). The obtained immune effector cells are genetically modified to express a CAR and are effectively redirected against specific antigens (e.g., ALK). Thus, in some embodiments, immune effector cells are obtained from a subject in need of CAR-T immunotherapy. In some embodiments, these autologous immune effector cells are cultured and modified shortly after they are obtained from the subject. In other embodiments, the autologous cells are obtained and then stored for future use. This practice may be advisable for individuals who may be undergoing parallel treatment that will diminish immune effector cell counts in the future. In allogeneic immune effector cell immunotherapy, immune effector cells can be obtained from a donor other than the subject who will be receiving treatment. The immune effector cells, after modification to express a CAR, are administered to a subject for treating a neoplasia (e.g., ALK-positive cancer (e.g., neuroblastoma)). In some embodiments, immune effector cells to be modified to express a CAR can be obtained from pre-existing stock cultures of immune effector cells.

Immune effector cells can be isolated or purified from a sample collected from a subject or a donor using standard techniques known in the art. For example, immune effector cells can be isolated or purified from a whole blood sample by lysing red blood cells and removing peripheral mononuclear blood cells by centrifugation. The immune effector cells can be further isolated or purified using a selective purification method that isolates the immune effector cells based on cell-specific markers such as CD25, CD3, CD4, CD8, CD28, CD45RA, or CD45RO. Another technique for isolating or purifying immune effector cells is flow cytometry. In fluorescence activated cell sorting a fluorescently labelled antibody with affinity for an immune effector cell marker is used to label immune effector cells in a sample. A gating strategy appropriate for the cells expressing the marker is used to segregate the cells. For example, T lymphocytes can be separated from other cells in a sample by using, for example, a fluorescently labeled antibody specific for an immune effector cell marker (e.g., CD4, CD8, CD28, CD45) and corresponding gating strategy. In one embodiment, a CD45 gating strategy is employed. In some embodiments, a gating strategy for other markers specific to an immune effector cell is employed instead of, or in combination with, the CD45 gating strategy.

In some embodiments, the immune effector cells contemplated in the invention are effector T cells. In some embodiments, the effector T cell is a naïve CD8⁺ T cell, a cytotoxic T cell, a natural killer T (NKT) cell, or a regulatory T (Treg) cell. In some embodiments, the effector T cells are thymocytes, immature T lymphocytes, mature T lymphocytes, resting T lymphocytes, or activated T lymphocytes. In some embodiments the immune effector cell is a CD4⁺ CD8⁺ T cell or a CD4⁻ CD8⁻ T cell. In some embodiments the immune effector cell is a T helper cell. In some embodiments the T helper cell is a T helper 1 (Th1), a T helper 2 (Th2) cell, or a helper T cell expressing CD4 (CD4+ T cell). In some embodiments, the immune effector cell is any other subset of T cells. The modified immune effector cell may express, in addition to the CAR, an exogenous cytokine, a different chimeric receptor, or any other agent that would enhance immune effector cell signaling or function. For example, coexpression of the chimeric antigen receptor and a cytokine may enhance the CAR-T cell's ability to lyse a target cell. Nonlimiting examples of cytokines include interleukin-1 (IL-1), interleukin-2 (IL-2), interleukin-4 (IL-4), interleukin-6 (IL-6), interleukin-7 (IL-7), interleukin-12 (IL-12), interleukin-15 (IL-15), interleukin-21 (IL-21), the protein memory T-cell attractant “Regulated on Activation; Normal T Expressed and Secreted” (RANTES), granulocyte-macrophage-colony stimulating factor (GM-CSF), tumor necrosis factor-alpha (TNF-α), or interferon-gamma (IFN-γ), macrophage inflammatory protein 1 alpha (MIP-1α). In some embodiments, the cytokines are of human origin (e.g., hIL-1, hIL-2, hIL-4, hIL-6, hIL-7, hIL-12, hIL-15, hIL-21, hRANTES, hGM-CSF, hTNF-α, hTNF-α, hIFNγ or hMIP-1α).

Disclosed herein are ALK CARs that are artificially constructed chimeric proteins including an extracellular antigen binding domain (e.g., single chain variable fragment (scFv)) that specifically binds to ALK), linked to a transmembrane domain, linked to one or more intracellular T-cell signaling domains. Characteristics of the disclosed ALK CARs include their ability to redirect T-cell specificity and reactivity towards ALK expressing cells in a non-MHC-restricted manner. The non-MHC-restricted ALK recognition gives T cells expressing a disclosed CAR the ability to recognize antigen independent of antigen processing, thus bypassing a major mechanism of tumor escape. Binding of an antigen (e.g., ALK) to the extracellular binding domain can activate the CAR-T cell and generate an effector response, which includes CAR-T cell proliferation, cytokine production, and other processes that lead to the death of the antigen expressing cell.

In some embodiments, the ALK CAR further comprises a linker. In some embodiments, the ALK CAR further comprises a signal peptide. In some embodiments, the ALK CAR further comprises a reporter gene (e.g., green fluorescent protein (GFP)). In some embodiments, the ALK CAR further comprises a splice donor and/or splice acceptor sequences (e.g., CMV and/or HTLV splice acceptor and donor sequences). In some embodiments, the ALK CAR further comprises a packaging signal.

Provided herein are nucleic acids that encode the ALK CARs described herein. In some embodiments, the nucleic acid is isolated or purified. Delivery of the nucleic acids ex vivo can be accomplished using methods known in the art. For example, immune effector cells obtained from a subject (e.g., mammal) may be transformed with a nucleic acid vector encoding the CAR. The vector may then be used to transform recipient immune effector cells so that these cells will then express the CAR. Efficient means of transforming immune effector cells include transfection and transduction. Such methods are well known in the art. For example, applicable methods for delivery the nucleic acid molecule encoding the chimeric antigen receptor can be found in International Application No. PCT/US2009/040040 and U.S. Pat. Nos. 8,450,112; 9,132,153; and 9,669,058, each of which is incorporated herein in its entirety.

The ALK CARs can be of any length, i.e., can comprise any number of amino acids (or nucleotides encoding amino acids), provided that the CARs retain their biological activity, e.g., the ability to specifically bind to an antigen (e.g., ALK), detect diseased cells in a mammal, or treat or prevent disease (e.g. ALK-positive cancer (e.g., neuroblastoma)) in a subject (e.g., mammal). In some embodiments, the CAR is about 50 to about 5000 amino acids long. In some embodiments, the CAR is about 50, 70, 75, 100, 125, 150, 175, 200, 300, 400, 500, 600, 700, 800, 900, 1000 or more amino acids in length.

In some embodiments, the CAR construct is derived from or comprises the m1928z CAR construct as provided in Davila et al., CD19 CAR-Targeted T Cells Induce Long-Term Remission and B Cell Aplasia in an Immunocompetent Mouse Model of B Cell Acute Lymphoblastic Leukemia, PLoS ONE (2013), which is incorporated by reference in its entirety herein.

### Extracellular Binding Domain

The ALK CARs contemplated herein include an extracellular binding domain. The extracellular binding domain of an ALK CAR contemplated herein comprises an amino acid sequence of an antibody, or an antigen binding fragment thereof, that has an affinity for a specific antigen (e.g., ALK). In some embodiments the ALK CAR comprises an amino acid sequence of an ALK antibody. In some embodiments, the ALK CAR comprises the amino acid sequence of an antigen binding fragment of an ALK antibody. The ALK antibody (or fragment thereof) portion of the extracellular binding domain recognizes and binds to an epitope of an antigen (e.g., ALK). In some embodiments, the antibody fragment portion of an ALK CAR receptor is a single chain variable fragment (scFv). An scFv comprises the light and heavy variable domains of a monoclonal antibody. In other embodiments, the antibody fragment portion of an ALK CAR is a multichain variable fragment, which can comprise more than one extracellular binding domain and therefore bind to more than one antigen simultaneously. In a multiple chain variable fragment embodiment, a hinge region may separate the different variable fragments, providing necessary spatial arrangement and flexibility.

In some embodiments, the antigen recognized and bound by the extracellular domain is a protein or peptide, a nucleic acid, a lipid, or a polysaccharide (e.g., ALK protein). Antigens can be heterologous, such as those expressed in a pathogenic bacteria or virus. Antigens can also be synthetic; for example, some individuals have extreme allergies to synthetic latex and exposure to this antigen can result in an extreme immune reaction. In some embodiments, the antigen is autologous, and is expressed on a diseased or otherwise altered cell. For example, in some embodiments, the antigen (e.g., ALK protein) is expressed in a neoplastic cell (e.g., ALK-positive cancer (e.g., neuroblastoma)). In some embodiments, the neoplastic cell is an ALK-positive cancer. In some embodiments, the ALK-positive cancer is non-small cell lung cancer (NSCLC), anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, and melanoma. In some embodiments, the ALK-positive cancer is neuroblastoma.

Antibody-antigen interactions are noncovalent interactions resulting from hydrogen bonding, electrostatic or hydrophobic interactions, or from van der Waals forces. The affinity of extracellular binding domain of the chimeric antigen receptor for an antigen can be calculated with the following formula:

K_{A}=[Antibody−Antigen]/[Antibody][Antigen], wherein
[Ab]=molar concentration of unoccupied binding sites on the antibody;
[Ag]=molar concentration of unoccupied binding sites on the antigen; and
[Ab−Ag]=molar concentration of the antibody-antigen complex.
   The antibody-antigen interaction can also be characterized based on the dissociation of the antigen from the antibody. The dissociation constant (K_{D}) is the ratio of the association rate to the dissociation rate and is inversely proportional to the affinity constant. Thus, K_{D}=1/K_{A}. Those skilled in the art will be familiar with these concepts and will know that traditional methods, such as ELISA assays, can be used to calculate these constants.

In some embodiments, the antibody portion of an ALK CAR comprises at least one heavy chain (H). In some embodiments, the antibody portion of an ALK CAR comprises at least one light chain (L). In some embodiments, the antibody portion of an ALK CAR comprises at least one heavy chain (H) and at least one light chain (L). In some embodiments, the antibody portion of an ALK CAR comprises two heavy chains, joined by disulfide bridges and two light chains, wherein the light chains are each joined to one of the heavy chains by disulfide bridges. In some embodiments, the light chain comprises a constant region (LC) and a variable region (VL). In some embodiments, the heavy chain comprises a constant region (HC) and a variable region (VH). Complementarity determining regions (CDRs) residing in the variable region of an antibody are responsible for the antibody's affinity for a particular antigen. Thus, antibodies that recognize different antigens comprise different CDRs. CDRs reside in the variable domains of the extracellular binding domain, and variable domains (i.e., the VH and VL) can be linked with a linker or, in some embodiments, with disulfide bridges.

In some embodiments, the extracellular binding domain of the ALK CAR includes sequences from an anti-ALK antibody. In some embodiments, the ALK CAR includes sequences from an anti-ALK antibody selected from ALK #1, ALK #2, ALK #3, ALK #4, ALK #5, ALK #6, or ALK #7. In some embodiments, the extracellular binding domain includes VH and/or VL sequences from an anti-ALK antibody. In some embodiments, the extracellular binding domain includes VH and/or VL CDR sequences from an anti-ALK antibody. In some embodiments, the extracellular binding domain can include a VL and/or VH of an antibody selected from ALK #1, ALK #2, ALK #3, ALK #4, ALK #5, ALK #6, or ALK #7 (e.g., as set forth in Table 1 and Table 2, respectively). In some embodiments, the extracellular binding domain can include the HCDR1, HCDR2, and HCDR3, and/or LCDR1, LCDR2, and LCDR3 of the VH and/or VL, respectively, of an antibody selected from ALK #1, ALK #2, ALK #3, ALK #4, ALK #5, ALK #6, or ALK #7 (e.g., as set forth in Table 4 and Table 3, respectively).

In some embodiments, the ALK CAR comprises at least one linker. The at least one linker joins, or links, a variable heavy (VH) region to a constant heavy (CH) region of the extracellular binding domain of the CAR. Linkers can also link a variable light (VL) region to a variable constant (VC) region of the extracellular binding domain. In some embodiments, the linker is a flexible protein linker. In some embodiments, the linker is a (Gly₄Ser)ₙ linker. In some embodiments, the linker is (Gly₄Ser₁)₃.

In some embodiments, the ALK CAR includes a signal peptide sequence, e.g., N-terminal to the antigen binding domain, that directs newly synthesized secretory or membrane proteins to and through membranes (e.g., the endoplasmic reticulum membrane). Signal peptide sequences typically contain three common structural features: N-terminal polar basic region (n-region), a hydrophobic core, and a hydrophilic c-region). The signal peptide sequence may comprise any suitable signal peptide sequence. While the signal peptide sequence may facilitate expression of the CAR on the surface of the cell, the presence of the signal peptide sequence in an expressed CAR is not necessary in order for the CAR to function. Upon expression of the CAR on the cell surface, the signal peptide sequence may be cleaved off of the CAR. Accordingly, in some embodiments, the CAR lacks a signal peptide sequence. In some embodiments, the signal peptide sequence is approximately 16-30 amino acids in length. In one embodiment, the signal peptide sequence is mCD8. In one embodiment the leader peptide is CD8α. In one embodiment, the signal peptide sequence is a human granulocyte-macrophage colony-stimulating factor (GM-CSF) receptor sequence.

### Transmembrane Domain

The ALK CARs contemplated herein include a transmembrane domain. The transmembrane domain of the ALK CARs described herein spans the CAR-T cells lipid bilayer cellular membrane and separates the extracellular binding domain and the intracellular signaling domain. The transmembrane domain may be derived either from a natural or from a synthetic source. In some embodiments, where the source is natural, the domain may be derived from any membrane-bound or transmembrane protein. In some embodiments, the transmembrane domain may be derived from a non-human transmembrane domain and, in some embodiments, humanized (i.e., having the sequence of the nucleic acid encoding the transmembrane domain optimized such that it is more reliably or efficiently expressed in a human subject). In some embodiments, the transmembrane domain is derived from another transmembrane protein expressed in a human immune effector cell. Examples of such proteins include, but are not limited to, subunits of the T cell receptor (TCR) complex, PD1, or any of the Cluster of Differentiation proteins, or other proteins, that are expressed in the immune effector cell and that have a transmembrane domain. Transmembrane domains for use in the disclosed ALK CARs can include at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some embodiments, the transmembrane domain will be synthetic, and such sequences will comprise many hydrophobic residues.

In some embodiments, the ALK CAR transmembrane domain is fused to the extracellular domain. In some embodiments, the ALK CAR comprises a spacer between the transmembrane domain and the extracellular binding domain, the intracellular domain, or both. Such spacers can be 1, 2, 3, 4, 5, 6, 7, 8, 9, 10, 11, 12, 13, 14, 15, 16, 17, 18, 19, or 20 amino acids in length. In some embodiments, the spacer can be 20, 30, 40, 50, 60, 70, 80, 90, or 100 amino acids in length. In still other embodiments the spacer can be between 100 and 500 amino acids in length. The spacer can be any polypeptide that links one domain to another and are used to position such linked domains to enhance or optimize CAR function. In some embodiments, the spacer domain can include an immunoglobulin domain, such as a human immunoglobulin sequence. In an embodiment, the immunoglobulin domain comprises an immunoglobulin CH2 and CH3 immunoglobulin G (IgG1) domain sequence (CH2CH3). The CH2CH3 domain extends the antigen binding domain of the CAR away from the membrane of CAR-expressing cells and may more accurately mimic the size and domain structure of a native TCR.

In some embodiments, a peptide linker, preferably between 2 and 10 amino acids in length, may form the linkage between the transmembrane domain and the intracellular T cell signaling domain and/or T cell costimulatory domain of the ALK CAR. In one embodiment, the linker sequence includes one or more glycine-serine doublets. In some embodiments, the linker is a flexible protein linker. In some embodiments, the linker is a (Gly₄Ser)ₙ linker. In some embodiments, the linker is (Gly₄Ser₁)₃.

In some embodiments, the transmembrane domain comprises the transmembrane domain of a T cell receptor, such as a CD8 transmembrane domain. In another embodiment, the transmembrane domain comprises the transmembrane domain of a T cell costimulatory molecule, such as CD137 (4-1BB) or CD28.

In some embodiments, the CD28 transmembrane domain is at least 85%, at least 90%, at least 95%, at least 98%, at least 99%, or 100% identical to an exemplary amino acid sequence as provided below:

IEVMYPPPYLDNEKSNGTIIHVKGKHLCPSPLFPGP
SKPEWVLVVVGGVLACYSLLVTVAFIIEWVR

In some embodiments, the CD8 transmembrane domain is at least 85%, at least 90%, at least 95%, at least 98%, at least 99%, or 100% identical to the CD8 transmembrane domain of the m1928z CAR construct (see Davila et al., PlosOne 2013).

In some embodiments, the CD8 transmembrane domain is at least 85%, at least 90%, at least 95%, at least 98%, at least 99%, or 100% identical to an exemplary amino acid sequence as provided below:

TTTPAPRPPTPAPTIASQPLSLRPEACRPAAGGAVH
TRGLDFACDIYIWAPLAGTCGVLLLSLVITLYC

### Intracellular Signaling Domain

The ALK CARs contemplated herein comprise one or more T cell signaling domains that are capable of transducing a T cell effector function signal (e.g., an activation signal) and directing the T cell to perform a specialized function. T cell activation can be induced by a number of factors, including binding of cognate antigen to the T cell receptor on the surface of T cells and binding of cognate ligand to costimulatory molecules on the surface of the T cell. A T cell co-stimulatory molecule is a cognate binding partner on a T cell that specifically binds with a co-stimulatory ligand, thereby mediating a co-stimulatory response by the T cell, such as, but not limited to, proliferation. Co-stimulatory molecules include, but are not limited to an MEW class I molecule. Activation of a T cell leads to immune response, Such as T cell proliferation and differentiation (see, e.g., Smith-Garvin et al., Annu. Rev. Immunol., 27:591-619, 2009). Exemplary T cell signaling domains are known in the art. Non-limiting examples include the CD3ζ, CD8, CD28, CD27, CD154, GITR (TNFRSF18), CD134 (OX40), and CD137 (4-1BB) signaling domains.

In some embodiments, the intracellular signaling domain of the ALK CAR contemplated herein comprises a primary signaling domain. In some embodiments, the chimeric antigen receptor comprises the primary signaling domain and a secondary, or co-stimulatory, signaling domain. In some embodiments, the primary signaling domain comprises one or more immunoreceptor tyrosine-based activation motifs or ITAMs. In some embodiments, the primary signaling domain comprises more than one ITAM. ITAMs incorporated into the chimeric antigen receptor may be derived from ITAMs from other cellular receptors. In some embodiments, the primary signaling domain comprising an ITAM may be derived from subunits of the TCR complex, such as CD3γ, CD3ε, CD3ζ, or CD3δ. In some embodiments, the primary signaling domain comprising an ITAM may be derived from FcRγ, FcRβ, CD5, CD22, CD79a, CD79b, or CD66d. The secondary signaling domain, in some embodiments, is derived from CD28. In other embodiments, the secondary signaling domain is derived from CD2, CD4, CDS, CD8α, CD83, CD134, CD137, ICOS, or CD154.

In some embodiments, the ALK CAR can include a ON signaling domain, a CD8 signaling domain, a CD28 signaling domain, a CD137 signaling domain or a combination of two or more thereof. In one embodiment, the cytoplasmic domain includes the signaling domain of CD3ζ and the signaling domain of CD28. In another embodiment, the cytoplasmic domain includes the signaling domain of CD3ζ and the signaling domain of CD137 (4-1BB). In yet another embodiment, the cytoplasmic domain includes the signaling domain of CD3-zeta and the signaling domain of CD28 and CD137. The order of the one or more T cell signaling domains on the CAR can be varied as needed by the person of ordinary skill in the art.

In some embodiments, the entire intracellular T cell signaling domain can be employed in an ALK CAR. In some embodiments, a truncated portion of the intracellular T cell signaling domain, which is still able to transduce T cell effector function, is used in an ALK CAR. In some embodiments, the cytoplasmic sequences of the T cell receptor (TCR) and co-stimulatory molecules that act in concert to initiate signal transduction following antigen receptor engagement are used in an ALK CAR.

In some embodiments, the CD3 ζ signaling domain is at least 85%, at least 90%, at least 95%, at least 98%, at least 99%, or 100% identical to the CD3ζ signaling domain of the m1928z CAR construct (see Davila et al., PlosOne 2013).

In some embodiments, the CD3ζ signaling domain is at least 85%, at least 90%, at least 95%, at least 98%, at least 99%, or 100% identical to an exemplary amino acid sequence as provided below:

RVKFSRSADAPAYQQGQNQLYNELNLGRREEYDVL
DKRRGRDPEMGGKPRRKNPQEGLYNELQKDKMAEA
YSEIGMKGERRRGKGHDGLYQGLSTATKDTYDALH
MQALPPR

In some embodiments, the CD8 signaling domain is at least 85%, at least 90%, at least 95%, at least 98%, at least 99%, or 100% identical to the CD8 signaling domain of the m1928z CAR construct (see Davila et al., PlosOne 2013).

In some embodiments, the CD8 signaling domain is at least 85%, at least 90%, at least 95%, at least 98%, at least 99%, or 100% identical to an exemplary amino acid sequence as provided below:

FVPVFLPARPTTTPAPRPPTPAPTIASQPLSLRPE
ACRPAAGGAVHTRGLDFACDIYIWAPLAGTCGVLL
LSLVITLYCNHRNR

In some embodiments, the CD28 signaling domain is at least 85%, at least 90%, at least 95%, at least 98%, at least 99%, or 100% identical to the CD28 signaling domain of the m1928z CAR construct (see Davila et al., PlosOne 2013).

In some embodiments, the CD28 signaling domain is at least 85%, at least 90%, at least 95%, at least 98%, at least 99%, or 100% identical to an exemplary amino acid sequence as provided below:

SKRSRLLHSDYMNMTPRRPGPTRKHYQPYAPPRDFAAYRS

In some embodiments, the CD137 (4-1BB) signaling domain is at least 85%, at least 90%, at least 95%, at least 98%, at least 99%, or 100% identical to an exemplary amino acid sequence as provided below:

KRGRKKLLYIFKQPEMRPVQTTQEEDGCSCRFPEEEEGGCEL

In some embodiments, the CD137 (4-1BB) signaling domain is at least 85%, at least 90%, at least 95%, at least 98%, at least 99%, or 100% identical to an exemplary amino acid sequence as provided below:

RFSVVKRGRKKLLYIFKQPEMRPVQTTQEEDGCSCRFPEEEEGGCEL

In some embodiments, the CD134 (OX40) signaling domain is at least 85%, at least 90%, at least 95%, at least 98%, at least 99%, or 100% identical to an exemplary amino acid sequence as provided below:

RRDQRLPPDAHKPPGGGSFRTPIQEEQADAHSTLAKI

### Anaplastic Lymphoma Kinase (ALK) Antibodies

The invention provides anaplastic lymphoma kinase chimeric antigen receptors (ALK CARs) that contain ALK antibody sequences that specifically bind to an ALK polypeptide or antibody-binding fragment thereof. The full-length ALK polypeptide includes an extracellular domain, a hydrophobic stretch corresponding to a single pass transmembrane region, and an intracellular kinase domain.

In some embodiments, the ALK polypeptide is at least 85%, at least 90%, at least 95%, at least 98%, at least 99%, or 100% identical to a full-length ALK protein. In some embodiments, the ALK polypeptide is at least 85%, at least 90%, at least 95%, at least 98%, at least 99%, or 100% identical to a full-length ALK protein in Homo Sapiens. In some embodiments, the ALK polypeptide is at least 85%, at least 90%, at least 95%, at least 98%, at least 99%, or 100% identical to a full-length murine ALK protein. In some embodiments, the ALK polypeptide comprises an ALK extracellular domain. In some embodiments, the ALK polypeptide is at least 85%, at least 90%, at least 95%, at least 98%, at least 99%, or 100% identical to an ALK extracellular domain in Homo Sapiens. In some embodiments, the ALK polypeptide is at least 85%, at least 90%, at least 95%, at least 98%, at least 99%, or 100% identical to a murine ALK extracellular domain. In some embodiments, the ALK polypeptide comprises an ALK intracellular domain. In some embodiments, the ALK polypeptide is at least 85%, at least 90%, at least 95%, at least 98%, at least 99%, or 100% identical to an ALK intracellular domain in Homo Sapiens. In some embodiments, the ALK polypeptide is at least 85%, at least 90%, at least 95%, at least 98%, at least 99%, or 100% identical to a murine ALK intracellular domain.

In some embodiments, the ALK polypeptide comprises an amino acid sequence that is at least 85%, at least 90%, at least 95%, at least 98%, at least 99%, or 100% identical to an ALK amino acid sequence associated with GenBank™ Accession NOs.: BAD92714.1, ACY79563, NP_004295, ACI47591, or EDL38401.1). Human and murine ALK protein sequences are publicly available. One of ordinary skill in the art can identify additional ALK protein sequences, including ALK variants.

An exemplary ALK full-length amino acid sequence from Homo Sapiens is provided below (ALK cytoplasmic portion in bold font):

TASSGGMGAIGLLWLLPLLLSTAAVGSGMGTGQRA
GSPAAGPPLQPREPLSYSRLQRKSLAVDEVVPSLF
RVYARDLLLPPSSSELKAGRPEARGSLALDCAPLL
RLLGPAPGVSWTAGSPAPAEARTLSRVLKGGSVRK
LRRAKQLVLELGEEAILEGCVGPPGEAAVGLLQFN
LSELFSWWIRQGEGRLRIRLMPEKKASEVGREGRL
SAAIRASQPRLLFQIFGTGHSSLESPTNMPSPSPD
YFTWNLTWIMKDSFPFLSHRSRYGLECSFDFPCEL
EYSPPLHDLRNQSWSWRRIPSEEASQMDLLDGPGA
ERSKEMPRGSFLLLNTSADSKHTILSPWMRSSSEH
CTLAVSVHRHLQPSGRYIAQLLPHNEAAREILLMP
TPGKHGWTVLQGRIGRPDNPFRVALEYISSGNRSL
SAVDFFALKNCSEGTSPGSKMALQSSFTCWNGTVL
QLGQACDFHQDCAQGEDESQMCRKLPVGFYCNFED
GFCGWTQGTLSPHTPQWQVRTLKDARFQDHQDHAL
LLSTTDVPASESATVTSATFPAPIKSSPCELRMSW
LIRGVLRGNVSLVLVENKTGKEQGRMVWHVAAYEG
LSLWQWMVLPLLDVSDRFWLQMVAWWGQGSRAIVA
FDNISISLDCYLTISGEDKILQNTAPKSRNLFERN
PNKELKPGENSPRQTPIFDPTVHWLFTTCGASGPH
GPTQAQCNNAYQNSNLSVEVGSEGPLKGIQIWKVP
ATDTYSISGYGAAGGKGGKNTMMRSHGVSVLGIFN
LEKDDMLYILVGQQGEDACPSTNQLIQKVCIGENN
VIEEEIRVNRSVHEWAGGGGGGGGATYVFKMKDGV
PVPLIIAAGGGGRAYGAKTDTFHPERLENNSSVLG
LNGNSGAAGGGGGWNDNTSLLWAGKSLQEGATGGH
SCPQAMKKWGWETRGGFGGGGGGCSSGGGGGGYIG
GNAASNNDPEMDGEDGVSFISPLGILYTPALKVME
GHGEVNIKHYLNCSHCEVDECHMDPESHKVICFCD
HGTVLAEDGVSCIVSPTPEPHLPLSLILSVVTSAL
VAALVLAFSGIMIVYRRKHQELQAMQMELQSPEYK
LSKLRTSTIMTDYNPNYCFAGKTSSISDLKEVPRK
NITLIRGLGHGAFGEVYEGQVSGMPNDPSPLQVAV
KTLPEVCSEQDELDFLMEALIISKFNHQNIVRCIG
VSLQSLPRFILLELMAGGDLKS
FLRETRPRPSQPSSIAMLDLLHVARDIACGCQYLE
ENHFIHRDIAARNCLLTCPGPGRVAKIGDFGMARD
IYRASYYRKGGCAMLPVKWMPPEAFMEGIFTSKTD
TWSFGVLLWEIFSLGYMPYPSKSNQEVLEFVTSGG
RMDPPKNCPGPVYRIMTQCWQHQPEDRPNFAIILE
RIEYCTQDPDVINTALPIEYGPLVEEEEKVPVRPK
DPEGVPPLLVSQQAKREEERSPAAPPPLPTTSSGK
AAKKPTAAEISVRVPRGPAVEGGHVNMAFSQSNPP
SELHKVHGSRNKPTSLWNPTYGSWFTEKPTKKNNP
IAKKEPHDRGNLGLEGSCTVPPNVATGRLPGASLL
LEPSSLTANMKEVPLFRLRHFPCGNVNYGYQQQGL
PLEAATAPGAGHYEDTILKSKNSMNQPGP

An exemplary full-length ALK amino acid sequence from Homo Sapiens is provided below:

G A A A V V A A G T S R R L C S E G R G
A P R C F P A A L W S A T Q S R G R * *
W V R R G R Q D F G R P C P E R P Q L L
P P G P L Q C L R T L R S R G A G E S K
D A A N L R S A G A G I H A Q K F S R Q
T V R S L P A A E R * L E G A Q D G S L
R P R F P P R P G R R A W R S Q K E R K
R R P G Q R A A A G S R R S Q P * K L Q
R L E A A P R G D R P Q L R L R G A G E
D G T Q L P P P F N H S S S S V P S A A
S Y R R G R G T R R G E R E A Q G P S Q
* A Q C A * V S L D S P L S F Q V C F I
* T P A R L R A V G G K Q E T C A H A Q
S S G D Q V E G A A G Y Q G L F R A S S
H L G E S E G * G W A R R A V * T A S S
G G M G A I G L L W L L P L L L S T A A
V G S G M G T G Q R A G S P A A G P P L
Q P R E P L S Y S R L Q R K S L A V D F
V V P S L F R V Y A R D L L L P P S S S
E L K A G R P E A R G S L A L D C A P L
L R L L G P A P G V S W T A G S P A P A
E A R T L S R V L K G G S V R K L R R A
K Q L V L E L G E E A I L E G C V G P P
G E A A V G L L Q F N L S E L F S W W I
R Q G E G R L R I R L M P E K K A S E V
G R E G R L S A A I R A S Q P R L L F Q
I F G T G H S S L E S P T N M P S P S P
D Y F T W N L T W I M K D S F P F L S H
R S R Y G L E C S F D F P C E L E Y S P
P L H D L R N Q S W S W R R I P S E E A
S Q M D L L D G P G A E R S K E M P R G
S F L L L N T S A D S K H T I L S P W M
R S S S E H C T L A V S V H R H L Q P S
G R Y I A Q L L P H N E A A R E I L L M
P T P G K H G W T V L Q G R I G R P D N
P F R V A L E Y I S S G N R S L S A V D
F F A L K N C S E G T S P G S K M A L Q
S S F T C W N G T V L Q L G Q A C D F H
Q D C A Q G E D E S Q M C R K L P V G F
Y C N F E D G F C G W T Q G T L S P H T
P Q W Q V R T L K D A R F Q D H Q D H A
L L L S T T D V P A S E S A T V T S A T
F P A P I K S S P C E L R M S W L I R G
V L R G N V S L V L V E N K T G K E Q G
R M V W H V A A Y E G L S L W Q W M V L
P L L D V S D R F W L Q M V A W W G Q G
S R A I V A F D N I S I S L D C Y L T I
S G E D K I L Q N T A P K S R N L F E R
N P N K E L K P G E N S P R Q T P I F D
P T V H W L F T T C G A S G P H G P T Q
A Q C N N A Y Q N S N L S V E V G S E G
P L K G I Q I W K V P A T D T Y S I S G
Y G A A G G K G G K N T M M R S H G V S
V L G I F N L E K D D M L Y I L V G Q Q
G E D A C P S T N Q L I Q K V C I G E N
N V I E E E I R V N R S V H E W A G G G
G G G G G A T Y V F K M K D G V P V P L
I I A A G G G G R A Y G A K T D T F H P
E R L E N N S S V L G L N G N S G A A G
G G G G W N D N T S L L W A G K S L Q E
G A T G G H S C P Q A M K K W G W E T R
G G F G G G G G G C S S G G G G G G Y I
G G N A A S N N D P E M D G E D G V S F
I S P L G I L Y T P A L K V M E G H G E
V N I K H Y L N C S H C E V D E C H M D
P E S H K V I C F C D H G T V L A E D G
V S C I V S P T P E P H L P L S L I L S
V V T S A L V A A L V L A F S G I M I V
Y R R K H Q E L Q A M Q M E L Q S P E Y
K L S K L R T S T I M T D Y N P N Y C F
A G K T S S I S D L K E V P R K N I T L
I R G L G H G A F G E V Y E G Q V S G M
P N D P S P L Q V A V K T L P E V C S E
Q D E L D F L M E A L I I S K F N H Q N
I V R C I G V S L Q S L P R F I L L E L
M A G G D L K S F L R E T R P R P S Q P
S S L A M L D L L H V A R D I A C G C Q
Y L E E N H F I H R D I A A R N C L L T
C P G P G R V A K I G D F G M A R D I Y
R A S Y Y R K G G C A M L P V K W M P P
E A F M E G I F T S K T D T W S F G V L
L W E I F S L G Y M P Y P S K S N Q E V
L E F V T S G G R M D P P K N C P G P V
Y R I M T Q C W Q H Q P E D R P N F A I
I L E R I E Y C T Q D P D V I N T A L P
I E Y G P L V E E E E K V P V R P K D P
E G V P P L L V S Q Q A K R E E E R S P
A A P P P L P T T S S G K A A K K P T A
A E I S V R V P R G P A V E G G H V N M
A F S Q S N P P S E L H K V H G S R N K
P T S L W N P T Y G S W F T E K P T K K
N N P I A K K E P H D R G N L G L E G S
C T V P P N V A T G R L P G A S L L L E
P S S L T A N M K E V P L F R L R H F P
C G N V N Y G Y Q Q Q G L P L E A A T A
P G A G H Y E D T I L K S K N S M N Q P
G P * A R S H T H F S S L G S L R P W R
R E R Q W L L H K P E T K C H V L F C A
N L F * S T T K K A V F * K C F R K V L
S M G S S Y S F E R R K Y H K N E * * I
Q G P D V V A * G F Y A C L L Y T S L C
F F Q I V C A L L Q C S Q N * L L L C F
I V G V I D V S L P C * C G H E P F E G
R G N G N K G V I C N D *

An exemplary Homo Sapiens ALK amino acid sequence from GenBank™ accession no. NP_004295 is provided below:

| | |
|---|---|
| 1 | MGAIGLLWLL PLLLSTAAVG SGMGTGQRAG |
| | SPAAGPPLQP REPLSYSRLQ RKSLAVDFVV |
| 61 | PSLFRVYARD LLLPPSSSEL KAGRPEARGS |
| | LALDCAPLLR LLGPAPGVSW TAGSPAPAEA |
| 121 | RTLSRVLKGG SVRKLRRAKQ LVLELGEEAI |
| | LEGCVGPPGE AAVGLLQFNL SELFSWWIRQ |
| 181 | GEGRLRIRLM PEKKASEVGR EGRLSAAIRA |
| | SQPRLLFQIF GTGHSSLESP TNMPSPSPDY |
| 241 | FTWNLTWIMK DSFPFLSHRS RYGLECSFDF |
| | PCELEYSPPL HDLRNQSWSW RRIPSEEASQ |
| 301 | MDLLDGPGAE RSKEMPRGSF LLLNTSADSK |
| | HTILSPWMRS SSEHCTLAVS VHRHLQPSGR |
| 361 | YIAQLLPHNE AAREILLMPT PGKHGWTVLQ |
| | GRIGRPDNPF RVALEYISSG NRSLSAVDFF |
| 421 | ALKNCSEGTS PGSKMALQSS FTCWNGTVLQ |
| | LGQACDFHQD CAQGEDESQM CRKLPVGFYC |
| 481 | NFEDGFCGWT QGTLSPHTPQ WQVRTLKDAR |
| | FQDHQDHALL LSTTDVPASE SATVTSATFP |
| 541 | APIKSSPCEL RMSWLIRGVL RGNVSLVLVE |
| | NKTGKEQGRM VWHVAAYEGL SLWQWMVLPL |
| 601 | LDVSDRFWLQ MVAWWGQGSR AIVAFDNISI |
| | SLDCYLTISG EDKILQNTAP KSRNLFERNP |
| 661 | NKELKPGENS PRQTPIFDPT VHWLFTTCGA |
| | SGPHGPTQAQ CNNAYQNSNL SVEVGSEGPL |
| 721 | KGIQIWKVPA TDTYSISGYG AAGGKGGKNT |
| | MMRSHGVSVL GIFNLEKDDM LYILVGQQGE |
| 781 | DACPSTNQLI QKVCIGENNV IEEEIRVNRS |
| | VHEWAGGGGG GGGATYVFKM KDGVPVPLII |
| 841 | AAGGGGRAYG AKTDTFHPER LENNSSVLGL |
| | NGNSGAAGGG GGWNDNTSLL WAGKSLQEGA |
| 901 | TGGHSCPQAM KKWGWETRGG FGGGGGGCSS |
| | GGGGGGYIGG NAASNNDPEM DGEDGVSFIS |
| 961 | PLGILYTPAL KVMEGHGEVN IKHYLNCSHC |
| | EVDECHMDPE SHKVICFCDH GTVLAEDGVS |
| 1021 | CIVSPTPEPH LPLSLILSVV TSALVAALVL |
| | AFSGIMIVYR RKHQELQAMQ MELQSPEYKL |
| 1081 | SKLRTSTIMT DYNPNYCFAG KTSSISDLKE |
| | VPRKNITLIR GLGHGAFGEV YEGQVSGMPN |
| 1141 | DPSPLQVAVK TLPEVCSEQD ELDFLMEALI |
| | ISKFNHQNIV RCIGVSLQSL PRFILLELMA |
| 1201 | GGDLKSFLRE TRPRPSQPSS LAMLDLLHVA |
| | RDIACGCQYL EENHFIHRDI AARNCLLTCP |
| 1261 | GPGRVAKIGD FGMARDIYRA SYYRKGGCAM |
| | LPVKWMPPEA FMEGIFTSKT DTWSFGVLLW |
| 1321 | EIFSLGYMPY PSKSNQEVLE FVTSGGRMDP |
| | PKNCPGPVYR IMTQCWQHQP EDRPNFAIIL |
| 1381 | ERIEYCTQDP DVINTALPIE YGPLVEEEEK |
| | VPVRPKDPEG VPPLLVSQQA KREEERSPAA |
| 1441 | PPPLPTTSSG KAAKKPTAAE ISVRVPRGPA |
| | VEGGHVNMAF SQSNPPSELH KVHGSRNKPT |
| 1501 | SLWNPTYGSW ETEKPTKKNN PIAKKEPHDR |
| | GNLGLEGSCT VPPNVATGRL PGASLLLEPS |
| 1561 | SLTANMKEVP LFRLRHFPCG NVNYGYQQQG |
| | LPLEAATAPG AGHYEDTILK SKNSMNQPGP |

An exemplary Homo Sapiens ALK polypeptide sequence from UniProt Accession No. Q9UM73 is provided below (extracellular domain (amino acids 19-1038) provided in bold font):

MGAIGLLWLLPLLLSTAAVGSGMGTGQRAGSPAAG
PPLQPREPLSYSRLQRKSLAVDFVVPSLFRVYARD
LLLPPSSSELKAGRPEARGSLALDCAPLLRLLGPA
PGVSWTAGSPAPAEARTLSRVLKGGSVRKLRRAKQ
LVLELGEEAILEGCVGPPGEAAVGLLQFNLSELFS
WWIRQGEGRLRIRLMPEKKASEVGREGRLSAAIRA
SQPRLLFQIFGTGHSSLESPTNMPSPSPDYFTWNL
TWIMKDSFPFLSHRSRYGLECSFDFPCELEYSPPL
HDLRNQSWSWRRIPSEEASQMDLLDGPGAERSKEM
PRGSFLLLNTSADSKHTILSPWMRSSSEHCTLAVS
VHRHLQPSGRYIAQLLPHNEAAREILLMPTPGKHG
WTVLQGRIGRPDNPFRVALEYISSGNRSLSAVDFF
ALKNCSEGTSPGSKMALQSSFTCWNGTVLQLGQAC
DFHQDCAQGEDESQMCRKLPVGFYCNFEDGFCGWT
QGTLSPHTPQWQVRTLKDARFQDHQDHALLLSTTD
VPASESATVTSATFPAPIKSSPCELRMSWLIRGVL
RGNVSLVLVENKTGKEQGRMVWHVAAYEGLSLWQW
MVLPLLDVSDRFWLQMVAWWGQGSRAIVAFDNISI
SLDCYLTISGEDKILQNTAPKSRNLFERNPNKELK
PGENSPRQTPIFDPTVHWLFTTCGASGPHGPTQAQ
CNNAYQNSNLSVEVGSEGPLKGIQIWKVPATDTYS
ISGYGAAGGKGGKNTMMRSHGVSVLGIFNLEKDDM
LYILVGQQGEDACPSTNQLIQKVCIGENNVIEEEI
RVNRSVHEWAGGGGGGGGATYVFKMKDGVPVPLII
AAGGGGRAYGAKTDTFHPERLENNSSVLGLNGNSG
AAGGGGGWNDNTSLLWAGKSLQEGATGGHSCPQAM
KKWGWETRGGFGGGGGGCSSGGGGGGYIGGNAASN
NDPEMDGEDGVSFISPLGILYTPALKVMEGHGEVN
IKHYLNCSHCEVDECHMDPESHKVICFCDHGTVLA
EDGVSCIVSPTPEPHLPLSLILSVVTSALVAALVL
AFSGIMIVYRRKHQELQAMQMELQSPEYKLSKLRT
STIMTDYNPNYCFAGKTSSISDLKEVPRKNITLIR
GLGHGAFGEVYEGQVSGMPNDPSPLQVAVKTLPEV
CSEQDELDFLMEALIISKFNHQNIVRCIGVSLQSL
PRFILLELMAGGDLKSFLRETRPRPSQPSSLAMLD
LLHVARDIACGCQYLEENHFIHRDIAARNCLLTCP
GPGRVAKIGDFGMARDIYRASYYRKGGCAMLPVKW
MPPEAFMEGIFTSKTDTWSFGVLLWEIFSLGYMPY
PSKSNQEVLEFVTSGGRMDPPKNCPGPVYRIMTQC
WQHQPEDRPNFAIILERIEYCTQDPDVINTALPIE
YGPLVEEEEKVPVRPKDPEGVPPLLVSQQAKREEE
RSPAAPPPLPTTSSGKAAKKPTAAEISVRVPRGPA
VEGGHVNMAFSQSNPPSELHKVHGSRNKPTSLWNP
TYGSWFTEKPTKKNNPIAKKEPHDRGNLGLEGSCT
VPPNVATGRLPGASLLLEPSSLTANMKEVPLFRLR
HFPCGNVNYGYQQQGLPLEAATAPGAGHYEDTILK
SKNSMNQPGP

An exemplary ALK full-length amino acid sequence from Mus musculus is provided below:

| | |
|---|---|
| 1 | MGQRQSILKR CPEGSFLLLN TSADSKHTIL |
| | SPWMRSSSDH CTLAVSVHRH LQPSGRYVAQ |
| 61 | LLPHNEAGRE ILLVPTPGKH GWTVLQGRVG |
| | RPANPFRVAL EYISSGNRSL SAVDFFALKN |
| 121 | CSEGTSPGSK MALQSSFTCW NGTVLQLGQA |
| | CDFHQDCAQG EDEGQLCSKL PAGFYCNFEN |
| 181 | GFCGWTQSPL SPHMPRWQVR TLRDAHSQGH |
| | QGRALLLSTT DILASEGATV TSATFPAPMK |
| 241 | NSPCELRMSW LIRGVLRGNV SLVLVENKTG |
| | KEQSRTVWHV ATDEGLSLWQ HTVLSLLDVT |
| 301 | DRFWLQIVTW WGPGSRATVG FDNISISLDC |
| | YLTISGEEKM SLNSVPKSRN LFEKNPNKES |
| 361 | KSWANISGPT PIFDPTVHWL FTTCGASGPH |
| | GPTQAQCNNA YQNSNLSVVV GSEGPLKGVQ |
| 421 | IWKVPATDTY SISGYGAAGG KGGKNTMMRS |
| | HGVSVLGIFN LEKGDTLYIL VGQQGEDACP |
| 481 | RANQLIQKVC VGENNVIEEE IRVNRSVHEW |
| | AGGGGGGGGA TYVFKMKDGV PVPLIIAAGG |
| 541 | GGRAYGAKTE TFHPERLESN SSVLGLNGNS |
| | GAAGGGGGWN DNTSLLWAGK SLLEGAAGGH |
| 601 | SCPQAMKKWG WETRGGFGGG GGGGAPQVEE |
| | AEDI |

In some embodiments, the ALK antigen is isolated and/or purified. In some embodiments, the amino acid sequence of the antigen, e.g., the ALK protein, is reverse translated and optimized for expression in mammalian cells. As will be appreciated by a skilled practitioner in the art, optimization of the nucleic acid sequence includes optimization of the codons for expression of a sequence in mammalian cells and RNA optimization (such as RNA stability).

In some embodiments, the ALK polypeptide or antibody-binding fragment thereof (e.g., antigen or antigen protein) is encoded by a polynucleotide.

In some embodiments, the ALK polynucleotide is at least 85%, at least 90%, at least 95%, at least 98%, at least 99%, or 100% identical to a polynucleotide encoding full-length ALK protein. In some embodiments, the ALK polynucleotide is at least 85%, at least 90%, at least 95%, at least 98%, at least 99%, or 100% identical to a polynucleotide encoding full-length ALK protein in Homo Sapiens. In some embodiments, the ALK polynucleotide is at least 85%, at least 90%, at least 95%, at least 98%, at least 99%, or 100% identical to a polynucleotide encoding a full-length murine ALK protein. In some embodiments, the ALK polynucleotide encodes an ALK extracellular domain. In some embodiments, the ALK polynucleotide is at least 85%, at least 90%, at least 95%, at least 98%, at least 99%, or 100% identical to a polypeptide encoding an ALK extracellular domain in Homo Sapiens. In some embodiments, the ALK polynucleotide is at least 85%, at least 90%, at least 95%, at least 98%, at least 99%, or 100% identical to a polypeptide encoding a murine ALK extracellular domain. In some embodiments, the ALK polynucleotide encodes an ALK intracellular domain. In some embodiments, the ALK polynucleotide is at least 85%, at least 90%, at least 95%, at least 98%, at least 99%, or 100% identical to a polynucleotide encoding an ALK intracellular domain in Homo Sapiens. In some embodiments, the ALK polynucleotide is at least 85%, at least 90%, at least 95%, at least 98%, at least 99%, or 100% identical to a polynucleotide encoding a murine ALK intracellular domain. In some embodiments, the ALK polynucleotide is at least 85%, at least 90%, at least 95%, at least 98%, at least 99%, or 100% identical to a polynucleotide encoding an ALK amino acid sequence associated with GenBank™ Accession Nos.: BAD92714.1, ACY79563, NP_004295, NM_007439.2, or ACI47591. Human and murine ALK polynucleotide sequences are publicly available. One of ordinary skill in the art can identify additional ALK polynucleotide sequences, including ALK variants.

An exemplary Homo Sapiens ALK amino acid sequence from GenBank′ accession no. NM_004304 is provided below:

| | |
|---|---|
| 1 | agatgcgatc cagcggctct gggggcggca gcggtggtag cagctggtac ctcccgccgc |
| 61 | ctctgttcgg agggtcgcgg ggcaccgagg tgctttccgg ccgccctctg gtcggccacc |
| 121 | caaagccgcg ggcgctgatg atgggtgagg agggggcggc aagatttcgg gcgcccctgc |
| 181 | cctgaacgcc ctcagctgct gccgccgggg ccgctccagt gcctgcgaac tctgaggagc |
| 241 | cgaggcgccg gtgagagcaa ggacgctgca aacttgcgca gcgcgggggc tgggattcac |
| 301 | gcccagaagt tcagcaggca gacagtccga agccttcccg cagcggagag atagcttgag |
| 361 | ggtgcgcaag acggcagcct ccgccctcgg ttcccgccca gaccgggcag aagagcttgg |
| 421 | aggagccaaa aggaacgcaa aaggcggcca ggacagcgtg cagcagctgg gagccgccgt |
| 481 | tctcagcctt aaaagttgca gagattggag gctgccccga gaggggacag accccagctc |
| 541 | cgactgcggg gggcaggaga ggacggtacc caactgccac ctcccttcaa ccatagtagt |
| 601 | tcctctgtac cgagcgcagc gagctacaga cgggggcgcg gcactcggcg cggagagcgg |
| 661 | gaggctcaag gtcccagcca gtgagcccag tgtgcttgag tgtctctgga ctcgcccctg |
| 721 | agcttccagg tctgtttcat ttagactcct gctcgcctcc gtgcagttgg gggaaagcaa |
| 781 | gagacttgcg cgcacgcaca gtcctctgga gatcaggtgg aaggagccgc tgggtaccaa |
| 841 | ggactgttca gagcctcttc ccatctcggg gagagcgaag ggtgaggctg ggcccggaga |
| 901 | gcagtgtaaa cggcctcctc cggcgggatg ggagccatcg ggctcctgtg gctcctgccg |
| 961 | ctgctgcttt ccacggcagc tgtgggctcc gggatgggga ccggccagcg cgcgggctcc |
| 1021 | ccagctgcgg ggccgccgct gcagccccgg gagccactca gctactcgcg cctgcagagg |
| 1081 | aagagtctgg cagttgactt cgtggtgccc tcgctcttcc gtgtctacgc ccgggaccta |
| 1141 | ctgctgccac catcctcctc ggagctgaag gctggcaggc ccgaggcccg cggctcgcta |
| 1201 | gctctggact gcgccccgct gctcaggttg ctggggccgg cgccgggggt ctcctggacc |
| 1261 | gccggttcac cagccccggc agaggcccgg acgctgtcca gggtgctgaa gggcggctcc |
| 1321 | gtgcgcaagc tccggcgtgc caagcagttg gtgctggagc tgggcgagga ggcgatcttg |
| 1381 | gagggttgcg tcgggccccc cggggaggcg gctgtggggc tgctccagtt caatctcagc |
| 1441 | gagctgttca gttggtggat tcgccaaggc gaagggcgac tgaggatccg cctgatgccc |
| 1501 | gagaagaagg cgtcggaagt gggcagagag ggaaggctgt ccgcggcaat tcgcgcctcc |
| 1561 | cagccccgcc ttctcttcca gatcttcggg actggtcata gctccttgga atcaccaaca |
| 1621 | aacatgcctt ctccttctcc tgattatttt acatggaatc tcacctggat aatgaaagac |
| 1681 | tccttccctt tcctgtctca tcgcagccga tatggtctgg agtgcagctt tgacttcccc |
| 1741 | tgtgagctgg agtattcccc tccactgcat gacctcagga accagagctg gtcctggcgc |
| 1801 | cgcatcccct ccgaggaggc ctcccagatg gacttgctgg atgggcctgg ggcagagcgt |
| 1861 | tctaaggaga tgcccagagg ctcctttctc cttctcaaca cctcagctga ctccaagcac |
| 1921 | accatcctga gtccgtggat gaggagcagc agtgagcact gcacactggc cgtctcggtg |
| 1981 | cacaggcacc tgcagccctc tggaaggtac attgcccagc tgctgcccca caacgaggct |
| 2041 | gcaagagaga tcctcctgat gcccactcca gggaagcatg gttggacagt gctccaggga |
| 2101 | agaatcgggc gtccagacaa cccatttcga gtggccctgg aatacatctc cagtggaaac |
| 2161 | cgcagcttgt ctgcagtgga cttctttgcc ctgaagaact gcagtgaagg aacatcccca |
| 2221 | ggctccaaga tggccctgca gagctccttc acttgttgga atgggacagt cctccagctt |
| 2281 | gggcaggcct gtgacttcca ccaggactgt gcccagggag aagatgagag ccagatgtgc |
| 2341 | cggaaactgc ctgtgggttt ttactgcaac tttgaagatg gcttctgtgg ctggacccaa |
| 2401 | ggcacactgt caccccacac tcctcaatgg caggtcagga ccctaaagga tgcccggttc |
| 2461 | caggaccacc aagaccatgc tctattgctc agtaccactg atgtccccgc ttctgaaagt |
| 2521 | gctacagtga ccagtgctac gtttcctgca ccgatcaaga gctctccatg tgagctccga |
| 2581 | atgtcctggc tcattcgtgg agtcttgagg ggaaacgtgt ccttggtgct agtggagaac |
| 2641 | aaaaccggga aggagcaagg caggatggtc tggcatgtcg ccgcctatga aggcttgagc |
| 2701 | ctgtggcagt ggatggtgtt gcctctcctc gatgtgtctg acaggttctg gctgcagatg |
| 2761 | gtcgcatggt ggggacaagg atccagagcc atcgtggctt ttgacaatat ctccatcagc |
| 2821 | ctggactgct acctcaccat tagcggagag gacaagatcc tgcagaatac agcacccaaa |
| 2881 | tcaagaaacc tgtttgagag aaacccaaac aaggagctga aacccgggga aaattcacca |
| 2941 | agacagaccc ccatctttga ccctacagtt cattggctgt tcaccacatg tggggccagc |
| 3001 | gggccccatg gccccaccca ggcacagtgc aacaacgcct accagaactc caacctgagc |
| 3061 | gtggaggtgg ggagcgaggg ccccctgaaa ggcatccaga tctggaaggt gccagccacc |
| 3121 | gacacctaca gcatctcggg ctacggagct gctggcggga aaggcgggaa gaacaccatg |
| 3181 | atgcggtccc acggcgtgtc tgtgctgggc atcttcaacc tggagaagga tgacatgctg |
| 3241 | tacatcctgg ttgggcagca gggagaggac gcctgcccca gtacaaacca gttaatccag |
| 3301 | aaagtctgca ttggagagaa caatgtgata gaagaagaaa tccgtgtgaa cagaagcgtg |
| 3361 | catgagtggg caggaggcgg aggaggaggg ggtggagcca cctacgtatt taagatgaag |
| 3421 | gatggagtgc cggtgcccct gatcattgca gccggaggtg gtggcagggc ctacggggcc |
| 3481 | aagacagaca cgttccaccc agagagactg gagaataact cctcggttct agggctaaac |
| 3541 | ggcaattccg gagccgcagg tggtggaggt ggctggaatg ataacacttc cttgctctgg |
| 3601 | gccggaaaat ctttgcagga gggtgccacc ggaggacatt cctgccccca ggccatgaag |
| 3661 | aagtgggggt gggagacaag agggggtttc ggagggggtg gaggggggtg ctcctcaggt |
| 3721 | ggaggaggcg gaggatatat aggcggcaat gcagcctcaa acaatgaccc cgaaatggat |
| 3781 | ggggaagatg gggtttcctt catcagtcca ctgggcatcc tgtacacccc agctttaaaa |
| 3841 | gtgatggaag gccacgggga agtgaatatt aagcattatc taaactgcag tcactgtgag |
| 3901 | gtagacgaat gtcacatgga ccctgaaagc cacaaggtca tctgcttctg tgaccacggg |
| 3961 | acggtgctgg ctgaggatgg cgtctcctgc attgtgtcac ccaccccgga gccacacctg |
| 4021 | ccactctcgc tgatcctctc tgtggtgacc tctgccctcg tggccgccct ggtcctggct |
| 4081 | ttctccggca tcatgattgt gtaccgccgg aagcaccagg agctgcaagc catgcagatg |
| 4141 | gagctgcaga gccctgagta caagctgagc aagctccgca cctcgaccat catgaccgac |
| 4201 | tacaacccca actactgctt tgctggcaag acctcctcca tcagtgacct gaaggaggtg |
| 4261 | ccgcggaaaa acatcaccct cattcggggt ctgggccatg gcgcctttgg ggaggtgtat |
| 4321 | gaaggccagg tgtccggaat gcccaacgac ccaagccccc tgcaagtggc tgtgaagacg |
| 4381 | ctgcctgaag tgtgctctga acaggacgaa ctggatttcc tcatggaagc cctgatcatc |
| 4441 | agcaaattca accaccagaa cattgttcgc tgcattgggg tgagcctgca atccctgccc |
| 4501 | cggttcatcc tgctggagct catggcgggg ggagacctca agtccttcct ccgagagacc |
| 4561 | cgccctcgcc cgagccagcc ctcctccctg gccatgctgg accttctgca cgtggctcgg |
| 4621 | gacattgcct gtggctgtca gtatttggag gaaaaccact tcatccaccg agacattgct |
| 4681 | gccagaaact gcctcttgac ctgtccaggc cctggaagag tggccaagat tggagacttc |
| 4741 | gggatggccc gagacatcta cagggcgagc tactatagaa agggaggctg tgccatgctg |
| 4801 | ccagttaagt ggatgccccc agaggccttc atggaaggaa tattcacttc taaaacagac |
| 4861 | acatggtcct ttggagtgct gctatgggaa atcttttctc ttggatatat gccatacccc |
| 4921 | agcaaaagca accaggaagt tctggagttt gtcaccagtg gaggccggat ggacccaccc |
| 4981 | aagaactgcc ctgggcctgt ataccggata atgactcagt gctggcaaca tcagcctgaa |
| 5041 | gacaggccca actttgccat cattttggag aggattgaat actgcaccca ggacccggat |
| 5101 | gtaatcaaca ccgctttgcc gatagaatat ggtccacttg tggaagagga agagaaagtg |
| 5161 | cctgtgaggc ccaaggaccc tgagggggtt cctcctctcc tggtctctca acaggcaaaa |
| 5221 | cgggaggagg agcgcagccc agctgcccca ccacctctgc ctaccacctc ctctggcaag |
| 5281 | gctgcaaaga aacccacagc tgcagagatc tctgttcgag tccctagagg gccggccgtg |
| 5341 | gaagggggac acgtgaatat ggcattctct cagtccaacc ctccttcgga gttgcacaag |
| 5401 | gtccacggat ccagaaacaa gcccaccagc ttgtggaacc caacgtacgg ctcctggttt |
| 5461 | acagagaaac ccaccaaaaa gaataatcct atagcaaaga aggagccaca cgacaggggt |
| 5521 | aacctggggc tggagggaag ctgtactgtc ccacctaacg ttgcaactgg gagacttccg |
| 5581 | ggggcctcac tgctcctaga gccctcttcg ctgactgcca atatgaagga ggtacctctg |
| 5641 | ttcaggctac gtcacttccc ttgtgggaat gtcaattacg gctaccagca acagggcttg |
| 5701 | cccttagaag ccgctactgc ccctggagct ggtcattacg aggataccat tctgaaaagc |
| 5761 | aagaatagca tgaaccagcc tgggccctga gctcggtcgc acactcactt ctcttccttg |
| 5821 | ggatccctaa gaccgtggag gagagagagg caatggctcc ttcacaaacc agagaccaaa |
| 5881 | tgtcacgttt tgttttgtgc caacctattt tgaagtacca ccaaaaaagc tgtattttga |
| 5941 | aaatgcttta gaaaggtttt gagcatgggt tcatcctatt ctttcgaaag aagaaaatat |
| 6001 | cataaaaatg agtgataaat acaaggccca gatgtggttg cataaggttt ttatgcatgt |
| 6061 | ttgttgtata cttccttatg cttctttcaa attgtgtgtg ctctgcttca atgtagtcag |
| 6121 | aattagctgc ttctatgttt catagttggg gtcatagatg tttccttgcc ttgttgatgt |
| 6181 | ggacatgagc catttgaggg gagagggaac ggaaataaag gagttatttg taatgactaa |

An exemplary full-length ALK nucleic acid sequence from Homo Sapiens is provided below:

| | |
|---|---|
| 2 | ggggcggcagcggtggtagcagctggtacctcccgccgcctctgttcggagggtcgcggg 61 |
| 62 | gcaccgaggtgctttccggccgccctctggtcggccacccaaagccgcgggcgctgatga 121 |
| 122 | tgggtgaggagggggcggcaagatttcgggcgcccctgccctgaacgccctcagctgctg 181 |
| 182 | ccgccggggccgctccagtgcctgcgaactctgaggagccgaggcgccggtgagagcaag 241 |
| 242 | gacgctgcaaacttgcgcagcgcgggggctgggattcacgcccagaagttcagcaggcag 301 |
| 302 | acagtccgaagccttcccgcagcggagagatagcttgagggtgcgcaagacggcagcctc 361 |
| 362 | cgccctcggttcccgcccagaccgggcagaagagcttggaggagccaaaaggaacgcaaa 421 |
| 422 | aggcggccaggacagcgtgcagcagctgggagccgccgttctcagccttaaaagttgcag 481 |
| 482 | agattggaggctgccccgagaggggacagaccccagctccgactgcggggggcaggagag 541 |
| 542 | gacggtacccaactgccacctcccttcaaccatagtagttcctctgtaccgagcgcagcg 601 |
| 602 | agctacagacgggggcgcggcactcggcgcggagagcgggaggctcaaggtcccagccag 661 |
| 662 | tgagcccagtgtgcttgagtgtctctggactcgcccctgagcttccaggtctgtttcatt 721 |
| 722 | tagactcctgctcgcctccgtgcagttgggggaaagcaagagacttgcgcgcacgcacag 781 |
| 782 | tcctctggagatcaggtggaaggagccgctgggtaccaaggactgttcagagcctcttcc 841 |
| 842 | catctcggggagagcgaagggtgaggctgggcccggagagcagtgtaaacggcctcctcc 901 |
| 902 | ggcgggatgggagccatcgggctcctgtggctcctgccgctgctgctttccacggcagct 961 |
| 962 | gtgggctccgggatggggaccggccagcgcgcgggctccccagctgcggggccgccgctg 1021 |
| 1022 | cagccccgggagccactcagctactcgcgcctgcagaggaagagtctggcagttgacttc 1081 |
| 1082 | gtggtgccctcgctcttccgtgtctacgcccgggacctactgctgccaccatcctcctcg 1141 |
| 1142 | gagctgaaggctggcaggcccgaggcccgcggctcgctagctctggactgcgccccgctg 1201 |
| 1202 | ctcaggttgctggggccggcgccgggggtctcctggaccgccggttcaccagccccggca 1261 |
| 1262 | gaggcccggacgctgtccagggtgctgaagggcggctccgtgcgcaagctccggcgtgcc 1321 |
| 1322 | aagcagttggtgctggagctgggcgaggaggcgatcttggagggttgcgtcgggcccccc 1381 |
| 1382 | ggggaggcggctgtggggctgctccagttcaatctcagcgagctgttcagttggtggatt 1441 |
| 1442 | cgccaaggcgaagggcgactgaggatccgcctgatgcccgagaagaaggcgtcggaagtg 1501 |
| 1502 | ggcagagagggaaggctgtccgcggcaattcgcgcctcccagccccgccttctcttccag 1561 |
| 1562 | atcttcgggactggtcatagctccttggaatcaccaacaaacatgccttctccttctcct 1621 |
| 1622 | gattattttacatggaatctcacctggataatgaaagactccttccctttcctgtctcat 1681 |
| 1682 | cgcagccgatatggtctggagtgcagctttgacttcccctgtgagctggagtattcccct 1741 |
| 1742 | ccactgcatgacctcaggaaccagagctggtcctggcgccgcatcccctccgaggaggcc 1801 |
| 1802 | tcccagatggacttgctggatgggcctggggcagagcgttctaaggagatgcccagaggc 1861 |
| 1862 | tcctttctccttctcaacacctcagctgactccaagcacaccatcctgagtccgtggatg 1921 |
| 1922 | aggagcagcagtgagcactgcacactggccgtctcggtgcacaggcacctgcagccctct 1981 |
| 1982 | ggaaggtacattgcccagctgctgccccacaacgaggctgcaagagagatcctcctgatg 2041 |
| 2042 | cccactccagggaagcatggttggacagtgctccagggaagaatcgggcgtccagacaac 2101 |
| 2102 | ccatttcgagtggccctggaatacatctccagtggaaaccgcagcttgtctgcagtggac 2161 |
| 2162 | ttctttgccctgaagaactgcagtgaaggaacatccccaggctccaagatggccctgcag 2221 |
| 2222 | agctccttcacttgttggaatgggacagtcctccagcttgggcaggcctgtgacttccac 2281 |
| 2282 | caggactgtgcccagggagaagatgagagccagatgtgccggaaactgcctgtgggtttt 2341 |
| 2342 | tactgcaactttgaagatggcttctgtggctggacccaaggcacactgtcaccccacact 2401 |
| 2402 | cctcaatggcaggtcaggaccctaaaggatgcccggttccaggaccaccaagaccatgct 2461 |
| 2462 | ctattgctcagtaccactgatgtccccgcttctgaaagtgctacagtgaccagtgctacg 2521 |
| 2522 | tttcctgcaccgatcaagagctctccatgtgagctccgaatgtcctggctcattcgtgga 2581 |
| 2582 | gtcttgaggggaaacgtgtccttggtgctagtggagaacaaaaccgggaaggagcaaggc 2641 |
| 2642 | aggatggtctggcatgtcgccgcctatgaaggcttgagcctgtggcagtggatggtgttg 2701 |
| 2702 | cctctcctcgatgtgtctgacaggttctggctgcagatggtcgcatggtggggacaagga 2761 |
| 2762 | tccagagccatcgtggcttttgacaatatctccatcagcctggactgctacctcaccatt 2821 |
| 2822 | agcggagaggacaagatcctgcagaatacagcacccaaatcaagaaacctgtttgagaga 2881 |
| 2882 | aacccaaacaaggagctgaaacccggggaaaattcaccaagacagacccccatctttgac 2941 |
| 2942 | cctacagttcattggctgttcaccacatgtggggccagcgggccccatggccccacccag 3001 |
| 3002 | gcacagtgcaacaacgcctaccagaactccaacctgagcgtggaggtggggagcgagggc 3061 |
| 3062 | cccctgaaaggcatccagatctggaaggtgccagccaccgacacctacagcatctcgggc 3121 |
| 3122 | tacggagctgctggcgggaaaggcgggaagaacaccatgatgcggtcccacggcgtgtct 3181 |
| 3182 | gtgctgggcatcttcaacctggagaaggatgacatgctgtacatcctggttgggcagcag 3241 |
| 3242 | ggagaggacgcctgccccagtacaaaccagttaatccagaaagtctgcattggagagaac 3301 |
| 3302 | aatgtgatagaagaagaaatccgtgtgaacagaagcgtgcatgagtgggcaggaggcgga 3361 |
| 3362 | ggaggagggggtggagccacctacgtatttaagatgaaggatggagtgccggtgcccctg 3421 |
| 3422 | atcattgcagccggaggtggtggcagggcctacggggccaagacagacacgttccaccca 3481 |
| 3482 | gagagactggagaataactcctcggttctagggctaaacggcaattccggagccgcaggt 3541 |
| 3542 | ggtggaggtggctggaatgataacacttccttgctctgggccggaaaatctttgcaggag 3601 |
| 3602 | ggtgccaccggaggacattcctgcccccaggccatgaagaagtgggggtgggagacaaga 3661 |
| 3662 | gggggtttcggagggggtggaggggggtgctcctcaggtggaggaggcggaggatatata 3721 |
| 3722 | ggcggcaatgcagcctcaaacaatgaccccgaaatggatggggaagatggggtttccttc 3781 |
| 3782 | atcagtccactgggcatcctgtacaccccagctttaaaagtgatggaaggccacggggaa 3841 |
| 3842 | gtgaatattaagcattatctaaactgcagtcactgtgaggtagacgaatgtcacatggac 3901 |
| 3902 | cctgaaagccacaaggtcatctgcttctgtgaccacgggacggtgctggctgaggatggc 3961 |
| 3962 | gtctcctgcattgtgtcacccaccccggagccacacctgccactctcgctgatcctctct 4021 |
| 4022 | gtggtgacctctgccctcgtggccgccctggtcctggctttctccggcatcatgattgtg 4081 |
| 4082 | taccgccggaagcaccaggagctgcaagccatgcagatggagctgcagagccctgagtac 4141 |
| 4142 | aagctgagcaagctccgcacctcgaccatcatgaccgactacaaccccaactactgcttt 4201 |
| 4202 | gctggcaagacctcctccatcagtgacctgaaggaggtgccgcggaaaaacatcaccctc 4261 |
| 4262 | attcggggtctgggccatggcgcctttggggaggtgtatgaaggccaggtgtccggaatg 4321 |
| 4322 | cccaacgacccaagccccctgcaagtggctgtgaagacgctgcctgaagtgtgctctgaa 4381 |
| 4382 | caggacgaactggatttcctcatggaagccctgatcatcagcaaattcaaccaccagaac 4441 |
| 4442 | attgttcgctgcattggggtgagcctgcaatccctgccccggttcatcctgctggagctc 4501 |
| 4502 | atggcggggggagacctcaagtccttcctccgagagacccgccctcgcccgagccagccc 4561 |
| 4562 | tcctccctggccatgctggaccttctgcacgtggctcgggacattgcctgtggctgtcag 4621 |
| 4622 | tatttggaggaaaaccacttcatccaccgagacattgctgccagaaactgcctcttgacc 4681 |
| 4682 | tgtccaggccctggaagagtggccaagattggagacttcgggatggcccgagacatctac 4741 |
| 4742 | agggcgagctactatagaaagggaggctgtgccatgctgccagttaagtggatgccccca 4801 |
| 4802 | gaggccttcatggaaggaatattcacttctaaaacagacacatggtcctttggagtgctg 4861 |
| 4862 | ctatgggaaatcttttctcttggatatatgccataccccagcaaaagcaaccaggaagtt 4921 |
| 4922 | ctggagtttgtcaccagtggaggccggatggacccacccaagaactgccctgggcctgta 4981 |
| 4982 | taccggataatgactcagtgctggcaacatcagcctgaagacaggcccaactttgccatc 5041 |
| 5042 | attttggagaggattgaatactgcacccaggacccggatgtaatcaacaccgctttgccg 5101 |
| 5102 | atagaatatggtccacttgtggaagaggaagagaaagtgcctgtgaggcccaaggaccct 5161 |
| 5162 | gagggggttcctcctctcctggtctctcaacaggcaaaacgggaggaggagcgcagccca 5221 |
| 5222 | gctgccccaccacctctgcctaccacctcctctggcaaggctgcaaagaaacccacagct 5281 |
| 5282 | gcagagatctctgttcgagtccctagagggccggccgtggaagggggacacgtgaatatg 5341 |
| 5342 | gcattctctcagtccaaccctccttcggagttgcacaaggtccacggatccagaaacaag 5401 |
| 5402 | cccaccagcttgtggaacccaacgtacggctcctggtttacagagaaacccaccaaaaag 5461 |
| 5462 | aataatcctatagcaaagaaggagccacacgacaggggtaacctggggctggagggaagc 5521 |
| 5522 | tgtactgtcccacctaacgttgcaactgggagacttccgggggcctcactgctcctagag 5581 |
| 5582 | ccctcttcgctgactgccaatatgaaggaggtacctctgttcaggctacgtcacttccct 5641 |
| 5642 | tgtgggaatgtcaattacggctaccagcaacagggcttgcccttagaagccgctactgcc 5701 |
| 5702 | cctggagctggtcattacgaggataccattctgaaaagcaagaatagcatgaaccagcct 5761 |
| 5762 | gggccctgagctcggtcgcacactcacttctcttccttgggatccctaagaccgtggagg 5821 |
| 5822 | agagagaggcaatggctccttcacaaaccagagaccaaatgtcacgttttgttttgtgcc 5881 |
| 5882 | aacctattttgaagtaccaccaaaaaagctgtattttgaaaatgctttagaaaggttttg 5941 |
| 5942 | agcatgggttcatcctattctttcgaaagaagaaaatatcataaaaatgagtgataaata 6001 |
| 6002 | caaggcccagatgtggttgcataaggtttttatgcatgtttgttgtatacttccttatgc 6061 |
| 6062 | ttctttcaaattgtgtgtgctctgcttcaatgtagtcagaattagctgcttctatgtttc 6121 |
| 6122 | atagttggggtcatagatgtttccttgccttgttgatgtggacatgagccatttgagggg 6181 |
| 6182 | agagggaacggaaataaaggagttatttgtaatgactaaaa 6222 |

An exemplary Mus musculus ALK nucleic acid sequence from GenBank™ accession no. NM_007439.2 is provided below:

| | |
|---|---|
| 1 | gtgttcacgc ccagaagttc agcgggcagg gtgatcgatc cgaagacttc ctgcagcgga |
| 61 | ggtcacttga gggggcgcta gaaagcagcc ccctccggtg gtccttgcct agacctggga |
| 121 | aggagcgcag aggaggtgac aggagcggag gacgtgggca agacagtgac cgactcggag |
| 181 | ccacggttca cagcctggaa agttgcagaa gattggaagc taagaggaga gctctggtcg |
| 241 | ccgagggctc cttgaacggt acctaattgc cacctccctg gtccctgagc aaaggcctct |
| 301 | acaaatgggg cgcagcacgg cgagaggcgc aggatccagc tgttgagccc agggtgtctc |
| 361 | actgtctccg aactaccccc tgactttgtc ttccgttttg ctgagaaccc ttctcgcctc |
| 421 | cttgtagctt gggaaaagca agggcgctct atagtgtaca cacagtccct gagatctagt |
| 481 | ggaaggagcc attcaggacc aaggactatt tggagccctt tcctgtttgg gggagagtga |
| 541 | agggcgaggc tggaccagca agggaaggga gactagtgta aactcgccct ccagcgggat |
| 601 | gggagctgct gggttcctgt ggctgctgcc tccactgctt ttggcagcag cctcgtactc |
| 661 | cggagctgca accgatcagc gcgcgggttc cccagcctca gggcctcctc tgcagccccg |
| 721 | ggagccgctc agttattcgc gcctgcagag gaagagtctg gcagtggact tcgttgtacc |
| 781 | ctcgctcttc cgcgtctatg cccgagacct gctgctaccg cagccacggt ccccctcgga |
| 841 | gcccgaggct ggcgggctgg aggcgcgggg atcactggcc ctggattgtg agcctctgct |
| 901 | caggctgctg gggccactgc ctggaatctc ctgggcagat ggagccagtt ctcctagtcc |
| 961 | cgaggcgggt ccgacgctgt ccagggtgct gaagggaggc tcggtgcgca agctcaggcg |
| 1021 | tgccaaacag ctggtgctgg agctgggcga ggagacgatt cttgaaggct gtattggtcc |
| 1081 | cccagaggag gtagcggctg tggggatact ccagttcaac ctcagcgagc tgttcagctg |
| 1141 | gtggattctc cacggcgaag ggaggctgag gatccgcctg atgcctgaga agaaggcatc |
| 1201 | ggaagtgggc agggagggaa ggctatccag tgcgatccga gcctcccagc cccgccttct |
| 1261 | cttccagatc ttcgggacgg gacacagctc catggagtca ccctcagaaa cgccttctcc |
| 1321 | tcctggtacc ttcatgtgga atctgacctg gacgatgaaa gactccttcc ctttcctttc |
| 1381 | ccaccgcagt cgatatggtc tggagtgcag ctttgacttc ccctgtgagc tggaatattc |
| 1441 | tcctcccctg cacaaccacg ggaatcagag ctggtcctgg cgccatgtgc cctccgagga |
| 1501 | ggcctcgagg atgaacttgc tggatgggcc agaggcagag cattctcaag agatgcccag |
| 1561 | aggctccttc ctcctcctga acacctctgc agattccaag cataccattc tgagcccatg |
| 1621 | gatgaggagc agtagtgatc actgcacact ggctgtctcc gtgcacagac atctacagcc |
| 1681 | ttcggggaga tatgttgccc agctcctacc ccacaacgaa gctggaagag agattctttt |
| 1741 | ggtgcccacc ccagggaagc atggctggac agtgctgcag gggagagtcg ggcgcccagc |
| 1801 | aaacccattt cgagtggctc tggaatacat ctccagtggc aaccggagct tgtcggcggt |
| 1861 | ggatttcttt gccctgaaga actgcagtga agggacatcc ccaggctcca agatggcatt |
| 1921 | gcagagttcc ttcacttgtt ggaatgggac cgtcctccag ctcgggcaag cctgtgattt |
| 1981 | ccaccaggac tgtgcccaag gagaagatga gggccagctg tgcagtaaac ttcctgctgg |
| 2041 | attttactgt aactttgaaa atggcttctg tggctggacc caaagtccac tctcacccca |
| 2101 | tatgccccgg tggcaagtga ggaccctaag agatgcccat tcccagggcc accaaggccg |
| 2161 | tgccctgttg ctcagcacca ctgacatcct cgcttctgaa ggtgcaacag tgaccagtgc |
| 2221 | caccttccct gcaccaatga aaaattctcc ttgtgagctc cgcatgtcct ggctcatccg |
| 2281 | cggggttttg agaggaaacg tatctctggt gctggtggag aacaaaaccg gaaaggagca |
| 2341 | aagccggact gtctggcatg tcgccactga cgaaggctta agcctgtggc agcatacagt |
| 2401 | gctgtccctc ctcgatgtga ctgacaggtt ctggctgcag atagtcacat ggtggggtcc |
| 2461 | aggatccagg gcaaccgtgg gatttgacaa catttccatc agcctcgact gctaccttac |
| 2521 | catcagtgga gaggagaaaa tgtccctgaa ttcagtaccc aaatctagaa atctgtttga |
| 2581 | gaaaaaccca aacaaggagt caaaatcctg ggcaaacata tcaggaccaa ctcccatctt |
| 2641 | cgaccctaca gttcactggc tgttcaccac gtgtggggcc agtggacctc atggccccac |
| 2701 | ccaggcacag tgcaacaacg cctaccagaa ttccaacttg agcgtggtgg tgggaagtga |
| 2761 | agggcccttg aagggagtcc agatttggaa agtgccagct actgacacct acagtatctc |
| 2821 | gggctacgga gcagctggcg ggaaaggtgg gaaaaacacc atgatgcggt cccatggcgt |
| 2881 | gtctgtcctg ggcatcttca atctggagaa aggtgacaca ctctacatcc ttgtcggtca |
| 2941 | gcaaggggag gatgcctgtc ccagggcaaa ccaactaatc cagaaagtct gtgtgggtga |
| 3001 | gaacaatgtc atagaagaag agatccgagt gaacagaagc gtgcacgagt gggcaggagg |
| 3061 | aggaggaggt gggggtggag ccacctacgt gtttaagatg aaagatggcg tgcctgtacc |
| 3121 | cctgatcatt gcagctggtg gtggtggcag ggcctatggg gccaagacag aaacgttcca |
| 3181 | cccagagaga ctggagagta actcctcggt tctagggctg aacggcaatt ccggagccgc |
| 3241 | aggtggtgga ggcggctgga atgataacac ttccttgctc tgggccggaa agtctttgct |
| 3301 | ggagggtgcc gccggaggac attcctgccc ccaggccatg aagaagtggg ggtgggagac |
| 3361 | aagagggggt ttcggagggg gtggaggggg gtgctcctca ggtggaggag gcggaggata |
| 3421 | tataggtggc aacgcagcat caaacaatga ccccgaaatg gatggggaag atggggtttc |
| 3481 | cttcatcagt ccattgggta tcctgtacac cccggcctta aaagtgatgg agggccacgg |
| 3541 | ggaagtgaat atcaagcatt atctaaactg cagtcactgc gaggtagacg aatgtcacat |
| 3601 | ggaccccgag agccacaaag tcatctgctt ctgtgatcat gggaccgtgc tggctgatga |
| 3661 | tggtgtctcc tgcattgtgt cacccacccc ggagccccac ctgccgctct cattgatcct |
| 3721 | ctccgtcgtg acctctgccc tggtggctgc ccttgttctg gcattctccg gcatcatgat |
| 3781 | tgtgtaccgt cggaagcacc aggagttgca ggctatgcag atggaactgc agagccccga |
| 3841 | gtataagctg agcaagctac ggacctcgac catcatgacc gactacaacc ccaactactg |
| 3901 | cttcgctggc aagacttcct ccatcagtga cctgaaagaa gtgccacgga aaaacatcac |
| 3961 | actcatccgg ggcctaggcc atggcgcatt tggggaggtg tatgaaggcc aggtgtctgg |
| 4021 | aatgcccaat gacccaagcc ctctacaagt ggctgtaaag acgctgccag aagtgtgttc |
| 4081 | agaacaagat gagctggact ttctcatgga agctctgatc atcagcaaat tcaaccacca |
| 4141 | gaatattgtt cgctgcatcg gggtgagtct acaagccctg ccccgcttca tcctgctgga |
| 4201 | actcatggct ggcggagacc tcaagtcctt cctcagggag acacgccctc gcccgaacca |
| 4261 | acccacctcc ctggccatgc tggaccttct gcatgtggct cgggacattg cctgtggctg |
| 4321 | tcagtaccta gaggagaatc actttatcca ccgggatatt gctgctagaa actgtctgtt |
| 4381 | gacctgccca ggagctggaa gaatagcaaa gattggagac tttgggatgg cccgagatat |
| 4441 | ctacagggcc agctactacc gaaagggagg ctgcgccatg ctgccggtca agtggatgcc |
| 4501 | ccctgaagcc ttcatggaag ggatatttac ctctaaaaca gacacatggt cttttggagt |
| 4561 | gttgctatgg gaaatatttt ctcttggata tatgccgtac cccagcaaga gcaaccagga |
| 4621 | agttctggag tttgtcacca gcggaggacg gatggacccg cctaagaact gccccgggcc |
| 4681 | tgtataccgg ataatgacgc agtgctggca gcatcagcct gaagacagac ccaacttcgc |
| 4741 | catcattttg gagaggatcg aatactgcac ccaggacccc gatgtgatca acacagctct |
| 4801 | gcccatcgaa tacggtccag tagtagaaga ggaggagaaa gtgcccatgc gccccaaaga |
| 4861 | ccccgagggg atgccacctt tgctggtgtc tccccagcct gcgaagcacg aggaggcgtc |
| 4921 | cgcagctccc cagcccgcag ccctgacggc accaggccca tcggtgaaga agcccccggg |
| 4981 | tgcgggtgcg ggcgcgggcg cgggtgcggg tgccggcccg gtgccccgag gtgcggccga |
| 5041 | tcggggccac gtgaacatgg ctttctctca gcccaaccct cccccggagc tgcacaaagg |
| 5101 | cccgggatcc agaaacaagc cgaccagcct gtggaacccc acctacggct cgtggttcac |
| 5161 | cgagaagcct gccaaaaaga cccatcctcc gccaggcgcc gagccgcagg cgcgggcagg |
| 5221 | agcggccgag ggtggctgga ccgggccggg cgcggggccc cgcagagccg aggcagcgct |
| 5281 | gctgctagag ccatcggcgc tgagcgccac catgaaggag gtgccgctgt tcaggctgcg |
| 5341 | ccacttcccc tgcggcaatg tcaactatgg ttaccagcaa cagggtctcc ccttggaagc |
| 5401 | cacagccgcg ccaggggaca ccatgctgaa aagcaagaat aaggtcaccc agccggggcc |
| 5461 | ctgagccctg tactccacta gcttctcctc ctggcggagc cggagcccac ccagagggag |
| 5521 | atggacagga tggctccacc acaaacccaa gaccaaaact ttcatttttg tgccaacttg |
| 5581 | ttttgaagtg ccacatttta aaaaaaggaa acttgtgttt ttaagatgtg ttagaaggtt |
| 5641 | ttttgagcat gggttcatct atcctctcaa aagaagaaaa tgccattctt taaaaaagaa |
| 5701 | aaaaaagcaa tcagtgcaag gcccagattg gttgcgccaa gttttcgtgc atggtctgct |
| 5761 | gtacagtccc ctaaggcttc tttccgattt ttgtgtgcgc tctgcttccg cgtagtcaga |
| 5821 | aatagctgct tccatgtctc atagggggag tcctaggtgt ttcctttgcc ttatgaatat |
| 5881 | gaaccactcg aggggcgggc gagggaacag aaataaag |

In some embodiments, fusion proteins comprising the ALK antigen polypeptides are described herein. In some embodiments, the ALK polypeptide can be fused to any heterologous amino acid sequence to form a fusion protein. For example, a fusion protein includes an ALK protein fused to a heterologous protein. In some embodiments, the fusion protein is an ALK protein fused to a nucleophosmin (NPM) protein. In some embodiments, the NPM-ALK fusion protein is at least 85%, at least 90%, at least 95%, at least 98%, at least 99%, or 100% identical to a NPM-ALK fusion protein in Homo Sapiens. In some embodiments, the NPM-ALK fusion protein is at least 85%, at least 90%, at least 95%, at least 98%, at least 99%, or 100% identical to an exemplary NPM-ALK fusion protein amino acid sequence from Homo Sapiens as provided below (ALK cytoplasmic portion in bold font):

MEDSMDMDMSPLRPQNYLFGCELKADKDYHFKVDN
DENEHQLSLRTVSLGAGAKDELHIVEAEAMNYEGS
PIKVTLATLKMSVQPTVSLGGFEITPPVVLRLKCG
SGPVHISGQHLVVYRRKHQELQAMOMELQSPEYKL
SKLRTSTIMTDYNPNYCFAGKTSSISDLKEVPRKN
NTLIRGLGHGAFGEVYEGQVSGMPNDPSPLQVAVK
TLPEVCSEQDELDFLMEALIISKFNHQNIVRCIGV
SLQSLPRFILLELMAGGDLKSFLRETRPRPSQPSS
IAMLDLLHVARDIACGCQYLEENHFIHRDIAARNC
LLTCPGPGRVAKIGDFGMARDIYRASYYRKGGCAM
LPVKWMPPEAFMEGIFTSKTDTWSFGVLLWEIFS
LGYMPYPSKSNQEVLEFVTSGGRMDPPKNCPGP
VYRIMTQCWQHQPEDRPNFAIILERIEYCTQDPDV
INTALPIEYGPLVEEEEKVPVRPKDPEGVPPLLVS
QQAKREEERSPAAPPPLPTTSSGKAAKKPTAAEVS
VRVPRGPAVEGGHVNMAFSQSNPPSELHRVHGSRN
KPTSLWNPTYGSWFTEKPTKKNNPIAKKEPHERGN
LGLEGSCTVPPNVATGRLPGASLLLEPSSLTANMK
EVPLFRLRHFPCGNVNYGYOOQGLPLEAATAPGAG
HYEDTILKSKNSMNQPGP

In some embodiments, the NPM-ALK fusion protein is at least 85%, at least 90%, at least 95%, at least 98%, at least 99%, or 100% identical to an exemplary NPM-ALK fusion protein amino acid sequence from Homo Sapiens (GenBank: AAA58698.1) as provided below:

| | |
|---|---|
| 1 | MEDSMDMDMS PLRPQNYLFG CELKADKDYH |
| | FKVDNDENEH QLSLRTVSLG AGAKDELHIV |
| 61 | EAEAMNYEGS PIKVTLATLK MSVQPTVSLG |
| | GFEITPPVVL RLKCGSGPVH ISGQHLVVYR |
| 121 | RKHQELQAMQ MELQSPEYKL SKLRTSTIMT |
| | DYNPNYCFAG KTSSISDLKE VPRKNITLIR |
| 181 | GLGHGAFGEV YEGQVSGMPN DPSPLQVAVK |
| | TLPEVCSEQD ELDFLMEALI ISKFNHQNIV |
| 241 | RCIGVSLQSL PRFILLELMA GGDLKSFLRE |
| | TRPRPSQPSS LAMLDLLHVA RDIACGCQYL |
| 301 | EENHFIHRDI AARNCLLTCP GPGRVAKIGD |
| | FGMARDIYRA SYYRKGGCAM LPVKWMPPEA |
| 361 | FMEGIFTSKT DTWSFGVLLW EIFSLGYMPY |
| | PSKSNQEVLE FVTSGGRMDP PKNCPGPVYR |
| 421 | IMTQCWQHQP EDRPNFAIIL ERIEYCTQDP |
| | DVINTALPIE YGPLVEEEEK VPVRPKDPEG |
| 481 | VPPLLVSQQA KREEERSPAA PPPLPTTSSG |
| | KAAKKPTAAE VSVRVPRGPA VEGGHVNMAF |
| 541 | SQSNPPSELH KVHGSRNKPT SLWNPTYGSW |
| | FTEKPTKKNN PIAKKEPHDR GNLGLEGSCT |
| 601 | VPPNVATGRL PGASLLLEPS SLTANMKEVP |
| | LFRLRHFPCG NVNYGYQQQG LPLEAATAPG |
| 661 | AGHYEDTILK SKNSMNQPGP |

In some embodiments, the NPM-ALK fusion protein is at least 85%, at least 90%, at least 95%, at least 98%, at least 99%, or 100% identical to an exemplary NPM-ALK fusion protein amino acid sequence from Homo Sapiens as provided below:

M E D S M D M D M S P L R P Q N Y L F G
C E L K A D K D Y H F K V D N D E N E H
Q L S L R T V S L G A G A K D E L H I V
E A E A M N Y E G S P I K V T L A T L K
M S V Q P T V S L G G F E I T P P V V L
R L K C G S G P V H I S G Q H L V V Y R
R K H Q E L Q A M Q M E L Q S P E Y K L
S K L R T S T I M T D Y N P N Y C F A G
K T S S I S D L K E V P R K N I T L I R
G L G H G A F G E V Y E G Q V S G M P N
D P S P L Q V A V K T L P E V C S E Q D
E L D F L M E A L I I S K F N H Q N I V
R C I G V S L Q S L P R F I L L E L M A
G G D L K S F L R E T R P R P S Q P S S
L A M L D L L H V A R D I A C G C Q Y L
E E N H F I H R D I A A R N C L L T C P
G P G R V A K I G D F G M A R D I Y R A
S Y Y R K G G C A M L P V K W M P P E A
F M E G I F T S K T D T W S F G V L L W
E I F S L G Y M P Y P S K S N Q E V L E
F V T S G G R M D P P K N C P G P V Y R
I M T Q C W Q H Q P E D R P N F A I I L
E R I E Y C T Q D P D V I N T A L P I E
Y G P L V E E E E K V P V R P K D P E G
V P P L L V S Q Q A K R E E E R S P A A
P P P L P T T S S G K A A K K P T A A E
V S V R V P R G P A V E G G H V N M A F
S Q S N P P S E L H K V H G S R N K P T
S L W N P T Y G S W F T E K P T K K N N
P I A K K E P H D R G N L G L E G S C T
V P P N V A T G R L P G A S L L L E P S
S L T A N M K E V P L F R L R H F P C G
N V N Y G Y Q Q Q G L P L E A A T A P G
A G H Y E D T I L K S K N S M N Q P G P

In some embodiments, the NPM-ALK fusion protein is encoded by a nucleic acid sequence that is at least 85%, at least 90%, at least 95%, at least 98%, at least 99%, or 100% identical to the exemplary nucleic acid sequence from Homo Sapiens as provided below:

| | |
|---|---|
| 1 | atggaagattcgatggacatggacatgagccccctgaggccccagaactatcttttcggt 60 |
| 61 | tgtgaactaaaggccgacaaagattatcactttaaggtggataatgatgaaaatgagcac 120 |
| 121 | cagttatctttaagaacggtcagtttaggggctggtgcaaaggatgagttgcacattgtt 180 |
| 181 | gaagcagaggcaatgaattacgaaggcagtccaattaaagtaacactggcaactttgaaa 240 |
| 241 | atgtctgtacagccaacggtttcccttgggggctttgaaataacaccaccagtggtctta 300 |
| 301 | aggttgaagtgtggttcagggccagtgcatattagtggacagcacttagtagtgtaccgc 360 |
| 361 | cggaagcaccaggagctgcaagccatgcagatggagctgcagagccctgagtacaagctg 420 |
| 421 | agcaagctccgcacctcgaccatcatgaccgactacaaccccaactactgctttgctggc 480 |
| 481 | aagacctcctccatcagtgacctgaaggaggtgccgcggaaaaacatcaccctcattcgg 540 |
| 541 | ggtctgggccatggcgcctttggggaggtgtatgaaggccaggtgtccggaatgcccaac 600 |
| 601 | gacccaagccccctgcaagtggctgtgaagacgctgcctgaagtgtgctctgaacaggac 660 |
| 661 | gaactggatttcctcatggaagccctgatcatcagcaaattcaaccaccagaacattgtt 720 |
| 721 | cgctgcattggggtgagcctgcaatccctgccccggttcatcctgctggagctcatggcg 780 |
| 781 | gggggagacctcaagtccttcctccgagagacccgccctcgcccgagccagccctcctcc 840 |
| 841 | ctggccatgctggaccttctgcacgtggctcgggacattgcctgtggctgtcagtatttg 900 |
| 901 | gaggaaaaccacttcatccaccgagacattgctgccagaaactgcctcttgacctgtcca 960 |
| 961 | ggccctggaagagtggccaagattggagacttcgggatggcccgagacatctacagggcg 1020 |
| 1021 | agctactatagaaagggaggctgtgccatgctgccagttaagtggatgcccccagaggcc 1080 |
| 1081 | ttcatggaaggaatattcacttctaaaacagacacatggtcctttggagtgctgctatgg 1140 |
| 1141 | gaaatcttttctcttggatatatgccataccccagcaaaagcaaccaggaagttctggag 1200 |
| 1201 | tttgtcaccagtggaggccggatggacccacccaagaactgccctgggcctgtataccgg 1260 |
| 1261 | ataatgactcagtgctggcaacatcagcctgaagacaggcccaactttgccatcattttg 1320 |
| 1321 | gagaggattgaatactgcacccaggacccggatgtaatcaacaccgctttgccgatagaa 1380 |
| 1381 | tatggtccacttgtggaagaggaagagaaagtgcctgtgaggcccaaggaccctgagggg 1440 |
| 1441 | gttcctcctctcctggtctctcaacaggcaaaacgggaggaggagcgcagcccagctgcc 1500 |
| 1501 | ccaccacctctgcctaccacctcctctggcaaggctgcaaagaaacccacagctgcagag 1560 |
| 1561 | gtctctgttcgagtccctagagggccggccgtggaagggggacacgtgaatatggcattc 1620 |
| 1621 | tctcagtccaaccctccttcggagttgcacaaggtccacggatccagaaacaagcccacc 1680 |
| 1681 | agcttgtggaacccaacgtacggctcctggtttacagagaaacccaccaaaaagaataat 1740 |
| 1741 | cctatagcaaagaaggagccacacgacaggggtaacctggggctggagggaagctgtact 1800 |
| 1801 | gtcccacctaacgttgcaactgggagacttccgggggcctcactgctcctagagccctct 1860 |
| 1861 | tcgctgactgccaatatgaaggaggtacctctgttcaggctacgtcacttcccttgtggg 1920 |
| 1921 | aatgtcaattacggctaccagcaacagggcttgcccttagaagccgctactgcccctgga 1980 |
| 1981 | gctggtcattacgaggataccattctgaaaagcaagaatagcatgaaccagcctgggccc 2040 |
| 2041 | tga 2043 |

In some embodiments, the fusion protein is an ALK protein fused to an echinoderm microtubule-associated protein-like 4 (EML4) protein. In some embodiments, the ELM4-ALK fusion protein is at least 85%, at least 90%, at least 95%, at least 98%, at least 99%, or 100% identical to a ELM4-ALK fusion protein in Homo Sapiens or a variant thereof. In some embodiments, the ELM4-ALK fusion protein is at least 85%, at least 90%, at least 95%, at least 98%, at least 99%, or 100% identical to an exemplary ELM4-ALK fusion protein amino acid sequence from Homo Sapiens (GenBank: BAM37627.1) as provided below:

| | |
|---|---|
| 1 | MDGFAGSLDD SISAASTSDV QDRLSALESR |
| | VQQQEDEITV LKAALADVLR RLAISEDHVA |
| 61 | SVKKSVSSKG QPSPRAVIPM SCITNGSGAN |
| | RKPSHTSAVS IAGKETLSSA AKSGTEKKKE |
| 121 | KPQGQREKKE ESHSNDQSPQ IRASPSPQPS |
| | SQPLQIHRQT PESKNATPTK SIKRPSPAEK |
| 181 | SHNSWENSDD SRNKLSKIPS TPKLIPKVTK |
| | TADKHKDVII NQEGEYIKMF MRGRPITMFI |
| 241 | PSDVDNYDDI RTELPPEKLK LEWAYGYRGK |
| | DCRANVYLLP TGEIVYFIAS VVVLFNYEER |
| 301 | TQRHYLGHTD CVKCLAIHPD KIRIATGQIA |
| | GVDKDGRPLQ PHVRVWDSVT LSTLQIIGLG |
| 361 | TFERGVGCLD FSKADSGVHL CVIDDSNEHM |
| | LTVWDWQRKA KGAEIKTTNE VVLAVEFHPT |
| 421 | DANTIITCGK SHIFFWTWSG NSLTRKQGIF |
| | GKYEKPKFVQ CLAFLGNGDV LTGDSGGVML |
| 481 | IWSKTTVEPT PGKGPKGVYQ ISKQIKAHDG |
| | SVFTLCQMRN GMLLTGGGKD RKIILWDHDL |
| 541 | NPEREIEFSA SRARLPGHVA ADHPPAVYRR |
| | KHQELQAMQM ELQSPEYKLS KLRTSTIMTD |
| 601 | YNPNYCFAGK TSSISDLKEV PRKNITLIRG |
| | LGHGAFGEVY EGQVSGMPND PSPLQVAVKT |
| 661 | LPEVCSEQDE LDFLMEALII SKFNHQNIVR |
| | CIGVSLQSLP RFILLELMAG GDLKSFLRET |
| 721 | RPRPSQPSSL AMLDLLHVAR DIACGCQYLE |
| | ENHFIHRDIA ARNCLLTCPG PGRVAKIGDF |
| 781 | GMARDIYRAS YYRKGGCAML PVKWMPPEAF |
| | MEGIFTSKTD TWSFGVLLWE IFSLGYMPYP |
| 841 | SKSNQEVLEF VTSGGRMDPP KNCPGPVYRI |
| | MTQCWQHQPE DRPNFAIILE RIEYCTQDPD |
| 901 | VINTALPIEY GPLVEEEEKV PVRPKDPEGV |
| | PPLLVSQQAK REEERSPAAP PPLPTTSSGK |
| 961 | AAKKPTAAEI SVRVPRGPAV EGGHVNMAFS |
| | QSNPPSELHK VHGSRNKPTS LWNPTYGSWF |
| 1021 | TEKPTKKNNP IAKKEPHDRG NLGLEGSCTV |
| | PPNVATGRLP GASLLLEPSS LTANMKEVPL |
| 1081 | FRLRHFPCGN VNYGYOOOGL PLEAATAPGA |
| | GHYEDTILKS KNSMNQPGP |

In some embodiments, the ELM4-ALK fusion protein is encoded by a nucleic acid sequence that is at least 85%, at least 90%, at least 95%, at least 98%, at least 99%, or 100% identical to an exemplary nucleic acid sequence from Homo Sapiens (GenBank: AB274722.1) as provided below:

| | |
|---|---|
| 1 | ggcggcgcgg cgcggcgctc gcggctgctg cctgggaggg aggccgggca ggcggctgag |
| 61 | cggcgcggct ctcaacgtga cggggaagtg gttcgggcgg ccgcggctta ctaccccagg |
| 121 | gcgaacggac ggacgacgga ggcgggagcc ggtagccgag ccgggcgacc tagagaacga |
| 181 | gcgggtcagg ctcagcgtcg gccactctgt cggtccgctg aatgaagtgc ccgcccctct |
| 241 | gagcccggag cccggcgctt tccccgcaag atggacggtt tcgccggcag tctcgatgat |
| 301 | agtatttctg ctgcaagtac ttctgatgtt caagatcgcc tgtcagctct tgagtcacga |
| 361 | gttcagcaac aagaagatga aatcactgtg ctaaaggcgg ctttggctga tgttttgagg |
| 421 | cgtcttgcaa tctctgaaga tcatgtggcc tcagtgaaaa aatcagtctc aagtaaaggc |
| 481 | caaccaagcc ctcgagcagt tattcccatg tcctgtataa ccaatggaag tggtgcaaac |
| 541 | agaaaaccaa gtcataccag tgctgtctca attgcaggaa aagaaactct ttcatctgct |
| 601 | gctaaaagtg gtacagaaaa aaagaaagaa aaaccacaag gacagagaga aaaaaaagag |
| 661 | gaatctcatt ctaatgatca aagtccacaa attcgagcat caccttctcc ccagccctct |
| 721 | tcacaacctc tccaaataca cagacaaact ccagaaagca agaatgctac tcccaccaaa |
| 781 | agcataaaac gaccatcacc agctgaaaag tcacataatt cttgggaaaa ttcagatgat |
| 841 | agccgtaata aattgtcgaa aataccttca acacccaaat taataccaaa agttaccaaa |
| 901 | actgcagaca agcataaaga tgtcatcatc aaccaagaag gagaatatat taaaatgttt |
| 961 | atgcgcggtc ggccaattac catgttcatt ccttccgatg ttgacaacta tgatgacatc |
| 1021 | agaacggaac tgcctcctga gaagctcaaa ctggagtggg catatggtta tcgaggaaag |
| 1081 | gactgtagag ctaatgttta ccttcttccg accggggaaa tagtttattt cattgcatca |
| 1141 | gtagtagtac tatttaatta tgaggagaga actcagcgac actacctggg ccatacagac |
| 1201 | tgtgtgaaat gccttgctat acatcctgac aaaattagga ttgcaactgg acagatagct |
| 1261 | ggcgtggata aagatggaag gcctctacaa ccccacgtca gagtgtggga ttctgttact |
| 1321 | ctatccacac tgcagattat tggacttggc acttttgagc gtggagtagg atgcctggat |
| 1381 | ttttcaaaag cagattcagg tgttcattta tgtgttattg atgactccaa tgagcatatg |
| 1441 | cttactgtat gggactggca gaagaaagca aaaggagcag aaataaagac aacaaatgaa |
| 1501 | gttgttttgg ctgtggagtt tcacccaaca gatgcaaata ccataattac atgcggtaaa |
| 1561 | tctcatattt tcttctggac ctggagcggc aattcactaa caagaaaaca gggaattttt |
| 1621 | gggaaatatg aaaagccaaa atttgtgcag tgtttagcat tcttggggaa tggagatgtt |
| 1681 | cttactggag actcaggtgg agtcatgctt atatggagca aaactactgt agagcccaca |
| 1741 | cctgggaaag gacctaaagt gtaccgccgg aagcaccagg agctgcaagc catgcagatg |
| 1801 | gagctgcaga gccctgagta caagctgagc aagctccgca cctcgaccat catgaccgac |
| 1861 | tacaacccca actactgctt tgctggcaag acctcctcca tcagtgacct gaaggaggtg |
| 1921 | ccgcggaaaa acatcaccct cattcggggt ctgggccatg gagcctttgg ggaggtgtat |
| 1981 | gaaggccagg tgtccggaat gcccaacgac ccaagccccc tgcaagtggc tgtgaagacg |
| 2041 | ctgcctgaag tgtgctctga acaggacgaa ctggatttcc tcatggaagc cctgatcatc |
| 2101 | agcaaattca accaccagaa cattgttcgc tgcattgggg tgagcctgca atccctgccc |
| 2161 | cggttcatcc tgctggagct catggcgggg ggagacctca agtccttcct ccgagagacc |
| 2221 | cgccctcgcc cgagccagcc ctcctccctg gccatgctgg accttctgca cgtggctcgg |
| 2281 | gacattgcct gtggctgtca gtatttggag gaaaaccact tcatccaccg agacattgct |
| 2341 | gccagaaact gcctcttgac ctgtccaggc cctggaagag tggccaagat tggagacttc |
| 2401 | gggatggccc gagacatcta cagggcgagc tactatagaa agggaggctg tgccatgctg |
| 2461 | ccagttaagt ggatgccccc agaggccttc atggaaggaa tattcacttc taaaacagac |
| 2521 | acatggtcct ttggagtgct gctatgggaa atcttttctc ttggatatat gccatacccc |
| 2581 | agcaaaagca accaggaagt tctggagttt gtcaccagtg gaggccggat ggacccaccc |
| 2641 | aagaactgcc ctgggcctgt ataccggata atgactcagt gctggcaaca tcagcctgaa |
| 2701 | gacaggccca actttgccat cattttggag aggattgaat actgcaccca ggacccggat |
| 2761 | gtaatcaaca ccgctttgcc gatagaatat ggtccacttg tggaagagga agagaaagtg |
| 2821 | cctgtgaggc ccaaggaccc tgagggggtt cctcctctcc tggtctctca acaggcaaaa |
| 2881 | cgggaggagg agcgcagccc agctgcccca ccacctctgc ctaccacctc ctctggcaag |
| 2941 | gctgcaaaga aacccacagc tgcagaggtc tctgttcgag tccctagagg gccggccgtg |
| 3001 | gaagggggac acgtgaatat ggcattctct cagtccaacc ctccttcgga gttgcacagg |
| 3061 | gtccacggat ccagaaacaa gcccaccagc ttgtggaacc caacgtacgg ctcctggttt |
| 3121 | acagagaaac ccaccaaaaa gaataatcct atagcaaaga aggagccaca cgagaggggt |
| 3181 | aacctggggc tggagggaag ctgtactgtc ccacctaacg ttgcaactgg gagacttccg |
| 3241 | ggggcctcac tgctcctaga gccctcttcg ctgactgcca atatgaagga ggtacctctg |
| 3301 | ttcaggctac gtcacttccc ttgtgggaat gtcaattacg gctaccagca acagggcttg |
| 3361 | cccttagaag ccgctactgc ccctggagct ggtcattacg aggataccat tctgaaaagc |
| 3421 | aagaatagca tgaaccagcc tgggccctga gctcggtcac acactcactt ctcttccttg |
| 3481 | ggatccctaa gaccgtggag gagagagagg caatcaatgg ctccttcaca aaccagagac |
| 3541 | caaatgtcac gttttgtttt gtgccaacct attttgaagt accaccaaaa aagctgtatt |
| 3601 | ttgaaaatgc tttagaaagg ttttgagcat gggttcatcc tattctttcg aaagaagaaa |
| 3661 | atatcataaa aatgagtgat aaatacaagg cccagatgtg gttgcataag gtttttatgc |
| 3721 | atgtttgttg tatacttcct tatgcttctt ttaaattgtg tgtgctctgc ttcaatgtag |
| 3781 | tcagaattag ctgcttctat gtttcatagt tggggtcata gatgtttcct tgccttgttg |
| 3841 | atgtggacat gagccatttg aggggagagg gaacggaaat aaaggagtta tttgtaatga |
| 3901 | aaaaaaaaaa aaaaaaaaaa aaaaaa |

In some embodiments, the ELM4-ALK fusion protein is at least 85%, at least 90%, at least 95%, at least 98%, at least 99%, or 100% identical to an exemplary ELM4-ALK variant 1 fusion protein amino acid sequence from Homo Sapiens (GenBank: BAF73611.1) as provided below:

| | |
|---|---|
| 1 | MDGFAGSLDD SISAASTSDV QDRLSALESR VQQQEDEITV LKAALADVLR RLAISEDHVA |
| 61 | SVKKSVSSKG QPSPRAVIPM SCITNGSGAN RKPSHTSAVS IAGKETLSSA AKSGTEKKKE |
| 121 | KPQGQREKKE ESHSNDQSPQ IRASPSPQPS SQPLQIHRQT PESKNATPTK SIKRPSPAEK |
| 181 | SHNSWENSDD SRNKLSKIPS TPKLIPKVTK TADKHKDVII NQEGEYIKMF MRGRPITMFI |
| 241 | PSDVDNYDDI RTELPPEKLK LEWAYGYRGK DCRANVYLLP TGEIVYFIAS VWVVFNYEER |
| 301 | TQRHYLGHTD CVKCLAIHPD KIRIATGQIA GVDKDGRPLQ PHVRVWDSVT LSTLQIIGLG |
| 361 | TFERGVGCLD FSKADSGVHL CVIDDSNEHM LTVWDWQKKA KGAEIKTTNE VVLAVEFHPT |
| 421 | DANTIITCGK SHIFFWTWSG NSLTRKQGIF GKYEKPKFVQ CLAFLGNGDV LTGDSGGVML |
| 481 | IWSKTTVEPT PGKGPKVYRR KHQELQAMQM ELQSPEYKLS KLRTSTIMTD YNPNYCFAGK |
| 541 | TSSISDLKEV PRKNITLIRG LGHGAFGEVY EGQVSGMPND PSPLQVAVKT LPEVCSEQDE |
| 601 | LDFLMEALII SKFNHQNIVR CIGVSLQSLP RFILLELMAG GDLKSFLRET RPRPSQPSSL |
| 661 | AMLDLLHVAR DIACGCQYLE ENHFIHRDIA ARNCLLTCPG PGRVAKIGDF GMARDIYRAS |
| 721 | YYRKGGCAML PVKWMPPEAF MEGIFTSKTD TWSFGVLLWE IFSLGYMPYP SKSNQEVLEF |
| 781 | VTSGGRMDPP KNCPGPVYRI MTQCWQHQPE DRPNFAIILE RIEYCTQDPD VINTALPIEY |
| 841 | GPLVEEEEKV PVRPKDPEGV PPLLVSQQAK REEERSPAAP PPLPTTSSGK AAKKPTAAEV |
| 901 | SVRVPRGPAV EGGHVNMAFS QSNPPSELHR VHGSRNKPTS LWNPTYGSWF TEKPTKKNNP |
| 961 | IAKKEPHERG NLGLEGSCTV PPNVATGRLP GASLLLEPSS LTANMKEVPL FRLRHFPCGN |
| 1021 | VNYGYOOOGL PLEAATAPGA GHYEDTILKS KNSMNQPGP |

In some embodiments, the ALK CAR of the present invention includes sequences from an anti-ALK antibody that specifically binds to a mammalian ALK protein or antigen. In some embodiments, the ALK CAR includes sequences from an anti-ALK antibody that binds to a murine ALK protein or an antibody-binding portion thereof. In some embodiments, the ALK CAR includes sequences from an anti-ALK antibody that binds to a human ALK protein or an antibody-binding portion thereof. In some embodiments, the ALK CAR includes sequences from an anti-ALK antibody that binds to a portion of the extracellular domain of the ALK receptor. In some embodiments, the ALK CAR includes sequences from an anti-ALK antibody that binds to a portion of the extracellular domain of a murine ALK receptor. In some embodiments, the ALK CAR includes sequences from an anti-ALK antibody that binds to a portion of the extracellular domain of a human ALK receptor. In some embodiments, the ALK CAR includes sequences from an anti-ALK antibody that is a murine antibody. In some embodiments, the ALK CAR includes sequences from an anti-ALK antibody that is a human antibody. In some embodiments, the ALK CAR includes sequences from an anti-ALK antibody that is a humanized antibody. In some embodiments, the ALK CAR includes sequences from an anti-ALK antibody that is a chimeric antibody.

In some embodiments, the ALK CAR includes sequences from an anti-ALK antibody that modulates ALK activity (e.g., ALK signaling) and/or ALK expression. In some embodiments, the ALK CAR includes sequences from an anti-ALK antibody that inhibits ALK signaling and/or ALK expression (e.g., inhibits ALK phosphorylation). In some embodiments, the ALK CAR includes sequences from an anti-ALK antibody that activates ALK signaling and/or ALK expression (e.g., agonist of ALK phosphorylation).

In some embodiments, the ALK CAR includes sequences from an anti-ALK antibody that is selected from ALK Antibody #1 (ALK #1), ALK Antibody #2 (ALK #2), ALK Antibody #3 (ALK #3), ALK Antibody #4 (ALK #4), ALK Antibody #5 (ALK #5), ALK Antibody #6 (ALK #6), or ALK Antibody #7 (ALK #7). In some embodiments, the ALK CAR includes sequences from ALK #1. In some embodiments, the ALK CAR includes sequences from ALK #2. In some embodiments, the ALK CAR includes sequences from ALK #3. In some embodiments, the ALK CAR includes sequences from ALK #4. In some embodiments, the ALK CAR includes sequences from ALK #5. In some embodiments, the ALK CAR includes sequences from ALK #6. In some embodiments, the ALK CAR includes sequences from ALK #7.

In some embodiments, the ALK CAR includes sequences from an anti-ALK antibody or an antigen binding fragment thereof comprising a VL region selected from ALK Antibody #1 (ALK #1), ALK Antibody #2 (ALK #2), ALK Antibody #3 (ALK #3), ALK Antibody #4 (ALK #4), ALK Antibody #5 (ALK #5), ALK Antibody #6 (ALK #6), or ALK Antibody #7 (ALK #7) (see Table 1). In some embodiments, the ALK CAR includes sequences from the VL region of ALK #1. In some embodiments, the ALK CAR includes sequences from the VL region of ALK #2. In some embodiments, the ALK CAR includes sequences from the VL region of ALK #3. In some embodiments, the ALK CAR includes sequences from the VL region of ALK #4. In some embodiments, the ALK CAR includes sequences from the VL region of ALK #5. In some embodiments, the ALK CAR includes sequences from the VL region of ALK #6. In some embodiments, the ALK CAR includes sequences from the VL region of ALK #7.

TABLE 1
Variable Light Chain (VL) ALK Antibody Sequences

| ALK | | |
|---|---|---|
| Antibody | VL Amino Acid Sequence | VL Nucleic Acid Sequence |
| ALK#1 | DIQMTQSPASLAASVGETVTITCRA | gacatccaga tgactcagtc tccagcctcc |
| | SENIYYSLAWYQQKQGKSPQLLIYN | ctggctgcat ctgtgggaga aactgtcacc |
| | ANSLEDGVPSRFSGSGSGTQYSMKI | atcacatgtc gagcaagtga gaacatttac |
| | NSMQPEDTATYFCKQAYDVPFTFGS | tacagtttag catggtatca gcagaagcaa |
| | GTKLEIKR | gggaaatctc ctcagctcct gatctataat |
| | | gcaaacagct tggaagatgg tgtcccatcg |
| | | aggttcagtg gcagtggatc tgggacacag |
| | | tattctatga agatcaacag catgcagcct |
| | | gaagataccg caacttattt ctgtaaacag |
| | | gcttatgacg ttccattcac gttcggctcg |
| | | gggacaaagt tggaaataaa acgg |
| ALK#2 | AIQMTQTTSSLSASLGDRVTISCSV | gctatccaga tgacacagac tacatcctcc |
| | SQGISNSLNWYQQKPDGTVKLLIYY | ctgtctgcct ctctgggaga cagagtcacc |
| | TSSLHSGVPSRFSGSGSGTDYSLTI | atcagttgca gtgtaagtca gggcattagc |
| | SNLEPEDIATYYCQQYSKLPLTFGA | aattctttaa actggtatca gcagaaacca |
| | GTKLELKR | gatggaactg ttaaactcct gatctattac |
| | | acatcaagtt tacactcagg agtcccatca |
| | | aggttcagtg gcagtgggtc tgggacagat |
| | | tattctctca ccatcagcaa cctggaacct |
| | | gaagatattg ccacttacta ttgtcagcag |
| | | tatagtaagc ttccgctcac gttcggtgct |
| | | gggaccaagc tggagctgaa acgg |
| ALK#3 | DIVMTQSQRFMSTSVGDRVSVT | gacattgtga tgacccagtc tcaaagattc |
| | CKASQNVGTNVAWYQQKPGQS | atgtccacat cagtaggaga cagggtcagc |
| | PKALIYSASYRYSGVPDRFTGS | gtcacctgca aggccagtca gaatgtgggt |
| | GSGTDFTLTVSNVQSEDLAEYF | actaatgtag cctggtatca acagaaacca |
| | CQQYNSYPYMYTFGGGTKLEIK | gggcaatctc ctaaagcact gatttactcg |
| | R | gcatcctacc ggtacagtgg agtccctgat |
| | | cgcttcacag gcagtggatc tgggacagat |
| | | ttcactctca ccgtcagcaa tgtgcagtct |
| | | gaagacttgg cagagtattt ctgtcagcaa |
| | | tataacagct atccgtacat gtacacgttc |
| | | ggagggggga ccaagctgga aataaaacgg |
| ALK#4 | DIVLTQSPASLAVSLGQRATISCRA | gacattgtgc tgacccaatc tccagcttct |
| | SESVDNYGISFMNWFQQKPGQPP | ttggctgtgt ctctagggca gagggccacc |
| | KLLIYAASNQGSGVPARFSGSGSG | atctcctgca gagccagcga aagtgttgat |
| | TDFSLNIHPMEEDDTAMYFCQQSK | aattatggca ttagttttat gaactggttc |
| | EVPWTFGGGTKLEIKR | caacagaaac caggacagcc acccaaactc |
| | | ctcatctatg ctgcatccaa ccaaggatcc |
| | | ggggtccctg ccaggtttag tggcagtggg |
| | | tctgggacag acttcagcct caacatccat |
| | | cctatggagg aggatgatac tgcaatgtat |
| | | ttctgtcagc aaagtaagga ggttccgtgg |
| | | acgttcggtg gaggcaccaa gctggaaatc |
| | | aaacgg |
| ALK#5 | DIVMTQSQRFMSTSVGDRVSVTCKA | gacattgtga tgacccagtc tcaaagattc |
| | SQNVGTNVAWYQQKPGQSPKALIYS | atgtccacat cagtaggaga cagggtcagc |
| | ASYRYSGVPDRFTGSGSGTDFTLTI | gtcacctgca aggccagtca gaatgtgggt |
| | SNVQSEDLAEYFCQQYNSYPYMYTF | actaatgtag cctggtatca acagaaacca |
| | GGGTKLEIKR | gggcaatctc ctaaagcact gatttactcg |
| | | gcatcctacc ggtacagtgg agtccctgat |
| | | cgcttcacag gcagtggatc tgggacagat |
| | | ttcactctca ccatcagcaa tgtgcagtct |
| | | gaagacttgg cagagtattt ctgtcaacaa |
| | | tataacagct atccgtacat gtacacgttc |
| | | ggagggggga ccaagctgga aataaaacgg |
| ALK#6 | DIVMTQSQKFMSTSVGDRVSITCKA | gacattgtga tgacccagtc tcaaaaattc |
| | SQNVGTAVAWYQLKPGQSPKLLIY | atgtccacat cagtaggaga cagggtcagc |
| | SASNRFTGVPDRFTGSGSGTDFTL | atcacctgta aggccagtca gaatgtgggt |
| | TISNMQSEDLADYFCQQYSSYPLTF | actgctgtag cctggtatca actgaaacca |
| | GSGTKLEIKR | ggacaatctc ctaaactact gatttactcg |
| | | gcatccaatc ggttcactgg agtccctgat |
| | | cgcttcacag gcagtggatc tgggacagat |
| | | ttcactctca ccatcagcaa tatgcagtct |
| | | gaagacctgg cagattattt ctgccagcaa |
| | | tatagcagct atcctctcac gttcggctcg |
| | | gggacaaagt tggaaataaa acgg |
| ALK#7 | DIVMTQSQKFMSTSVGDRVSVTCK | gacattgtga tgacccagtc tcaaaaattc |
| | ASQNVGTNVAWYQQKPGHSPKALI | atgtccacat cagtaggaga cagggtcagc |
| | YSASYRYSGVPDRFTGSGSGTDFT | gtcacctgca aggccagtca gaatgtgggt |
| | LTISNVQSEDLAEYFCQRYNSYPY | actaatgtag cctggtatca acagaaacca |
| | MFTFGGGTKLEIKR | gggcactctc ctaaagcact gatttactcg |
| | | gcatcctacc ggtacagtgg agtccctgat |
| | | cgcttcacag gcagtggatc tgggacagat |
| | | ttcactctca ccatcagcaa tgtgcagtct |
| | | gaagacttgg cagagtattt ctgtcagcga |
| | | tataacagct atccgtacat gttcacgttc |
| | | ggagggggga ccaagctgga aataaaacgg |

In some embodiments, the ALK CAR includes a sequence that is at least 85%, at least 90%, at least 95%, at least 98%, at least 99%, or 100% identical to an exemplary anti-ALK antibody VL amino acid sequence as provided below:

DIQMTQSPASLAASVGETVTITCRASENIYYSLAW
YQQKQGKSPQLLIYNANSLEDGVPSRFSGSGSGTQ
YSMKINSMQPEDTATYFCKQAYDVPFTFGSGTKLE
IKR

In some embodiments, the ALK CAR includes a sequence that is at least 85%, at least 90%, at least 95%, at least 98%, at least 99%, or 100% identical to an exemplary anti-ALK antibody VL amino acid sequence as provided below:

AIQMTQTTSSLSASLGDRVTISCSVSQGISNSLNW
YQQKPDGTVKLLIYYTSSLHSGVPSRFSGSGSGTD
YSLTISNLEPEDIATYYCQQYSKLPLTFGAGTKLE
LKR

In some embodiments, the ALK CAR includes a sequence that is at least 85%, at least 90%, at least 95%, at least 98%, at least 99%, or 100% identical to an exemplary anti-ALK antibody VL amino acid sequence as provided below:

DIVMTQSQRFMSTSVGDRVSVTCKASQNVGTNVAW
YQQKPGQSPKALIYSASYRYSGVPDRFTGSGSGTD
FTLTVSNVQSEDLAEYFCQQYNSYPYMYTFGGGTK
LEIKR

In some embodiments, the ALK CAR includes a sequence that is at least 85%, at least 90%, at least 95%, at least 98%, at least 99%, or 100% identical to an exemplary anti-ALK antibody VL amino acid sequence as provided below:

DIVLTQSPASLAVSLGQRATISCRASESVDNYGIS
FMNWFQQKPGQPPKLLIYAASNQGSGVPARFSGSG
SGTDFSLNIHPMEEDDTAMYFCQQSKEVPWTFGGG
TKLEIKR

In some embodiments, the ALK CAR includes a sequence that is at least 85%, at least 90%, at least 95%, at least 98%, at least 99%, or 100% identical to an exemplary anti-ALK antibody VL amino acid sequence as provided below:

DIVMTQSQREMSTSVGDRVSVTCKASQNVGTNVAW
YQQKPGQSPKALIYSASYRYSGVPDRETGSGSGTD
FTLTISNVQSEDLAEYFCQQYNSYPYMYTFGGGTK
LEIKR

In some embodiments, the ALK CAR includes a sequence that is at least 85%, at least 90%, at least 95%, at least 98%, at least 99%, or 100% identical to an exemplary anti-ALK antibody VL amino acid sequence as provided below:

DIVMTQSQKFMSTSVGDRVSITCKASQNVGTAVAW
YQLKPGQSPKLLIYSASNRFTGVPDRFTGSGSGTD
FTLTISNMQSEDLADYFCQQYSSYPLTFGSGTKLE
IKR

In some embodiments, the ALK CAR includes a sequence that is at least 85%, at least 90%, at least 95%, at least 98%, at least 99%, or 100% identical to an exemplary anti-ALK antibody VL amino acid sequence as provided below:

DIVMTQSQKFMSTSVGDRVSVTCKASQNVGTNVAW
YQQKPGHSPKALIYSASYRYSGVPDRFTGSGSGTD
FTLTISNVQSEDLAEYFCQRYNSYPYMFTFGGGTK
LEIKR

In some embodiments, the ALK CAR includes an anti-ALK antibody VL region that is encoded by a polynucleotide that is at least 85%, at least 90%, at least 95%, at least 98%, at least 99%, or 100% identical to an exemplary nucleic acid sequence as provided below:

gacatccaga tgactcagtc tccagcctcc ctggctgcat ctgtgggaga aactgtcacc 60
atcacatgtc gagcaagtga gaacatttac tacagtttag catggtatca gcagaagcaa 120
gggaaatctc ctcagctcct gatctataat gcaaacagct tggaagatgg tgtcccatcg 180
aggttcagtg gcagtggatc tgggacacag tattctatga agatcaacag catgcagcct 240
gaagataccg caacttattt ctgtaaacag gcttatgacg ttccattcac gttcggctcg 300
gggacaaagt tggaaataaa acgg 324

In some embodiments, the ALK CAR includes an anti-ALK antibody VL region that is encoded by a polynucleotide that is at least 85%, at least 90%, at least 95%, at least 98%, at least 99%, or 100% identical to an exemplary nucleic acid sequence as provided below:

gctatccaga tgacacagac tacatcctcc ctgtctgcct ctctgggaga cagagtcacc 60
atcagttgca gtgtaagtca gggcattagc aattctttaa actggtatca gcagaaacca 120
gatggaactg ttaaactcct gatctattac acatcaagtt tacactcagg agtcccatca 180
aggttcagtg gcagtgggtc tgggacagat tattctctca ccatcagcaa cctggaacct 240
gaagatattg ccacttacta ttgtcagcag tatagtaagc ttccgctcac gttcggtgct 300
gggaccaagc tggagctgaa acgg 324

In some embodiments, the ALK CAR includes an anti-ALK antibody VL region that is encoded by a polynucleotide that is at least 85%, at least 90%, at least 95%, at least 98%, at least 99%, or 100% identical to an exemplary nucleic acid sequence as provided below:

gacattgtga tgacccagtc tcaaagattc atgtccacat cagtaggaga cagggtcagc 60
gtcacctgca aggccagtca gaatgtgggt actaatgtag cctggtatca acagaaacca 120
gggcaatctc ctaaagcact gatttactcg gcatcctacc ggtacagtgg agtccctgat 180
cgcttcacag gcagtggatc tgggacagat ttcactctca ccgtcagcaa tgtgcagtct 240
gaagacttgg cagagtattt ctgtcagcaa tataacagct atccgtacat gtacacgttc 300
ggagggggga ccaagctgga aataaaacgg 330

In some embodiments, the ALK CAR includes an anti-ALK antibody VL region that is encoded by a polynucleotide that is at least 85%, at least 90%, at least 95%, at least 98%, at least 99%, or 100% identical to an exemplary nucleic acid sequence as provided below:

gacattgtgc tgacccaatc tccagcttct ttggctgtgt ctctagggca gagggccacc 60
atctcctgca gagccagcga aagtgttgat aattatggca ttagttttat gaactggttc 120
caacagaaac caggacagcc acccaaactc ctcatctatg ctgcatccaa ccaaggatcc 180
ggggtccctg ccaggtttag tggcagtggg tctgggacag acttcagcct caacatccat 240
cctatggagg aggatgatac tgcaatgtat ttctgtcagc aaagtaagga ggttccgtgg 300
acgttcggtg gaggcaccaa gctggaaatc aaacgg 336

In some embodiments, the ALK CAR includes an anti-ALK antibody VL region that is encoded by a polynucleotide that is at least 85%, at least 90%, at least 95%, at least 98%, at least 99%, or 100% identical to an exemplary nucleic acid sequence as provided below:

gacattgtga tgacccagtc tcaaagattc atgtccacat cagtaggaga cagggtcagc 60
gtcacctgca aggccagtca gaatgtgggt actaatgtag cctggtatca acagaaacca 120
gggcaatctc ctaaagcact gatttactcg gcatcctacc ggtacagtgg agtccctgat 180
cgcttcacag gcagtggatc tgggacagat ttcactctca ccatcagcaa tgtgcagtct 240
gaagacttgg cagagtattt ctgtcaacaa tataacagct atccgtacat gtacacgttc 300
ggagggggga ccaagctgga aataaaacgg 330

In some embodiments, the ALK CAR includes an anti-ALK antibody VL region that is encoded by a polynucleotide that is at least 85%, at least 90%, at least 95%, at least 98%, at least 99%, or 100% identical to an exemplary nucleic acid sequence as provided below:

gacattgtga tgacccagtc tcaaaaattc atgtccacat cagtaggaga cagggtcagc 60
atcacctgta aggccagtca gaatgtgggt actgctgtag cctggtatca actgaaacca 120
ggacaatctc ctaaactact gatttactcg gcatccaatc ggttcactgg agtccctgat 180
cgcttcacag gcagtggatc tgggacagat ttcactctca ccatcagcaa tatgcagtct 240
gaagacctgg cagattattt ctgccagcaa tatagcagct atcctctcac gttcggctcg 300
gggacaaagt tggaaataaa acgg 324

In some embodiments, the ALK CAR includes an anti-ALK antibody VL region that is encoded by a polynucleotide that is at least 85%, at least 90%, at least 95%, at least 98%, at least 99%, or 100% identical to an exemplary nucleic acid sequence as provided below:

gacattgtga tgacccagtc tcaaaaattc atgtccacat cagtaggaga cagggtcagc 60
gtcacctgca aggccagtca gaatgtgggt actaatgtag cctggtatca acagaaacca 120
gggcactctc ctaaagcact gatttactcg gcatcctacc ggtacagtgg agtccctgat 180
cgcttcacag gcagtggatc tgggacagat ttcactctca ccatcagcaa tgtgcagtct 240
gaagacttgg cagagtattt ctgtcagcga tataacagct atccgtacat gttcacgttc 300
ggagggggga ccaagctgga aataaaacgg 330

In some embodiments, the ALK CAR includes an anti-ALK antibody or an antigen binding fragment thereof comprising a VH region selected from ALK Antibody #1 (ALK #1), ALK Antibody #2 (ALK #2), ALK Antibody #3 (ALK #3), ALK Antibody #4 (ALK #4), ALK Antibody #5 (ALK #5), ALK Antibody #6 (ALK #6), or ALK Antibody #7 (ALK #7) (see Table 2). In some embodiments, the ALK CAR includes the VH region selected from ALK #1. In some embodiments, the ALK CAR includes the VH region selected from ALK #2. In some embodiments, the ALK CAR includes the VH region selected from ALK #3. In some embodiments, the ALK CAR includes the VH region selected from ALK #4. In some embodiments, the ALK CAR includes the VH region selected from ALK #5. In some embodiments, the ALK CAR includes the VH region selected from ALK #6. In some embodiments, the ALK CAR includes the VH region selected from ALK #7.

TABLE 2
Variable Heavy Chain (VH) ALK Antibody Sequences

| ALK | | |
|---|---|---|
| Antibody | VH Amino Acid Sequence | VH Nucleic Acid Sequence |
| ALK#1 | QVQLQQSGAELVKPGASVKISC | caggttcagc tgcagcagtc tggggctgag |
| | KASGYAFSSYWMNWWKQRPG | ctggtgaagc ctggggcctc agtgaagatt |
| | KGLEWIGQIYPGDGDTNYNGKF | tcctgcaaag cttctggcta cgcattcagt |
| | KGKATLTADKSSSTAYMQLSSL | agctactgga tgaactgggt gaagcagagg |
| | TSEDSAVYFCASYYYGSKAYW | cctggaaagg gtcttgagtg gattggacag |
| | GQGTLVTVSA | atttatcctg gagatggtga tactaactac |
| | | aacggaaagt tcaagggcaa ggccacactg |
| | | actgcagaca aatcctccag cacagcctac |
| | | atgcagctca gcagcctgac ctctgaggac |
| | | tctgcggtct atttctgtgc ctcttattac |
| | | tacggtagta aggcttactg gggccaaggg |
| | | actctggtca ctgtctctgc a |
| ALK#2 | QVQLQQPGAEFVKPGASVKLS | caggtccaac tgcagcagcc tggggctgag |
| | CKASGYTFTSYWMHWWKQRP | tttgtgaagc ctggggcttc agtgaagctg |
| | GRGLEWIGRIDPNSGGTKYNEK | tcctgcaagg cttctggcta caccttcacc |
| | FKSKATLTVDKPSSTAYMQLSS | agctactgga tgcactgggt gaagcagagg |
| | LTSEDSAVYYCARDYYGSSYRF | cctggacgag gccttgagtg gattggaagg |
| | AYWGQGTLVTVSA | attgatccta atagtggtgg tactaagtac |
| | | aatgagaagt tcaagagcaa ggccacactg |
| | | actgtagaca aaccctccag cacagcctac |
| | | atgcagctca gcagcctgac atctgaggac |
| | | tctgcggtct attattgtgc aagagattac |
| | | tacggtagta gctaccggtt tgcttactgg |
| | | ggccaaggga ctctggtcac tgtctctgca |
| ALK#3 | QVQLQQSGAELAKPGASVKLS | caggtccagc tgcagcagtc tggggctgaa |
| | CKASGYTFTNYWM H WWKQRP | ctggcaaaac ctggggcctc agtgaagctg |
| | GQGLEWIGYINPSSGYTKYNQK | tcctgcaagg cttctggcta cacctttact |
| | FKDKATLTADKSSSTAYMQLSS | aactactgga tgcactgggt aaaacagagg |
| | LTYEDSAVYYCARDYYGSSSW | cctggacagg gtctggaatg gattggatac |
| | FAYWGQGTLVTVSA | attaatccta gcagtggtta tactaagtac |
| | | aatcagaagt tcaaggacaa ggccacattg |
| | | actgcagaca aatcctccag cacagcctac |
| | | atgcagctga gcagcctgac atatgaggac |
| | | tctgcagtct attactgtgc aagagattac |
| | | tacggtagta gctcctggtt tgcttactgg |
| | | ggccaaggga ctctggtcac tgtctctgca |
| ALK#4 | QVQLQQSGAELVKPGASVKISC | caggttcagc tgcaacagtc tggggctgag |
| | KASGYAFSSYWVNWVKQRPGK | ctggtgaagc ctggggcctc agtgaagatt |
| | GLEWIGQIYPGDGDTNYNGKFK | tcctgcaaag cttctggcta cgcattcagt |
| | GKATLTADKSSSTAYMQLSSLT | agctactggg tgaactgggt gaagcagagg |
| | SEDSAVYFCARSRGYFYGSTY | cctggaaagg gtcttgagtg gattggacag |
| | DSWGQGTTLTVSS | atttatcctg gagatggtga tactaactac |
| | | aacggaaagt tcaagggcaa ggccacactg |
| | | actgcagaca aatcctccag cacagcctac |
| | | atgcagctca gcagcctgac ctctgaggac |
| | | tctgcggtct atttctgtgc aagatcaaga |
| | | gggtatttct acggtagtac ctacgactcc |
| | | tggggccaag gcaccactct cacagtctcc |
| | | tca |
| ALK#5 | QVQLQQSGAELAKPGASVKLS | caggtccagc tgcagcagtc tggggctgaa |
| | CKASGYTFTSYWMHWVKQRP | ctggcaaaac ctggggcctc agtgaagctg |
| | GQGLEWIGYIKPSSGYTKYNQK | tcctgcaagg cttctggcta cacctttact |
| | FKDKATLTADKSSSTAYMQLSS | agctactgga tgcactgggt aaaacagagg |
| | LTYEDSAVYYCARDYYGSSSW | cctggacagg gtctggaatg gattggatac |
| | FAYWGQGTLVTVSA | attaagccta gcagtggtta tactaagtac |
| | | aatcagaagt tcaaggacaa ggccacattg |
| | | actgcagaca aatcctccag cacagcctac |
| | | atgcagctga gcagcctgac atatgaggac |
| | | tctgcagtct attactgtgc aagagattac |
| | | tacggtagta gctcctggtt tgcttactgg |
| | | ggccaaggga ctctggtcac tgtctctgca |
| ALK#6 | DVKLVESGEGLVKPGGSLKLSC | gacgtgaagc tggtggagtc tggggaaggc |
| | AASGFTFSSYAMSWVRQTPEK | ttagtgaagc ctggagggtc cctgaaactc |
| | RLEWVTYISSGGDYIYYADTVK | tcctgtgcag cctctggatt cactttcagt |
| | GRFTISRDNARNTLYLQMSSLK | agctatgcca tgtcttgggt tcgccagact |
| | SEDTAMYYCTRERIWLRRFFDV | ccagagaaga ggctggagtg ggtcacatac |
| | WGTGTTVTVSS | attagtagtg gtggtgatta catctactat |
| | | gcagacactg tgaagggccg attcaccatc |
| | | tccagagaca atgccaggaa caccctgtac |
| | | ctgcaaatga gcagtctgaa gtctgaggac |
| | | acagccatgt attactgtac aagagagcgg |
| | | atatggttac gacggttctt cgatgtctgg |
| | | ggcacaggga ccacggtcac cgtctcctca |
| ALK#7 | QVQLQQSGAELAKPGASVKLS | caggtccagc tgcagcagtc tggggctgaa |
| | CKASGYTFTSYWMHVWKQRP | ctggcaaaac ctggggcctc agtgaagctg |
| | GQGLEWIGYINPSSGYTKYNQK | tcctgcaagg cttctggcta cacctttact |
| | FKDKATLTADKSSSTAYMQLSS | agctactgga tgcactgggt aaaacagagg |
| | LTFEDSAVYYCARDYYGSSSW | cctggacagg gtctggaatg gattggatac |
| | FAYWGQGTLVTVSA | attaatccta gcagtggtta tactaagtac |
| | | aatcagaagt tcaaggacaa ggccacattg |
| | | actgcagaca aatcctccag cacagcctac |
| | | atgcagctga gcagcctgac atttgaggac |
| | | tctgcagtct attactgtgc aagagattac |
| | | tacggtagta gctcctggtt tgcttactgg |
| | | ggccaaggga ctctggtcac tgtctctgca |

In some embodiments, the ALK CAR includes a sequence that is at least 85%, at least 90%, at least 95%, at least 98%, at least 99%, or 100% identical to an exemplary anti-ALK antibody VH amino acid sequence as provided below:

QVQLQQSGAELVKPGASVKISCKASGYAFSSYWMN
WKQRPGKGLEWIGQIYPGDGDTNYNGKFKGKATLT
ADKSSSTAYMQLSSLTSEDSAVYFCASYYYGSKAY
WGQGTLVTVSA

In some embodiments, the ALK CAR includes a sequence that is at least 85%, at least 90%, at least 95%, at least 98%, at least 99%, or 100% identical to an exemplary anti-ALK antibody VH amino acid sequence as provided below:

QVQLQQPGAEFVKPGASVKLSCKASGYTFTSYWMH
WKQRPGRGLEWIGRIDPNSGGTKYNEKFKSKATLT
VDKPSSTAYMQLSSLTSEDSAVYYCARDYYGSSYR
FAYWGQGTLVTVSA

In some embodiments, the ALK CAR includes a sequence that is at least 85%, at least 90%, at least 95%, at least 98%, at least 99%, or 100% identical to an exemplary anti-ALK antibody VH amino acid sequence as provided below:

QVQLQQSGAELAKPGASVKLSCKASGYTFTNYWMH
WKQRPGQGLEWIGYINPSSGYTKYNQKFKDKATLT
ADKSSSTAYMQLSSLTYEDSAVYYCARDYYGSSSW
FAYWGQGTLVTVSA

In some embodiments, the ALK CAR includes a sequence that is at least 85%, at least 90%, at least 95%, at least 98%, at least 99%, or 100% identical to an exemplary anti-ALK antibody VH amino acid sequence as provided below:

QVQLQQSGAELVKPGASVKISCKASGYAFSSYWVN
WKQRPGKGLEWIGQIYPGDGDTNYNGKFKGKATLT
ADKSSSTAYMQLSSLTSEDSAVYFCARSRGYFYGS
TYDSWGQGTTLTVSS

In some embodiments, the ALK CAR includes a sequence that is at least 85%, at least 90%, at least 95%, at least 98%, at least 99%, or 100% identical to an exemplary anti-ALK antibody VH amino acid sequence as provided below:

QVQLQQSGAELAKPGASVKLSCKASGYTFTSYWMH
WVKQRPGQGLEWIGYIKPSSGYTKYNQKFKDKATL
TADKSSSTAYMQLSSLTYEDSAVYYCARDYYGSSS
WFAYWGQGTLVTVSA

In some embodiments, the ALK CAR includes a sequence that is at least 85%, at least 90%, at least 95%, at least 98%, at least 99%, or 100% identical to an exemplary anti-ALK antibody VH amino acid sequence as provided below:

DVKLVESGEGLVKPGGSLKLSCAASGFTFSSYAMS
WRQTPEKRLEWTYISSGGDYIYYADTVKGRFTISR
DNARNTLYLQMSSLKSEDTAMYYCTRERIWLRRFF
DVWGTGTTVTVSS

In some embodiments, the ALK CAR includes a sequence that is at least 85%, at least 90%, at least 95%, at least 98%, at least 99%, or 100% identical to an exemplary anti-ALK antibody VH amino acid sequence as provided below:

QVQLQQSGAELAKPGASVKLSCKASGYTFTSYWMH
WVKQRPGQGLEWIGYINPSSGYTKYNQKFKDKATL
TADKSSSTAYMQLSSLTFEDSAVYYCARDYYGSSS
WFAYWGQGTLVTVSA

In some embodiments, the ALK CAR includes an anti-ALK antibody VH region that is encoded by a polynucleotide that is at least 85%, at least 90%, at least 95%, at least 98%, at least 99%, or 100% identical to an exemplary nucleic acid sequence as provided below:

caggttcagc tgcagcagtc tggggctgag ctggtgaagc ctggggcctc agtgaagatt 60
tcctgcaaag cttctggcta cgcattcagt agctactgga tgaactgggt gaagcagagg 120
cctggaaagg gtcttgagtg gattggacag atttatcctg gagatggtga tactaactac 180
aacggaaagt tcaagggcaa ggccacactg actgcagaca aatcctccag cacagcctac 240
atgcagctca gcagcctgac ctctgaggac tctgcggtct atttctgtgc ctcttattac 300
tacggtagta aggcttactg gggccaaggg actctggtca ctgtctctgc a 351

In some embodiments, the ALK CAR includes an anti-ALK antibody VH region that is encoded by a polynucleotide that is at least 85%, at least 90%, at least 95%, at least 98%, at least 99%, or 100% identical to an exemplary nucleic acid sequence as provided below:

caggtccaac tgcagcagcc tggggctgag tttgtgaagc ctggggcttc agtgaagctg 60
tcctgcaagg cttctggcta caccttcacc agctactgga tgcactgggt gaagcagagg 120
cctggacgag gccttgagtg gattggaagg attgatccta atagtggtgg tactaagtac 180
aatgagaagt tcaagagcaa ggccacactg actgtagaca aaccctccag cacagcctac 240
atgcagctca gcagcctgac atctgaggac tctgcggtct attattgtgc aagagattac 300
tacggtagta gctaccggtt tgcttactgg ggccaaggga ctctggtcac tgtctctgca 360

In some embodiments, the ALK CAR includes an anti-ALK antibody VH region that is encoded by a polynucleotide that is at least 85%, at least 90%, at least 95%, at least 98%, at least 99%, or 100% identical to an exemplary nucleic acid sequence as provided below:

caggtccagc tgcagcagtc tggggctgaa ctggcaaaac ctggggcctc agtgaagctg 60
tcctgcaagg cttctggcta cacctttact aactactgga tgcactgggt aaaacagagg 120
cctggacagg gtctggaatg gattggatac attaatccta gcagtggtta tactaagtac 180
aatcagaagt tcaaggacaa ggccacattg actgcagaca aatcctccag cacagcctac 240
atgcagctga gcagcctgac atatgaggac tctgcagtct attactgtgc aagagattac 300
tacggtagta gctcctggtt tgcttactgg ggccaaggga ctctggtcac tgtctctgca 360

In some embodiments, the ALK CAR includes an anti-ALK antibody VH region that is encoded by a polynucleotide that is at least 85%, at least 90%, at least 95%, at least 98%, at least 99%, or 100% identical to an exemplary nucleic acid sequence as provided below:

caggttcagc tgcaacagtc tggggctgag ctggtgaagc ctggggcctc agtgaagatt 60
tcctgcaaag cttctggcta cgcattcagt agctactggg tgaactgggt gaagcagagg 120
cctggaaagg gtcttgagtg gattggacag atttatcctg gagatggtga tactaactac 180
aacggaaagt tcaagggcaa ggccacactg actgcagaca aatcctccag cacagcctac 240
atgcagctca gcagcctgac ctctgaggac tctgcggtct atttctgtgc aagatcaaga 300
gggtatttct acggtagtac ctacgactcc tggggccaag gcaccactct cacagtctcc 360
tca 363

In some embodiments, the ALK CAR includes an anti-ALK antibody VH region that is encoded by a polynucleotide that is at least 85%, at least 90%, at least 95%, at least 98%, at least 99%, or 100% identical to an exemplary nucleic acid sequence as provided below:

caggtccagc tgcagcagtc tggggctgaa ctggcaaaac ctggggcctc agtgaagctg 60
tcctgcaagg cttctggcta cacctttact agctactgga tgcactgggt aaaacagagg 120
cctggacagg gtctggaatg gattggatac attaagccta gcagtggtta tactaagtac 180
aatcagaagt tcaaggacaa ggccacattg actgcagaca aatcctccag cacagcctac 240
atgcagctga gcagcctgac atatgaggac tctgcagtct attactgtgc aagagattac 300
tacggtagta gctcctggtt tgcttactgg ggccaaggga ctctggtcac tgtctctgca 360

In some embodiments, the ALK CAR includes an anti-ALK antibody VH region that is encoded by a polynucleotide that is at least 85%, at least 90%, at least 95%, at least 98%, at least 99%, or 100% identical to an exemplary nucleic acid sequence as provided below:

gacgtgaagc tggtggagtc tggggaaggc ttagtgaagc ctggagggtc cctgaaactc 60
tcctgtgcag cctctggatt cactttcagt agctatgcca tgtcttgggt tcgccagact 120
ccagagaaga ggctggagtg ggtcacatac attagtagtg gtggtgatta catctactat 180
gcagacactg tgaagggccg attcaccatc tccagagaca atgccaggaa caccctgtac 240
ctgcaaatga gcagtctgaa gtctgaggac acagccatgt attactgtac aagagagcgg 300
atatggttac gacggttctt cgatgtctgg ggcacaggga ccacggtcac cgtctcctca 360

In some embodiments, the ALK CAR includes an anti-ALK antibody VH region that is encoded by a polynucleotide that is at least 85%, at least 90%, at least 95%, at least 98%, at least 99%, or 100% identical to an exemplary nucleic acid sequence as provided below:

caggtccagc tgcagcagtc tggggctgaa ctggcaaaac ctggggcctc agtgaagctg 60
tcctgcaagg cttctggcta cacctttact agctactgga tgcactgggt aaaacagagg 120
cctggacagg gtctggaatg gattggatac attaatccta gcagtggtta tactaagtac 180
aatcagaagt tcaaggacaa ggccacattg actgcagaca aatcctccag cacagcctac 240
atgcagctga gcagcctgac atttgaggac tctgcagtct attactgtgc aagagattac 300
tacggtagta gctcctggtt tgcttactgg ggccaaggga ctctggtcac tgtctctgca 360

In some embodiments, the ALK CAR includes an anti-ALK antibody provided herein, or an antigen-binding fragment thereof, comprising a VL region and a VH region that is at least 85%, at least 90%, at least 95%, at least 98%, at least 99%, or 100% identical to the VL and VH amino acid sequences of any one of antibodies ALK #1, ALK #2, ALK #3, ALK #4, ALK #5, ALK #6, or ALK #7. In some embodiments, the ALK CAR includes an anti-ALK antibody provided herein, or an antigen-binding fragment thereof, comprising a VL region and a VH region of any one of antibodies ALK #1, ALK #2, ALK #3, ALK #4, ALK #5, ALK #6, or ALK #7.

The CDRs are primarily responsible for binding to an epitope of an antigen. The amino acid sequence positions of a given CDR can be readily determined using any methods known in the art, including those described by Kabat et al. (“Sequences of Proteins of Immunological Interest, 5th Ed. Public Health Service, National Institutes of Health, Bethesda, Md., 1991; “Kabat” numbering scheme), Al-Lazikani et al., (JMB 273,927-948, 1997: “Chothia” numbering scheme), and Lefranc et al. (“IMGT unique numbering for immunoglobulin and T cell receptor variable domains and Ig superfamily V-like domains.” Dev. Comp. Immunol., 27:55-77, 2003: “IMGT” numbering scheme). The CDRs of each chain are typically referred to as CDR1, CDR2, and CDR3 (from the N-terminus to C-terminus), and are also typically identified by the chain in which the particular CDR is located. Thus, herein a VH-CDR3 is the CDR3 from the variable domain of the heavy chain of the antibody in which it is found, and a VL-CDR1 is the CDR1 from the variable domain of the light chain of the antibody in which it is found. Light chain CDRs are referred herein as LCDR1, LCDR2, and LCDR3. Heavy chain CDRs are referred herein as HCDR1, HCDR2, and HCDR3.

In some embodiments, the ALK CAR includes CDRs of an anti-ALK antibody that specifically binds ALK (e.g., human ALK). In some embodiments, the ALK CAR includes the CDRs of an anti-ALK antibody that specifically binds the ECD of ALK (e.g., human ALK ECD). In some embodiments, the ALK CAR includes one or more CDRs of a VL region selected from ALK Antibody #1 (ALK #1), ALK Antibody #2 (ALK #2), ALK Antibody #3 (ALK #3), ALK Antibody #4 (ALK #4), ALK Antibody #5 (ALK #5), ALK Antibody #6 (ALK #6), or ALK Antibody #7 (ALK #7) (see Table 3). In some embodiments, the ALK CAR includes one or more CDRs of a VL region selected from ALK #1. In some embodiments, the ALK CAR includes one or more CDRs of a VL region selected from ALK #2. In some embodiments, the ALK CAR includes one or more CDRs of a VL region selected from ALK #3. In some embodiments, the ALK CAR includes one or more CDRs of a VL region selected from ALK #4. In some embodiments, the ALK CAR includes one or more CDRs of a VL region selected from ALK #5. In some embodiments, the ALK CAR includes one or more CDRs of a VL region selected from ALK #6. In some embodiments, the ALK CAR includes one or more CDRs of a VL region selected from ALK #7.

TABLE 3
Variable Light Chain (VL)
Complementary Determining Region
(CDR) ALK
Antibody Sequences (Kabat)

| ALK | | VL CDR Amino Acid |
|---|---|---|
| Antibody | CDR | Sequence |
| ALK#1 | LCDR1 | RASENIYYSLA |
| | LCDR2 | NANSLED |
| | LCDR3 | KQAYDVPFT |
| ALK#2 | LCDR1 | SVSQGISNSLN |
| | LCDR2 | YTSSLHS |
| | LCDR3 | QQYSKLPLT |
| ALK#3 | LCDR1 | KASQNVGTNVA |
| | LCDR2 | SASYRYS |
| | LCDR3 | QQYNSYPYMYT |
| ALK#4 | LCDR1 | RASESVDNYGISFMN |
| | LCDR2 | AASNQGS |
| | LCDR3 | QQSKEVPWT |
| ALK#5 | LCDR1 | KASQNVGTNVA |
| | LCDR2 | SASYRYS |
| | LCDR3 | QQYNSYPYMYT |
| ALK#6 | LCDR1 | KASQNVGTAVA |
| | LCDR2 | SASNRFT |
| | LCDR3 | QQYSSYPLT |
| ALK#7 | LCDR1 | KASQNVGTNVA |
| | LCDR2 | SASYRYS |
| | LCDR3 | QRYNSYPYMFT |

In some embodiments, the ALK CAR includes an anti-ALK antibody LCDR1 that is at least 85%, at least 90%, at least 95%, at least 98%, at least 99%, or 100% identical to an exemplary amino acid sequence as provided below:

RASENIYYSLA

In some embodiments, the ALK CAR includes an anti-ALK antibody LCDR2 that is at least 85%, at least 90%, at least 95%, at least 98%, at least 99%, or 100% identical to an exemplary amino acid sequence as provided below:

NANSLED

In some embodiments, the ALK CAR includes an anti-ALK antibody LCDR3 that is at least 85%, at least 90%, at least 95%, at least 98%, at least 99%, or 100% identical to an exemplary amino acid sequence as provided below:

KQAYDVPFT

In some embodiments, the ALK CAR includes an anti-ALK antibody LCDR1 that is at least 85%, at least 90%, at least 95%, at least 98%, at least 99%, or 100% identical to an exemplary amino acid sequence as provided below:

SVSQGISNSLN

In some embodiments, the ALK CAR includes an anti-ALK antibody LCDR2 that is at least 85%, at least 90%, at least 95%, at least 98%, at least 99%, or 100% identical to an exemplary amino acid sequence as provided below:

YTSSLHS

In some embodiments, the ALK CAR includes an anti-ALK antibody LCDR3 that is at least 85%, at least 90%, at least 95%, at least 98%, at least 99%, or 100% identical to an amino acid sequence as provided below:

QQYSKLPLT

In some embodiments, the ALK CAR includes an anti-ALK antibody LCDR1 that is at least 85%, at least 90%, at least 95%, at least 98%, at least 99%, or 100% identical to an exemplary amino acid sequence as provided below:

KASQNVGTNVA

In some embodiments, the ALK CAR includes an anti-ALK antibody LCDR2 that is at least 85%, at least 90%, at least 95%, at least 98%, at least 99%, or 100% identical to an exemplary amino acid sequence as provided below:

SASYRYS

In some embodiments, the ALK CAR includes an anti-ALK antibody LCDR3 that is at least 85%, at least 90%, at least 95%, at least 98%, at least 99%, or 100% identical to an exemplary amino acid sequence as provided below:

QQYNSYPYMYT

In some embodiments, the ALK CAR includes an anti-ALK antibody LCDR1 that is at least 85%, at least 90%, at least 95%, at least 98%, at least 99%, or 100% identical to an exemplary amino acid sequence as provided below:

RASESVDNYGISFMN

In some embodiments, the ALK CAR includes an anti-ALK antibody LCDR2 that is at least 85%, at least 90%, at least 95%, at least 98%, at least 99%, or 100% identical to an exemplary amino acid sequence as provided below:

AASNQGS

In some embodiments, the ALK CAR includes an anti-ALK antibody LCDR3 that is at least 85%, at least 90%, at least 95%, at least 98%, at least 99%, or 100% identical to an exemplary amino acid sequence as provided below:

QQSKEVPWT

In some embodiments, the ALK CAR includes an anti-ALK antibody LCDR1 that is at least 85%, at least 90%, at least 95%, at least 98%, at least 99%, or 100% identical to an exemplary amino acid sequence as provided below:

KASQNVGTAVA

In some embodiments, the ALK CAR includes an anti-ALK antibody LCDR2 that is at least 85%, at least 90%, at least 95%, at least 98%, at least 99%, or 100% identical to an exemplary amino acid sequence as provided below:

SASNRFT

In some embodiments, the ALK CAR includes an anti-ALK antibody LCDR3 that is at least 85%, at least 90%, at least 95%, at least 98%, at least 99%, or 100% identical to an exemplary amino acid sequence as provided below:

QQYSSYPLT

In some embodiments, the ALK CAR includes an anti-ALK antibody LCDR3 that is at least 85%, at least 90%, at least 95%, at least 98%, at least 99%, or 100% identical to an exemplary amino acid sequence as provided below:

QRYNSYPYMFT

In some embodiments, the ALK CAR includes one or more CDRs of a VH region selected from ALK Antibody #1 (ALK #1), ALK Antibody #2 (ALK #2), ALK Antibody #3 (ALK #3), ALK Antibody #4 (ALK #4), ALK Antibody #5 (ALK #5), ALK Antibody #6 (ALK #6), or ALK Antibody #7 (ALK #7) (see Table 4). In some embodiments, the ALK CAR includes one or more CDRs of a VH region selected from ALK #1. In some embodiments, the ALK CAR includes one or more CDRs of a VH region selected from ALK #2. In some embodiments, the ALK CAR includes one or more CDRs of a VH region selected from ALK #3. In some embodiments, the ALK CAR includes one or more CDRs of a VH region selected from ALK #4. In some embodiments, the ALK CAR includes one or more CDRs of a VH region selected from ALK #5. In some embodiments, the ALK CAR includes one or more CDRs of a VH region selected from ALK #6. In some embodiments, the ALK CAR includes one or more CDRs of a VH region selected from ALK #7.

TABLE 4
Variable Heavy Chain (VH) Complementary
Determining Region (CDR) ALK
Antibody Sequences (Kabat)

| ALK | | VH CDR Amino Acid |
|---|---|---|
| Antibody | CDR | Sequence |
| ALK#1 | HCDR1 | SYWMN |
| | HCDR2 | QIYPGDGDTNYNGKFKG |
| | HCDR3 | YYYGSKAY |
| ALK#2 | HCDR1 | SYWMH |
| | HCDR2 | RIDPNSGGTKYNEKFKS |
| | HCDR3 | DYYGSSYRFAY |
| ALK#3 | HCDR1 | NYWMH |
| | HCDR2 | YINPSSGYTKYNQKFKD |
| | HCDR3 | DYYGSSSWFAY |
| ALK#4 | HCDR1 | SYWVN |
| | HCDR2 | QIYPGDGDTNYNGKFKG |
| | HCDR3 | SRGYFYGSTYDS |
| ALK#5 | HCDR1 | SYWMH |
| | HCDR2 | YIKPSSGYTKYNQKFKD |
| | HCDR3 | DYYGSSSWFAY |
| ALK#6 | HCDR1 | SYAMS |
| | HCDR2 | YISSGGDYIYYADTVKG |
| | HCDR3 | ERIWLRRFFDV |
| ALK#7 | HCDR1 | SYWMH |
| | HCDR2 | YINPSSGYTKYNQKFKD |
| | HCDR3 | DYYGSSSWFAY |

In some embodiments, the ALK CAR includes an anti-ALK antibody HCDR1 that is at least 85%, at least 90%, at least 95%, at least 98%, at least 99%, or 100% identical to an exemplary amino acid sequence as provided below:

SYWMN

In some embodiments, the ALK CAR includes an anti-ALK antibody HCDR2 that is at least 85%, at least 90%, at least 95%, at least 98%, at least 99%, or 100% identical to an exemplary amino acid sequence as provided below:

QIYPGDGDTNYNGKFKG

In some embodiments, the ALK CAR includes an anti-ALK antibody HCDR3 that is at least 85%, at least 90%, at least 95%, at least 98%, at least 99%, or 100% identical to an exemplary amino acid sequence as provided below:

YYYGSKAY

In some embodiments, the ALK CAR includes an anti-ALK antibody HCDR1 that is at least 85%, at least 90%, at least 95%, at least 98%, at least 99%, or 100% identical to an exemplary amino acid sequence as provided below:

SYWMH

In some embodiments, the ALK CAR includes an anti-ALK antibody HCDR2 that is at least 85%, at least 90%, at least 95%, at least 98%, at least 99%, or 100% identical to an exemplary amino acid sequence as provided below:

RIDPNSGGTKYNEKFKS

In some embodiments, the ALK CAR includes an anti-ALK antibody HCDR3 that is at least 85%, at least 90%, at least 95%, at least 98%, at least 99%, or 100% identical to an exemplary amino acid sequence as provided below:

DYYGSSYRFAY

In some embodiments, the ALK CAR includes an anti-ALK antibody HCDR1 that is at least 85%, at least 90%, at least 95%, at least 98%, at least 99%, or 100% identical to an exemplary amino acid sequence as provided below:

NYWMH

In some embodiments, the ALK CAR includes an anti-ALK antibody HCDR2 that is at least 85%, at least 90%, at least 95%, at least 98%, at least 99%, or 100% identical to an exemplary amino acid sequence as provided below:

YINPSSGYTKYNQKFKD

In some embodiments, the ALK CAR includes an anti-ALK antibody HCDR3 that is at least 85%, at least 90%, at least 95%, at least 98%, at least 99%, or 100% identical to an exemplary amino acid sequence as provided below:

DYYGSSSWFAY

In some embodiments, the ALK CAR includes an anti-ALK antibody HCDR1 that is at least 85%, at least 90%, at least 95%, at least 98%, at least 99%, or 100% identical to an exemplary amino acid sequence as provided below:

SYWVN

In some embodiments, the ALK CAR includes an anti-ALK antibody HCDR3 that is at least 85%, at least 90%, at least 95%, at least 98%, at least 99%, or 100% identical to an exemplary amino acid sequence as provided below:

SRGYFYGSTYDS

In some embodiments, the ALK CAR includes an anti-ALK antibody HCDR1 that is at least 85%, at least 90%, at least 95%, at least 98%, at least 99%, or 100% identical to an exemplary amino acid sequence as provided below:

SYWMH

In some embodiments, the ALK CAR includes an anti-ALK antibody HCDR2 that is at least 85%, at least 90%, at least 95%, at least 98%, at least 99%, or 100% identical to an exemplary amino acid sequence as provided below:

YIKPSSGYTKYNQKFKD

In some embodiments, the ALK CAR includes an anti-ALK antibody HCDR1 that is at least 85%, at least 90%, at least 95%, at least 98%, at least 99%, or 100% identical to an exemplary amino acid sequence as provided below:

SYAMS

In some embodiments, the ALK CAR includes an anti-ALK antibody HCDR2 that is at least 85%, at least 90%, at least 95%, at least 98%, at least 99%, or 100% identical to an exemplary amino acid sequence as provided below:

YISSGGDYIYYADTVKG

In some embodiments, the ALK CAR includes an anti-ALK antibody HCDR3 that is at least 85%, at least 90%, at least 95%, at least 98%, at least 99%, or 100% identical to an exemplary amino acid sequence as provided below:

ERIWLRRFFDV

In some embodiments, the ALK CAR includes an anti-ALK antibody HCDR1 that is at least 85%, at least 90%, at least 95%, at least 98%, at least 99%, or 100% identical to an exemplary amino acid sequence as provided below:

SYWMH

In some embodiments, the ALK CAR comprises one or more CDRs from a VL region and one or more CDRs from a VH region that are at least 85%, at least 90%, at least 95%, at least 98%, at least 99%, or 100% identical to the CDRs of the VL and VH amino acid sequences of any one of antibodies ALK #1, ALK #2, ALK #3, ALK #4, ALK #5, ALK #6, or ALK #7. In some embodiments, the ALK CAR comprises one or more CDRs from a VL region and one or more CDRs from a VH region of any one of antibodies ALK #1, ALK #2, ALK #3, ALK #4, ALK #5, ALK #6, or ALK #7. In some embodiments, the ALK CAR comprises three CDRs from a VL region of any one of antibodies ALK #1, ALK #2, ALK #3, ALK #4, ALK #5, ALK #6, or ALK #7. In some embodiments, the ALK CAR comprises three CDRs from a VH region of any one of antibodies ALK #1, ALK #2, ALK #3, ALK #4, ALK #5, ALK #6, or ALK #7. In some embodiments, the ALK CAR comprises three CDRs from a VL region and three CDRs from a VH region of any one of antibodies ALK #1, ALK #2, ALK #3, ALK #4, ALK #5, ALK #6, or ALK #7.

### Vectors

CAR-T cells may be produced by using genome-integrating vectors, including but not limited to viral vectors, including retrovirus, lentivirus or transposon, or non-genome-integrating (episomal) DNA/RNA vectors, such as plasmids or mRNA. Production of CARs and CAR-T cells is known in the art (see e.g., U.S. Pat. Nos. 7,446,190, 7,741,465, 9,181,527; Kalos et al. Sci Transl Med. 2011, 3(95):95ra73, Milone et al. Mol Ther. 2009, 17(8):1453-64, and Maude et al. N Engl J Med. 2014, 371(16):1507-17, which are incorporated herein in their entirety).

Vectors containing a nucleotide sequence encoding an ALK CAR are provided. The vectors used to express an ALK CAR as described herein may be any suitable expression vector known and used in the art. In some embodiments, the vector is a prokaryotic or eukaryotic vector. In some embodiments, the vector is an expression vector, such as a eukaryotic (e.g., mammalian) expression vector. In another embodiment, the vector is a plasmid (prokaryotic or bacterial) vector. In another embodiment, the vector is a viral vector (e.g., lentiviral vector). In some embodiments, the vector further includes a promoter operably linked to the nucleotide sequence encoding the ALK CAR. In a particular embodiment, the promoter is a cytomegalovirus (CMV) promoter.

In some embodiments, the vectors comprise a nucleotide sequence encoding a VH and/or VL amino acid sequence that is at least 95%, at least 96%, at least 97%, at least 98%, at least 99%, or 100% identical to a VH and/or VL amino acid sequence of ALK Antibody #1 (ALK #1), ALK Antibody #2 (ALK #2), ALK Antibody #3 (ALK #3), ALK Antibody #4 (ALK #4), ALK Antibody #5 (ALK #5), ALK Antibody #6 (ALK #6), or ALK Antibody #7 (ALK #7). In some embodiments, the vectors comprise a nucleotide sequence encoding the VH and VL of ALK #1. In some embodiments, the vectors comprise a nucleotide sequence encoding the VH and VL of ALK #2. In some embodiments, the vectors comprise a nucleotide sequence encoding the VH and VL of ALK #3. In some embodiments, the vectors comprise a nucleotide sequence encoding the VH and VL of ALK #4. In some embodiments, the vectors comprise a nucleotide sequence encoding the VH and VL of ALK #5. In some embodiments, the vectors comprise a nucleotide sequence encoding the VH and VL of ALK #6. In some embodiments, the vectors comprise a nucleotide sequence encoding the VH and VL of ALK #7.

In some embodiments, the vectors comprise a nucleotide sequence encoding one or more CDRs of a VH and/or VL amino acid sequence that is at least 95%, at least 96%, at least 97%, at least 98%, at least 99%, or 100% identical to the CDR amino acid sequences of ALK Antibody #1 (ALK #1), ALK Antibody #2 (ALK #2), ALK Antibody #3 (ALK #3), ALK Antibody #4 (ALK #4), ALK Antibody #5 (ALK #5), ALK Antibody #6 (ALK #6), or ALK Antibody #7 (ALK #7). In some embodiments, the vectors comprise a nucleotide sequence encoding the CDRs of ALK #1. In some embodiments, the vectors comprise a nucleotide sequence encoding the CDRs of ALK #2. In some embodiments, the vectors comprise a nucleotide sequence encoding the CDRs of ALK #3. In some embodiments, the vectors comprise a nucleotide sequence encoding the CDRs of ALK #4. In some embodiments, the vectors comprise a nucleotide sequence encoding the CDRs of ALK #5. In some embodiments, the vectors comprise a nucleotide sequence encoding the CDRs of ALK #6. In some embodiments, the vectors comprise a nucleotide sequence encoding the CDRs of ALK #7.

Provided herein are ALK CAR-T cells produced by transfecting a host cell (e.g., T cell, natural killer (NK) cell, cytotoxic T lymphocyte (CTL) cell, or regulatory T cell) with an expression vector containing a polynucleotide encoding an ALK CAR, as described herein, as known and used in the art under conditions sufficient to allow for expression of the ALK CAR, thereby producing the CAR-T cell. Isolated cells (e.g., T cells, NK cells, CTL cells, or regulatory T cells) containing the vectors are also provided. Collections of plasmids (vectors) are also contemplated. In certain embodiments, the collection of plasmids includes plasmid encoding an ALK CAR as described herein.

Methods of generating chimeric antigen receptors and T cells including such receptors are known in the art and further described herein (see, e.g., Brentjens et al., 2010, Molecular Therapy, 18:4, 666-668; Morgan et al., 2010, Molecular Therapy, published online Feb. 23, 2010, pages 1-9; Till et al., 2008, Blood, 1 12:2261-2271; Park et al., Trends Biotechnol., 29:550-557, 2011; Grupp et al., N Engl J Med., 368:1509-1518, 2013; Han et al., J. Hematol Oncol., 6:47, 2013; Tumaini et al., Cytotherapy, 15, 1406-1417, 2013; Haso et al., (2013) Blood, 121, 1165-1174; PCT Pubs. WO2012/079000, WO2013/126726; and U.S. Pub. 2012/0213783, each of which is incorporated by reference herein in its entirety).

### Pharmaceutical Compositions

Compositions comprising at least one ALK CAR, polynucleotide encoding an ALK CAR, or engineered immune cell comprising an ALK CAR, as described herein are provided. In some embodiments, the compositions further comprise a pharmaceutically acceptable carrier, diluent, excipient, or vehicle. In some embodiments, an adjuvant (a pharmacological or immunological agent that modifies or boosts an immune response, e.g., to produce more antibodies that are longer-lasting) is also employed. For example, without limitation, the adjuvant can be an inorganic compound, such as alum, aluminum hydroxide, or aluminum phosphate; mineral or paraffin oil; squalene; detergents such as Quil A; plant saponins; Freund's complete or incomplete adjuvant, a biological adjuvant (e.g., cytokines such as IL-1, IL-2, IL-12, or IL-15); bacterial products such as killed Bordetella pertussis, or toxoids; or immunostimulatory oligonucleotides (such as CpG oligonucleotides).

Compositions and preparations (e.g., physiologically or pharmaceutically acceptable compositions) containing ALK CARs, polynucleotides encoding an ALK CAR, or engineered immune cells comprising an ALK CARs for parenteral administration include, without limitation, sterile aqueous or non-aqueous solutions, suspensions, and emulsions. Nonlimiting examples of non-aqueous solvents include propylene glycol, polyethylene glycol, vegetable oils, such as olive oil and canola oil, and injectable organic esters, such as ethyl oleate. Aqueous carriers include water, alcoholic/aqueous solutions, emulsions or suspensions, including saline and buffered media. Parenteral vehicles include, for example, sodium chloride solution, Ringer's dextrose, dextrose and sodium chloride, lactated Ringer's, or fixed oils. Intravenous vehicles include, for example, fluid and nutrient replenishers, electrolyte replenishers (such as those based on Ringer's dextrose), and the like. Preservatives and other additives may also be present in such compositions and preparations, such as, for example, antimicrobials, antioxidants, chelating agents, colorants, stabilizers, inert gases and the like.

Some of the compositions may potentially be administered as a pharmaceutically acceptable acid- or base-addition salt, formed by reaction with inorganic acids, such as hydrochloric acid, hydrobromic acid, perchloric acid, nitric acid, thiocyanic acid, sulfuric acid, and phosphoric acid, and organic acids, such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, tri-alkyl and aryl amines and substituted ethanolamines.

Provided herein are pharmaceutical compositions which include a therapeutically effective amount of an ALK CAR, polynucleotide encoding an ALK CAR, or engineered immune cell comprising an ALK CAR, alone, or in combination with a pharmaceutically acceptable carrier. Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid or aqueous solution, suspension, emulsion, dispersion, tablet, pill, capsule, powder, or sustained release formulation. A liquid or aqueous composition can be lyophilized and reconstituted with a solution or buffer prior to use. The composition can be formulated as a suppository, with traditional binders and carriers such as triglycerides. Oral formulations can include standard carriers, such as pharmaceutical grades of mannitol, lactose, starch, magnesium stearate, sodium saccharine, cellulose, and magnesium carbonate. Any of the commonly known pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives and the like. Other media that can be used in the compositions and administration methods as described are normal saline and sesame oil.

### Methods of Treatment, Administration and Delivery

Methods of treating a disease (e.g., ALK-positive cancers (e.g., neuroblastoma)), or symptoms thereof, are provided. The methods comprise administering a therapeutically effective amount of an ALK CAR, a polynucleotide encoding an ALK CAR, or engineered immune cell comprising an ALK CAR, as described herein, or a pharmaceutical composition thereof, as described herein, to a subject (e.g., a mammal), in particular, a human subject. The invention provides methods of treating a subject suffering from, or at risk of, or susceptible to disease, or a symptom thereof, or delaying the progression of a disease (e.g., ALK-positive cancer (e.g., neuroblastoma)). In some embodiments, the method includes administering to the subject (e.g., a mammalian subject), an amount or a therapeutic amount of an ALK CAR, a polynucleotide encoding an ALK CAR, or engineered immune cell comprising an ALK CAR, or pharmaceutical composition thereof, sufficient to treat the disease, delay the growth of, or treat the symptoms thereof (e.g., ALK-positive cancers (e.g., neuroblastoma)).

In some embodiments, the methods herein include administering to the subject (including a human subject identified as in need of such treatment) an effective amount of an ALK CAR, a polynucleotide encoding an ALK CAR, or engineered immune cell comprising an ALK CAR, or a pharmaceutical composition thereof, as described herein to produce such effect. The treatment methods are suitably administered to subjects, particularly humans, suffering from, are susceptible to, or at risk of having a disease, or symptoms thereof, namely, cancer (e.g., ALK-positive cancers (e.g., neuroblastoma)). Nonlimiting examples of ALK-positive cancers include non-small cell lung cancer (NSCLC), anaplastic large cell lymphoma (ALCL), neuroblastoma, B-cell lymphoma, thyroid cancer, colon cancer, breast cancer, inflammatory myofibroblastic tumors (IMT), renal carcinoma, esophageal cancer, melanoma, or a combination thereof. In some embodiments, the ALK-positive cancer is neuroblastoma.

The ALK-positive cancer may be caused by an oncogenic ALK gene that either forms a fusion gene with other genes, gains additional gene copies, or is genetically mutated. In some embodiments, the ALK-positive cancer is caused by an ALK fusion gene encoding an ALK fusion protein. In some embodiments, the ALK-positive cancer is caused by a fusion between the ALK gene and the nucleophosmin (NPM) gene encoding a NPM-ALK fusion protein. In some embodiments, the ALK-positive cancer is caused by a fusion between the ALK gene and the echinoderm microtubule-associated protein-like 4 (EML4) gene encoding an ELM4-ALK fusion protein. In some embodiments, the ALK-positive cancer is caused by a point mutation. In some embodiments, the point mutation is F1174L (ALK^{F1174L}).

Identifying a subject in need of such treatment can be based on the judgment of the subject or of a health care professional and can be subjective (e.g., opinion) or objective (e.g., measurable by a test or diagnostic method). Briefly, the determination of those subjects who are in need of treatment or who are “at risk” or “susceptible” can be made by any objective or subjective determination by a diagnostic test (e.g., blood sample, biopsy, genetic test, enzyme or protein marker assay), marker analysis, family history, and the like, including an opinion of the subject or a health care provider. The ALK CAR, a polynucleotide encoding an ALK CAR, or engineered immune cell comprising an ALK CAR, or pharmaceutical compositions thereof, as described herein, may also be used in the treatment of any other disorders in which disease caused by oncogenic ALK gene fusions, rearrangements, duplications or mutations may be implicated. A subject undergoing treatment can be a non-human mammal, such as a veterinary subject, or a human subject (also referred to as a “patient”).

In addition, prophylactic methods of preventing or protecting against a disease (e.g., ALK-positive cancers (e.g., neuroblastoma)), or symptoms thereof, are provided. Such methods comprise administering a therapeutically effective amount of a pharmaceutical composition comprising an ALK CAR, a polynucleotide encoding an ALK CAR, or engineered immune cell comprising an ALK CAR as described herein to a subject (e.g., a mammal, such as a human), in particular, prior to development or onset of a disease (e.g., ALK-positive cancers (e.g., neuroblastoma)).

In another embodiment, a method of monitoring the progress of a disease (e.g., ALK-positive cancers (e.g., neuroblastoma)), or monitoring treatment of the disease is provided. The method includes a diagnostic measurement (e.g., CT scan, screening assay or detection assay) in a subject suffering from or susceptible to disease or symptoms thereof (e.g., ALK-positive cancers (e.g., neuroblastoma)), in which the subject has been administered an amount (e.g., a therapeutic amount) of an ALK CAR, a polynucleotide encoding an ALK CAR, or engineered immune cell comprising an ALK CAR, or a pharmaceutical composition thereof, as described herein, sufficient to treat the disease or symptoms thereof. The diagnostic measurement in the method can be compared to samples from healthy, normal controls; in a pre-disease sample of the subject; or in other afflicted/diseased patients to establish the treated subject's disease status. For monitoring, a second diagnostic measurement may be obtained from the subject at a time point later than the determination of the first diagnostic measurement, and the two measurements can be compared to monitor the course of disease or the efficacy of the therapy/treatment. In certain embodiments, a pre-treatment measurement in the subject (e.g., in a sample or biopsy obtained from the subject or CT scan) is determined prior to beginning treatment as described; this measurement can then be compared to a measurement in the subject after the treatment commences and/or during the course of treatment to determine the efficacy of (monitor the efficacy of) the disease treatment.

The ALK CAR, a polynucleotide encoding an ALK CAR, or engineered immune cell comprising an ALK CAR, or pharmaceutical compositions thereof, can be administered to a subject by any of the routes normally used for introducing a recombinant protein or composition containing the recombinant protein into a subject. Routes and methods of administration include, without limitation, intradermal, intramuscular, intraperitoneal, intrathecal, parenteral, such as intravenous (IV) or subcutaneous (SC), vaginal, rectal, intranasal, inhalation, intraocular, intracranial, or oral. Parenteral administration, such as subcutaneous, intravenous or intramuscular administration, is generally achieved by injection (immunization). Injectables can be prepared in conventional forms and formulations, either as liquid solutions or suspensions, solid forms (e.g., lyophilized forms) suitable for solution or suspension in liquid prior to injection, or as emulsions. Injection solutions and suspensions can be prepared from sterile powders, granules, and tablets. Administration can be systemic or local.

The ALK CAR, a polynucleotide encoding an ALK CAR, or engineered immune cell comprising an ALK CAR, or pharmaceutical compositions thereof, can be administered in any suitable manner, such as with pharmaceutically acceptable carriers, diluents, or excipients as described supra. Pharmaceutically acceptable carriers are determined in part by the particular composition being administered, as well as by the particular method used to administer the composition. Accordingly, a pharmaceutical composition comprising the ALK CAR, a polynucleotide encoding an ALK CAR, or engineered immune cell comprising an ALK CAR, can be prepared using a wide variety of suitable and physiologically and pharmaceutically acceptable formulations. In some embodiments, the disclosed methods include isolating T cells from a subject, transducing the T cells with an expression vector (e.g., a lentiviral vector) encoding the ALK CAR, and administering the ALK CAR-expressing T cells to the subject for treatment of a disease ((e.g., ALK-positive cancers (e.g., neuroblastoma)) in the subject.

Administration of the ALK CAR, a polynucleotide encoding an ALK CAR, or engineered immune cell comprising an ALK CAR, or pharmaceutical compositions thereof, can be accomplished by single or multiple doses. The dose administered to a subject should be sufficient to induce a beneficial therapeutic response in a subject over time, such as to inhibit, block, reduce, ameliorate, protect against, or prevent disease (e.g., ALK-positive cancers (e.g., neuroblastoma)). The dose required will vary from subject to subject depending on the species, age, weight and general condition of the subject, by the severity of the cancer being treated, by the particular composition being used and by the mode of administration. An appropriate dose can be determined by a person skilled in the art, such as a clinician or medical practitioner, using only routine experimentation. One of skill in the art is capable of determining therapeutically effective amounts of ALK CAR, a polynucleotide encoding an ALK CAR, or engineered immune cell comprising an ALK CAR, or pharmaceutical compositions, that provide a therapeutic effect or protection against disease (e.g., ALK-positive cancers (e.g., neuroblastoma)) suitable for administering to a subject in need of treatment or protection.

In some embodiments, an ALK CAR, a polynucleotide encoding an ALK CAR, or engineered immune cell comprising an ALK CAR, or a pharmaceutical composition thereof, is administered as a maximum-tolerated dose (MTD). In some embodiments, MTD is the dose with estimated probability of dose limiting toxicity (DLT) closest to the target toxicity rate of 20%. In some embodiments, an ALK CAR, a polynucleotide encoding an ALK CAR, or engineered immune cell comprising an ALK CAR, or a pharmaceutical composition thereof, is administered in a therapeutically effective dose for a mammal. In some embodiments, the mammal is a mouse. In some embodiments, a mouse is administered a dose of 0.5 million to 15 million ALK CAR-T cells. In some embodiments, the mammal is a human. In some embodiments, a human is administered a dose of at least about 0.25×10⁶ CAR⁺ cells/kg, at least about 0.5×10⁶ CAR⁺ cells/kg, at least about 1×10⁶ CAR⁺ cells/kg, or at least about 1.5×10⁶ CAR⁺ cells/kg.

### Combination Therapies

The anaplastic lymphoma kinase chimeric antigen receptor (ALK CAR) or engineered immune cell comprising an ALK CAR as described herein can be administered alone or in combination with other therapeutic agents in a subject for the treatment of cancer (e.g., ALK-positive cancer (e.g., neuroblastoma)). For example, the ALK CAR or engineered immune cell comprising an ALK CAR can be administered with an adjuvant, such as alum, Freund's incomplete adjuvant, Freund's complete adjuvant, biological adjuvant, or immunostimulatory oligonucleotides (such as CpG oligonucleotides). The adjuvant may be conjugated to an amphiphile as previously described (H. Liu et al., Structure-based programming of lymph-node targeting in molecular vaccines. Nature 507, 5199522 (2014)). In some embodiments, the amphiphile conjugated to the adjuvant is N-hydroxy succinimidyl ester-end-functionalized poly(ethylene glycol)-lipid (NHS-PEG2KDa-DSPE).

One or more cytokines, including but not limited to, interleukin-1 (IL-1), interleukin-2 (IL-2), interleukin-4 (IL-4), interleukin-6 (IL-6), interleukin-7 (IL-7), interleukin-12 (IL-12), interleukin-15 (IL-15), interleukin-21 (IL-21), the protein memory T-cell attractant “Regulated Activation, on Normal T Expressed and Secreted” (RANTES), granulocyte-macrophage-colony stimulating factor (GM-CSF), tumor necrosis factor-alpha (TNF-α), or interferon-gamma (IFN-γ), macrophage inflammatory protein 1 alpha (MIP-1α); one or more molecules such as the TNF ligand superfamily member 4 ligand (OX40L) or the type 2 transmembrane glycoprotein receptor belonging to the TNF superfamily (4-1BBL), or combinations of these molecules, can be used as biological adjuvants, if desired or warranted (see, e.g., Salgaller et al., 1998, J. Surg. Oncol. 68(2):122-38; Lotze et al., 2000, Cancer J. Sci. Am. 6(Suppl 1):561-6; Cao et al., 1998, Stem Cells 16(Suppl 1):251-60; Kuiper et al., 2000, Adv. Exp. Med. Biol. 465:381-90). These molecules can be administered systemically (or locally) to a subject.

The ALK CAR or engineered immune cell comprising an ALK CAR can also be administered as a combination therapy with one or more other therapeutic agents, such as an ALK peptide or fusion protein, ALK peptide vaccine, ALK inhibitors, tyrosine kinase inhibitors (TKIs), and/or immune checkpoint inhibitors. Non-limiting examples of ALK inhibitors include lorlatinib (Lobrena®). Non-limiting examples of checkpoint inhibitors include programmed cell death protein 1 (PD-1) inhibitors, programmed death-ligand 1 (PD-L1), and cytotoxic T-lymphocyte-associated antigen-4 (CTLA-4) inhibitors. Nonlimiting examples of PD-1 inhibitors include pembrolizumab (Keytruda®) and nivolumab)(Opdivo®). Nonlimiting examples of CTLA-4 inhibitors include ipilimumab (Yervoy®). Non-limiting examples of TKI inhibitors include crizotinib, ceritinib, alectinib, brigatinib, and lorlatinib. In some embodiments, one or more ALK peptides or fusion proteins, ALK peptide vaccines, ALK inhibitors, immune checkpoint inhibitors, and/or TKI inhibitors is/are administered simultaneously or sequentially with ALK CAR or engineered immune cell comprising an ALK CAR to a subject (e.g., human).

In some embodiments, the ALK CAR or engineered immune cell comprising an ALK CAR is administered simultaneously or sequentially with an ALK peptide vaccine. In particular embodiments, the ALK peptide vaccine contains antigenic determinants that serve to elicit an immune response in a subject (e.g., the production of activated T-cells) that can treat and/or protect a subject against disease caused by oncogenic ALK gene fusions, rearrangements, duplications or mutations (e.g., ALK-positive cancers) and symptoms thereof. In some embodiments, the immune response includes producing T-lymphocytes. In some embodiments, the ALK peptide vaccine contains at least one ALK antigen or peptide or fragment thereof. In some embodiments, the ALK peptide vaccine contains two or more ALK peptides or antigens or fragments thereof. In some embodiments, the ALK peptides or antigens or fragments thereof are fragments of the cytoplasmic portion of an ALK protein, which bind a human leukocyte antigen (HLA). In some embodiments the ALK peptides or antigens or fragments thereof are modified with an amphiphilic conjugate to increase T-cell expansion and greatly enhance anti-tumor efficacy. In some embodiments, the amphiphile is N-hydroxy succinimidyl ester-end-functionalized poly(ethylene glycol)-lipid (NHS-PEG2KDa-DSPE).

### Kits

Also provided are kits containing the anaplastic lymphoma kinase chimeric antigen receptor (ALK CAR) or engineered immune cell comprising an ALK CAR as described, or a pharmaceutically acceptable composition containing the ALK CAR and a pharmaceutically acceptable carrier, diluent, or excipient, for administering to a subject, for example. In some embodiments, the kit is provided for treating cancer (e.g., ALK-positive cancer (e.g., neuroblastoma)) in a subject (e.g., human). In some embodiments, the kit is provided for making an ALK CAR as provided herein. In some embodiments, the kit will contain one or more of an ALK antibody or antigen binding fragment thereof, nucleic acid molecule encoding for an ALK peptide, ALK CAR or T cell expressing an ALK CAR as disclosed herein. The ALK CAR may be in the form of a polypeptide or a polynucleotide encoding an ALK CAR, as described herein. In some embodiments, the kit comprises a vector containing a nucleotide sequence encoding an ALK CAR as disclosed herein. As will be appreciated by the skilled practitioner in the art, such a kit may contain one or more containers, labels, carriers, diluents or excipients, as necessary, and instructions for use.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Useful techniques for particular embodiments will be discussed in the sections that follow.

The following examples are put forth to provide those of ordinary skill in the art with a complete disclosure and description of how to make and use the assay, screening, and therapeutic methods of the invention, and are not intended to limit the scope of what the inventors regard as their invention.

### EXAMPLES

The following examples are provided to illustrate certain particular features and/or embodiments. The examples should not be construed to limit the disclosure to the particular features or embodiments described.

### Example 1: Generation of ALK CAR-T Constructs

More than 70% of neuroblastoma express the Anaplastic Lymphoma Kinase (ALK) receptor (Chiarle R, et al., The anaplastic lymphoma kinase in the pathogenesis of cancer. Nat Rev Cancer 2008 January; 8(1):11-23). About 10% of neuroblastoma cases have an activating point mutation in the ALK protein (e.g., ALK^{F1174L}), correlating to advanced disease stage and poor prognosis (Passoni L, et al., Mutation-independent anaplastic lymphoma kinase overexpression in poor prognosis neuroblastoma patients. Cancer Res 2009 Sep. 15; 69(18):7338-46; Mosse Y P, et al., Identification of ALK as a major familial neuroblastoma predisposition gene. Nature 2008 Oct. 16; 455(7215):930-5). Promising antitumor effects have been obtained with ALK tyrosine kinase inhibitors (TKIs), but almost invariably disease progresses (Mosse Y P, Anaplastic Lymphoma Kinase as a Cancer Target in Pediatric Malignancies. Clin Cancer Res 2016 Feb. 1; 22(3):546-52). Thus, while ALK remains a promising target in neuroblastoma, it is clinically evident that alternative strategies to TKIs must be implemented to target ALK. In particular, ALK protein has several features suitable to be targeted by immunotherapy. For instance, ALK has almost no expression in normal tissues and is naturally immunogenic in humans (Blasco R B, et al., Comment on “ALK is a therapeutic target for lethal sepsis,” Sci Transl Med 2018 Dec. 12; 10(471)). Patients with ALK-rearranged lymphoma and lung cancer can indeed spontaneously develop immune responses against ALK (Awad M M, et al., Epitope mapping of spontaneous autoantibodies to anaplastic lymphoma kinase (ALK) in non-small cell lung cancer. Oncotarget 2017 Nov. 3; 8(54):92265-74; Ait-Tahar K, et al., Correlation of the autoantibody response to the ALK oncoantigen in pediatric anaplastic lymphoma kinase-positive anaplastic large cell lymphoma with tumor dissemination and relapse risk. Blood 2010 Apr. 22; 115(16):3314-9). Importantly, ALK is a potent driver oncogene required for tumor survival and growth, which minimizes the chances of escape of ALK-negative tumor cells (Chiarle R, et al., The anaplastic lymphoma kinase in the pathogenesis of cancer. Nat Rev Cancer 2008 January; 8(1):11-23; Voena C, et al., Efficacy of a Cancer Vaccine against ALK-Rearranged Lung Tumors. Cancer Immunol Res 2015 December; 3(12):1333-43). Specifically, in neuroblastoma, therapeutic effects are achieved by ALK knock-down (Di P D, et al., Neuroblastoma-targeted nanoparticles entrapping siRNA specifically knockdown ALK. Mol Ther 2011 June; 19(6):1131-40), inhibition (Infarinato N R, et al., The ALK/ROS1 Inhibitor PF-06463922 Overcomes Primary Resistance to Crizotinib in ALK-Driven Neuroblastoma. Cancer Discov 2016 January; 6(1):96-107) or antibody-mediated drug delivery (Sano R, et al., An antibody-drug conjugate directed to the ALK receptor demonstrates efficacy in preclinical models of neuroblastoma. Sci Transl Med 2019 Mar. 13; 11(483)). ALK-specific cancer immunotherapy based on CAR-Ts may represent an opportunity to increase clinical benefits. Accordingly, a series of ALK-specific CARs (ALK CARs) were developed from ALK antibodies that recognize both human and murine ALK and validated in preclinical models of neuroblastoma.

Seven (7) ALK-specific antibodies (ALK Antibodies #1-#7) directed against the extracellular domain of human ALK receptor were evaluated for use in CAR-based immunotherapy. These antibodies demonstrated specificity to the ALK extracellular domain (ECD) with various activities on ALK signaling (Table 5). ALK Antibodies #4 and #7 were agonistic of ALK signaling, ALK Antibodies #2, #3, #5, and #6 inhibited ALK signaling, and ALK Antibody #1 had no effect on ALK signaling. These antibodies also demonstrated different biology affinity and bound to various portions of human ALK receptor. For example, ALK antibodies #5, #6 and #7) recognize both the human and murine ALK, and can thus be utilized for toxicity studies in mice.

TABLE 5
Characterization of ALK Antibodies

| ALK | ALK | K_{D}^{app} | ALK | Mouse-ALK |
|---|---|---|---|---|
| Antibody | Phosphorylation | [nM] | Turnover | Binding |
| 1 | No activity | 0.35 | no | moderate |
| 2 | Inhibitor | 0.2 | no | moderate |
| 3 | Weak inhibitor | 0.5 | no | no |
| 4 | Strong agonist | 0.5 | strong | no |
| 5 | Inhibitor | 0.5 | no | strong |
| 6 | Inhibitor | 0.4 | strong | strong |
| 7 | Weak agonist | 0.5 | strong | strong |

To develop a chimeric antigen receptor (CAR)-based immunotherapy for the treatment of neuroblastoma, CAR-T cells were constructed by fusing each of the seven ALK antibodies to T cell receptor intracellular domains for the activation of T cells. Specifically, the VH and VL regions were cloned from each of the seven antibodies to generate scFvs. The ALK scFvs were cloned into a murine CAR backbone, i.e., SFG-m1928z-GFP CAR-T retroviral construct. The SFG-m1928z-GFP CAR construct has been shown to be very efficacious in targeting CD19+ cells in mouse models (Dr. M Sadelain (MSKCC, NY)).

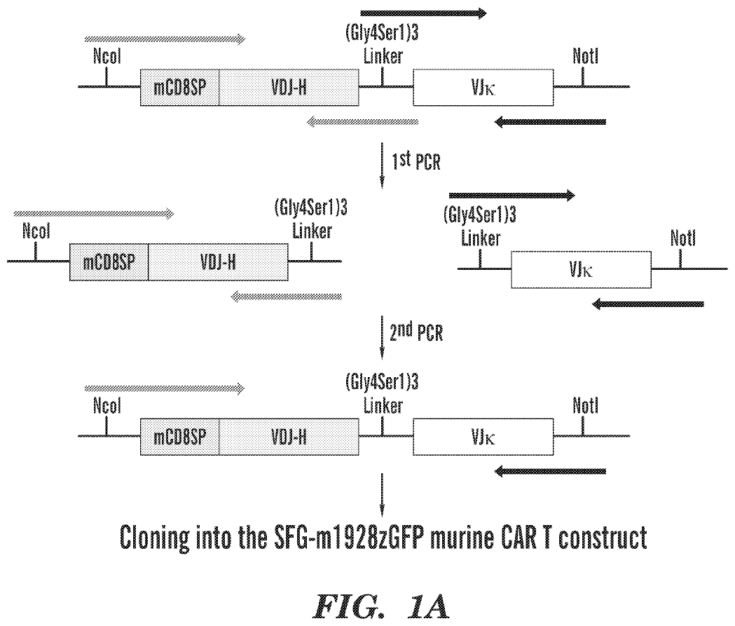

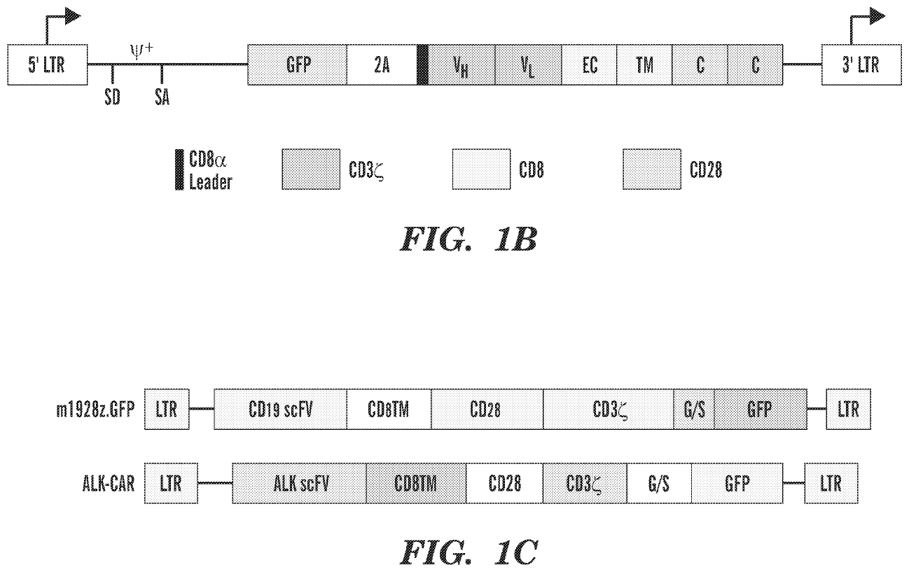

The cloning strategy is shown in FIG. 1A. Overlap PCR was used to create a VDJ-H followed by mCD8 signal peptide and VJκ followed by partial (Gly₄Ser₁)₃ linker sequence. After a second-round of PCR, mCD8SP, VDJ-H, linker, and VJκ were fused. The efficacy of gene transfer was evaluated by GFP expression. FIG. 1B shows a schematic representation of the mouse CAR genetic construct backbone GL-2A-m1928z for the reporter gene (GFP) and CAR (m1928z) using a 2A peptide sequence. Depicted are the packaging signal, splice donor (SD), splice acceptor (SA), the VH and VL regions of the scFv, the extracellular (EC) domain (e.g., CD8), transmembrane (TM) domain (e.g., CD8), and cytosolic (C) domains (e.g., CD28, CD3ζ). The m1928z CAR construct was created as provided in Davila et al., CD19 CAR-Targeted T Cells Induce Long-Term Remission and B Cell Aplasia in an Immunocompetent Mouse Model of B Cell Acute Lymphoblastic Leukemia, PLoS ONE (2013), which is incorporated herein by reference in its entirety. The resulting ALK CAR constructs contain at least the scFv (i.e., VH and VL) of ALK (e.g., ALK #1, ALK #1, ALK #2, ALK #3, ALK #4, ALK #5, ALK #6, or ALK #7), a CD8 transmembrane domain, a CD28 intracellular signaling domain, and a CD3ζ intracellular signaling domain (FIG. 1C).

### Example 2: Transduction of ALK CAR-T Constructs into Mouse T Cells

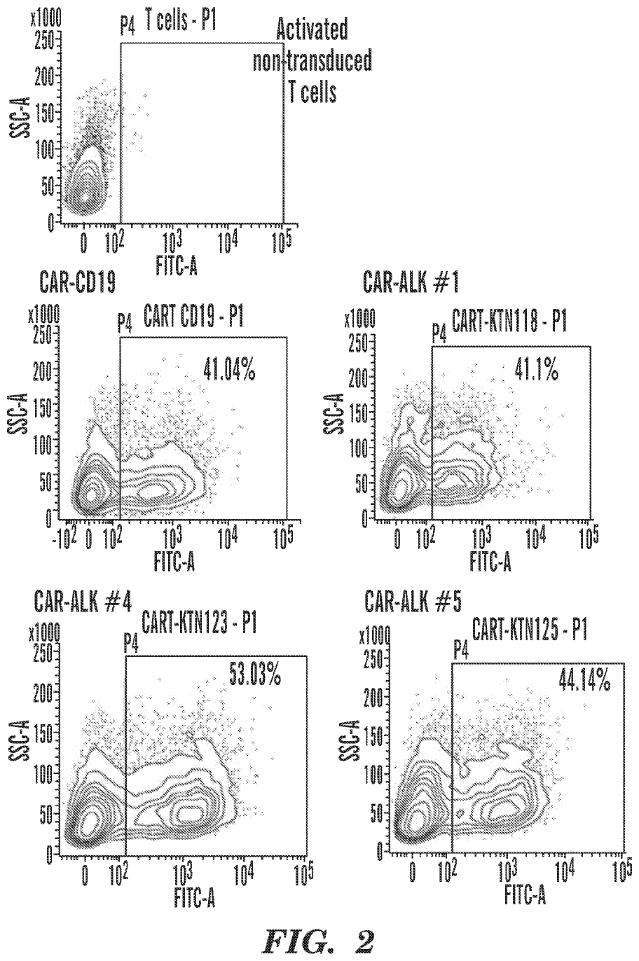

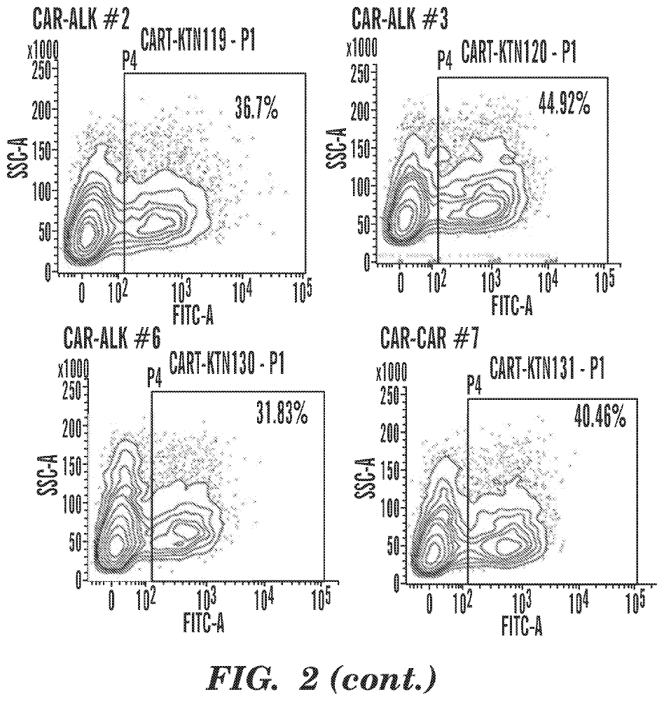

To generate CAR-T cells with the ALK CAR constructs, retrovirus vectors expressing CD19 (m1928Z-GFP) scFv or ALK CARs were transduced into T cell splenocytes from C57BL/6J mice. Mouse T cells were purified from spleen, activated with anti-CD3/CD28+IL2, and transduced with a CAR retroviral construct containing GFP as a reporter. Efficiency of transduction was evaluated 48 hours after viral infection by GFP reporter expression. Transduction efficiency was evaluated by measuring the percentage of GFP-positive T cells (FIG. 2). Activated non-transduced T cells were used as a negative control and CD19-directed CAR-T cells were used as a positive control (FIG. 2).

### Example 3: ALK-Specific Cytolytic Activity and Cytokine Release of ALK CAR-T Cell Constructs In Vitro

Cytokine release by ALK-specific CAR constructs was evaluated. Specifically, IFNγ and GM-CSF production by ALK CAR-T cells was measured. Retrovirally transduced CAR-T cells were incubated at a 1:1 effector cell (GFP+CAR-T cells) to target cell ratio (E:T ratio). Target cells included NIH3T3, Eμ-myc, SH-SY5Y (expresses normal low levels of mutated ALK^{F1174L}) and SK-N-BE (expresses high levels of amplified wild-type ALK).

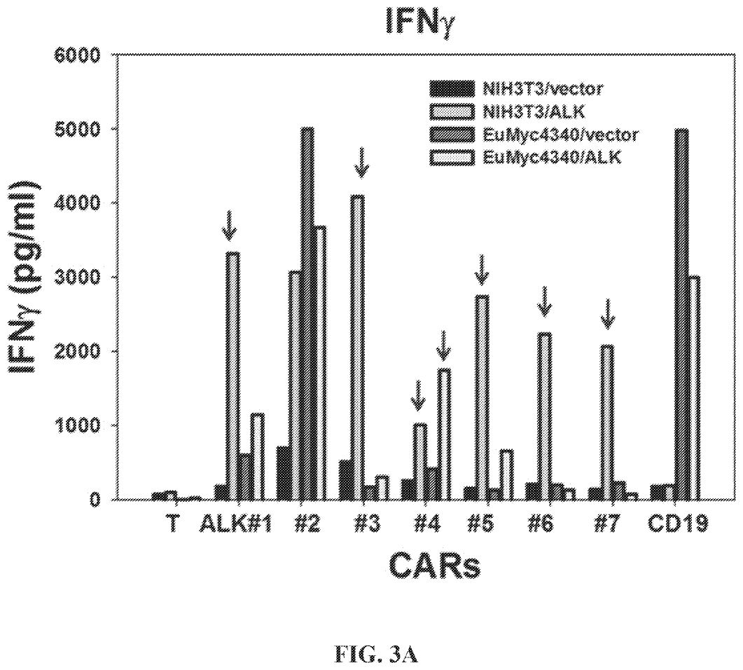

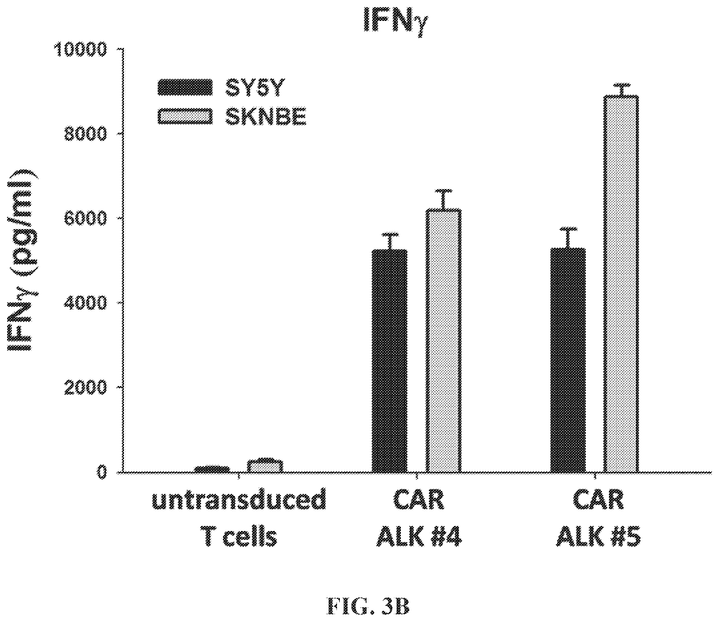

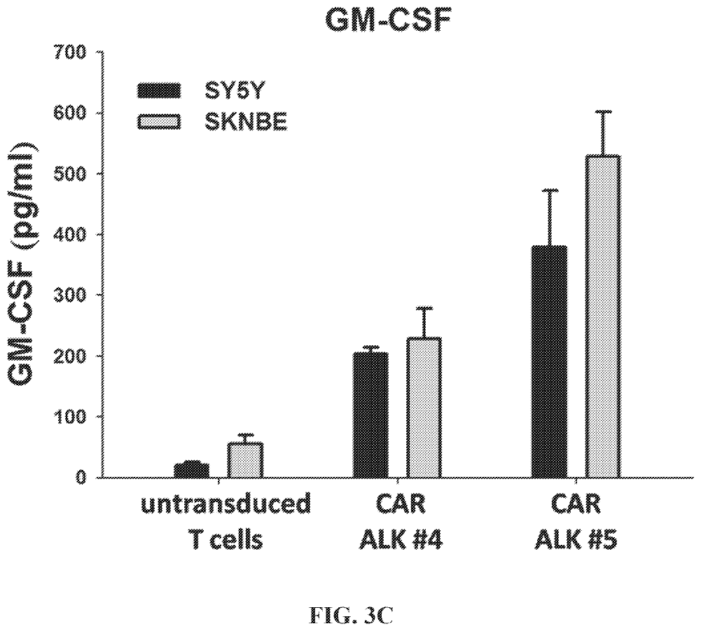

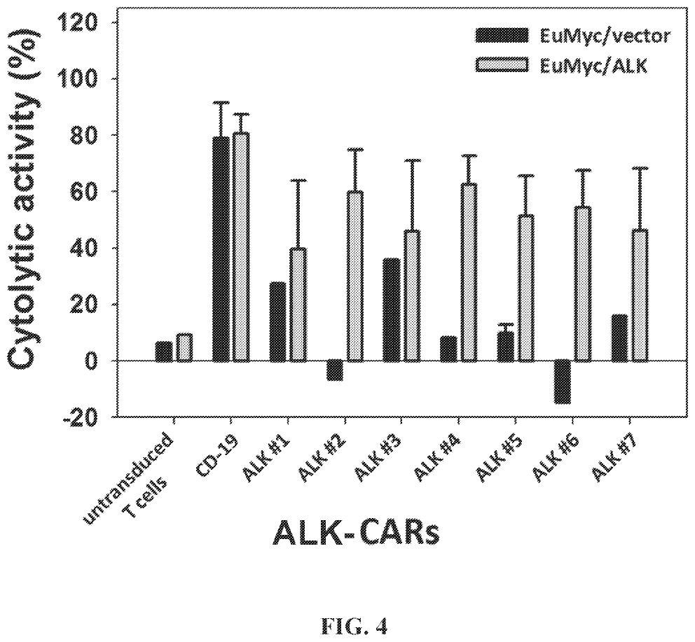

IFNγ production was measured in NIH3T3 and Eμ-myc cells transduced with full-length human ALK retroviral vector or mock vector in the presence of CAR-T cells with ALK #1, ALK #2, ALK #3, ALK #4, ALK #5, ALK #6, or ALK #7 (FIG. 3A). CD19 CAR-T cells were co-incubated as a positive control because human neuroblastoma cells do not express CD19, and untransduced T cells were co-incubated as a negative control. IFNγ production (FIG. 3B) and GM-CSF production (FIG. 3C) were measured in human neuroblastoma cells SH-SY5Y and SK-N-BE in the presence of CAR-T cells with ALK #4 or ALK #5. Untransduced T cells were used as a negative control. ELISA was used to evaluate production of IFNγ and GM-CSF in the supernatant of cells after 24 hours co-incubation. Cytokine production was observed in human neuroblastoma cells, SY5Y and SK-N-BE, and NIH3T3 and Eμ-myc cells overexpressing human full-length ALK.

The in vitro killing activity of ALK CAR-T cells was also evaluated. Eμ-myc cells overexpressing mock vector or full-length human ALK were stained with CFSE and co-incubated with effector ALK CAR-T cells at E:T ratios of 1:1, 5:1 or 10:1. The cell numbers of CAR-T cells were normalized based on the percentage of GFP positive cells transduced with the CAR construct ALK #1, ALK #2, ALK #3, ALK #4, ALK #5, ALK #6, or ALK #7. After 18 and 24 hours, the cytolytic activity was calculated by determining the fraction of alive target cells with the formula: cytolytic activity=100−% of CSFE+/CD19+ alive cells. CD19 CAR-T cells were used as golden standard control as they efficiently target CD19+Eμ-Myc cells (see Davila et al., PlosOne 2013). Eμ-Myc vector (ALK−) cells were used as control to determine the specificity of ALK-directed cytolytic activity. Strong cytolytic activity of CAR-T cells against Eμ-myc/ALK cells was found in in the CFSE assay (FIG. 4). Several ALK CARs showed a cytolytic activity 2 to 6 times higher than a recently published ALK CAR-T construct generated from a different antibody (Sotillo E, et al., Convergence of Acquired Mutations and Alternative Splicing of CD19 Enables Resistance to CART-19 Immunotherapy. Cancer Discov 2015 December; 5(12):1282-95).

### Example 4: Validation of ALK CAR Constructs In Vivo

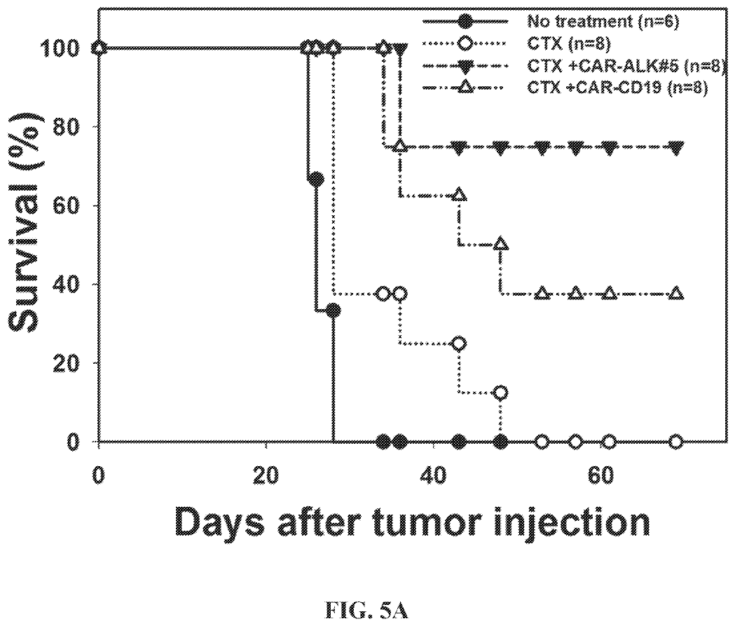

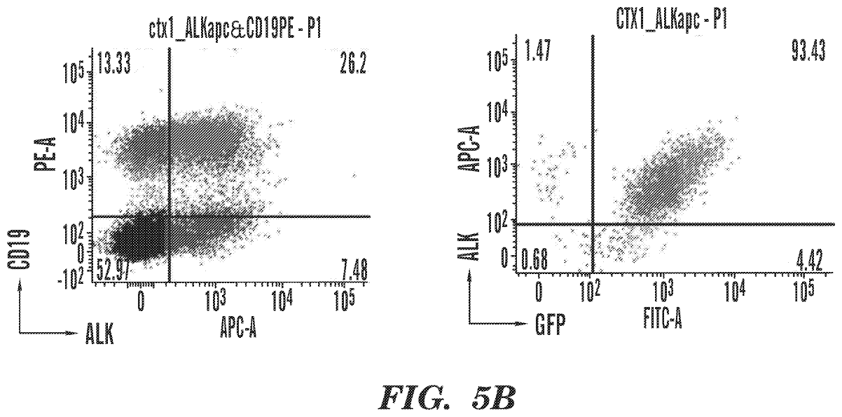

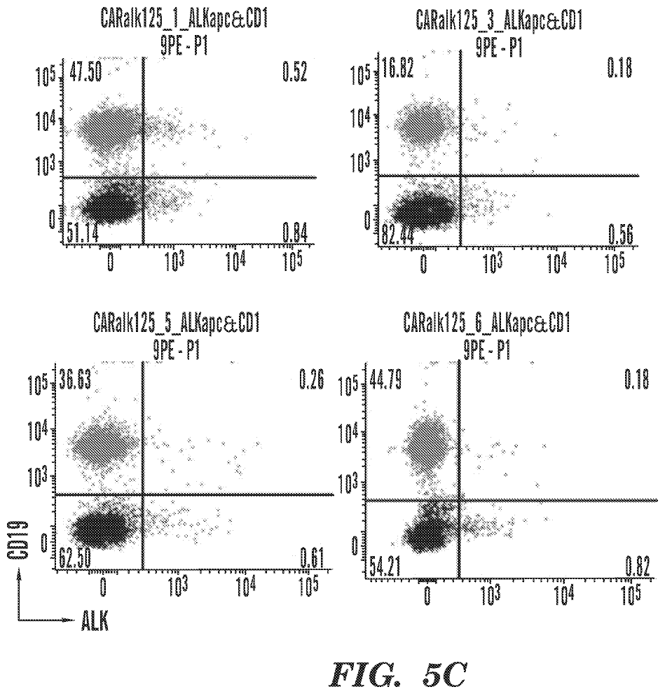

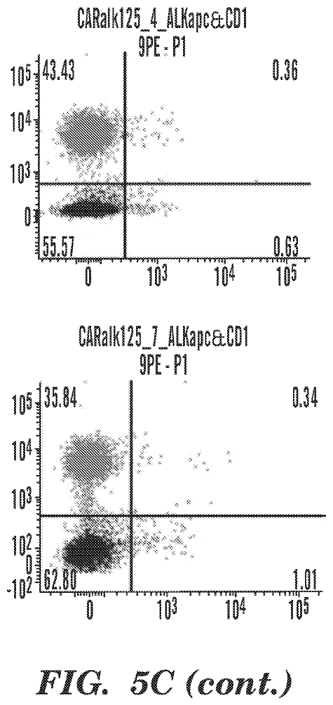

The CD19+/ALK+systemic leukemia model was used to validate and rank cytolytic activity of ALK CARs in vivo. Adoptive transfer of ALK CAR-T cells was conducted in mice with Eμ-myc/ALK systemic tumors. Mice were treated with cytophspamide (CTX, 100 mg/kg) alone (n=8), cytophosphamide plus CAR-CD19 (15×10⁶ based on GFP+) (n=8), or cytophosphamide plus CAR-ALK #5 (15×10⁶ based on GFP+) (n=8). Untreated mice (n=6) were used as a negative control. FIG. 5A provides a survival curve of these treated mice. Mice treated with immunosuppressor, cyclophosphamide, and CAR-ALK #5 (n=6 out of 8) exhibited a prolonged survival over two months (FIG. 5A). In contrast, untreated mice or mice treated with cyclophosphamide alone died within one month due to aggressive B cell malignancies.

Using FACS analysis, CD19+/ALK+ cells were found in one mouse treated with CTX alone. Circulating CD19+/ALK+ tumor cells were found in peripheral blood (FIG. 5B, Left). An ALK+ tumor mass was isolated close to lymph node (FIG. 5B, Right). FACS analysis was also conducted on the 6 out of 8 mice that survived more than two months. No tumor cells were found in peripheral blood of these mice (FIG. 5C).

### Example 5: Treatment of Transgenic Mice with ALK CAR-T Constructs

The generated ALK CARs were used for studies in both in immunocompetent and immunodeficient mice. ALK CAR-T constructs were investigated in two transplantable neuroblastoma mouse models: i) ALK^{F1174L}/MYCN (Brentjens R J, et al., CD19-targeted T cells rapidly induce molecular remissions in adults with chemotherapy-refractory acute lymphoblastic leukemia. Sci Transl Med 2013 Mar. 20; 5(177):177ra38) transgenic mice; and ii) NSG immunodeficient mice with orthotopic grafts of human neuroblastoma cells.

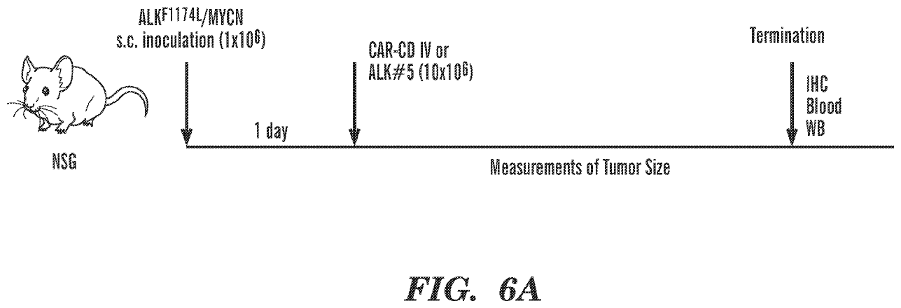

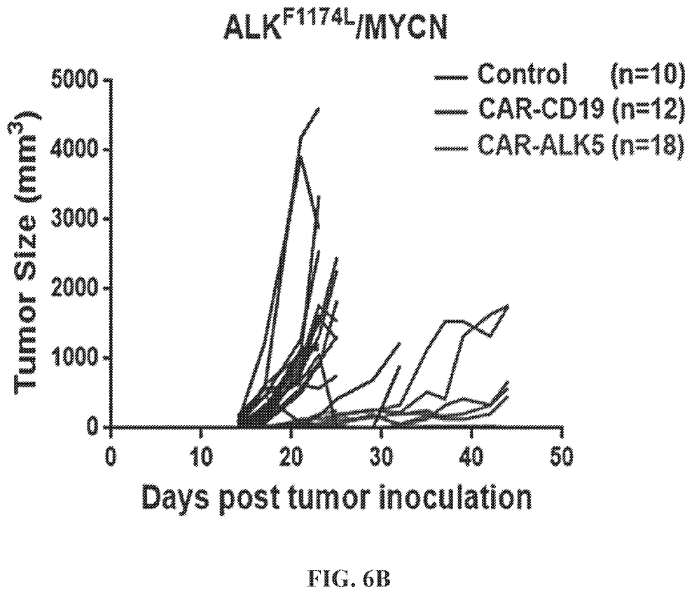

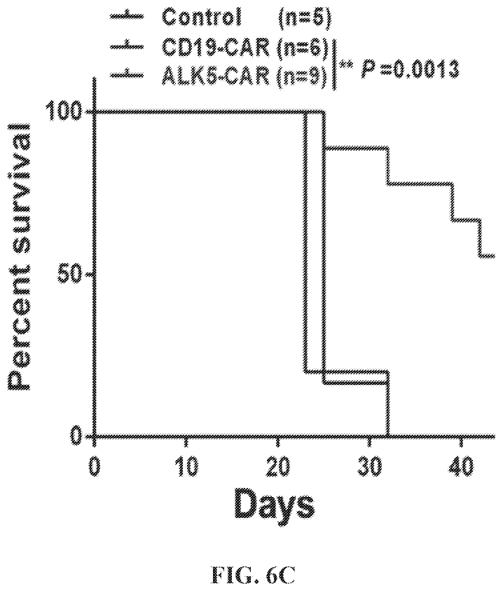

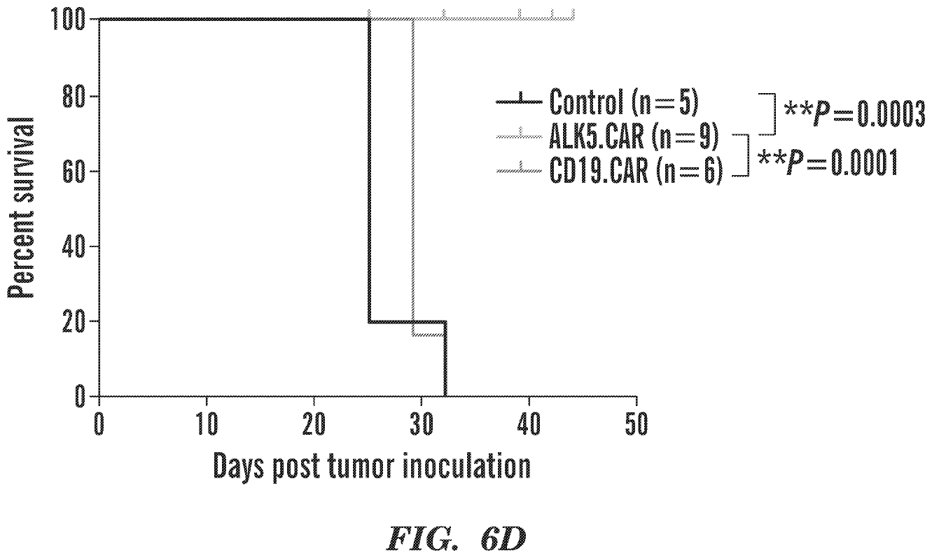

Neuroblastoma in ALK^{F1174L}/MYCN transgenic mice is driven by overexpression of human mutant ALK^{F1174L}. ALK^{F1174L}/MYCN transgenic mice express ALK at levels comparable to the human neuroblastoma cell line SH-SY5Y, a line expressing low ALK levels (Heczey A, et al., CAR T Cells Administered in Combination with Lymphodepletion and PD-1 Inhibition to Patients with Neuroblastoma. Mol Ther 2017 Sep. 6; 25(9):2214-24). FIG. 6A shows the experimental design for evaluating the anti-tumor efficacy of ALK CAR-Ts in a subcutaneous ALK^{F1174L}/MYCN neuroblastoma model. NSG mice were transplanted s.c. with 1×10⁶ALK^{F1174L}/MYCN cells in both flanks. The mice were then injected with 10×10⁶ ALK #5 CAR-T cells or 10×10⁶ CD19 CAR-T cells as a positive control. Neuroblastoma growth delay induced by ALK CAR-Ts with tumor volume was measured daily (two-tailed p value <0.0001, unpaired t test) (FIG. 6B). Negative Control mice were measured until day 23. Treatment with ALK #5 CAR-T cells was shown to extend the survival of neuroblastoma-bearing mice (FIGS. 6C, 6D).

NSG immunodeficient mice with orthotopic grafts of human neuroblastoma cells were implanted s.c. with 1×10⁶SH-SY5Y cells in the kidney capsule to model orthotopic human neuroblastoma. The mice were then injected with 10×10⁶ ALK #5 CAR-T cells or 10×10⁶ CD19 CAR-T cells as a positive control. Neuroblastoma growth delay induced by ALK CAR-Ts with tumor volume was measured daily (two-tailed p value <0.0001, unpaired t test).

### Example 6: Comparison of Anti-Tumor Activity of ALK CAR-T Cells to Lorlatinib in an Immunocompetent Model of Metastatic Neuroblastoma

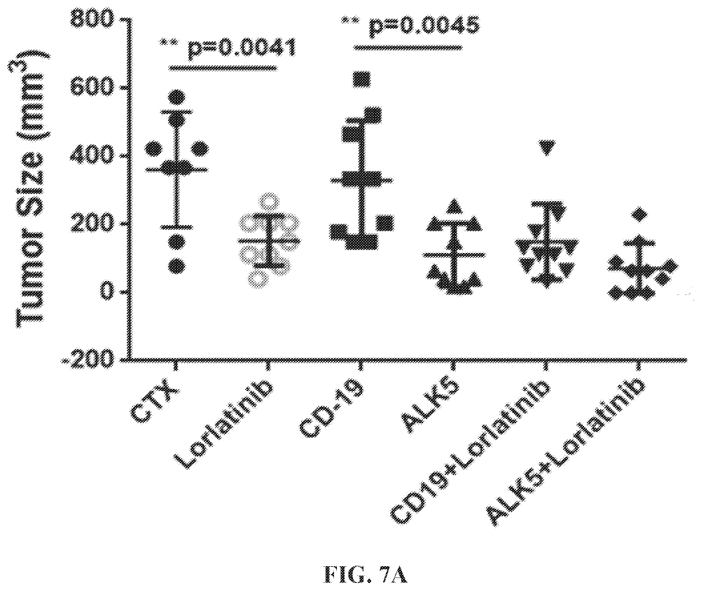

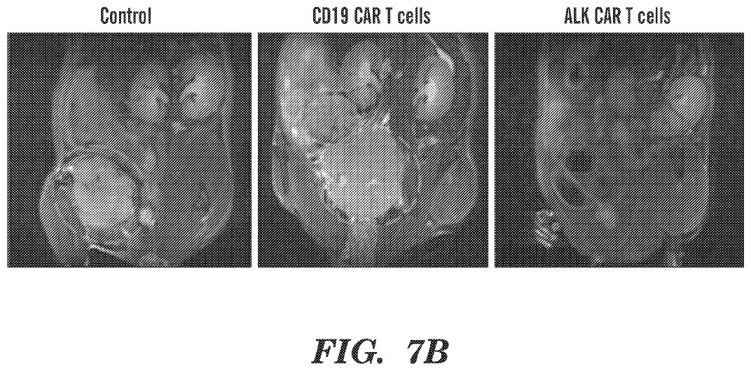

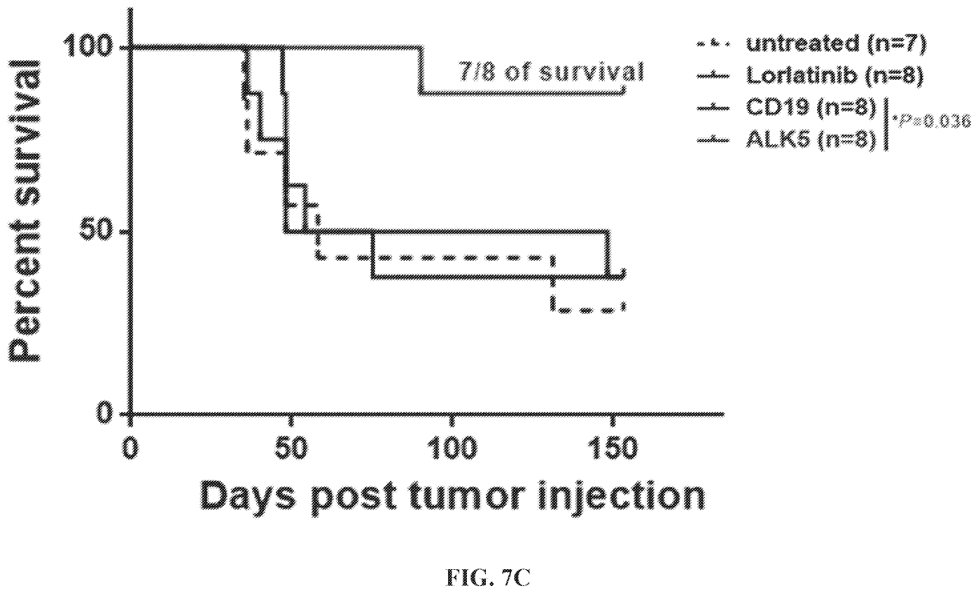

The antitumor effect of ALK CAR-T cells was evaluated in a fully syngeneic neuroblastoma model. ALK^{F1174L}/MYCN neuroblastoma was transplanted s.c. into BALB/c mice. ALK #5 CAR-T cells or CD19 CAR-T cells were generated from BALB/c purified T cells and injected i.v. weekly for three weeks. Lorlatinib was administered by oral gavage (4 mg/kg/day) for three weeks. Tumor volumes were measured at day 23. As shown in FIG. 7A, the combination of ALK #5 CAR-T cells with lorlatinib eradicated neuroblastoma in 30% of mice. Immunocompetent mice were injected i.v. with 1×10⁶ ALK^{F1174L}/MYCN neuroblastoma cells to induce multiple metastatic tumor formation, and were treated with CD19 CAR-T cells or ALK CAR-T cells. Metastatic tumors are shown in FIG. 7B as dashed circles. FIG. 7C shows the survival of immunocompetent mice in a metastatic model of neuroblastoma treated with the indicated CAR-T cells or lorlatinib.

The potential toxicity of ALK #5 CAR-T cells was also evaluated. In normal cells low ALK expression is confined to few neurons in the brain and in the testicle (Kabir T F, et al., Immune Checkpoint Inhibitors in Pediatric Solid Tumors: Status in 2018. Ochsner J 2018; 18(4):370-6). No signs of toxicity induced by ALK #5 CAR-T cells were detected as measured by weight loss, temperature changes and IL-6 release following injection of ALK #5 CAR-T cells. Histology examination showed no evidence of brain inflammation and mice did not show any obvious neurological symptoms.

### Example 7: In Vitro Validation of Human ALK CAR-T Cells

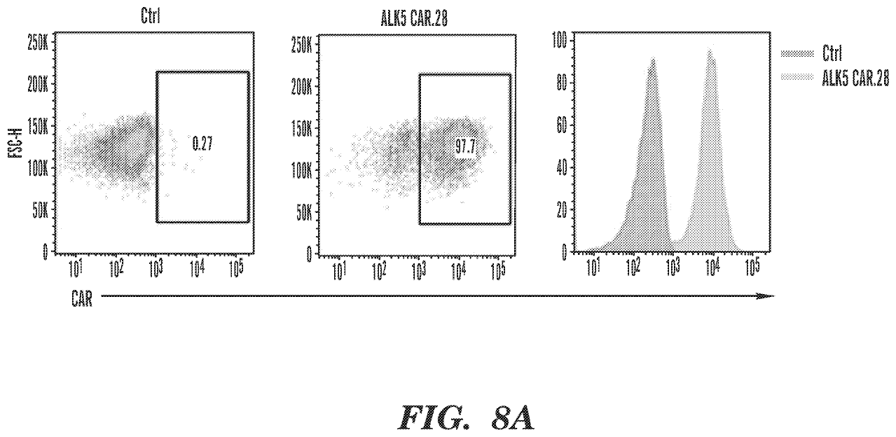

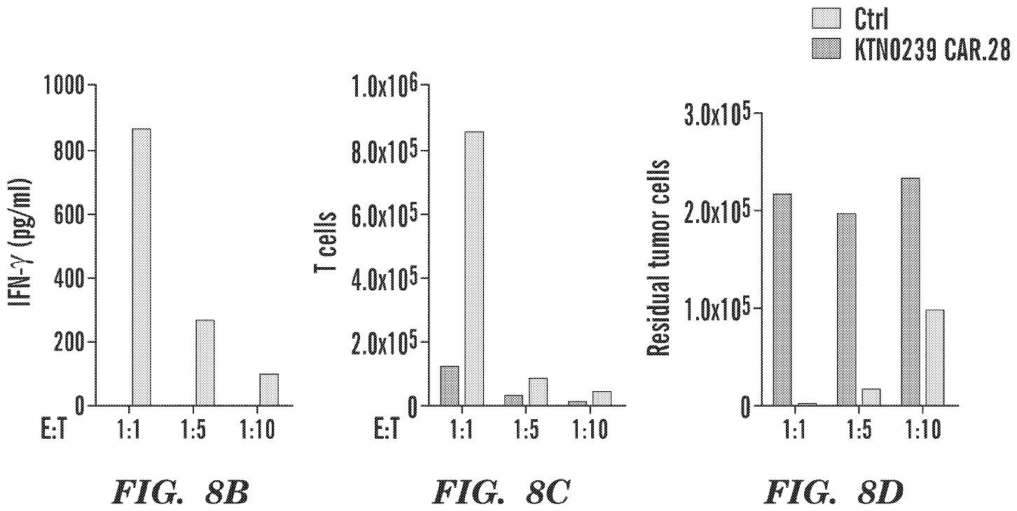

In order to validate hALK CAR-T cells in vitro, fully human ALK #5 CAR (hALK #5 CAR) based on a humanized version of the ALK #5 scFv was generated. Human T cells were transduced with the hALK #5 CAR and neuroblastoma tumor cells were targeted in vitro. ALK CAR expression was measured in human T cells at day 4 after transduction evaluated by flow cytometry (FIG. 8A). IFN-γ released by human T cells in co-culture with the human neuroblastoma cells IMR-32 at the indicated ration of effector:target cells is shown in FIG. 8B. Proliferation by human T cells in co-culture with the human neuroblastoma cells IMR-32 at the indicated ration of effector:target cells is shown in FIG. 8C. As shown in FIG. 8D, in vitro killing activity of ALK CAR-T cells was quantified by the number of residual IMR-32 neuroblastoma cells after 3 days of co-culture at the indicated ratio.

### Example 8: Validation of ALK CARs Against Human Neuroblastoma Lines with Different ALK Expression Levels In Vitro and In Vivo

The level of expression of the target molecule on cancer cells is a critical determinant of CAR-T cell anti-tumor activity. ALK is expressed at variable levels on the surface of neuroblastoma cells, ranging from cases with low expression of the wild-type ALK receptor, to cases with moderate or high expression of a wild-type or mutated ALK receptor, including in some cases cells with ALK gene amplification (Heczey A, et al., CAR T Cells Administered in Combination with Lymphodepletion and PD-1 Inhibition to Patients with Neuroblastoma. Mol Ther 2017 Sep. 6; 25(9):2214-24). Several neuroblastoma cell lines are representative of the various genetic mutations and varying ALK expression: cell lines with high ALK expression, i.e. NB-1 (ALK WT amplified) and Felix (mutated ALK^{F1245C}); cell lines with moderate ALK expression, i.e. IMR-32 (ALK WT), NBL-S(ALK WT) and COG-N-453 (mutated ALK^{F1174L}); cell lines with low ALK expression, i.e. SH-SY5Y (mutated ALK^{F1174L}) and COG-N-424x (ALK WT). All these lines grow well in vitro and engraft in NSG mice (Heczey A, et al., CAR T Cells Administered in Combination with Lymphodepletion and PD-1 Inhibition to Patients with Neuroblastoma. Mol Ther 2017 Sep. 6; 25(9):2214-24).

The antitumor activity of human T cells expressing the hALK #5 CAR was tested in vitro by measuring cytotoxic activity, cytokine release and T cell proliferation as previously described (Chen Y, et al., Eradication of Neuroblastoma by T Cells Redirected with an Optimized GD2-Specific Chimeric Antigen Receptor and Interleukin-15. Clin Cancer Res 2019 Jan. 7).

The anti-tumor activity of hALK #5 CAR-Ts in vivo in models of neuroblastoma in NSG mice was validated by implanting four patient-derived xenografts (PDXs) (SJNBL013762, SJNBL013761, SJNBL046148 and SJNBL046) (St. Jude Children's Research Hospital), which express the luciferase reporter (FFLuc), into NSG mice and injecting the mice with the hALK #5 CAR-Ts.

### Example 9: Testing NK Cells as a Cell Platform for CAR Expression

Genetic modification of NK cells remains challenging using either γ-retrovirus or lentivirus vectors. The use of α-retroviruses has a novel gene delivery system in NK cells. A split packaging design was developed for α-retrovirus based vectors in which viral coding sequences (gag/pol, env) were integrated into virus packaging cells without packaging sequences and without sequence overlaps (Awad M M, et al., Epitope mapping of spontaneous autoantibodies to anaplastic lymphoma kinase (ALK) in non-small cell lung cancer. Oncotarget 2017 Nov. 3; 8(54):92265-74). To improve viral titers, codon optimized α-virus packaging sequences are available with enhanced titers of several orders of magnitude due to enhanced gag/pol expression. Resulting α-virus vectors pseudotyped for infection of murine cells transduce hematopoietic stem cells murine (HSCs) with high efficiency (Ait-Tahar K, et al., Correlation of the autoantibody response to the ALK oncoantigen in pediatric anaplastic lymphoma kinase-positive anaplastic large cell lymphoma with tumor dissemination and relapse risk. Blood 2010 Apr. 22; 115(16):3314-9). Transduction of NK cells with an α-retroviral vector containing a CD19 CAR expression cassette selectively enhanced NK cell cytotoxicity towards CD19-expressing leukemia cells (Voena C, et al., Efficacy of a Cancer Vaccine against ALK-Rearranged Lung Tumors. Cancer Immunol Res 2015 December; 3(12):1333-43).

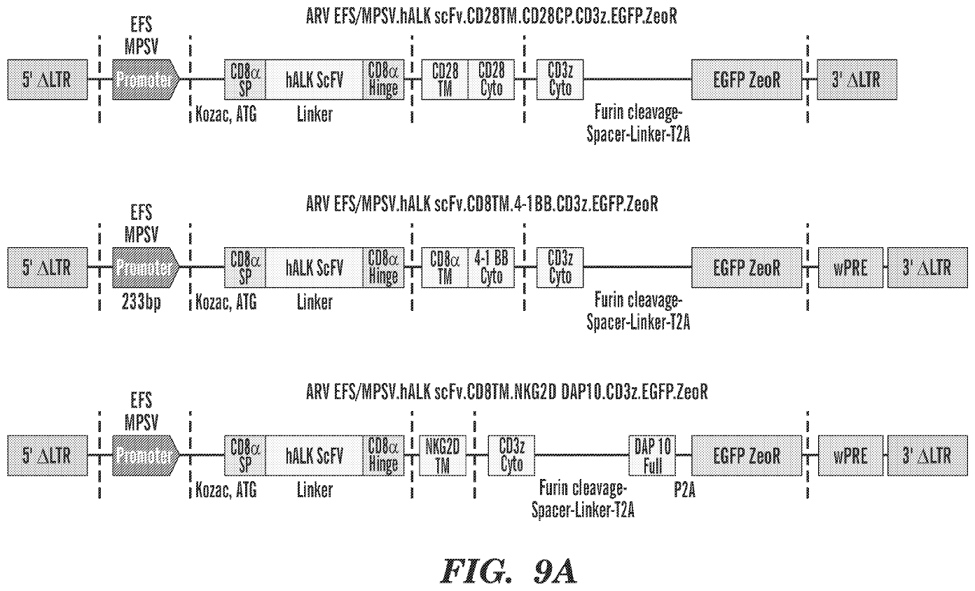

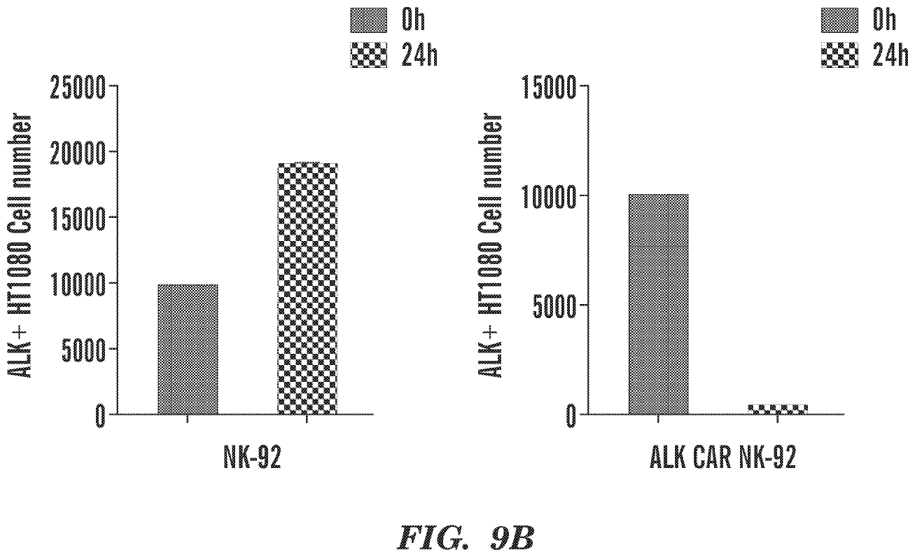

The use of α-retrovirus system was used to facilitate the manufacturing of ALK CAR expressing NK cells for targeting ALK-positive cells. An avian α-retroviral vector backbone was used to more effectively mediate CAR delivery to NK cells. As shown in FIG. 9A, a total of 6 vectors with differences in the strength of the promoter was used to express the hALK #5 CAR (strong promoter: MPSV; weaker promoter: EFS) and differences in the T cell or NK signaling components (CD28, 4-1BB, as in Aim 1, or NKG2D with DAP10).

For all these 6 vectors, stable producer clones were generated and NK-92 cells were transduced using the RD114/TR-pseudotyped α-retroviral particles. About 50 clones were screened in order to isolate a producer line with titers >1×10⁶/ml of infectious particles (tittered on HT1080, a standard human cell line used for such tittering).

Quantification of in vitro killing activity of NK-92 cells transduced with an hALK CAR construct was conducted after 24 hrs incubation with HT1080 cells expressing the human ALK receptor. NK-92 cells transduced with an MPSV.ALK5.CAR construct efficiently and specifically killed target cells expressing the ALK receptor (FIG. 9B). The 6 different α-retroviral ALK #5 CAR constructs were compared for their efficiency to transduce NK-92 cells and their potency to kill target neuroblastoma cells with different levels of ALK expression. The constructs identified from this screen were used to transduce human NK cells. The killing activity of α-retroviral ALK #5 CAR NK-92 or primary NK cells was compared to ALK #5 CAR-T cells. ALK #5 CAR NK cells were evaluated as to whether they possess a killing activity superior to ALK #5 CAR-T cells against cells with low surface ALK expression. From the in vitro studies, the most potent ALK #5 CAR NK constructs were identified for testing in vivo. Both donor-derived NK cells and NK-92 cells were tested. ALK5.CAR NK or ALK5.CAR-T cells were injected (5×10⁶ cells) in NSG mice bearing metastatic neuroblastoma. Additionally, both ALK #5 CAR NK cells and ALK #5 CAR-T cells were co-injected (5×10⁶ cells) in NSG mice bearing metastatic neuroblastoma to evaluate whether a more potent anti-tumor effect was produced by the combination.

### Example 10: Combination of ALK CAR-T Cells and ALK Blockade

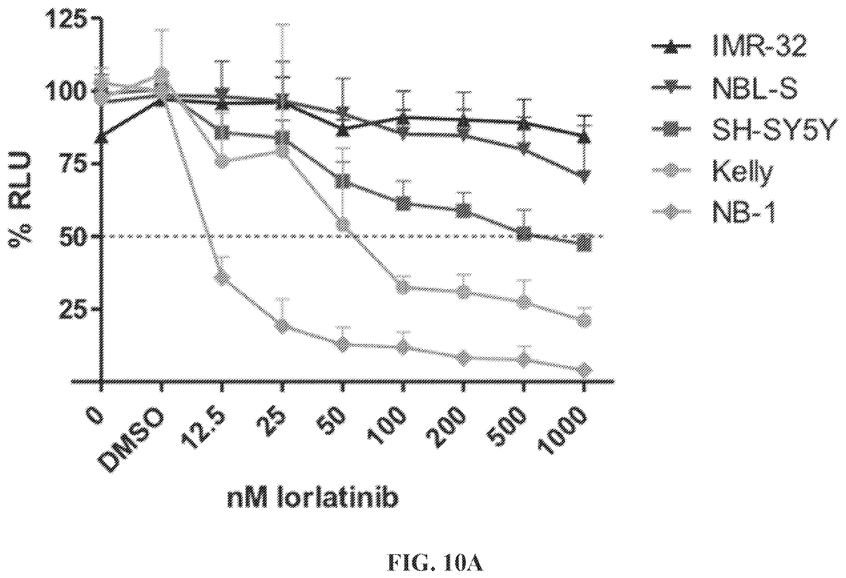

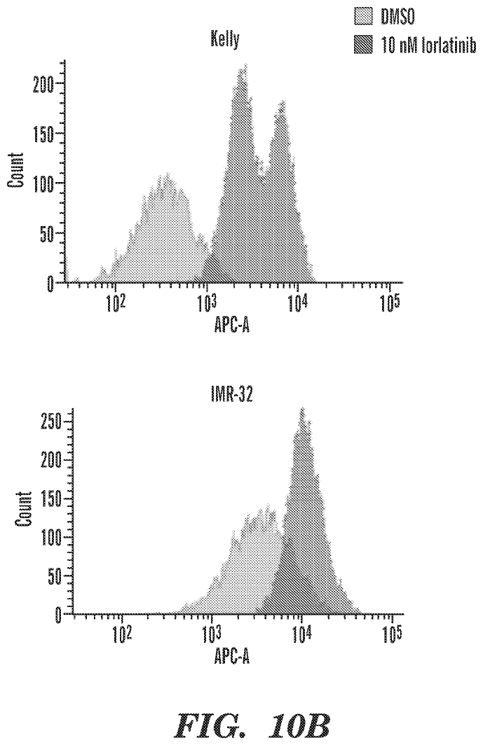

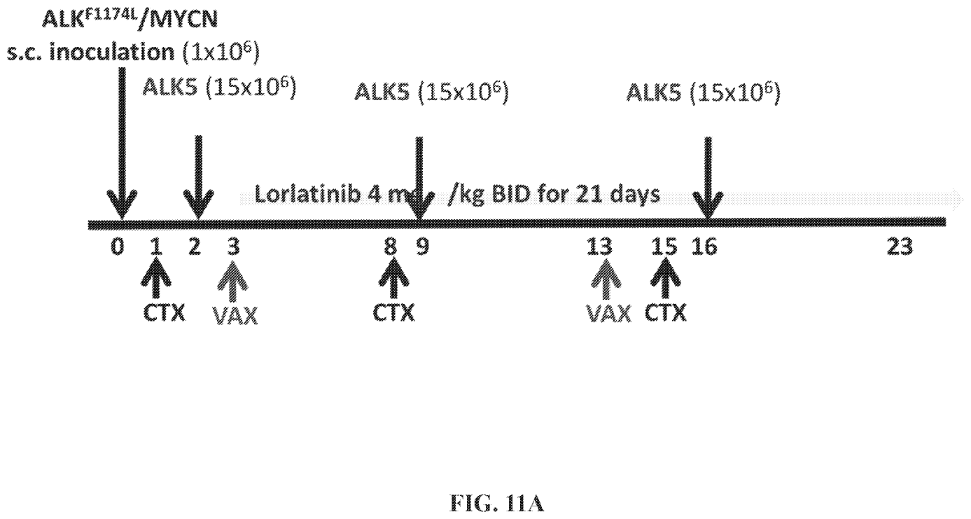

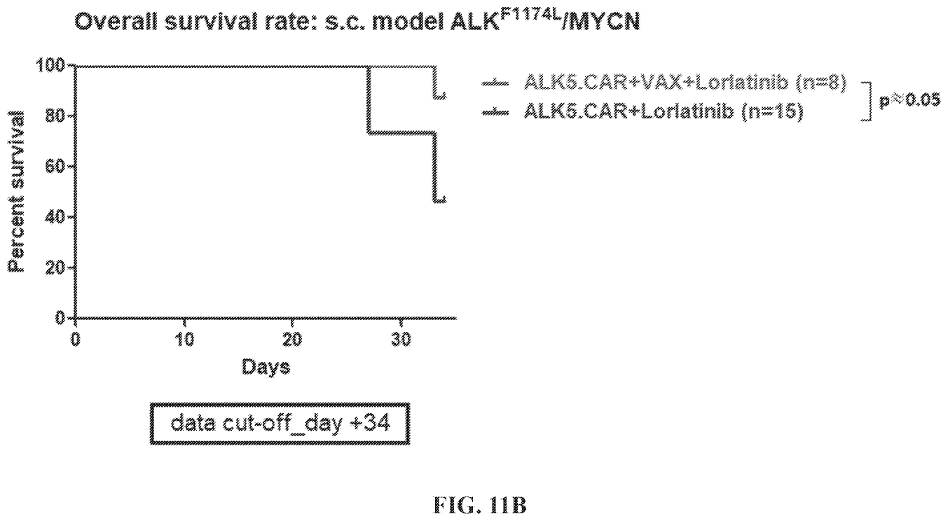

First generation ALK tyrosine kinase inhibitors (TKIs), such as crizotinib, have limited therapeutic efficacy in neuroblastoma, while the third generation ALK TKI, lorlatinib, is effective against mutated neuroblastoma (Infarinato N R, et al., The ALK/ROS1 Inhibitor PF-06463922 Overcomes Primary Resistance to Crizotinib in ALK-Driven Neuroblastoma. Cancer Discov 2016 January; 6(1):96-107). The effects of lorlatinib on ALK viability and expression in neuroblastoma cells was evaluated. Several neuroblastoma cell lines with various ALK genetic alterations, including NB-1 (ALK WT amplified), IMR-32 (ALK WT), NBL-S(ALK WT), SH-SY5Y (mutated ALK^{F1174L}), Kelly (mutated ALK^{F1174L}), were treated with increasing doses of lorlatinib (FIG. 10A). Viability was measured at 48 hours. Lorlatinib alone at 100 nM showed only modest anti-proliferative activity and reduced proliferation by less than 50% in Kelly or SH-SY5Y cells (FIG. 10A).

Expression of surface ALK was measured on Kelly and IMR-32 cells by flow cytometry on Kelly and IMR-32 cells treated with 10 nM lorlatinib for 24 hours (FIG. 10B). Lorlatinib not only reduced tumor growth, in particular, ALK-mutated neuroblastoma cells, but also greatly enhanced the expression of ALK on the surface of neuroblastoma cells likely by reducing its internalization or increasing stability of the mutated protein.

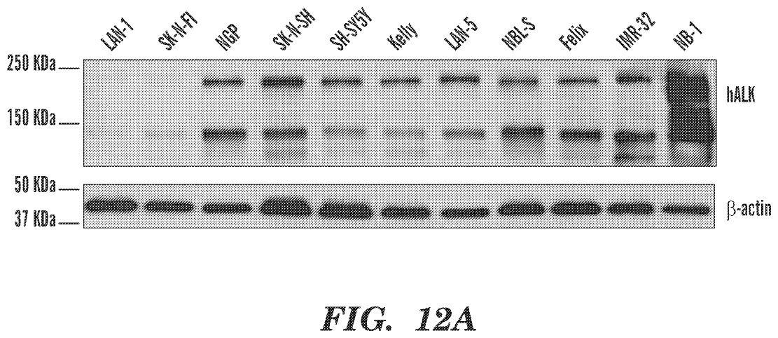

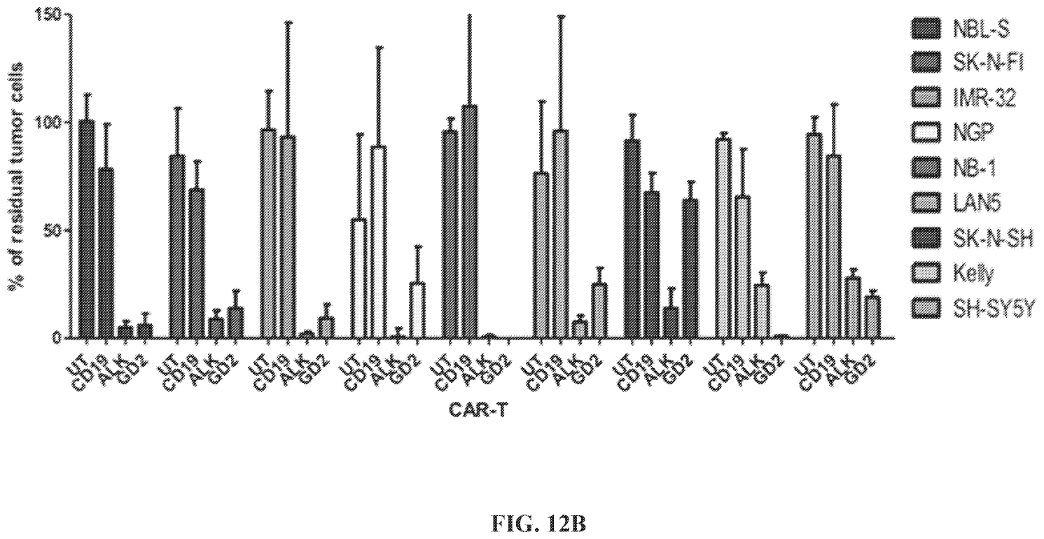

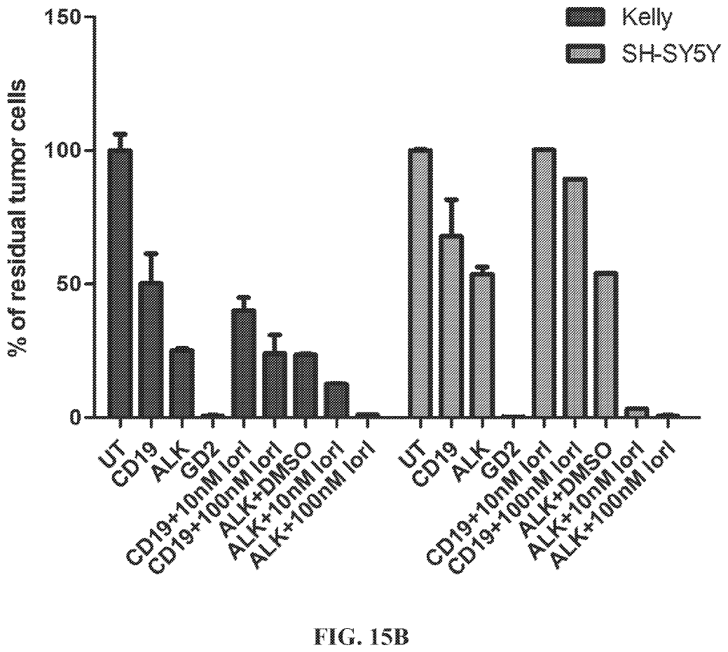

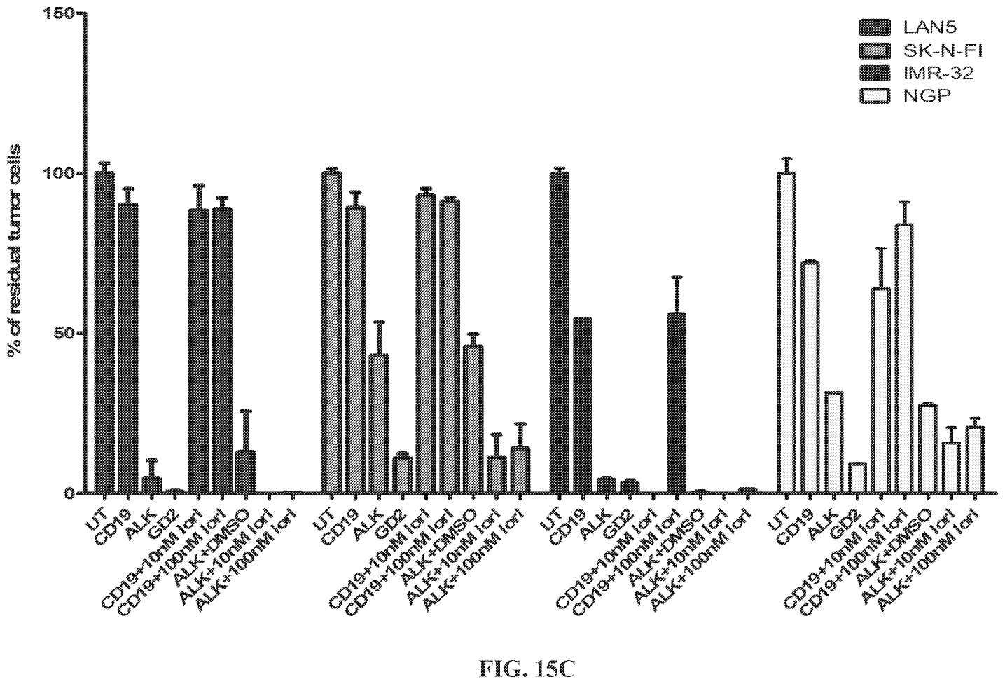

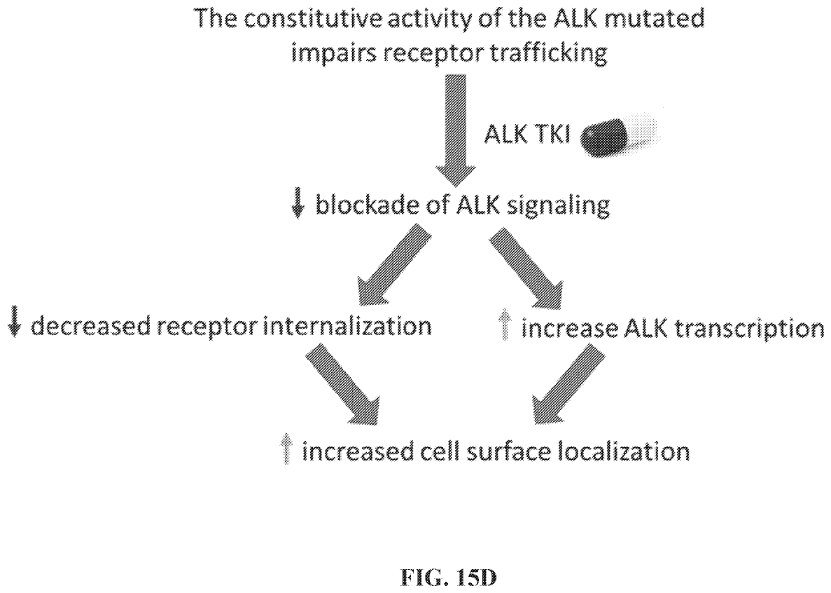

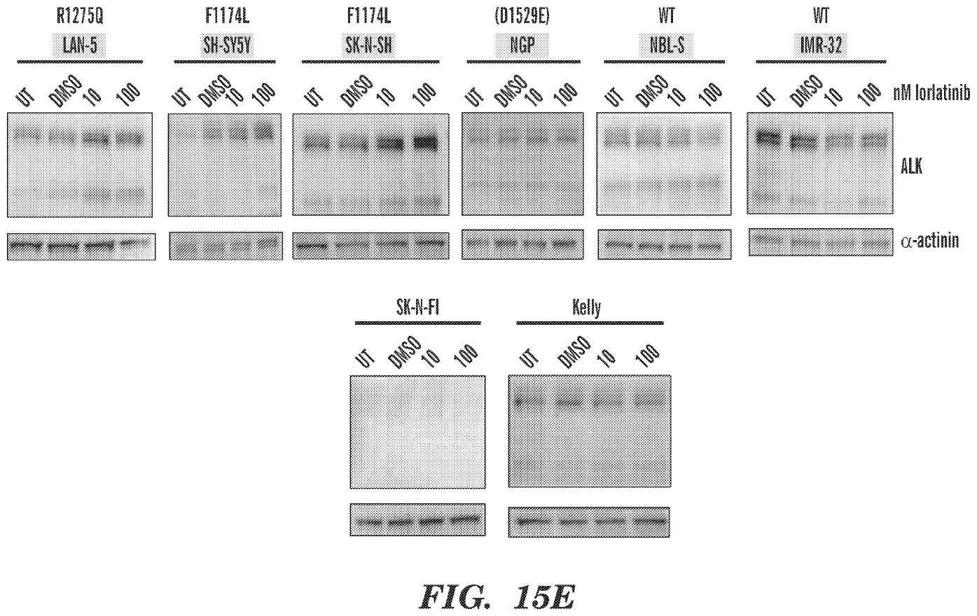

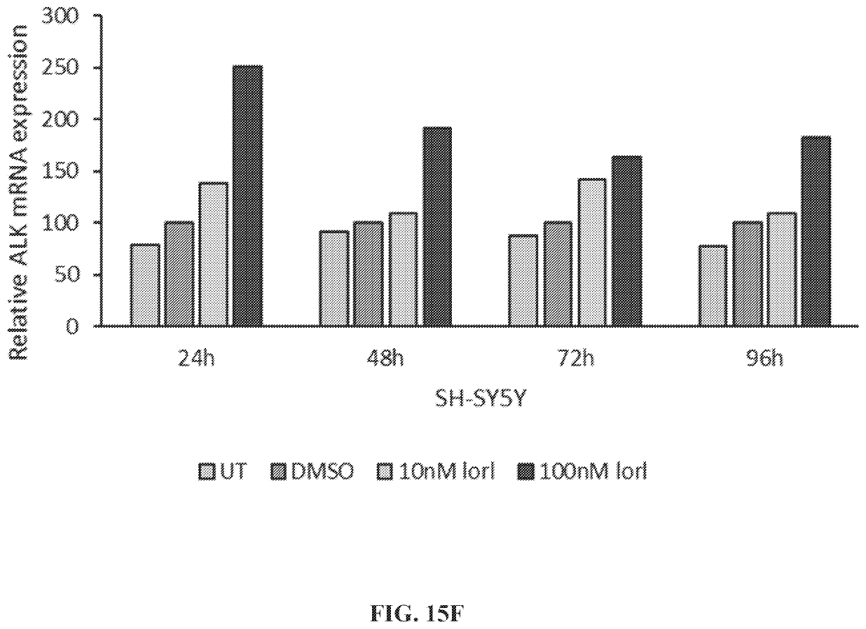

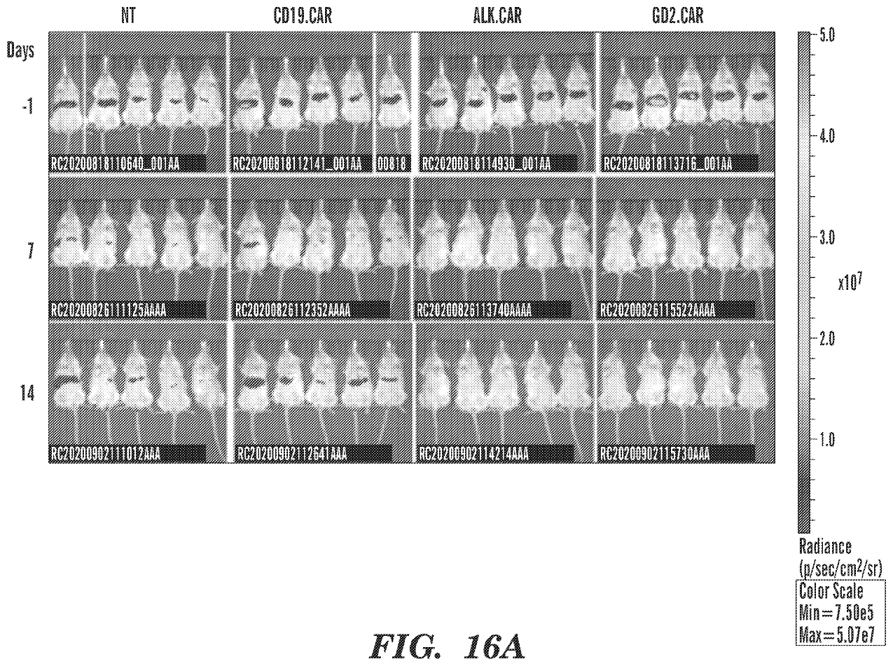

To examine the synergic killing effect of hALK CAR-T cells in combination with lorlatinib, hALK CAR-T cells alone or in combination with lorlatinib were administered against neuroblastoma cell lines. First, hALK CAR-T cells alone or in combination with lorlatinib at 10 nM and 100 nM were administered against two ALK^{F1174L} mutated neuroblastoma cell lines (Kelly and SH-SY5Y), which express relatively low levels of ALK (FIGS. 15A, 15B). CD19 CAR-T cells, CD19 CAR-T cells in combination with lorlatinib at 10 nM and 100 nM, ALK CAR-T cells in combination with DMSO, GD2 CAR-T cells, and untransduced T cells were used as controls. ALK^{F1174L} mutated Kelly or SH-SY5Y cell lines had previously shown incomplete killing by hALK CAR-T cells alone (FIG. 12B). Second, hALK CAR-T cells alone or in combination with lorlatinib at 10 nM and 100 nM were administered against LANS, SK-N-FI, IMR-32, and NGP human neuroblastoma cell lines (FIG. 15C). CD19 CAR-T cells, CD19 CAR-T cells in combination with lorlatinib at 10 nM and 100 nM, ALK CAR-T cells in combination with DMSO, GD2 CAR-T cells, and untransduced T cells were used as controls. As shown in FIGS. 15A-15C, the addition of lorlatinib maximized the killing activity of hALK CAR-T cells.

Lorlatinib was evaluated for potentiating the activity of ALK CAR-T cells, not only by affecting the viability of tumor cells, but also by increasing ALK expression. The mechanisms by which lorlatinib enhances expression of ALK on the surface of neuroblastoma cells and increases the targeting by ALK CAR-T cells is shown in FIG. 15D. Western blot analysis was conducted to evaluate the expression of ALK in neuroblastoma cells that have a mutation of the ALK gene (LAN-5 (R1275Q), SH-SY5Y (F1174L), SK-N-SH (F1174L), NGP (D1529E), NBL-S (WT), IMR-32 (WT), SK-N-FI (WT), Kelly (WT)) when used in combination with 10 nM and 100 nM of lorlatinib (FIG. 15C). DMSO treated and untransduced cells were used as controls. As shown in FIG. 15C, lorlatinib increased expression of ALK in neuroblastoma cells that have a mutation in the ALK gene.

Western blot analysis was further conducted to evaluate the expression of ALK in LAN-5 (R1275Q), SH-SY5Y (F1174L), SK-N-SH (F1174L), NGP (D1529E), NBL-S(WT), IMR-32 (WT), SK-N-FI (WT), Kelly (WT) neuroblastoma cells when used in combination with 10 nM and 100 nM of lorlatinib (FIG. 15E). DMSO treated and untransduced cells were used as controls. The relative ALK mRNA expression in SH-SY5Y neuroblastoma cells after treatment with 10 nM and 100 nM of lorlatinib at 24, 48, 72, and 96 hours is shown in FIG. 15F. DMSO treated and untransduced cells were used as controls.

To test whether the increased expression of surface ALK during treatment with lorlatinib enhances the killing activity of ALK CAR-Ts, IMR-32 and Kelly cell lines, that upregulate ALK expression without significant effects on cell viability at 10 nM lorlatinib, were incubated with increasing amounts of ALK #5 CAR-T cells or control CD19 CAR-T cells (10:1, 1:1, 1:5, 1:10 tumor:T cell ratios) for 3 days. The neuroblastoma cell viability was then measured by flow cytometry.

To test whether the anti-proliferative effect, combined with the increased expression of surface ALK induced by lorlatinib, enhanced the killing activity of hALK CAR-Ts in vitro and in vivo, neuroblastoma cell lines were incubated with 10 nM or 100 nM lorlatinib, plus increasing amounts of hALK5 CAR-T cells or control CD19 CAR-T cells (10:1, 1:1, 1:5, 1:10 tumor:T cell ratios) for 5 days, and then NB cell viability and ALK surface expression of the residual tumor cells was measured by flow cytometry.

For in vivo therapeutic experiments, immunocompetent BALB/c mice were injected i.v. with 1×10⁶ ALK^{F1174L}/MYCN syngeneic neuroblastoma cells and immunodeficient NSG mice were injected i.v. with NB-1 (ALK WT amplified), IMR-32 (ALK WT), or Kelly or SH-SY5Y (mutated ALK^{F1174L}) cells. One week after tumor injection lorlatinib was administered by oral gavage (4 mg/kg/day-10 mg/kg/day) for three weeks. hALK CAR-T cells or control CD19 CAR-T cells were injected one week after the first lorlatinib treatment. Tumor growth was measured by luciferase activity and survival was compared in mice treated with CAR-T cells alone or in combination with lorlatinib.

### Example 11: Combination Therapy Using ALK CAR-T Cells with an ALK Vaccine

ALK^{F1174L}/MYCN transgenic mice were used to evaluate combination therapy with CAR-T cells and an ALK vaccine. Immunocompetent BALB/c mice were injected s.c. with 1×10⁶ ALK^{F1174L}/MYCN syngeneic neuroblastoma cells. After tumor injection, ALK CAR-T cells were injected in combination with an ALK vaccine as shown in the administration schedule of FIG. 11A. ALK vaccines with unconjugated ALK peptides or ALK peptides conjugated to an amphiphile, such as N-hydroxy succinimidyl ester-end-functionalized poly(ethylene glycol)-lipid (NHS-PEG2KDa-DSPE) were evaluated. CD19 CAR-T cells were used as a control. Tumor growth was measured by luciferase activity and survival was compared in mice treated with CAR-T cells alone or ALK vaccine alone.

ALK^{F1174L}/MYCN transgenic mice were used to evaluate combination therapy with ALK CAR-T cells, ALK vaccine and lorlatinib. BALB/c mice were injected s.c. with 1×10⁶ cells of syngeneic ALK^{F1174L}/MYCN neuroblastoma cells. Mice were vaccinated with an ALK vaccine and injected with 15×10⁶ ALK CAR-T cells at the indicated times as shown in FIG. 11A. The ALK TKI lorlatinib was administered at 4 mg/Kg BID for the indicated time as shown in FIG. 11A. The mice were also treated with or without the immunosuppressor, cyclophosphamide (CTX) at the indicated times as shown in FIG. 11A. Survival of mice treated with the combination of ALK vaccine, ALK CAR-T cells, and lorlatinib was compared to mice treated with the combination of ALK CAR-T cells and lorlatinib (FIG. 11B). Follow-up curves were assessed up to a cut-off of 34 days. The addition of the ALK vaccine to ALK CAR-T cells further increased the percent survival of mice.

### Example 12: Validation of hALK5 CAR-T Cells Against Human Neuroblastoma Cell Lines Showing Different Levels of ALK Expression

The level of expression of the target molecule on cancer cells is a critical determinant of CAR-T cell anti-tumor activity. ALK is expressed at variable levels on the surface of neuroblastoma cells, and it is thus critical to assess the antitumor effects of hALK CAR-Ts in neuroblastoma cells expressing different levels of ALK. Several neuroblastoma cell lines representative of the various genetic mutations and ALK expression levels are shown in Table 6. ALK expression in human neuroblastoma cell lines, LAN-1, SK-N-FI, NGP, SK-N-SH, SH-SY5Y, Kelly, LAN-5, NBL-S, Felix, IMR-32, and NB-1, is shown in FIG. 12A. All these lines grow in vitro and engraft in NSG mice (Heczey A, et al., CAR T Cells Administered in Combination with Lymphodepletion and PD-1 Inhibition to Patients with Neuroblastoma. Mol. Ther. 2017; 25:2214-2224).

TABLE 6
List of Human Neuroblastoma (NB) Cells Lines

| NB Cell Line | ALK | MYCN |
|---|---|---|
| LAN-1 | F1174L | Amplified |
| SK-N-FI | R1275Q | Not amplified* |
| NGP | Wild-type | Amplified |
| SK-N-BE(2)C | Wild-type | Amplified |
| SK-N-SH | F1174L | Not amplified |
| SH-SY5Y | F1174L | Not amplified |
| Kelly | F1174L | Amplified |
| LAN-5 | R1275Q | Amplified |
| NBL-S | Wild-type | Not amplified |
| Felix | F1245C | Not amplified |
| IMR-32 | Wild-type | Amplified |
| NB-1 | Amplified | Amplified |

To measure the in vitro killing activity of hALK CAR-T cells, residual tumor cells from two independent donors were measured after administration of hALK CAR-T cells against NBL-S, SK-N-FI, IMR-32, NGP, NB-1, LAN-5, SK-N-SH, Kelly, SH-SY5Y neuroblastoma cell lines (FIG. 12B). CD19 CAR-T cells and untransduced T cells were used as negative controls, and GD2 CAR-T cells were used as a positive control. The CAR-T cells and target cells were incubated for 5 days at a 1:1 effector target ratio. As shown in FIG. 12B, hALK CAR-T cells showed almost complete elimination of all human neuroblastoma cells in vitro. the in vitro killing activity of hALK CAR-T cells was comparable or superior to the positive control GD2 CAR-T cells and approached the maximum killing for most cell lines (FIG. 12B).

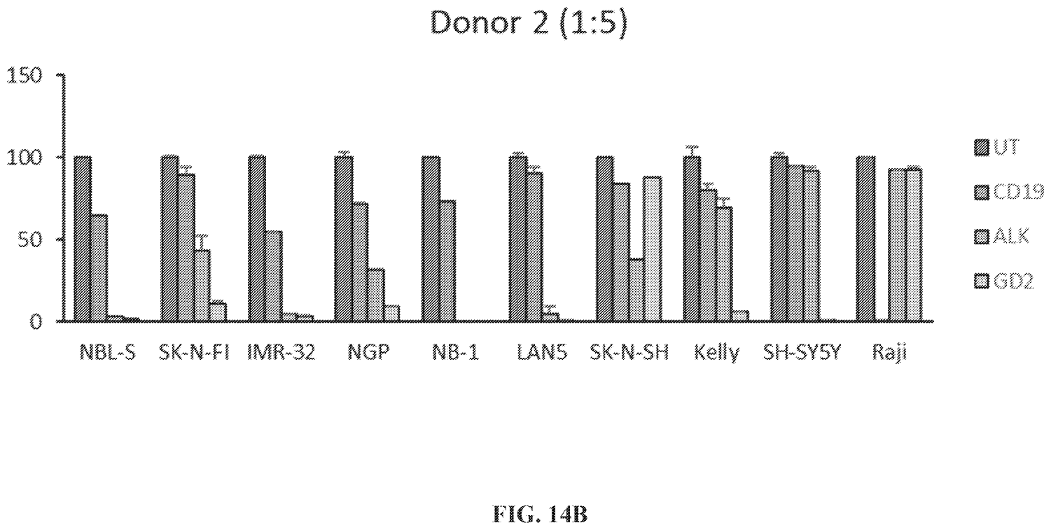

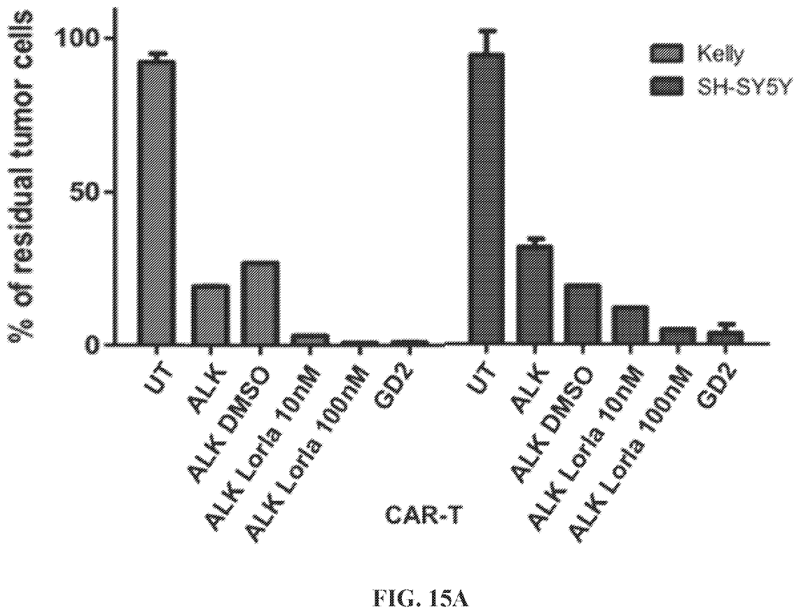

The killing activity of human ALK CAR-T cells against several cell lines (NBL-S, SK-N-FI, IMR-32, NGP, NB-1, LANS, SK-N-SH, Kelly, SH-SY5Y, Raji) of human neuroblastoma was also examined at a 1:1 tumor:CAR-T ratio (FIG. 14A) or a 1:5 tumor:CAR-T ratio (FIG. 14B). CD19 CAR-T cells and untransduced T cells were used as negative control, and GD2 CAR-T cells were used as a positive control. The in vitro killing activity of hALK CAR-T cells was comparable or superior to the positive control GD2 CAR-T cells and approached the maximum killing for most cell lines (FIGS. 14A, 14B).

Antitumor activity of hALK5 CAR-Ts in vivo in NSG mice was examined by injecting by i.v. 10⁶ luciferase-transduced neuroblastoma cell lines with different ALK expression levels (NB-1, IMR-32, Kelly and SH-SY5Y). hALK CAR-T cells and CD19 CAR-T cells (5×10⁶ cells/mouse) were injected 2 weeks after neuroblastoma injection. Tumor growth was assessed by luciferase monitoring via IVIS instrumentation. Four patient-derived xenografts (PDXs) (SJNBL013762, SJNBL013761, SJNBL046148 and SJNBL046) were obtained from the PDX bank of the St. Jude Children's Research Hospital (stjude.org/research/resources-data/childhood-solid-tumor-network/available-resources.html#xenografts). PDXs also express FFLuc and can be implanted in NSG mice. Neuroblastoma PDXs can also be injected orthotopically in the kidney capsule.

### Example 13: Testing of ALK CAR-T Cell Toxicity

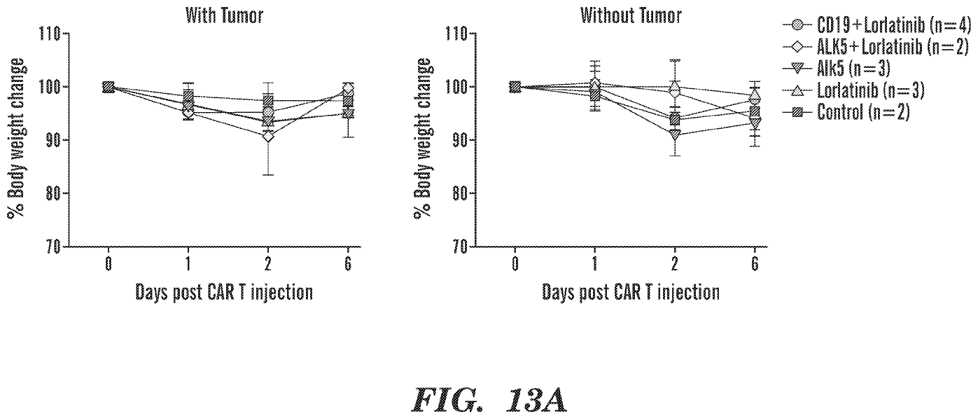

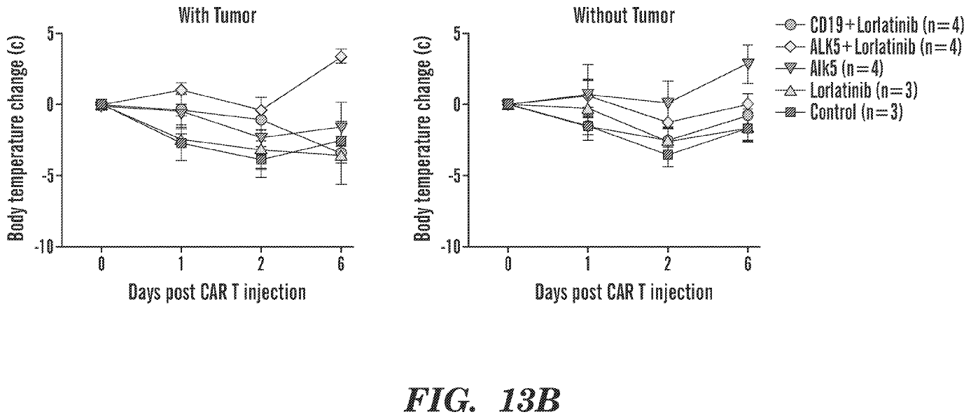

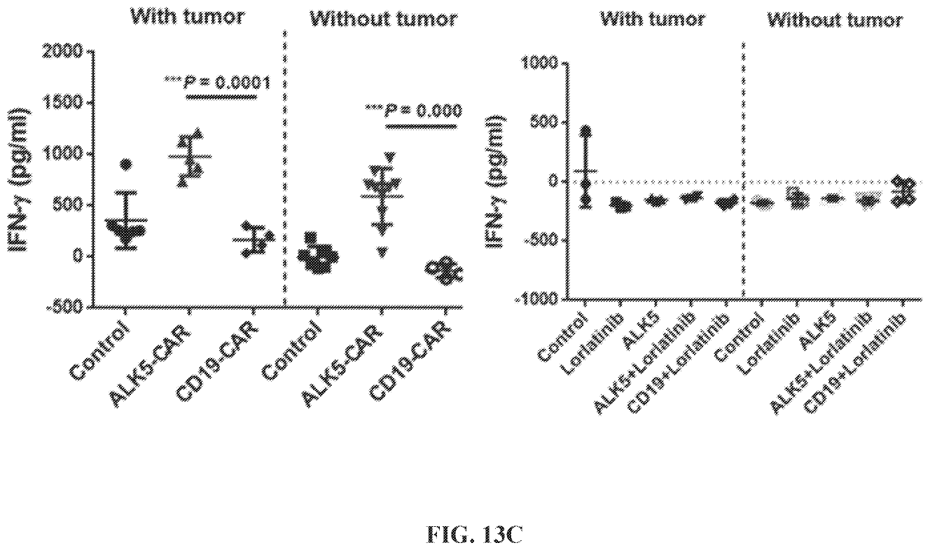

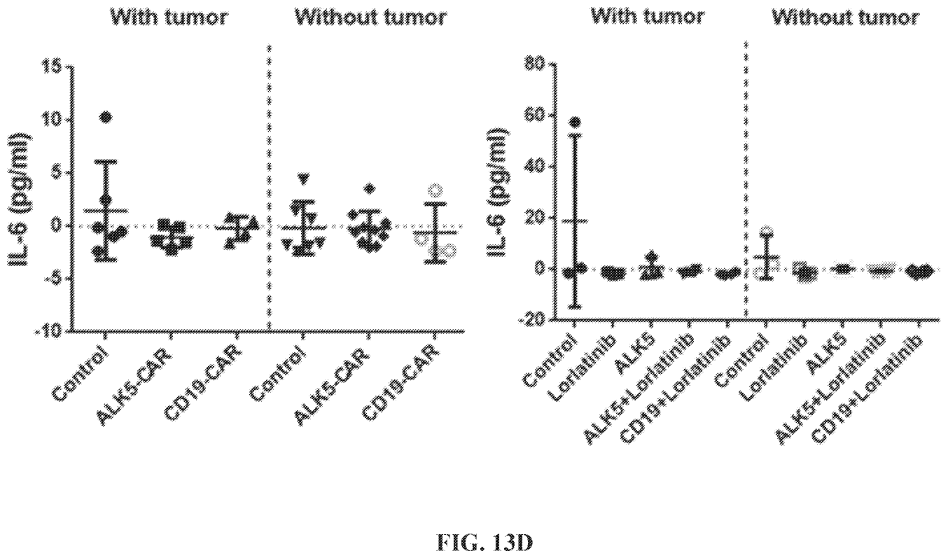

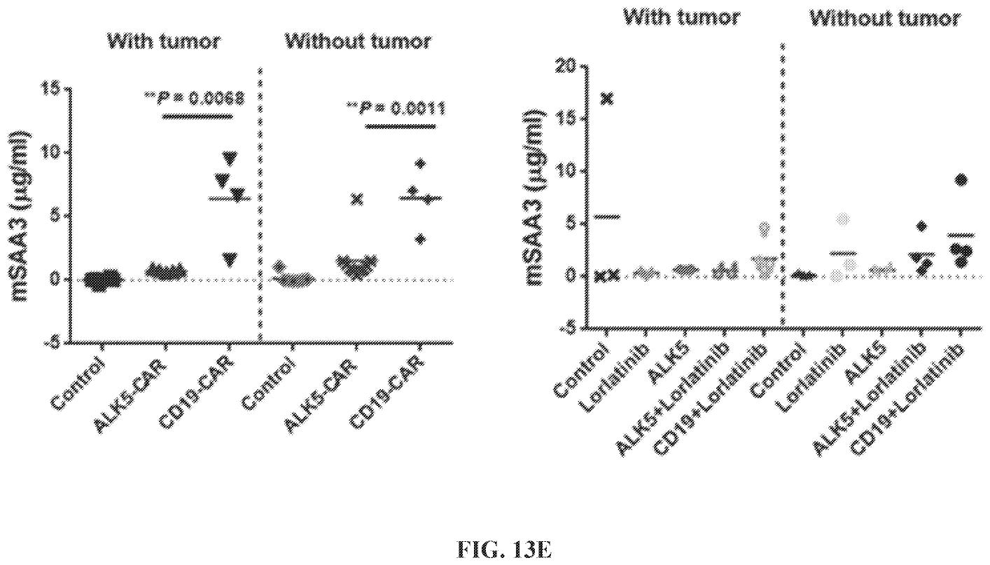

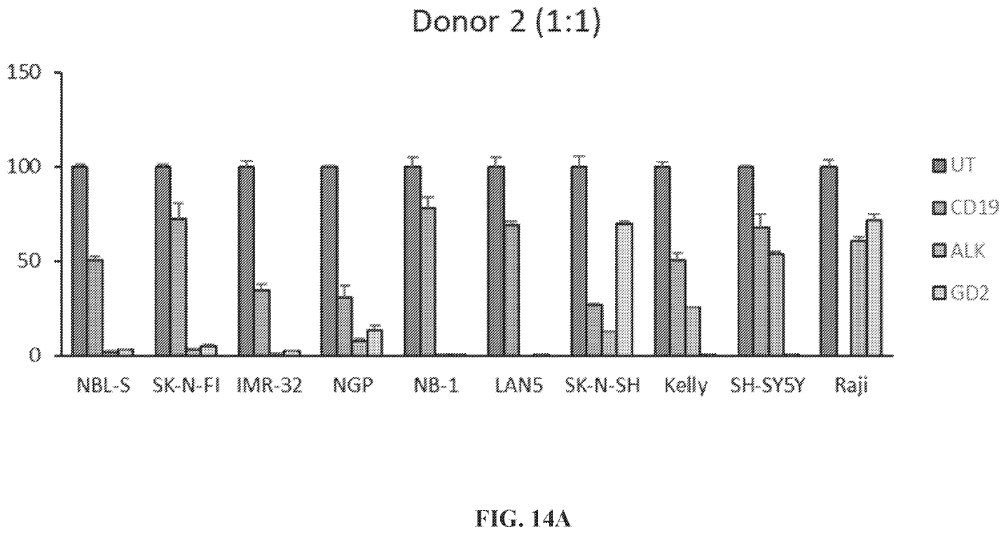

To examine the toxicity of ALK CAR-T cells, changes in body weight, body temperature, interferon gamma (IFNγ) production, interleukin 6 (IL-6) production, and serum amyloid A 3 (mSAA3) production was measured in mice with and without tumors injected with ALK5 CAR T cells alone and in combination with lorlatinib (FIGS. 13A-13E). CD19 CAR-T cells, CD19 CAR-T cells in combination with lorlatinib, lorlatinib alone, and untransduced T cells were used as controls. No significant changes in body weight, body temperature, and interleukin 6 (IL-6) production following injection of ALK CAR-T cells was observed.

### Example 14: In Vivo Anti-Tumor Activity of Human ALK CAR-T Cells

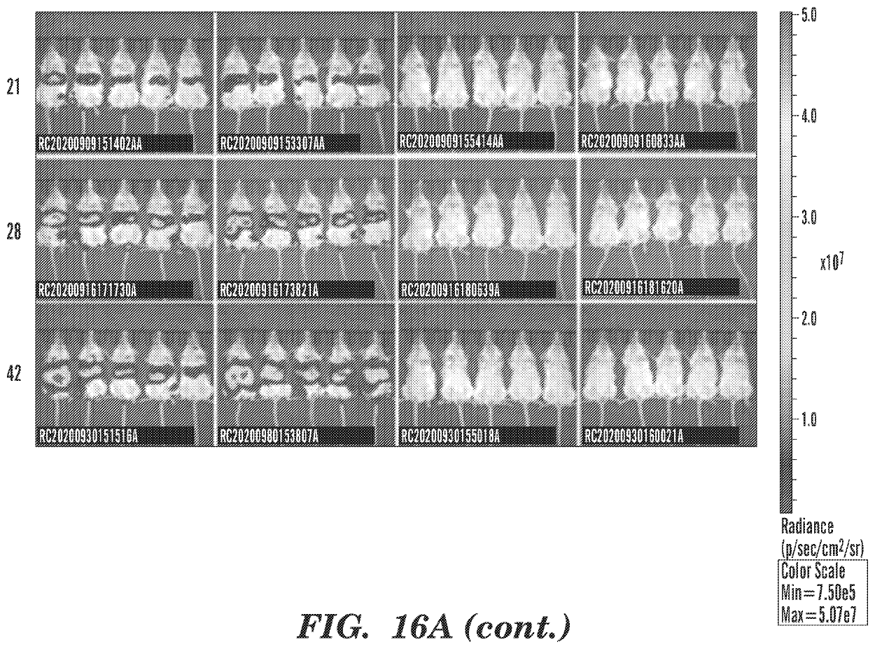

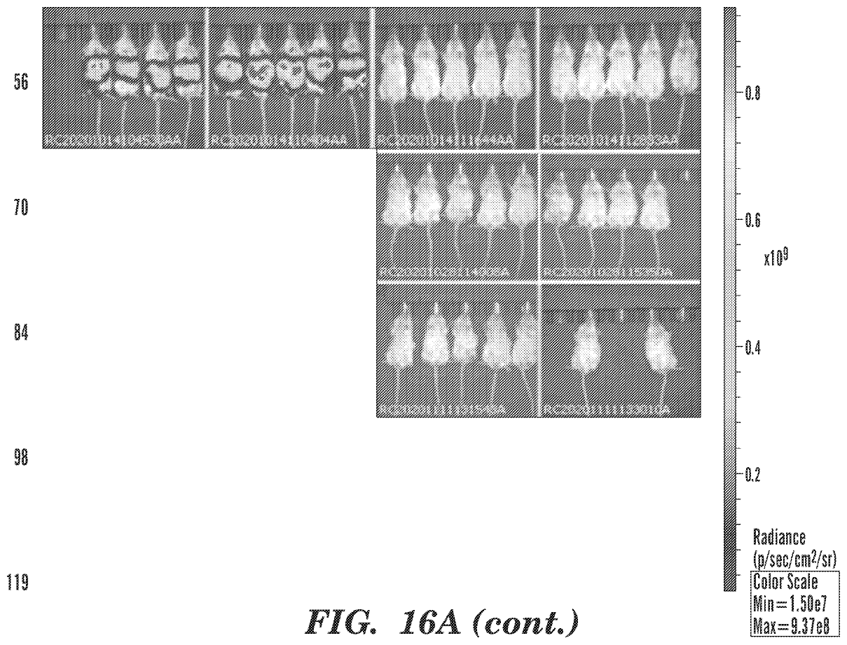

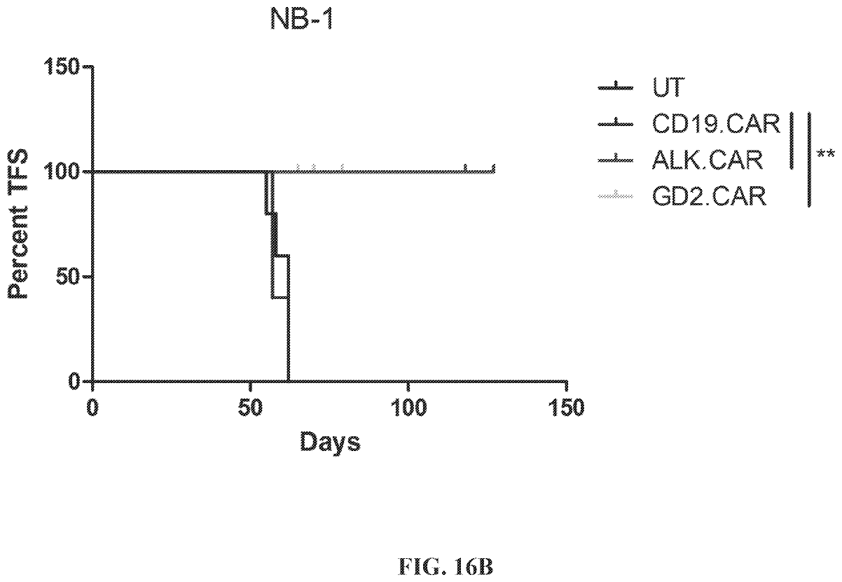

The anti-tumor activity of hALK CAR-T cells was evaluated in vivo. NSG mice were injected with NB-1 cells, which express high levels of ALK, and then treated with one single injection of ALK CAR-T cells (FIG. 16A). CD19 CAR-T cells and non-transduced (NT) cells were used as negative controls, and GD2 CAR-T cells were used as a positive control. Tumor growth was monitored over time by luciferase luminescence detected with IVIS instrumentation. The treatment-free survival (TFS) of these mice was monitored as shown in FIG. 16B. In vivo efficacy was shown in NSG mice of ALK CAR T cells against neuroblastoma cells expressing high levels of wild-type ALK.

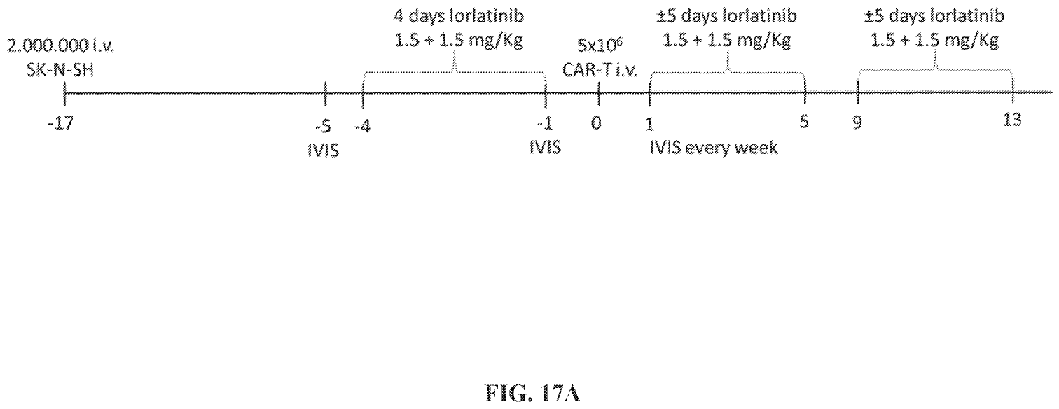

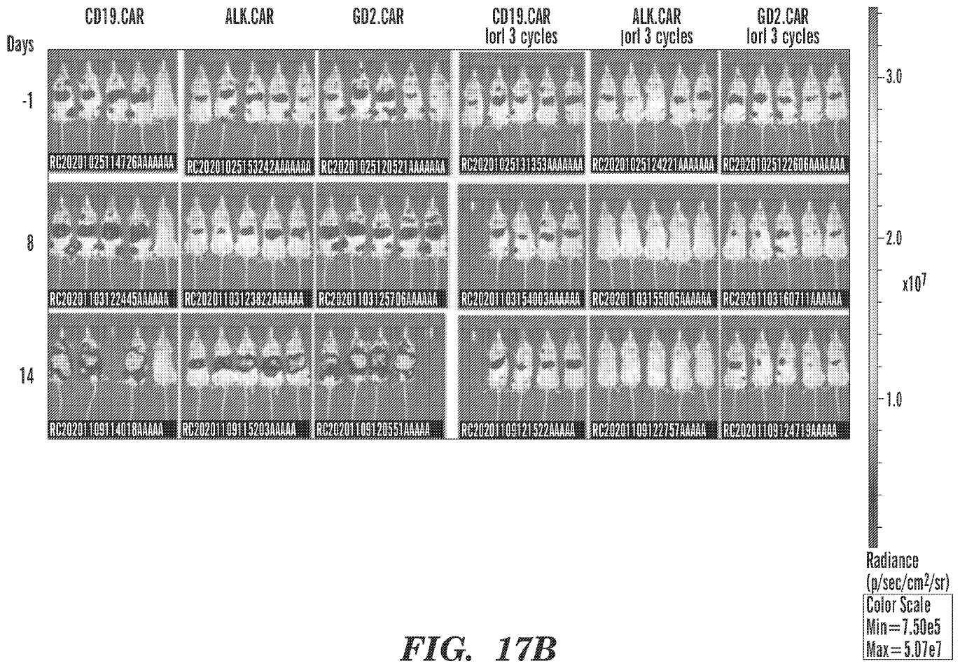

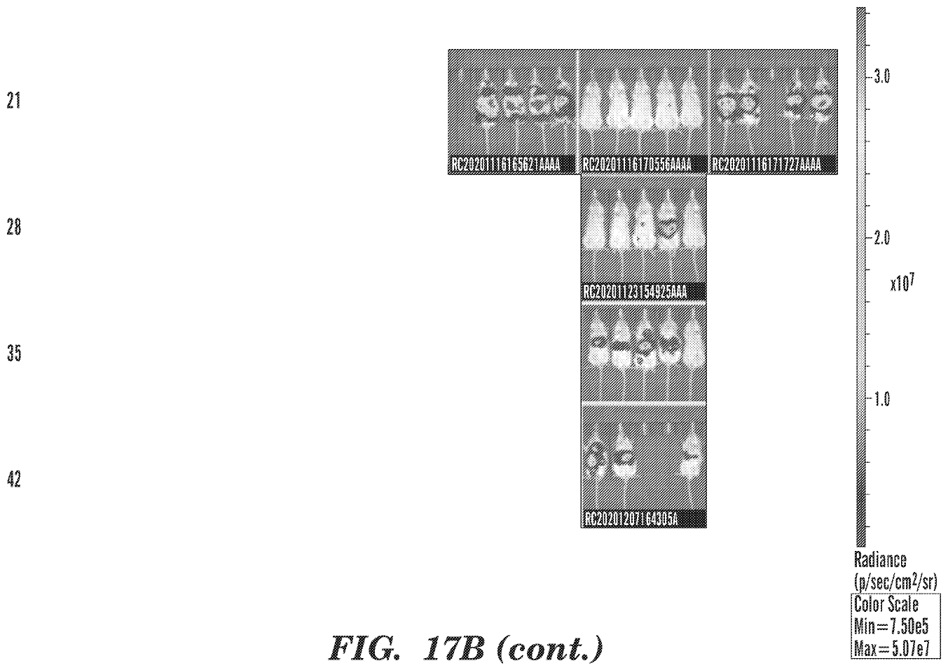

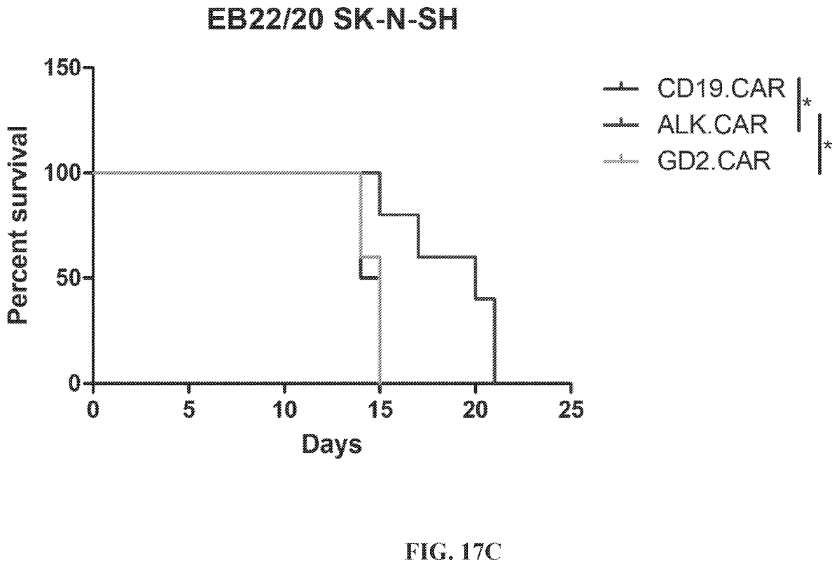

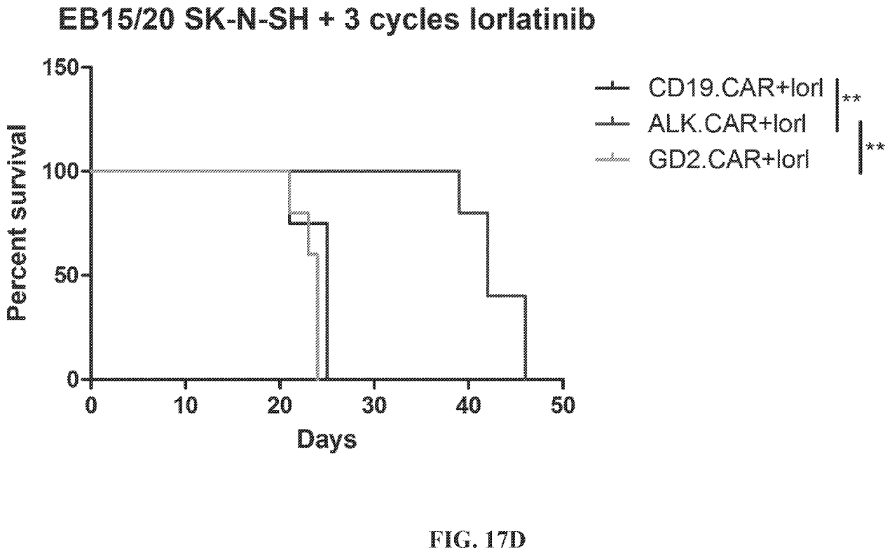

An experimental procedure was further developed for combining ALK CAR-T cells with three cycles of lorlatinib in vivo in NSG mice (FIG. 17A). NSG mice were injected with SK-N-SH cells, which express low levels of mutated ALK, and then treated with one single injection of ALK CAR-T cells (FIG. 17B). Mice either had no lorlatinib treatment or were treated with 3 cycles of lorlatinib according to the procedure depicted in FIG. 17A. CD19 CAR-T cells were used as negative control, and GD2 CAR-T cells were used as positive control. Tumor growth was monitored by luciferase luminescence detected with IVIS instrumentation. Survival of the NSG mice without lorlatinib treatment is shown in FIG. 17C, and the survival of the NSG mice with the 3 cycles of lorlatinib is shown in FIG. 17D. In vivo efficacy was shown in NSG mice of ALK CAR-T cells against neuroblastoma cells expressing low levels of mutated ALK alone or in combination with lorlatinib.

### Example 15: Dose-Escalation with Autologous hALK CAR-T Cells in Patients with Relapsed/Refractory Neuroblastoma

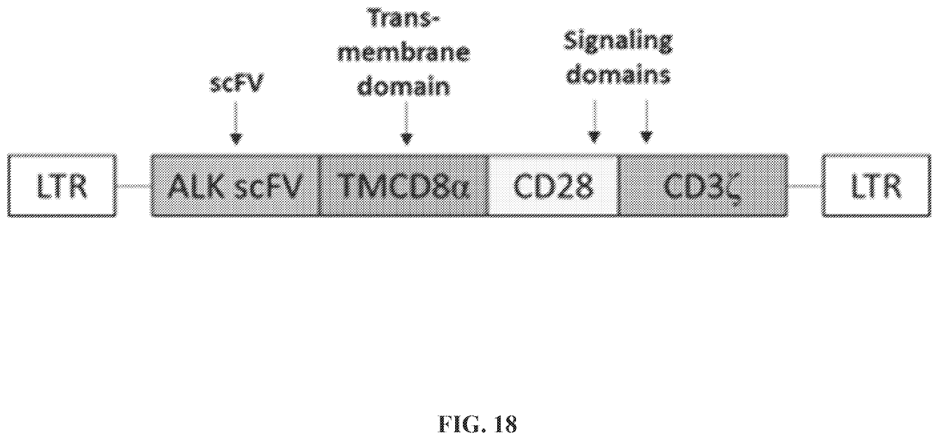

Autologous T cells expressing hALK CAR can be evaluated without any additional gene modification such as IL-15 delivery. Presence of surface ALK expression by immunohistochemistry (IHC) can be used as an eligibility criterion. About >80% of neuroblastoma patients are expected to express detectable ALK levels by IHC. Using a hALK CAR transgene as shown in FIG. 18, the clinical grade retroviral vector can be generated (UNC Advanced Cellular Therapeutics (ACT) facility) and hALK CAR-Ts can be manufactured (UNC ATC) in accordance with validated SOPs. Selected patients with relapsed/refractory neuroblastoma can be administered with the manufactured hALK CAR-T Cells to test safety and antitumor activity of escalating doses of autologous hALK CAR-T Cells.

### Patient Eligibility

Eligible subjects will have: 1) written HIPAA authorization signed by legal guardian; 2) age greater than 18 months and less than 18 years at the time of consent; 3) adequate performance status as defined by Lansky or Karnofsky performance status of >60 (Lansky for <16 years of age); 4) life expectancy 12 weeks; 5) histological confirmation of neuroblastoma or ganglioneuroblastoma at initial diagnosis. Bone marrow samples are acceptable as confirmation of neuroblastoma. 6) high risk neuroblastoma with persistent or relapsed disease, defined as: first or greater relapse of neuroblastoma following completion of aggressive multi-drug frontline therapy; first episode of progressive NB during aggressive multi-drug frontline therapy; persistent/refractory neuroblastoma as defined by less than a complete response (by the revised INRC) at the conclusion of at least 4 cycles of aggressive multidrug induction chemotherapy on or according to a high-risk NB protocol (such as A3973 or ANBL0532); 7) measurable or evaluable disease per Revised International Neuroblastoma Response Criteria; 8) adequate central nervous system function (no known CNS disease, no seizure disorder requiring antiepileptic drug therapy); 9) adequate cardiac function (shortening fraction of >27% by echocardiogram); and 10) adequate pulmonary function (no chronic oxygen requirement and room air pulse oximetry >94%).

### Treatment Plan

All patients will receive lymphodepleting chemotherapy before CAR-T cell infusion (Heczey A, et al., CAR T Cells Administered in Combination with Lymphodepletion and PD-1 Inhibition to Patients with Neuroblastoma. Mol. Ther. 2017; 25:2214-2224). Lymphodepletion will consist of cyclophosphamide 500 mg/m²/day IV on days 1-2 and fludarabine 30 mg/m²/day IV on days 1-4. The continual reassessment method (CRM) will be used to estimate the maximum-tolerated dose (MTD) of cells that can be administered in dose escalation cohorts comprised of 2-6 subjects. The final MTD will be the dose with estimated probability of dose limiting toxicity (DLT) closest to the target toxicity rate of 20%. Three cell doses will be evaluated: D1: 0.5×10⁶ CAR⁺ cells/kg; D2: 1×10⁶ CAR⁺ cells/kg; D3: 1.5×10⁶ CAR⁺ cells/kg. Cohort enrollment will be staggered and each subject must complete at least 2 weeks of cell treatment without incident of DLT before another subject can be enrolled at that dose level. A minimum of two subjects must complete the 4-week post-infusion DLT safety assessment period before cohort enrollment of subjects at the next higher dose level will be considered. If dose level 1 is determined to be above a tolerable dose, de-escalation would occur to dose level −1 where subjects would receive 0.25×10⁶ CAR⁺ cells/kg. Rimiducid (aka AP1903) (0.4 mg/kg), a dimerizing agent that is designed to engage and activate the iC9 to trigger T cell death, will be used to alleviate Grade 3 or 4 neurotoxicity or grade 3 pain symptoms unresponsive to standard of care (Di Stasi A., et al., Inducible apoptosis as a safety switch for adoptive cell therapy. N. Engl. J. Med. 2011; 365:1673-1683). In the dose expansion portion of the study, subjects may receive a second cell infusion (with prior lymphodepletion). Risk assessment will be evaluated per SOPs. Dose-limiting toxicity (DLT) will be evaluated per NCI CTCAE criteria v 5.0 or CRS and ICANS grading criteria if it occurs within the DLT reporting period (i.e., 4 weeks following CAR-T cell infusions).

### Clinical Monitoring of Patients

Patient follow-up is directed by SOPs including a history and physical examination and routine laboratory investigations performed preinfusion and at 4 hours and 1, 2, 3, 4, 6 weeks, and months 3, 6, 9, and 12 post T-cell infusion, then every 6 months for 4 years. Patients are monitored for tumor progression or recurrence using standard criteria. Patients are evaluated at week 6 post-CAR-T cell infusion. Additional imaging obtained as part of standard clinical care will also be evaluated. Clinical response will be assessed using the revised International Neuroblastoma Response Criteria (INRC). Progression free survival (PFS) and overall survival (OS) will be estimated using the Kaplan-Meier method. Imaging will be obtained before and 6 weeks after CAR-T cell infusion. Imaging will then be performed at months 3, 6, 9, and 12 for study purposes. Patients will have bilateral bone marrow aspirates and biopsies obtained before and 6 weeks after CAR-T cell infusion. Repeat bone marrows will then be performed at months 3, 6, 9, and 12 for study purposes. If other tissue is obtained for clinical indications during the first year, a portion will be used to assess for presence of transduced T cells. If the patient dies, an autopsy will be requested and tissues assessed for the presence of CAR-T cells.

### Example 16: Materials and Methods

### Cell Lines and Cell Culture

Human neuroblastoma (NB) tumor cell lines IMR-32, NBL-S, NGP, LAN-5, LAN-1, SK-N-SH, SK-N-FI, SH-SY5Y and Felix, and human Burkitt's lymphoma cell line Raji were purchased from American Type Culture Collection (ATCC). NBL-S, NGP, LAN-5, LAN-1, SK-N-SH, SK-N-FI, SH-SY5Y, NB-1 and Raji were maintained in RPMI 1640 (Corning) supplemented with 10% fetal bovine serum (FBS)(Gibco), 100 U/mL of penicillin, 100 μg/mL of streptomycin (Corning), and 2 mM of L-glutamine (Corning). Felix were maintained in RPMI 1640 (Corning) supplemented with 10% fetal bovine serum (FBS)(Gibco), 100 U/mL of penicillin, 100 μg/mL of streptomycin (Corning), 2 mM of L-glutamine (Corning), and 1% Insulin/Transferrin/Selenium (ITS)(Corning). Phoenix-ECO and 293T packaging cells were obtained from DSMZ and cultured in Dulbecco's modified Eagle's medium (DMEM) (Corning) supplemented with 10% FBS (Gibco), 100 U/mL of penicillin, 100 μg/mL of streptomycin (Corning), and 2 mM of L-glutamine (Corning).

Cells were maintained at 37° C. in humidified atmosphere with 5% CO₂. NB cell lines were transduced with a retroviral vector encoding the GFP-Firefly-Luciferase (GFP-FFluc) gene, kindly provided by Prof. Giampietro Dotti (Vera et al., 2006). All cell lines were mycoplasma free and validated by flow cytometry for surface markers and functional readouts as needed. Lorlatinib was obtained from Pfizer.

### CAR-T Plasmid Construction

The variable regions of the heavy and light chains of the ALK1, ALK2, ALK3, ALK4, ALK5, ALK6, and ALK7 mAbs were cloned from mouse hybridoma and then cloned as an scFv fragment into previously validated CAR formats that include the murine CD8α hinge and transmembrane domain, CD28 intracellular costimulatory domain, and CD3ζ intracellular signaling domain. The ALK CAR cassettes were cloned into the retroviral vector SFG. For the human version of the ALK5 CAR, murine CD8α, CD28 and CD3ζ were replaced by human CD8α, CD28 and CD3ζ, and the ALK5 scFv was modified to generate a humanized version (hALK5 CAR). The scFv specific for CD19 and GD2 were previously reported (Kochenderfer et al., Blood, 116(20): 4099-4102 (2010); Du H, et al., Antitumor Responses in the Absence of Toxicity in Solid Tumors by Targeting B7-H3 via Chimeric Antigen Receptor T Cells. Cancer Cell 2019; 35:221-237).

### Retrovirus Production

Retroviral supernatants used for the transduction of murine T cells were generated by cotransfecting Phoenix-ECO packaging cells. Phoenix-ECO cells were plated in a 10 cm dish. The following day, cells were transfected with the retroviral vectors and the pCL-Eco plasmid using the Xfect Transfection Reagent (Takara) according to the manufacturer's instruction. The media was changed 6 hours post-transfection. The viral supernatant was collected 48 hours after transfection, and filtered with 0.45 μm filters.

For the preparation of retroviral supernatants used for the transduction of human T cells, 2×10⁶ 293 T cells were seeded in a 10 cm cell culture dish and transfected with the plasmid mixture of the retroviral vector, the Peg-Pam-e plasmid encoding MoMLV gag-pol, and the RDF plasmid encoding the RD114 envelope, using the GeneJuice transfection reagent (Merck Millipore) according to the manufacturer's instructions. Supernatants containing the retrovirus were collected 48 and 72 hours after transfection, and filtered with 0.45 μm filters.

### Generation of Murine CAR-T Cells

Murine T cells were isolated using EasySep Mouse T Cell Isolation Kit (Stemcell) from splenocytes obtained from C57BL/6J mice and stimulated with 100 U/mL IL-2 and Dynabeads Mouse T-Activator CD3/CD28 (Gibco), according to the manufacturer's instructions, for 24 hours. Activated murine T lymphocytes were transduced with retroviral supernatants plus 6 μg/mL polybrene via spinfection at 2,000 rpm for 80 minutes, and expanded in complete medium (RPMI-1640 (Corning), 15% FBS (Gibco), 100 U/mL of penicillin, 100 μg/mL of streptomycin (Corning), 2 mM of L-glutamine (Corning), 55 μM β-mercaptoethanol (Gibco), 1 mM Sodium Pyruvate (Corning), 10 mM Hepes (Corning), 1×MEM Nonessential Amino Acids (Corning)) with rhIL-2 (100 U/mL; R&D systems) changing medium every 2 days. On days 4-6, T cells were collected and used for functional assays in vitro and in vivo.

Co-Culture Experiments with Murine CAR-T Cells

Eμ-myc cells labeled with 0.5 μM carboxyfluorescein diacetate succinimidyl ester (CFSE; Invitrogen) were seeded in 24-well plates at a concentration of 1×10⁵ cells/well, T cells were added to the culture at different ratios (E:T of 1:1, 2.5:1; 5:1; 10:1, or 20:1) without the addition of exogenous cytokines. Cells were analyzed 18 hours later to measure residual tumor cells by FACS. Target cells were identified by the expression of murine CD19-APC (130-102-546, Miltenyi Biotec) and their viability by the expression of CFSE.

### Transduction and Expansion of Human T Cells

Apheresis leukoreduction collars from healthy donors were obtained from the Boston Children's Hospital Blood Donor Center, Boston, Mass. On day 0, lymphocytes were isolated with Ficoll-Paque Plus density separation (GE Healthcare), T cells were isolated with EasySep Human T Cell Isolation Kit (Stemcell) and activated with Dynabeads Human T-Activator CD3/CD28 (Gibco) according to the manufacturer's instructions. The day after, plates for transduction were prepared: non-tissue culture treated 24-well plates were coated overnight with 7 μg/mL retronectin (500 μL/well) (Takara Bio Inc., Shiga, Japan) in the cold room. On day 2, T cells were transduced. Briefly, non-tissue culture treated 24-well plates coated overnight with 7 mg/mL retronectin in the cold room were washed once with 1 mL medium, coated with 1 mL of the retroviral supernatant per well and centrifuged at 2000 g for 90 min. After removal of the supernatant, 5×10⁵ activated T cells were plated, and centrifuged at 1000 g for 10 min. Three days later, T cells were collected and expanded in complete medium (45% RPMI-1640 and 45% Click's medium (Irvine Scientific), 10% FBS (Gibco), 2 mM GlutaMAX (Gibco), 100 unit/mL of penicillin and 100 μg/mL of streptomycin (Corning) with rhIL-7 (10 ng/mL; PeproTech) and rhIL-15 (5 ng/mL; PeproTech), changing medium every 2-3 days. On day 12-14, cells were collected for in vitro and in vivo experiments. T cells were cultured in rhIL-7/IL-15 depleted medium for two days prior to being used in in vitro functional assays.

Co-culture Experiments with hCAR-T Cells

Tumor cells were seeded in 24-well plates at a concentration of 5×10⁵ cells/well 24 hours before co-cloture. T cells were added to the culture at different ratios (E:T of 1:1; 1:5, or 1:10) without the addition of exogenous cytokines. Cells were analyzed at day 5 to measure residual tumor cells and T cells by FACS. Dead cells were gated out by Zombie Aqua Dye (Biolegend) staining while T cells were identified by the expression of CD3 and tumor cells by the expression of GFP (NB cell lines) or CD19 (Raji cell line).

### Flow Cytometry

Flow cytometry was performed using the following antibodies: human CD3 PerCP-cy5.5 (340948, BD Biosciences), human CD3 FITC (IM1281U, Beckman Coulter), human CD19 APC (IM2470U, Beckman Coulter), murine CD19 APC (130-102-546, Miltenyi Biotec). Expression of ALK in tumor cell lines was assessed with the ALK5 mAb conjugated with Alexa Fluor 647 using the Alexa Fluor Antibody Labeling Kit (Life technologies) according to manufacturer's instructions. Expression of the ALK CAR-T Cells was detected using F(ab′)2-Goat anti-Mouse IgG (H+L) Alexa Fluor 647 (Invitrogen). Samples were acquired with BD FACSCelesta flow cytometer using the BD Diva software (BD Biosciences). For each sample, a minimum of 10,000 events were acquired and data was analyzed using FlowJo 10.

### ELISA

T cells (5×10⁴, 1×10⁵ or 5×10⁵) were co-cultured with tumor cells (5×10⁵) in 24-well plates without the addition of exogenous cytokines. After 24 hours, supernatant was collected and IFNγ and GM-CSF cytokines were measured in duplicate using specific ELISA kits (BioLegend or R&D system) following manufacturer's instructions.

### T Cells Proliferation Assay

T cells were labeled with 1.5 mM carboxyfluorescein diacetate succinimidyl ester (CFSE; Invitrogen) and plated with tumor cells at an E:T ratio of 1:1. CFSE dilution was measured on gated T cells on day 5 using flow cytometry.

### Western Blot

Whole cell extracts were obtained using GST-FISH buffer (10 mM MgCl₂, 150 mM NaCl, 1% NP-40, 2% Glycerol, 1 mM EDTA, 25 mM HEPES pH 7.5) supplemented with Protease Inhibitor Cocktail (Roche), 1 mM phenylmethanesulfonylfluoride (PMSF), 10 mM NaF and 1 mM Na₃VO₄. Extracts were cleared by centrifugation at 15,000 rpm for 20 min. The supernatants were collected and assayed for protein concentration using BCA protein assay method (Sigma). Equal amounts of protein lysates were resolved by Mini-PROTEAN TGX gels (BIO-RAD), transferred on nitrocellulose membrane (GE Healthcare), and probed with the following primary antibodies: ALK, rabbit ALK (D5F3) XP (Cell Signaling Technology, #3633), rabbit GFP (Cell Signaling Technology, #2555), rabbit polyclonal anti-β-actin (Sigma, #A5316), rabbit α-actinin (D6F6) XP (Cell Signaling Technology, #6487). Membranes were developed with ECL solution (GE Healthcare).

NB Cell Proliferation and Apoptosis Assays after Lorlatinib Treatment

In white 96-well plates, 3×10⁴ cells/mL were grown in triplicates. The treatment with lorlatinib was done after 24 hour. Cell growth was analyzed 5 days after treatment using Cell Titer-GloMax assay (Promega, Fitchburg, Wis., USA), according to the manufacturer's instructions. In 24-well plates, 5×10⁴ cells/mL were grown in triplicates. The treatment with lorlatinib was done after 24 hours. Apoptosis was measured 48 hours after treatment by flow cytometry after staining with the FITC Annexin V and propidium iodide (PI) Staining Solution Apoptosis Detection Kit I (BD Pharmingen) according to the manufacturer's instructions.

### Quantification and Statistical Analysis

The unpaired and nonparametric Mann Whitney test with two tailed p value calculation was used to measure differences between two groups. For multiple group comparisons, one-way ANOVA or two-way ANOVA was used to determine statistically significant differences between samples. Holm-Sidak test adjusted p value <0.05 indicates a significant difference. Measurements were summarized as mean±SD. Difference between the survival curves were analyzed by the Chi-square test using GraphPad Prism v5. Graph generation and statistical analyses were performed using the GraphPad Prism software (GraphPad, La Jolla, Calif.).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of some embodiments herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. Particularly, WO 2017/035430, WO 2017/147383, U.S. Ser. No. 62/902,096, and Davila et al., CD19 CAR-Targeted T Cells Induce Long-Term Remission and B Cell Aplasia in an Immunocompetent Mouse Model of B Cell Acute Lymphoblastic Leukemia, PLoS ONE (2013), are incorporated herein by reference.

1. An anaplastic lymphoma kinase chimeric antigen receptor (ALK CAR) comprising:
an extracellular binding domain comprising (a) or (b)
(a) a heavy chain complementarity determining region 1 (HCDR1), a heavy chain complementarity determining region 2 (HCDR2), and a heavy chain complementarity determining region 3 (HCDR3) each comprising an amino acid sequence that is at least 80% identical to the HCDR1, HCDR2, and HCDR3 sequences of an anti-ALK antibody in Table 4, wherein the extracellular binding domain specifically binds to an anaplastic lymphoma kinase (ALK) polypeptide or antibody-binding fragment thereof; or
(b) a heavy chain variable region (VH) comprising an amino acid sequence that is at least 80% identical to the VH of an anti-ALK antibody in Table 2, wherein the extracellular binding domain specifically binds to an anaplastic lymphoma kinase (ALK) polypeptide or antibody-binding fragment thereof;
a transmembrane domain; and
at least one signaling domain.

2. The ALK CAR of claim 1, wherein the extracellular binding domain comprises the HCDR1, HCDR2, and HCDR3 amino acid sequences of an anti-ALK antibody in Table 4 or the VH of an anti-ALK antibody in Table 2.

3. The ALK CAR of claim 1, wherein the extracellular binding domain further comprising a light chain complementarity determining region 1 (LCDR1), a light chain complementarity determining region 2 (LCDR2), and a light chain complementarity determining region 3 (LCDR3) each comprising an amino acid sequence that is at least 80% identical to the LCDR1, LCDR2 and LCDR3 sequences of an anti-ALK antibody in Table 3.

4-15. (canceled)

16. The ALK CAR of claim 1, wherein the extracellular binding domain is an scFv.

17. The ALK CAR of claim 1, wherein the anti-ALK antibody comprises VH CDR amino acid sequences SYWMN, QIYPGDGDTNYNGKFKG, and YYYGSKAY, and VL CDR amino acid sequences RASENIYYSLA, NANSLED, KQAYDVPFT,
VH CDR amino acid sequences
SYWMH, RIDPNSGGTKYNEKFKS,
and
DYYGSSYRFAY,
and VL CDR amino acid sequences
SVSQGISNSLN, YTSSLHS
and
QQYSKLPLT;
VH CDR amino acid sequences
NYWMH, YINPSSGYTKYNQKFKD,
and
DYYGSSSWFAY,
and VL CDR amino acid sequences
KASQNVGTNVA, SASYRYS,
and
QQYNSYPYMYT;
VH CDR amino acid sequences
SYWVN, QIYPGDGDTNYNGKFKG,
and
SRGYFYGSTYDS,
and VL CDR amino acid sequences
RASESVDNYGISFMN, AASNQGS.
and
QQSKEVPWT;
VH CDR amino acid sequences
SYWMH, YIKPSSGYTKYNQKFKD,
and
DYYGSSSWFAY,
and VL CDR amino acid sequences
KASQNVGTNVA, SASYRYS,
and
QQYNSYPYMYT;
VH CDR amino acid sequences
SYAMS, YISSGGDYIYYADTVKG,
and
ERIWLRRFFDV,
and VL CDR amino acid sequences
KASQNVGTAVA, SASNRFT,
and
QQYSSYPLT;
VH CDR amino acid sequences
SYWMH, YINPSSGYTKYNQKFKD,
and
DYYGSSSWFAY.
and VL CDR amino acid sequence
KASQNVGTNVA, SASYRYS.
and
QRYNSYPYMFT.

18-23. (canceled)

24. The ALK CAR of claim 1, wherein the anti-ALK antibody comprises VH amino acid sequence
QVQLQQSGAELVKPGASVKISCKASGYAFSSYWMNWV
KQRPGKGLEWIGQIYPGDGDTNYNGKFKGKATLTADK
SSSTAYMQLSSLTSEDSAVYFCASYYYGSKAYWGQGT
LVTVSA,
and VL amino acid sequence
DIQMTQSPASLAASVGETVTITCRASENIYYSLAWYQQ
KQGKSPQLLIYNANSLEDGVPSRFSGSGSGTQYSMKIN
SMQPEDTATYFCKQAYDVPFTFGSGTKLEIKR.

25. The ALK CAR of claim 1, wherein the anti-ALK antibody comprises
VH amino acid sequence
QVQLQQPGAEFVKPGASVKLSCKASGYTFTSYWMHWV
KQRPGRGLEWIGRIDPNSGGTKYNEKFKSKATLTVDK
PSSTAYMQLSSLTSEDSAVYYCARDYYGSSYRFAYWG
QGTLVTVSA,
and VL amino acid sequence
AIQMTQTTSSLSASLGDRVTISCSVSQGIS
NSLNWYQQKPDGTVKLLIYYTSSLHSGVPS
RFSGSGSGTDYSLTISNLEPEDIATYYCQQ
YSKLPLTFGAGTKLELKR;
VH amino acid sequence
QVQLQQSGAELAKPGASVKLSCKASGYT
FTNYWMHWVKQRPGQGLEWIGYINPSSG
YTKYNQKFKDKATLTADKSSSTAYMQLS
SLTYEDSAVYYCARDYYGSSSWFAYWGQ
GTLVTVSA,
and VL amino acid sequence
DIVMTQSQRFMSTSVGDRVSVTCKASQNV
GTNVAWYQQKPGQSPKALIYSASYRYSGV
PDRFTGSGSGTDFTLTVSNVQSEDLAEYF
CQQYNSYPYMYTFGGGTKLEIKR;
VH amino acid sequence
QVQLQQSGAELVKPGASVKISCKASGYAF
SSYWVNWVKQRPGKGLEWIGQIYPGDGDT
NYNGKFKGKATLTADKSSSTAYMQLSSLT
SEDSAVYFCARSRGYFYGSTYDSWGQGTT
LTVSS,
and VL amino acid sequence
DIVLTQSPASLAVSLGQRATISCRASESVDNYGISFM
NWFQQKPGQPPKLLIYAASNQGSGVPARFSGSGSGTD
FSLNIHPMEEDDTAMYFCQQSKEVPWTFGGGTKLEIK
R;
VH amino acid sequence
QVQLQQSGAELAKPGASVKLSCKASGYTFTSYWMHWV
KQRPGQGLEWIGYIKPSSGYTKYNQKFKDKATLTADK
SSSTAYMQLSSLTYEDSAVYYCARDYYGSSSWFAYWG
QGTLVTVSA,
and VL amino acid sequence
DIVMTQSQRFMSTSVGDRVSVTCKASQNVGTNVAWYQQ
KPGQSPKALIYSASYRYSGVPDRFTGSGSGTDFTLTIS
NVQSEDLAEYFCQQYNSYPYMYTFGGGTKLEIKR;
VH amino acid sequence
DVKLVESGEGLVKPGGSLKLSCAASGFTFSSYAMSWVR
QTPEKRLEWVTYISSGGDYIYYADTVKGRFTISRDNAR
NTLYLQMSSLKSEDTAMYYCTRERIWLRRFFDVWGTGT
TVTVSS.
and VL amino acid sequence
DIVMTQSQKFMSTSVGDRVSITCKASQNVGTAVAWYQL
KPGQSPKLLIYSASNRFTGVPDRFTGSGSGTDFTLTIS
NMQSEDLADYFCQQYSSYPLTFGSGTKLEIKR;
or
VH amino acid sequence
QVQLQQSGAELAKPGASVKLSCKASGYT
FTSYWMHWVKQRPGQGLEWIGYINPSSG
YTKYNQKFKDKATLTADKSSSTAYMQLS
SLTFEDSAVYYCARDYYGSSSWFAYWGQ
GTLVTVSA,
and VL amino acid sequence
DIVMTQSQKFMSTSVGDRVSVTCKASQNV
GTNVAWYQQKPGHSPKALIYSASYRYSGV
PDRFTGSGSGTDFTLTISNVQSEDLAEYF
CQRYNSYPYMFTFGGGTKLEIKR.

26-30. (canceled)

31. The ALK CAR of claim 1, wherein the transmembrane domain is selected from the group consisting of CD8, CD137 (4-1BB), and CD28.

32. (canceled)

33. The ALK CAR of claim 1, wherein the at least one signaling domain is selected from the group consisting of CD8, CD28, CD134 (OX40), CD137 (4-1BB), and CD3ζ.

34-41. (canceled)

42. A polynucleotide encoding the ALK CAR of claim 1.

43. A vector comprising the polynucleotide of claim 42.

44-46. (canceled)

47. An engineered immune cell expressing at the cell surface membrane an ALK CAR according to claim 1.

48. An engineered immune cell produced by transforming an immune cell with the polynucleotide of claim 42.

49-55. (canceled)

56. The engineered immune cell of claim 48, wherein the ALK-positive cancer has an ALK^{F1174L} activating point mutation.

57. A method of engineering an immune cell comprising:
providing an immune cell; and
expressing at the surface of the immune cell at least one ALK CAR according to claim 1.

58. A method of engineering an immune cell comprising:
providing an immune cell;
introducing into the immune cell the polynucleotide according to claim 42; and
expressing the polynucleotide in the immune cell.

59-60. (canceled)

61. A pharmaceutical composition comprising the ALK CAR of claim 1, and a pharmaceutically acceptable carrier, diluent, or excipient.

62. (canceled)

63. A method of treating a subject with an ALK-positive cancer comprising administrating the pharmaceutical composition of claim 62 to the subject.

64. A method of treating a subject with an ALK-positive cancer comprising administering to the subject the ALK CAR of claim 1.

65. A method of treating a subject with an ALK-positive cancer, the method comprising:
transforming immune cells with the vector of claim 43 to obtain an engineered immune cell; and
administering an effective amount of the engineered immune cell to the subject.

66. The method of claim 65, wherein the immune cells are derived from the subject or a donor.

67-68. (canceled)

69. A method of treating a subject with an ALK-positive cancer, the method comprising administering an effective amount of an engineered immune cell comprising an ALK CAR and an effective amount of an ALK vaccine comprising at least one isolated ALK polypeptide or polynucleotide to the subject.

70-81. (canceled)

82. The method of claim 63, wherein the ALK-positive cancer has an ALK^{F1174L} activating point mutation.

83. A kit comprising an agent for administration to a subject, wherein the agent comprises the ALK CAR of claim 1, and instructions for using the kit.


SEQUENCE LISTING (119 sequences)

SEQ ID NO: 1 - protein, 5 aa, Artificial Sequence
  Description of Artificial Sequence Synthetic peptide
GGGGS

SEQ ID NO: 2 - protein, 5 aa, Artificial Sequence
  Description of Artificial Sequence Synthetic peptide
SYWMN

SEQ ID NO: 3 - protein, 17 aa, Artificial Sequence
  Description of Artificial Sequence Synthetic peptide
QIYPGDGDTNYNGKFKG

SEQ ID NO: 4 - protein, 8 aa, Artificial Sequence
  Description of Artificial Sequence Synthetic peptide
YYYGSKAY

SEQ ID NO: 5 - protein, 11 aa, Artificial Sequence
  Description of Artificial Sequence Synthetic peptide
RASENIYYSLA

SEQ ID NO: 6 - protein, 7 aa, Artificial Sequence
  Description of Artificial Sequence Synthetic peptide
NANSLED

SEQ ID NO: 7 - protein, 9 aa, Artificial Sequence
  Description of Artificial Sequence Synthetic peptide
KQAYDVPFT

SEQ ID NO: 8 - protein, 5 aa, Artificial Sequence
  Description of Artificial Sequence Synthetic peptide
SYWMH

SEQ ID NO: 9 - protein, 17 aa, Artificial Sequence
  Description of Artificial Sequence Synthetic peptide
RIDPNSGGTKYNEKFKS

SEQ ID NO: 10 - protein, 11 aa, Artificial Sequence
  Description of Artificial Sequence Synthetic peptide
DYYGSSYRFAY

SEQ ID NO: 11 - protein, 11 aa, Artificial Sequence
  Description of Artificial Sequence Synthetic peptide
SVSQGISNSLN

SEQ ID NO: 12 - protein, 7 aa, Artificial Sequence
  Description of Artificial Sequence Synthetic peptide
YTSSLHS

SEQ ID NO: 13 - protein, 9 aa, Artificial Sequence
  Description of Artificial Sequence Synthetic peptide
QQYSKLPLT

SEQ ID NO: 14 - protein, 5 aa, Artificial Sequence
  Description of Artificial Sequence Synthetic peptide
NYWMH

SEQ ID NO: 15 - protein, 17 aa, Artificial Sequence
  Description of Artificial Sequence Synthetic peptide
YINPSSGYTKYNQKFKD

SEQ ID NO: 16 - protein, 11 aa, Artificial Sequence
  Description of Artificial Sequence Synthetic peptide
DYYGSSSWFAY

SEQ ID NO: 17 - protein, 11 aa, Artificial Sequence
  Description of Artificial Sequence Synthetic peptide
KASQNVGTNVA

SEQ ID NO: 18 - protein, 7 aa, Artificial Sequence
  Description of Artificial Sequence Synthetic peptide
SASYRYS

SEQ ID NO: 19 - protein, 11 aa, Artificial Sequence
  Description of Artificial Sequence Synthetic peptide
QQYNSYPYMYT

SEQ ID NO: 20 - protein, 5 aa, Artificial Sequence
  Description of Artificial Sequence Synthetic peptide
SYWVN

SEQ ID NO: 21 - protein, 12 aa, Artificial Sequence
  Description of Artificial Sequence Synthetic peptide
SRGYFYGSTYDS

SEQ ID NO: 22 - protein, 15 aa, Artificial Sequence
  Description of Artificial Sequence Synthetic peptide
RASESVDNYGISFMN

SEQ ID NO: 23 - protein, 7 aa, Artificial Sequence
  Description of Artificial Sequence Synthetic peptide
AASNQGS

SEQ ID NO: 24 - protein, 9 aa, Artificial Sequence
  Description of Artificial Sequence Synthetic peptide
QQSKEVPWT

SEQ ID NO: 25 - protein, 17 aa, Artificial Sequence
  Description of Artificial Sequence Synthetic peptide
YIKPSSGYTKYNQKFKD

SEQ ID NO: 26 - protein, 5 aa, Artificial Sequence
  Description of Artificial Sequence Synthetic peptide
SYAMS

SEQ ID NO: 27 - protein, 17 aa, Artificial Sequence
  Description of Artificial Sequence Synthetic peptide
YISSGGDYIYYADTVKG

SEQ ID NO: 28 - protein, 11 aa, Artificial Sequence
  Description of Artificial Sequence Synthetic peptide
ERIWLRRFFDV

SEQ ID NO: 29 - protein, 11 aa, Artificial Sequence
  Description of Artificial Sequence Synthetic peptide
KASQNVGTAVA

SEQ ID NO: 30 - protein, 7 aa, Artificial Sequence
  Description of Artificial Sequence Synthetic peptide
SASNRFT

SEQ ID NO: 31 - protein, 9 aa, Artificial Sequence
  Description of Artificial Sequence Synthetic peptide
QQYSSYPLT

SEQ ID NO: 32 - protein, 11 aa, Artificial Sequence
  Description of Artificial Sequence Synthetic peptide
QRYNSYPYMFT

SEQ ID NO: 33 - protein, 117 aa, Artificial Sequence
  Description of Artificial Sequence Synthetic polypeptide
QVQLQQSGAELVKPGASVKISCKASGYAFSSYWMNWVKQRPGKGLEWIGQIYPGDGDTNYNGKFKGKATLTADKSSSTAYMQLSSLTSEDSAVYFCASYYYGSKAYWGQGTLVTVSA

SEQ ID NO: 34 - protein, 108 aa, Artificial Sequence
  Description of Artificial Sequence Synthetic polypeptide
DIQMTQSPASLAASVGETVTITCRASENIYYSLAWYQQKQGKSPQLLIYNANSLEDGVPSRFSGSGSGTQYSMKINSMQPEDTATYFCKQAYDVPFTFGSGTKLEIKR

SEQ ID NO: 35 - protein, 120 aa, Artificial Sequence
  Description of Artificial Sequence Synthetic polypeptide
QVQLQQPGAEFVKPGASVKLSCKASGYTFTSYWMHWVKQRPGRGLEWIGRIDPNSGGTKYNEKFKSKATLTVDKPSSTAYMQLSSLTSEDSAVYYCARDYYGSSYRFAYWGQGTLVTVSA

SEQ ID NO: 36 - protein, 108 aa, Artificial Sequence
  Description of Artificial Sequence Synthetic polypeptide
AIQMTQTTSSLSASLGDRVTISCSVSQGISNSLNWYQQKPDGTVKLLIYYTSSLHSGVPSRFSGSGSGTDYSLTISNLEPEDIATYYCQQYSKLPLTFGAGTKLELKR

SEQ ID NO: 37 - protein, 120 aa, Artificial Sequence
  Description of Artificial Sequence Synthetic polypeptide
QVQLQQSGAELAKPGASVKLSCKASGYTFTNYWMHWVKQRPGQGLEWIGYINPSSGYTKYNQKFKDKATLTADKSSSTAYMQLSSLTYEDSAVYYCARDYYGSSSWFAYWGQGTLVTVSA

SEQ ID NO: 38 - protein, 110 aa, Artificial Sequence
  Description of Artificial Sequence Synthetic polypeptide
DIVMTQSQRFMSTSVGDRVSVTCKASQNVGTNVAWYQQKPGQSPKALIYSASYRYSGVPDRFTGSGSGTDFTLTVSNVQSEDLAEYFCQQYNSYPYMYTFGGGTKLEIKR

SEQ ID NO: 39 - protein, 121 aa, Artificial Sequence
  Description of Artificial Sequence Synthetic polypeptide
QVQLQQSGAELVKPGASVKISCKASGYAFSSYWVNWVKQRPGKGLEWIGQIYPGDGDTNYNGKFKGKATLTADKSSSTAYMQLSSLTSEDSAVYFCARSRGYFYGSTYDSWGQGTTLTVSS

SEQ ID NO: 40 - protein, 112 aa, Artificial Sequence
  Description of Artificial Sequence Synthetic polypeptide
DIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIKR

SEQ ID NO: 41 - protein, 120 aa, Artificial Sequence
  Description of Artificial Sequence Synthetic polypeptide
QVQLQQSGAELAKPGASVKLSCKASGYTFTSYWMHWVKQRPGQGLEWIGYIKPSSGYTKYNQKFKDKATLTADKSSSTAYMQLSSLTYEDSAVYYCARDYYGSSSWFAYWGQGTLVTVSA

SEQ ID NO: 42 - protein, 110 aa, Artificial Sequence
  Description of Artificial Sequence Synthetic polypeptide
DIVMTQSQRFMSTSVGDRVSVTCKASQNVGTNVAWYQQKPGQSPKALIYSASYRYSGVPDRFTGSGSGTDFTLTISNVQSEDLAEYFCQQYNSYPYMYTFGGGTKLEIKR

SEQ ID NO: 43 - protein, 120 aa, Artificial Sequence
  Description of Artificial Sequence Synthetic polypeptide
DVKLVESGEGLVKPGGSLKLSCAASGFTFSSYAMSWVRQTPEKRLEWVTYISSGGDYIYYADTVKGRFTISRDNARNTLYLQMSSLKSEDTAMYYCTRERIWLRRFFDVWGTGTTVTVSS

SEQ ID NO: 44 - protein, 108 aa, Artificial Sequence
  Description of Artificial Sequence Synthetic polypeptide
DIVMTQSQKFMSTSVGDRVSITCKASQNVGTAVAWYQLKPGQSPKLLIYSASNRFTGVPDRFTGSGSGTDFTLTISNMQSEDLADYFCQQYSSYPLTFGSGTKLEIKR

SEQ ID NO: 45 - protein, 120 aa, Artificial Sequence
  Description of Artificial Sequence Synthetic polypeptide
QVQLQQSGAELAKPGASVKLSCKASGYTFTSYWMHWVKQRPGQGLEWIGYINPSSGYTKYNQKFKDKATLTADKSSSTAYMQLSSLTFEDSAVYYCARDYYGSSSWFAYWGQGTLVTVSA

SEQ ID NO: 46 - protein, 110 aa, Artificial Sequence
  Description of Artificial Sequence Synthetic polypeptide
DIVMTQSQKFMSTSVGDRVSVTCKASQNVGTNVAWYQQKPGHSPKALIYSASYRYSGVPDRFTGSGSGTDFTLTISNVQSEDLAEYFCQRYNSYPYMFTFGGGTKLEIKR

SEQ ID NO: 47 - protein, 1626 aa, Homo sapiens
TASSGGMGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 48 - protein, 38 aa, Homo sapiens
GAAAVVAAGTSRRLCSEGRGAPRCFPAALWSATQSRGR

SEQ ID NO: 49 - protein, 70 aa, Homo sapiens
WVRRGRQDFGRPCPERPQLLPPGPLQCLRTLRSRGAGESKDAANLRSAGAGIHAQKFSRQTVRSLPAAER

SEQ ID NO: 50 - protein, 45 aa, Homo sapiens
LEGAQDGSLRPRFPPRPGRRAWRSQKERKRRPGQRAAAGSRRSQP

SEQ ID NO: 51 - protein, 63 aa, Homo sapiens
KLQRLEAAPRGDRPQLRLRGAGEDGTQLPPPFNHSSSSVPSAASYRRGRGTRRGEREAQGPSQ

SEQ ID NO: 52 - protein, 4 aa, Homo sapiens
AQCA

SEQ ID NO: 53 - protein, 14 aa, Homo sapiens
VSLDSPLSFQVCFI

SEQ ID NO: 54 - protein, 46 aa, Homo sapiens
TPARLRAVGGKQETCAHAQSSGDQVEGAAGYQGLFRASSHLGESEG

SEQ ID NO: 55 - protein, 7 aa, Homo sapiens
GWARRAV

SEQ ID NO: 56 - protein, 1626 aa, Homo sapiens; identical to SEQ ID NO: 47

SEQ ID NO: 57 - protein, 40 aa, Homo sapiens
ARSHTHFSSLGSLRPWRRERQWLLHKPETKCHVLFCANLF

SEQ ID NO: 58 - protein, 8 aa, Homo sapiens
STTKKAVF

SEQ ID NO: 59 - protein, 24 aa, Homo sapiens
KCFRKVLSMGSSYSFERRKYHKNE

SEQ ID NO: 60 - protein, 8 aa, Homo sapiens
IQGPDVVA

SEQ ID NO: 61 - protein, 26 aa, Homo sapiens
GFYACLLYTSLCFFQIVCALLQCSQN

SEQ ID NO: 62 - protein, 16 aa, Homo sapiens
LLLCFIVGVIDVSLPC

SEQ ID NO: 63 - protein, 20 aa, Homo sapiens
CGHEPFEGRGNGNKGVICND

SEQ ID NO: 64 - protein, 1620 aa, Homo sapiens
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 65 - protein, 1620 aa, Homo sapiens; identical to SEQ ID NO: 64

SEQ ID NO: 66 - protein, 634 aa, Mus musculus
MGQRQSILKRCPEGSFLLLNTSADSKHTILSPWMRSSSDHCTLAVSVHRHLQPSGRYVAQLLPHNEAGREILLVPTPGKHGWTVLQGRVGRPANPFRVALEYISSGNRSLSAVDFFALKNCSEGTSPGSKMALQSSFTCWNGTVLQLGQACDFHQDCAQGEDEGQLCSKLPAGFYCNFENGFCGWTQSPLSPHMPRWQVRTLRDAHSQGHQGRALLLSTTDILASEGATVTSATFPAPMKNSPCELRMSWLIRGVLRGNVSLVLVENKTGKEQSRTVWHVATDEGLSLWQHTVLSLLDVTDRFWLQIVTWWGPGSRATVGFDNISISLDCYLTISGEEKMSLNSVPKSRNLFEKNPNKESKSWANISGPTPIFDPTVHWLFTTCGASGPHGPTQAQCNNAYQNSNLSVVVGSEGPLKGVQIWKVPATDTYSISGYGAAGGKGGKNTMMRSHGVSVLGIFNLEKGDTLYILVGQQGEDACPRANQLIQKVCVGENNVIEEEIRVNRSVHEWAGGGGGGGGATYVFKMKDGVPVPLIIAAGGGGRAYGAKTETFHPERLESNSSVLGLNGNSGAAGGGGGWNDNTSLLWAGKSLLEGAAGGHSCPQAMKKWGWETRGGFGGGGGGGAPQVEEAEDI

SEQ ID NO: 67 - DNA, 6240 bp, Homo sapiens
agatgcgatccagcggctctgggggcggcagcggtggtagcagctggtacctcccgccgcctctgttcggagggtcgcggggcaccgaggtgctttccggccgccctctggtcggccacccaaagccgcgggcgctgatgatgggtgaggagggggcggcaagatttcgggcgcccctgccctgaacgccctcagctgctgccgccggggccgctccagtgcctgcgaactctgaggagccgaggcgccggtgagagcaaggacgctgcaaacttgcgcagcgcgggggctgggattcacgcccagaagttcagcaggcagacagtccgaagccttcccgcagcggagagatagcttgagggtgcgcaagacggcagcctccgccctcggttcccgcccagaccgggcagaagagcttggaggagccaaaaggaacgcaaaaggcggccaggacagcgtgcagcagctgggagccgccgttctcagccttaaaagttgcagagattggaggctgccccgagaggggacagaccccagctccgactgcggggggcaggagaggacggtacccaactgccacctcccttcaaccatagtagttcctctgtaccgagcgcagcgagctacagacgggggcgcggcactcggcgcggagagcgggaggctcaaggtcccagccagtgagcccagtgtgcttgagtgtctctggactcgcccctgagcttccaggtctgtttcatttagactcctgctcgcctccgtgcagttgggggaaagcaagagacttgcgcgcacgcacagtcctctggagatcaggtggaaggagccgctgggtaccaaggactgttcagagcctcttcccatctcggggagagcgaagggtgaggctgggcccggagagcagtgtaaacggcctcctccggcgggatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaa

SEQ ID NO: 68 - DNA, 6221 bp, Homo sapiens
ggggcggcagcggtggtagcagctggtacctcccgccgcctctgttcggagggtcgcggggcaccgaggtgctttccggccgccctctggtcggccacccaaagccgcgggcgctgatgatgggtgaggagggggcggcaagatttcgggcgcccctgccctgaacgccctcagctgctgccgccggggccgctccagtgcctgcgaactctgaggagccgaggcgccggtgagagcaaggacgctgcaaacttgcgcagcgcgggggctgggattcacgcccagaagttcagcaggcagacagtccgaagccttcccgcagcggagagatagcttgagggtgcgcaagacggcagcctccgccctcggttcccgcccagaccgggcagaagagcttggaggagccaaaaggaacgcaaaaggcggccaggacagcgtgcagcagctgggagccgccgttctcagccttaaaagttgcagagattggaggctgccccgagaggggacagaccccagctccgactgcggggggcaggagaggacggtacccaactgccacctcccttcaaccatagtagttcctctgtaccgagcgcagcgagctacagacgggggcgcggcactcggcgcggagagcgggaggctcaaggtcccagccagtgagcccagtgtgcttgagtgtctctggactcgcccctgagcttccaggtctgtttcatttagactcctgctcgcctccgtgcagttgggggaaagcaagagacttgcgcgcacgcacagtcctctggagatcaggtggaaggagccgctgggtaccaaggactgttcagagcctcttcccatctcggggagagcgaagggtgaggctgggcccggagagcagtgtaaacggcctcctccggcgggatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaaaa

SEQ ID NO: 69 - DNA, 5918 bp, Mus musculus
gtgttcacgcccagaagttcagcgggcagggtgatcgatccgaagacttcctgcagcggaggtcacttgagggggcgctagaaagcagccccctccggtggtccttgcctagacctgggaaggagcgcagaggaggtgacaggagcggaggacgtgggcaagacagtgaccgactcggagccacggttcacagcctggaaagttgcagaagattggaagctaagaggagagctctggtcgccgagggctccttgaacggtacctaattgccacctccctggtccctgagcaaaggcctctacaaatggggcgcagcacggcgagaggcgcaggatccagctgttgagcccagggtgtctcactgtctccgaactaccccctgactttgtcttccgttttgctgagaacccttctcgcctccttgtagcttgggaaaagcaagggcgctctatagtgtacacacagtccctgagatctagtggaaggagccattcaggaccaaggactatttggagccctttcctgtttgggggagagtgaagggcgaggctggaccagcaagggaagggagactagtgtaaactcgccctccagcgggatgggagctgctgggttcctgtggctgctgcctccactgcttttggcagcagcctcgtactccggagctgcaaccgatcagcgcgcgggttccccagcctcagggcctcctctgcagccccgggagccgctcagttattcgcgcctgcagaggaagagtctggcagtggacttcgttgtaccctcgctcttccgcgtctatgcccgagacctgctgctaccgcagccacggtccccctcggagcccgaggctggcgggctggaggcgcggggatcactggccctggattgtgagcctctgctcaggctgctggggccactgcctggaatctcctgggcagatggagccagttctcctagtcccgaggcgggtccgacgctgtccagggtgctgaagggaggctcggtgcgcaagctcaggcgtgccaaacagctggtgctggagctgggcgaggagacgattcttgaaggctgtattggtcccccagaggaggtagcggctgtggggatactccagttcaacctcagcgagctgttcagctggtggattctccacggcgaagggaggctgaggatccgcctgatgcctgagaagaaggcatcggaagtgggcagggagggaaggctatccagtgcgatccgagcctcccagccccgccttctcttccagatcttcgggacgggacacagctccatggagtcaccctcagaaacgccttctcctcctggtaccttcatgtggaatctgacctggacgatgaaagactccttccctttcctttcccaccgcagtcgatatggtctggagtgcagctttgacttcccctgtgagctggaatattctcctcccctgcacaaccacgggaatcagagctggtcctggcgccatgtgccctccgaggaggcctcgaggatgaacttgctggatgggccagaggcagagcattctcaagagatgcccagaggctccttcctcctcctgaacacctctgcagattccaagcataccattctgagcccatggatgaggagcagtagtgatcactgcacactggctgtctccgtgcacagacatctacagccttcggggagatatgttgcccagctcctaccccacaacgaagctggaagagagattcttttggtgcccaccccagggaagcatggctggacagtgctgcaggggagagtcgggcgcccagcaaacccatttcgagtggctctggaatacatctccagtggcaaccggagcttgtcggcggtggatttctttgccctgaagaactgcagtgaagggacatccccaggctccaagatggcattgcagagttccttcacttgttggaatgggaccgtcctccagctcgggcaagcctgtgatttccaccaggactgtgcccaaggagaagatgagggccagctgtgcagtaaacttcctgctggattttactgtaactttgaaaatggcttctgtggctggacccaaagtccactctcaccccatatgccccggtggcaagtgaggaccctaagagatgcccattcccagggccaccaaggccgtgccctgttgctcagcaccactgacatcctcgcttctgaaggtgcaacagtgaccagtgccaccttccctgcaccaatgaaaaattctccttgtgagctccgcatgtcctggctcatccgcggggttttgagaggaaacgtatctctggtgctggtggagaacaaaaccggaaaggagcaaagccggactgtctggcatgtcgccactgacgaaggcttaagcctgtggcagcatacagtgctgtccctcctcgatgtgactgacaggttctggctgcagatagtcacatggtggggtccaggatccagggcaaccgtgggatttgacaacatttccatcagcctcgactgctaccttaccatcagtggagaggagaaaatgtccctgaattcagtacccaaatctagaaatctgtttgagaaaaacccaaacaaggagtcaaaatcctgggcaaacatatcaggaccaactcccatcttcgaccctacagttcactggctgttcaccacgtgtggggccagtggacctcatggccccacccaggcacagtgcaacaacgcctaccagaattccaacttgagcgtggtggtgggaagtgaagggcccttgaagggagtccagatttggaaagtgccagctactgacacctacagtatctcgggctacggagcagctggcgggaaaggtgggaaaaacaccatgatgcggtcccatggcgtgtctgtcctgggcatcttcaatctggagaaaggtgacacactctacatccttgtcggtcagcaaggggaggatgcctgtcccagggcaaaccaactaatccagaaagtctgtgtgggtgagaacaatgtcatagaagaagagatccgagtgaacagaagcgtgcacgagtgggcaggaggaggaggaggtgggggtggagccacctacgtgtttaagatgaaagatggcgtgcctgtacccctgatcattgcagctggtggtggtggcagggcctatggggccaagacagaaacgttccacccagagagactggagagtaactcctcggttctagggctgaacggcaattccggagccgcaggtggtggaggcggctggaatgataacacttccttgctctgggccggaaagtctttgctggagggtgccgccggaggacattcctgcccccaggccatgaagaagtgggggtgggagacaagagggggtttcggagggggtggaggggggtgctcctcaggtggaggaggcggaggatatataggtggcaacgcagcatcaaacaatgaccccgaaatggatggggaagatggggtttccttcatcagtccattgggtatcctgtacaccccggccttaaaagtgatggagggccacggggaagtgaatatcaagcattatctaaactgcagtcactgcgaggtagacgaatgtcacatggaccccgagagccacaaagtcatctgcttctgtgatcatgggaccgtgctggctgatgatggtgtctcctgcattgtgtcacccaccccggagccccacctgccgctctcattgatcctctccgtcgtgacctctgccctggtggctgcccttgttctggcattctccggcatcatgattgtgtaccgtcggaagcaccaggagttgcaggctatgcagatggaactgcagagccccgagtataagctgagcaagctacggacctcgaccatcatgaccgactacaaccccaactactgcttcgctggcaagacttcctccatcagtgacctgaaagaagtgccacggaaaaacatcacactcatccggggcctaggccatggcgcatttggggaggtgtatgaaggccaggtgtctggaatgcccaatgacccaagccctctacaagtggctgtaaagacgctgccagaagtgtgttcagaacaagatgagctggactttctcatggaagctctgatcatcagcaaattcaaccaccagaatattgttcgctgcatcggggtgagtctacaagccctgccccgcttcatcctgctggaactcatggctggcggagacctcaagtccttcctcagggagacacgccctcgcccgaaccaacccacctccctggccatgctggaccttctgcatgtggctcgggacattgcctgtggctgtcagtacctagaggagaatcactttatccaccgggatattgctgctagaaactgtctgttgacctgcccaggagctggaagaatagcaaagattggagactttgggatggcccgagatatctacagggccagctactaccgaaagggaggctgcgccatgctgccggtcaagtggatgccccctgaagccttcatggaagggatatttacctctaaaacagacacatggtcttttggagtgttgctatgggaaatattttctcttggatatatgccgtaccccagcaagagcaaccaggaagttctggagtttgtcaccagcggaggacggatggacccgcctaagaactgccccgggcctgtataccggataatgacgcagtgctggcagcatcagcctgaagacagacccaacttcgccatcattttggagaggatcgaatactgcacccaggaccccgatgtgatcaacacagctctgcccatcgaatacggtccagtagtagaagaggaggagaaagtgcccatgcgccccaaagaccccgaggggatgccacctttgctggtgtctccccagcctgcgaagcacgaggaggcgtccgcagctccccagcccgcagccctgacggcaccaggcccatcggtgaagaagcccccgggtgcgggtgcgggcgcgggcgcgggtgcgggtgccggcccggtgccccgaggtgcggccgatcggggccacgtgaacatggctttctctcagcccaaccctcccccggagctgcacaaaggcccgggatccagaaacaagccgaccagcctgtggaaccccacctacggctcgtggttcaccgagaagcctgccaaaaagacccatcctccgccaggcgccgagccgcaggcgcgggcaggagcggccgagggtggctggaccgggccgggcgcggggccccgcagagccgaggcagcgctgctgctagagccatcggcgctgagcgccaccatgaaggaggtgccgctgttcaggctgcgccacttcccctgcggcaatgtcaactatggttaccagcaacagggtctccccttggaagccacagccgcgccaggggacaccatgctgaaaagcaagaataaggtcacccagccggggccctgagccctgtactccactagcttctcctcctggcggagccggagcccacccagagggagatggacaggatggctccaccacaaacccaagaccaaaactttcatttttgtgccaacttgttttgaagtgccacattttaaaaaaaggaaacttgtgtttttaagatgtgttagaaggttttttgagcatgggttcatctatcctctcaaaagaagaaaatgccattctttaaaaaagaaaaaaaagcaatcagtgcaaggcccagattggttgcgccaagttttcgtgcatggtctgctgtacagtcccctaaggcttctttccgatttttgtgtgcgctctgcttccgcgtagtcagaaatagctgcttccatgtctcatagggggagtcctaggtgtttcctttgccttatgaatatgaaccactcgaggggcgggcgagggaacagaaataaag

SEQ ID NO: 70 - DNA, 324 bp, Artificial Sequence
  Description of Artificial Sequence Synthetic polynucleotide
gacatccagatgactcagtctccagcctccctggctgcatctgtgggagaaactgtcaccatcacatgtcgagcaagtgagaacatttactacagtttagcatggtatcagcagaagcaagggaaatctcctcagctcctgatctataatgcaaacagcttggaagatggtgtcccatcgaggttcagtggcagtggatctgggacacagtattctatgaagatcaacagcatgcagcctgaagataccgcaacttatttctgtaaacaggcttatgacgttccattcacgttcggctcggggacaaagttggaaataaaacgg

SEQ ID NO: 71 - DNA, 324 bp, Artificial Sequence
  Description of Artificial Sequence Synthetic polynucleotide
gctatccagatgacacagactacatcctccctgtctgcctctctgggagacagagtcaccatcagttgcagtgtaagtcagggcattagcaattctttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaagcttccgctcacgttcggtgctgggaccaagctggagctgaaacgg

SEQ ID NO: 72 - DNA, 330 bp, Artificial Sequence
  Description of Artificial Sequence Synthetic polynucleotide
gacattgtgatgacccagtctcaaagattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccgtcagcaatgtgcagtctgaagacttggcagagtatttctgtcagcaatataacagctatccgtacatgtacacgttcggaggggggaccaagctggaaataaaacgg

SEQ ID NO: 73 - DNA, 336 bp, Artificial Sequence
  Description of Artificial Sequence Synthetic polynucleotide
gacattgtgctgacccaatctccagcttctttggctgtgtctctagggcagagggccaccatctcctgcagagccagcgaaagtgttgataattatggcattagttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaagtaaggaggttccgtggacgttcggtggaggcaccaagctggaaatcaaacgg

SEQ ID NO: 74 - DNA, 330 bp, Artificial Sequence
  Description of Artificial Sequence Synthetic polynucleotide
gacattgtgatgacccagtctcaaagattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcaacaatataacagctatccgtacatgtacacgttcggaggggggaccaagctggaaataaaacgg

SEQ ID NO: 75 - DNA, 324 bp, Artificial Sequence
  Description of Artificial Sequence Synthetic polynucleotide
gacattgtgatgacccagtctcaaaaattcatgtccacatcagtaggagacagggtcagcatcacctgtaaggccagtcagaatgtgggtactgctgtagcctggtatcaactgaaaccaggacaatctcctaaactactgatttactcggcatccaatcggttcactggagtccctgatcgcttcacaggcagtggatctgggacagatttcactctcaccatcagcaatatgcagtctgaagacctggcagattatttctgccagcaatatagcagctatcctctcacgttcggctcggggacaaagttggaaataaaacgg

SEQ ID NO: 76 - DNA, 330 bp, Artificial Sequence
  Description of Artificial Sequence Synthetic polynucleotide
gacattgtgatgacccagtctcaaaaattcatgtccacatcagtaggagacagggtcagcgtcacctgcaaggccagtcagaatgtgggtactaatgtagcctggtatcaacagaaaccagggcactctcctaaagcactgatttactcggcatcctaccggtacagtggagtccctgatcgcttcacaggcagtggatctgggacagatttcactctcaccatcagcaatgtgcagtctgaagacttggcagagtatttctgtcagcgatataacagctatccgtacatgttcacgttcggaggggggaccaagctggaaataaaacgg

SEQ ID NO: 77 - DNA, 351 bp, Artificial Sequence
  Description of Artificial Sequence Synthetic polynucleotide
caggttcagctgcagcagtctggggctgagctggtgaagcctggggcctcagtgaagatttcctgcaaagcttctggctacgcattcagtagctactggatgaactgggtgaagcagaggcctggaaagggtcttgagtggattggacagatttatcctggagatggtgatactaactacaacggaaagttcaagggcaaggccacactgactgcagacaaatcctccagcacagcctacatgcagctcagcagcctgacctctgaggactctgcggtctatttctgtgcctcttattactacggtagtaaggcttactggggccaagggactctggtcactgtctctgca

SEQ ID NO: 78 - DNA, 360 bp, Artificial Sequence
  Description of Artificial Sequence Synthetic polynucleotide
caggtccaactgcagcagcctggggctgagtttgtgaagcctggggcttcagtgaagctgtcctgcaaggcttctggctacaccttcaccagctactggatgcactgggtgaagcagaggcctggacgaggccttgagtggattggaaggattgatcctaatagtggtggtactaagtacaatgagaagttcaagagcaaggccacactgactgtagacaaaccctccagcacagcctacatgcagctcagcagcctgacatctgaggactctgcggtctattattgtgcaagagattactacggtagtagctaccggtttgcttactggggccaagggactctggtcactgtctctgca

SEQ ID NO: 79 - DNA, 360 bp, Artificial Sequence
  Description of Artificial Sequence Synthetic polynucleotide
caggtccagctgcagcagtctggggctgaactggcaaaacctggggcctcagtgaagctgtcctgcaaggcttctggctacacctttactaactactggatgcactgggtaaaacagaggcctggacagggtctggaatggattggatacattaatcctagcagtggttatactaagtacaatcagaagttcaaggacaaggccacattgactgcagacaaatcctccagcacagcctacatgcagctgagcagcctgacatatgaggactctgcagtctattactgtgcaagagattactacggtagtagctcctggtttgcttactggggccaagggactctggtcactgtctctgca

SEQ ID NO: 80 - DNA, 363 bp, Artificial Sequence
  Description of Artificial Sequence Synthetic polynucleotide
caggttcagctgcaacagtctggggctgagctggtgaagcctggggcctcagtgaagatttcctgcaaagcttctggctacgcattcagtagctactgggtgaactgggtgaagcagaggcctggaaagggtcttgagtggattggacagatttatcctggagatggtgatactaactacaacggaaagttcaagggcaaggccacactgactgcagacaaatcctccagcacagcctacatgcagctcagcagcctgacctctgaggactctgcggtctatttctgtgcaagatcaagagggtatttctacggtagtacctacgactcctggggccaaggcaccactctcacagtctcctca

SEQ ID NO: 81 - DNA, 360 bp, Artificial Sequence
  Description of Artificial Sequence Synthetic polynucleotide
caggtccagctgcagcagtctggggctgaactggcaaaacctggggcctcagtgaagctgtcctgcaaggcttctggctacacctttactagctactggatgcactgggtaaaacagaggcctggacagggtctggaatggattggatacattaagcctagcagtggttatactaagtacaatcagaagttcaaggacaaggccacattgactgcagacaaatcctccagcacagcctacatgcagctgagcagcctgacatatgaggactctgcagtctattactgtgcaagagattactacggtagtagctcctggtttgcttactggggccaagggactctggtcactgtctctgca

SEQ ID NO: 82 - DNA, 360 bp, Artificial Sequence
  Description of Artificial Sequence Synthetic polynucleotide
gacgtgaagctggtggagtctggggaaggcttagtgaagcctggagggtccctgaaactctcctgtgcagcctctggattcactttcagtagctatgccatgtcttgggttcgccagactccagagaagaggctggagtgggtcacatacattagtagtggtggtgattacatctactatgcagacactgtgaagggccgattcaccatctccagagacaatgccaggaacaccctgtacctgcaaatgagcagtctgaagtctgaggacacagccatgtattactgtacaagagagcggatatggttacgacggttcttcgatgtctggggcacagggaccacggtcaccgtctcctca

SEQ ID NO: 83 - DNA, 360 bp, Artificial Sequence
  Description of Artificial Sequence Synthetic polynucleotide
caggtccagctgcagcagtctggggctgaactggcaaaacctggggcctcagtgaagctgtcctgcaaggcttctggctacacctttactagctactggatgcactgggtaaaacagaggcctggacagggtctggaatggattggatacattaatcctagcagtggttatactaagtacaatcagaagttcaaggacaaggccacattgactgcagacaaatcctccagcacagcctacatgcagctgagcagcctgacatttgaggactctgcagtctattactgtgcaagagattactacggtagtagctcctggtttgcttactggggccaagggactctggtcactgtctctgca

SEQ ID NO: 84 - protein, 680 aa, Homo sapiens [sequence omitted]

SEQ ID NO: 85 - protein, 680 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEVSVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 86 - protein, 680 aa, Homo sapiens; identical to SEQ ID NO: 85

SEQ ID NO: 87 - DNA, 2043 bp, Homo sapiens
atggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctga

SEQ ID NO: 88 - protein, 1129 aa, Homo sapiens [sequence omitted]

SEQ ID NO: 89 - DNA, 3926 bp, Homo sapiens
ggcggcgcggcgcggcgctcgcggctgctgcctgggagggaggccgggcaggcggctgagcggcgcggctctcaacgtgacggggaagtggttcgggcggccgcggcttactaccccagggcgaacggacggacgacggaggcgggagccggtagccgagccgggcgacctagagaacgagcgggtcaggctcagcgtcggccactctgtcggtccgctgaatgaagtgcccgcccctctgagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggagcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagaggtctctgttcgagtccctagagggccggccgtggaagggggacacgtgaatatggcattctctcagtccaaccctccttcggagttgcacagggtccacggatccagaaacaagcccaccagcttgtggaacccaacgtacggctcctggtttacagagaaacccaccaaaaagaataatcctatagcaaagaaggagccacacgagaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcacacactcacttctcttccttgggatccctaagaccgtggaggagagagaggcaatcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttcttttaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgaaaaaaaaaaaaaaaaaaaaaaaaaaa

SEQ ID NO: 90 - protein, 1059 aa, Homo sapiens [sequence omitted]

SEQ ID NO: 91 - protein, 15 aa, Artificial Sequence
  Description of Artificial Sequence Synthetic peptide
GGGGSGGGGSGGGGS

SEQ ID NO: 92 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 84 aa, Artificial Sequence
  Description of Artificial Sequence Synthetic polypeptide
FVPVFLPARPTTTPAPRPPTPAPTIASQPLSLRPEACRPAAGGAVHTRGLDFACDIYIWAPLAGTCGVLLLSLVITLYCNHRNR

SEQ ID NO: 94 - protein, 40 aa, Artificial Sequence
  Description of Artificial Sequence Synthetic polypeptide
SKRSRLLHSDYMNMTPRRPGPTRKHYQPYAPPRDFAAYRS

SEQ ID NO: 95 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 96 - protein, 47 aa, Artificial Sequence
  Description of Artificial Sequence Synthetic polypeptide
RFSVVKRGRKKLLYIFKQPFMRPVQTTQEEDGCSCRFPEEEEGGCEL

SEQ ID NO: 97 - protein, 37 aa, Artificial Sequence
  Description of Artificial Sequence Synthetic polypeptide
RRDQRLPPDAHKPPGGGSFRTPIQEEQADAHSTLAKI

SEQ ID NO: 98 - protein, 67 aa, Artificial Sequence
  Description of Artificial Sequence Synthetic polypeptide
IEVMYPPPYLDNEKSNGTIIHVKGKHLCPSPLFPGPSKPFWVLVVVGGVLACYSLLVTVAFIIFWVR

SEQ ID NO: 99 - protein, 69 aa, Artificial Sequence
  Description of Artificial Sequence Synthetic polypeptide
TTTPAPRPPTPAPTIASQPLSLRPEACRPAAGGAVHTRGLDFACDIYIWAPLAGTCGVLLLSLVITLYC

SEQ ID NO: 100 - protein, 634 aa, Mus musculus; identical to SEQ ID NO: 66

SEQ ID NO: 101 - DNA, 6240 bp, Homo sapiens; identical to SEQ ID NO: 67

SEQ ID NO: 102 - DNA, 5918 bp, Mus musculus; identical to SEQ ID NO: 69

SEQ ID NO: 103 - DNA, 2043 bp, Homo sapiens; identical to SEQ ID NO: 87

SEQ ID NO: 104 - protein, 1129 aa, Homo sapiens [sequence omitted]

SEQ ID NO: 105 - protein, 1059 aa, Homo sapiens [sequence omitted]

SEQ ID NO: 106 - DNA, 324 bp, Artificial Sequence; identical to SEQ ID NO: 70
  Description of Artificial Sequence Synthetic polynucleotide

SEQ ID NO: 107 - DNA, 324 bp, Artificial Sequence; identical to SEQ ID NO: 71
  Description of Artificial Sequence Synthetic polynucleotide

SEQ ID NO: 108 - DNA, 330 bp, Artificial Sequence; identical to SEQ ID NO: 72
  Description of Artificial Sequence Synthetic polynucleotide

SEQ ID NO: 109 - DNA, 336 bp, Artificial Sequence; identical to SEQ ID NO: 73
  Description of Artificial Sequence Synthetic polynucleotide

SEQ ID NO: 110 - DNA, 330 bp, Artificial Sequence; identical to SEQ ID NO: 74
  Description of Artificial Sequence Synthetic polynucleotide

SEQ ID NO: 111 - DNA, 324 bp, Artificial Sequence; identical to SEQ ID NO: 75
  Description of Artificial Sequence Synthetic polynucleotide

SEQ ID NO: 112 - DNA, 330 bp, Artificial Sequence; identical to SEQ ID NO: 76
  Description of Artificial Sequence Synthetic polynucleotide

SEQ ID NO: 113 - DNA, 351 bp, Artificial Sequence; identical to SEQ ID NO: 77
  Description of Artificial Sequence Synthetic polynucleotide

SEQ ID NO: 114 - DNA, 360 bp, Artificial Sequence; identical to SEQ ID NO: 78
  Description of Artificial Sequence Synthetic polynucleotide

SEQ ID NO: 115 - DNA, 360 bp, Artificial Sequence; identical to SEQ ID NO: 79
  Description of Artificial Sequence Synthetic polynucleotide

SEQ ID NO: 116 - DNA, 363 bp, Artificial Sequence; identical to SEQ ID NO: 80
  Description of Artificial Sequence Synthetic polynucleotide

SEQ ID NO: 117 - DNA, 360 bp, Artificial Sequence; identical to SEQ ID NO: 81
  Description of Artificial Sequence Synthetic polynucleotide

SEQ ID NO: 118 - DNA, 360 bp, Artificial Sequence; identical to SEQ ID NO: 82
  Description of Artificial Sequence Synthetic polynucleotide

SEQ ID NO: 119 - DNA, 360 bp, Artificial Sequence; identical to SEQ ID NO: 83
  Description of Artificial Sequence Synthetic polynucleotide